(19) United States
(12) Patent Application Publication
(10) Pub. No.: US 2025/0243488 A1
(43) Pub. Date: Jul. 31, 2025

(54) METHODS OF MODULATING PROGRANULIN EXPRESSION USING ANTISENSE OLIGONUCLEOTIDES TARGETING REGULATORY RNAS

(71) Applicant: Camp4 Therapeutics Corporation, Cambridge, MA (US)

(72) Inventors: Yeliz Yuva-Aydemir, Grafton, MA (US); Alfica Sehgal, Belmont, MA (US); Yuichi Nishi, Ayer, MA (US); Ali Al Abdullatif, Somerville, MA (US); Gokul Ramaswami, Somerville, MA (US); Justin Andrew Caravella, Cambridge, MA (US); Rutuja Sudhakar Pai, Charlestown, MA (US)

(21) Appl. No.: 18/973,877

(22) Filed: Dec. 9, 2024

Related U.S. Application Data
(63) Continuation of application No. PCT/US2023/068254, filed on Jun. 9, 2023.
(60) Provisional application No. 63/381,910, filed on Nov. 1, 2022.
(60) Provisional application No. 63/369,907, filed on Jul. 29, 2022.
(60) Provisional application No. 63/351,263, filed on Jun. 10, 2022.

(51) Int. Cl. C12N 15/113; A61K 38/17; A61P 5/00

(52) U.S. Cl. CPC C12N 15/113; A61K 38/1709; A61P 5/00; C12N 2310/113; C12N 2310/3341

(57) ABSTRACT

Described herein are methods of modulating GRN gene transcription using antisense oligonucleotides (ASOs) targeting GRN regulatory RNAs, such as promoter-associated RNAs and enhancer RNAs. These methods are useful for modulating the levels of GRN gene products, for example, increasing expression of progranulin (PGRN), to thereby treat diseases and disorders in a subject.

### CROSS REFERENCE TO RELATED APPLICATIONS

This application is a continuation of International Application No. PCT/US2023/068254, filed Jun. 9, 2023, which claims the benefit of U.S. Provisional Application No. 63/351,263, filed Jun. 10, 2022, U.S. Provisional Application No. 63/369,907, filed Jul. 29, 2022, and U.S. Provisional Application No. 63/381,910, filed Nov. 1, 2022, each of which are hereby incorporated in their entirety by reference.

### SEQUENCE LISTING

The instant application contains a Sequence Listing which has been submitted electronically in XML format and is hereby incorporated by reference in its entirety. Said XML file, created on Aug. 16, 2023, is named CTC-027WO_SL.xml, and is 11,597,640 bytes in size.

### FIELD OF THE INVENTION

The invention relates to methods of upregulating or downregulating GRN gene transcription using antisense oligonucleotides (ASOs) targeting GRN regulatory RNAs, such as promoter-associated RNAs and enhancer RNAs.

### BACKGROUND

Transcription factors bind specific sequences in promoter and enhancer DNA elements to regulate gene transcription. It was recently reported that active promoters and enhancer elements are themselves transcribed, generating noncoding regulatory RNAs (regRNAs) such as promoter-associated RNAs (paRNAs) and enhancer RNAs (eRNAs) (see Sartorelli and Lauberth, Nat. Struct. Mol. Biol. (2020) 27:521-28). Unlike coding RNAs, regRNAs are transcribed bi-directionally. Various models have been proposed for the functions of regRNAs, including nucleosome remodeling (see Mousavi et al., Mol. Cell (2013) 51(5):606-17), modulation of enhancer-promoter looping (see Lai et al., Nature (2013) 494(7438):497-501), and direct interaction with transcription regulators (see Sigova et al., Science (2015) 350:978-81).

Progranulin (PGRN) is encoded by the human GRN gene and is the precursor of granulin peptides. PGRN is a highly conserved secreted protein that is expressed in multiple cell types, including the central nervous system (CNS) and peripheral tissues. There is growing evidence that PGRN and its proteolytic granulin peptide products are involved in lysosomal function and have trophic and neuroprotective effects.

Deficiencies of PGRN and mutations in GRN may lead to a variety of neurological diseases and disorders, including frontotemporal dementia. Frontotemporal dementia (FTD) is a progressive neurodegenerative disease where patients progress rapidly to severe dementia and death. Approximately 40% of patients with FTD have familial FTD, with about 30% (10% overall) having a heterozygous GRN mutation resulting in haploinsufficiency. In general, PGRN protein levels stay constant over a patient's life, with non-prognostic factors for the time of initial onset (typically 55-65 years of age). GRN-frontotemporal dementia (GRN-FTD, also known as FTD-GRN) disease onset for FTD patients is subtle; behavioral and language symptoms begin a few years before diagnosis. Within 3-4 years of diagnosis patients have progressed to late-stage dementia, requiring full caregiver support. Life expectancy after the diagnosis of GRN-FTD is typically about 7 years. However, only symptomatic therapies for FTD exist today and there are no available disease-reversing or disease-modifying agents.

Gene expression has been generally known as an undruggable biological process. Despite on-going efforts into understanding the biology of gene transcription and regRNAs, clinically suitable methods of modulating gene expression are limited. There remains a need for new and useful methods for treating diseases associated with aberrant (e.g., reduced) expression of PGRN, such as FTD.

### SUMMARY

In one aspect, provided herein are antisense oligonucleotides (ASO) complementary to at least 5 contiguous nucleotides of a regulatory RNA of progranulin (pGRN), wherein the regulatory RNA has a nucleotide sequence selected from the group consisting of SEQ ID NOs: 1-6.

In some embodiments, the ASO is complementary to a sequence in the regRNA that is no more than 200 nucleotides from the 3′ end of the regRNA.

In some embodiments, the ASO is complementary to a sequence in the regRNA that is no more than 200 nucleotides from the 5′ end of the regRNA.

In some embodiments, the ASO comprises a nucleotide sequence selected from Table 17, 18, or 19.

In some embodiments, the ASO comprises a nucleotide sequence of any one of SEQ ID NOs: 1369-4738

In some embodiments, the regulatory RNA has a nucleotide sequence of SEQ ID NO: 1, and the ASO comprises a nucleotide sequence selected from the group consisting of SEQ ID NOs: 10-268, 691, 991-1368, or 4743-4915.

In some embodiments, the regulatory RNA has a nucleotide sequence of SEQ ID NO: 2, and the ASO comprises the nucleotide sequence of SEQ ID NO: 269-279.

In some embodiments, the regulatory RNA has a nucleotide sequence of SEQ ID NO: 3, and the ASO comprises the nucleotide sequence of SEQ ID NO: 280-291 or 336-359.

In some embodiments, the regulatory RNA has a nucleotide sequence of SEQ ID NO: 4, and the ASO comprises the nucleotide sequence of SEQ ID NO: 292-313 or 360-380.

In some embodiments, the regulatory RNA has a nucleotide sequence of SEQ ID NO: 5, and the ASO comprises the nucleotide sequence of SEQ ID NO: 314-335 or 381-416.

In some embodiments, the regulatory RNA has a nucleotide sequence of SEQ ID NO: 6, and the ASO comprises the nucleotide sequence of SEQ ID NO: 417-442.

In some embodiments, the ASO is no more than 50, 40, 30, 25, or 20 nucleotides in length.

In some embodiments, the ASO comprises a RNA polynucleotide comprising one or more chemical modifications.

In some embodiments, each nucleotide in the ASO comprises ribonucleotides with one or more chemical modifications

In some embodiments, at least 3, 4, or 5 nucleotides at the 5′ end and at least 3, 4, or 5 nucleotides at the 3′ end of the ASO comprise ribonucleotides with one or more chemical modifications.

In some embodiments, the one or more chemical modifications comprise 2′-O-methoxyethyl, 5-methyl on cytidine, locked nucleic acid (LNA), and phosphorothioate internucleotide bond.

In some embodiments, the ASO does not comprise 10 or more contiguous nucleotides of unmodified DNA.

In some embodiments, the ASO does not comprise a deoxyribonucleotide.

In some embodiments, the ASO does not comprise an unmodified ribonucleotide.

In some embodiments, each ribonucleotide of the ASO is modified by 2′-O-methoxyethyl.

In some embodiments, the length of the ASO is 3×n+10 nucleotides (n is an integer of 4 or greater), wherein the nucleotides at positions 3×m are ribonucleotides modified by LNA (m is an integer from 1 to n) and the nucleotides at the remaining positions are ribonucleotides modified by 2′-O-methoxyethyl.

In some embodiments, the length of the ASO is 2×n+4 nucleotides (n is an integer of 8 or greater), wherein the nucleotides at positions 2×m are ribonucleotides modified by LNA (m is an integer from 1 to n) and the nucleotides at the remaining positions are ribonucleotides modified by 2′-O-methoxyethyl.

In some embodiments, the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

In some embodiments, the length of the ASO is 4×n+4 nucleotides (n is an integer of 4 or greater), wherein the nucleotides at positions 4×m are ribonucleotides modified by LNA (m is an integer from 1 to n) and the nucleotides at the remaining positions are ribonucleotides modified by 2′-O-methoxyethyl.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the length of the ASO is 2×n+8 nucleotides (n is an integer of 8 or greater), wherein the nucleotides at positions 2×m are ribonucleotides modified by LNA (m is an integer from 1 to n) and the nucleotides at positions 2×m+1 are ribonucleotides modified by 2′-O-methoxyethyl.

In some embodiments, the length of the ASO is 2×n+8 nucleotides (n is an integer of 8 or greater), wherein the nucleotides at positions 2×m+1 are ribonucleotides modified by LNA (m is an integer from 1 to n) and the nucleotides at positions 2×m are ribonucleotides modified by 2′-O-methoxyethyl.

In some embodiments, the ASO comprises at least one phosphodiester bond.

In some embodiments, the ASO comprises 10 or more contiguous nucleotides of unmodified DNA flanked by at least 3 nucleotides of modified ribonucleotides at each of the 5′ end and the 3′ end.

In some embodiments, each cytidine in the ASO is modified by 5-methyl.

In some embodiments, the ASO comprises 2 or more contiguous nucleotides of unmodified DNA flanked by at least 3 nucleotides of modified ribonucleotides at each of the 5′ end and the 3′ end.

In some embodiments, the regRNA is an eRNA.

In some embodiments, the regRNA is a paRNA.

In one aspect, provided herein are pharmaceutical compositions comprising the ASO disclosed herein and a pharmaceutically acceptable carrier.

In one aspect, provided herein are methods of increasing transcription of pGRN in a human cell, the method comprising contacting the cell with the ASO disclosed herein or the pharmaceutical composition disclosed herein.

In some embodiments, the cell is a neuron.

In some embodiments, the ASO increases the amount of the regulatory RNA in the cell.

In some embodiments, the ASO increases the stability of the regulatory RNA in the cell.

In some embodiments, the ASO increases the amount of pGRN mRNA in the cell.

In some embodiments, the ASO increases the amount of pGRN protein in the cell.

In one aspect, provided herein are methods of treating frontotemporal dementia (FTD), the method comprising administering to a subject in need thereof an effective amount of the ASO disclosed herein or the pharmaceutical composition disclosed herein.

In some embodiments, the ASO increases the amount of the regulatory RNA in a cell of the subject.

In some embodiments, the ASO increases the stability of the regulatory RNA in a cell of the subject.

In some embodiments, the ASO increases the amount of pGRN mRNA in the cell.

In some embodiments, the ASO increases the amount of pGRN protein in the cell.

In some embodiments, the cell is a neuron.

### BRIEF DESCRIPTION OF THE DRAWINGS

FIG. 1A shows an illustrative schematic of eRNA, paRNA, mRNA, and natural antisense transcript (NAT) of a gene on a chromosome. The eRNA, paRNA, and NAT are all non-coding RNAs. The eRNA is transcribed bidirectionally from an enhancer of the gene. The paRNA is transcribed from the promoter of the gene, same as the mRNA, but in the antisense direction. The NAT is transcribed from a downstream promoter of its own in the antisense direction, such that the transcript overlaps at least partially with the mRNA. Generally, eRNAs and paRNAs upregulate gene expression whereas NATs downregulates gene expression. FIG. 1B shows an illustrative schematic of the interaction of regRNA with enhancer and promoter regions to recruit transcription factors and regulators that modulate gene expression.

FIGS. 2A and 2B shows that human GRN (hGRN) mRNA (FIG. 2A) and hGRN paRNA (FIG. 2B) was detected in human induced pluripotent stem cell (iPSC)-derived neurons and induced microglia-like (iMGL) cells following exposure 1 μM or 3 μM vorinostat or to DMSO alone (vehicle control), as determined using qPCR.

FIG. 3 shows that hGRN paRNA was detected in human cortex tissue from three donors (#1, #2 and #3), as determined using qPCR.

FIG. 4 shows the relative hGRN mRNA levels as determined using qPCR in human SK-N-AS cells after treatment with the indicated concentration of the indicated ASO.

FIG. 5A shows the relative hGRN mRNA levels in SK-N-AS cells after treatment with the indicated concentration of the indicated ASO, as determined using qPCR. FIG. 5B shows the relative hGRN mRNA levels in SK-N-AS cells after treatment with the indicated concentration of the indicated ASO, as determined using qPCR.

FIG. 6 shows the relative human PGRN (hGRN) protein levels in SK-N-AS cells after treatment with the indicated ASOs, as determined by ELISA.

FIG. 7A shows the relative hGRN mRNA levels in iMGL cells after treatment with the indicated dose of the indicated ASO, as determined using qPCR. FIG. 7B shows the fold change (FC) in secreted PGRN protein levels in iMGL cells after treatment with the indicated ASOs, as determined using ELISA.

FIGS. 8A and 8B show the relative hGRN mRNA levels in wild type human iPSC-derived neurons (FIG. 8A) and FTD-GRN patient-derived GRN^{M1L} neurons after treatment with the indicated ASOs at the indicated concentration, as determined using qPCR.

FIG. 9A shows the assay timeline for the staurosporine rescue assay. FIG. 9B shows the percent cytotoxicity levels in GRN-FTD patient derived neurons after treatment with the indicated ASO, PGRN protein, or BDNF protein, and either DMSO or staurosporine at the indicated concentrations.

FIG. 10 shows the murine mGrn mRNA and paRNA expression in the indicated mouse brain tissue from a C57/BL6, as determined using qPCR. (RT=reverse transcriptase).

FIGS. 11A, 11B and 11C show the PGRN protein levels in serum samples (FIG. 11A), cerebrospinal fluid (CSF) samples (FIG. 11B), and brain tissue lysates derived from the cortex or mixed brain regions (FIG. 11C), at the indicated dilutions, as determined using ELISA.

FIG. 12 shows mouse GRN paRNA expression levels in Neuro2a cells after exposure to 0.3 μM, 1 μM, or 3 μM of vorinostat, or DMSO control, as determined using qPCR with two different primer sets (1F/1R and 3F/3R).

FIG. 13 shows the relative mGrn mRNA levels in Neuro2a cells after treatment with the indicated ASOs at the indicated concentration or a no transfection control, as determined using qPCR.

FIG. 14 shows the relative mGrn mRNA levels in Neuro2a cells after treatment with the indicated ASO at the indicated concentration or untreated cells (“no ASO”), as determined using qPCR.

FIG. 15 shows the relative mGrn mRNA levels in Neuro2a cells after treatment with the indicated ASO in mouse primary neurons at the indicated concentration, as determined using qPCR.

FIG. 16 shows the relative mGrn mRNA levels in the cortex, hippocampus and striatum mouse brain regions from C57/BL6 mice after in vivo treatment with the indicated ASO or PBS control.

FIG. 17 provides the sequences and chemical modifications of human GRN ASOs. Medium gray shading indicates MOE; * indicates 5Me-C; dark gray indicates LNA; light gray indicates 2′OMe (2′-O-methyl); black line indicates phosphodiester (PO) bond; white indicates DNA.

FIG. 18 provides the sequences and chemical modifications of mouse GRN ASOs. Medium gray shading indicates 2′-MOE; light gray indicates O-methyl; * indicates 5Me-C; dark gray indicates LNA; black line indicates phosphodiester bond (PO); white indicates DNA.

FIG. 19 shows increased PGRN protein secretion and reduced protein secretion of IL-8 and CCL4, as well as reduced gene expression of IL-6, CCL4, and CCL2 in iMGL cells treated with PBS or IFNγ and the indicated ASOs.

FIG. 20A provides hGRN mRNA levels in hGRNT^{Tg} mice after ICV injection of CO-8178 or aCSF (control), as determined using qPCR. FIG. 20B provides hPGRN protein quantification in hGRNT^{Tg} mice after ICV injection of CO-8178 or aCSF (control), as determined by ELISA. FIG. 20C provides the fold change (FC) in hPGRN protein in CSF of mice post-ICV injection of CO-8178 or aCSF (control). Fold change was calculated by normalizing the levels of hPGRN protein CO-8178 treated samples to the aCSF treated samples.

FIG. 21A provides hGRN mRNA levels in hGRNT^{Tg} mice brain tissues following ICV injection of the indicated ASOs or aCSF (control), as determined using qPCR. FIG. 21B provides hPGRN protein levels in hGRNT^{Tg} mice brain tissues after ICV injection of the indicated ASOs or aCSF (control), as determined using ELISA.

FIG. 22A shows mGrn mRNA levels in heterozygous Grn^{tm1.1Far} mice brain tissues after ICV injection of the indicated ASO or aCSF (control), as determined using qPCR. FIG. 22B shows mPgrn protein quantifications heterozygous Grn^{tm1.1Far} mice brain tissues after ICV injection of the indicated ASO or aCSF (control), as determined using ELISA.

FIGS. 23A, 23B, 23C, and 23D show mGm mRNA levels in Grn^{tm1.1Far} mice brain tissues: cortex (FIG. 23A), hippocampus (FIG. 23B), cerebellum (FIG. 23C), and striatum (FIG. 23D) after ICV injection of the indicated ASOs or aCSF (control), as determined using qPCR.

FIG. 24 shows mPgrn protein levels in Grn^{tm1.1Far} mice brain tissues (cortex and cerebellum) after ICV injection of the indicated ASOs or aCSF (control), as determined using ELISA.

FIGS. 25A and 25B show the upregulation of secreted hPGRN protein (FIG. 25A) and intracellular hPGRN protein (FIG. 25B) in SK-NA-S cells after treatment with the indicated ASO, as determined using ELISA.

FIGS. 26A and 26B show dose dependent upregulation of hGRN mRNA in SK-NA-S cells after treatment with the indicated ASOs at the indicated concentration, as determined using qPCR.

FIG. 27 shows dose dependent upregulation of hGRN mRNA in human iPSC-derived neuron cells after treatment with the indicated ASO, as determined using qPCR.

FIG. 28A shows upregulation of GRN mRNA expression in iMGL cells treated with either PBS or IFNγ and the indicated ASOs, as determined using qPCR. FIGS. 28B and 28C show reduced IFNγ-induced chemokine (CCL3 and CCL4) expression in iMGL cells treated with either PBS or IFNγ and the indicated ASOs, as determined using ELISA.

### DETAILED DESCRIPTION

The present disclosure provides antisense oligonucleotides (ASOs) targeting regulatory RNAs, such as promoter-associated RNAs and enhancer RNAs, and methods using these ASOs to regulate gene expression. These methods are useful for modulating the levels of gene products, for example, modulating expression levels of granulin precursor or progranulin (PGRN, encoded by the GRN gene), to thereby treat diseases associated with aberrant GRN gene expression in a subject, such as, but not limited to, frontotemporal dementia (FTD), frontotemporal lobar degeneration (FTLD), neuroinflammation, myopathy, familial frontotemporal dementia with neuropathologic frontotemporal lobal degeneration associated with accumulation of TDP-43 inclusions (FTLD-TDP), Down syndrome, Huntington's disease, hippocampal sclerosis dementia, spinocerebellar ataxia 3, chronic traumatic encephalopathy, Alzheimer's disease (AD), amyotrophic lateral sclerosis (ALS), Gaucher disease (GD), Parkinson's disease (PD), neuronal ceroid lipofuscinosis (NCL) type11 (CLN11), limbic-predominant age-related TDP-43 encephalopathy (LATE) Gaucher disease, autism, ischemia-reperfusion injury in the brain, a lysosomal storage disease (LSD), rheumatoid arthritis (RA), inflammatory bowel disease (IBD), multiple sclerosis (MS), ischemic heart disease, intervertebral disc ceneration, and acute kidney injury.

In some embodiments, the ASOs of the disclosure may be used to restore progranulin or granulin expression in cells, such as cells which exhibit progranulin haploinsufficiency, or to enhance the expression of progranulin in cells (e.g., neurons). In some embodiments, the ASOs of the disclosure may be used to restorle or increase the levels of secreted progranulin or granulin in a subject (e.g., a subject having a progranulin haploinsufficiency).

Various aspects of the multi-specific binding proteins described in the present application are set forth below in sections.

### I. Definitions

To facilitate an understanding of the present application, a number of terms and phrases are defined below.

The terms “a” and “an” as used herein mean “one or more” and include the plural unless the context is inappropriate.

As used herein, the terms “granulin precursor,” or “progranulin,” or “PGRN” or refer to the protein of UniProt Accession No. P28799 (human) when used in reference to a human version of the protein, and to the protein of UniProt Accession No. P28798 (mouse) when used in reference to a mouse version of the protein, and related isoforms and orthologs.

As used herein, the terms “regulatory RNA” and “regRNA” are used interchangeably to refer to a noncoding RNA transcribed from a regulatory element of a gene (e.g., a protein-coding gene), wherein the gene is not the noncoding RNA itself. Exemplary regulatory elements include but are not limited to promoters, enhancers, and super-enhancers. A noncoding RNA transcribed from a promoter, in the antisense direction, is also called “promoter RNA” or “paRNA.” A noncoding RNA transcribed from an enhancer or super-enhancer, in either the sense direction or the anti-sense direction, is also called “enhancer RNA” or “eRNA.” It is understood that a natural antisense transcript (NAT) complementary with at least a portion of the transcript of the gene is not a regulatory RNA as used herein.

As used herein, the term “nascent RNA” refers to an RNA that is still being transcribed or has just been transcribed by RNA polymerase and remains tethered to the DNA from which it is transcribed. An RNA that has dissociated from the DNA from which it is transcribed is also called an “untethered RNA.”

As used herein, the term “antisense oligonucleotide” or “ASO” refers to a single-stranded oligonucleotide having a nucleotide sequence that hybridizes with a target nucleic acid under suitable conditions or a conjugate comprising such single-stranded oligonucleotide. In some embodiments, the disclosure encompasses pharmaceutically acceptable salts of any of the ASOs described herein. Suitable pharmaceutically acceptable salts include, ut are not limited to, sodium, potassium, calcium, and magnesium salts. In some embodiments, the ASOs provided herein are lyophilized and isolated as salts (e.g., sodium salts).

As used herein, in some embodiments, the stability of a regRNA is reversely correlated with the degradation rate of the regRNA. In some embodiments, where an ASO increases the stability of a regRNA, it reduces the degradation rate of the regRNA. In some embodiments, where an ASO decreases the stability of a regRNA, it increases the degradation rate of the regRNA. In some embodiments, the degradation rate of a regRNA can be measured by blocking synthesis of new regRNA and assessing the half-life of the existing regRNA.

As used herein, the terms “subject” and “patient” refer to an organism to be treated by the methods and compositions described herein. Such organisms preferably include, but are not limited to, mammals (e.g., rodents, primates, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### II. Antisense Oligonucleotides

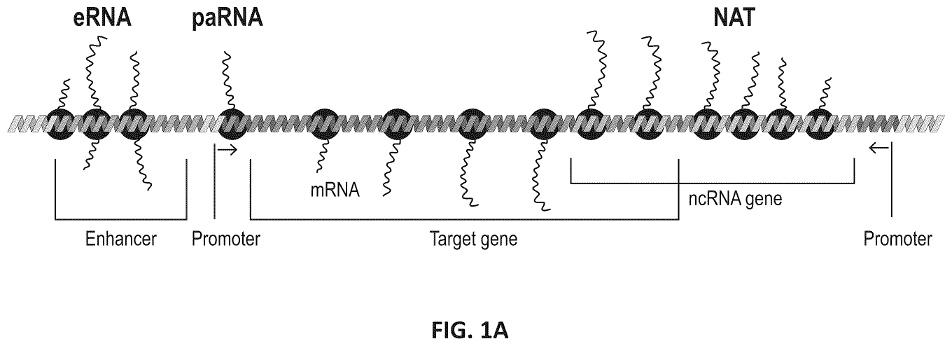

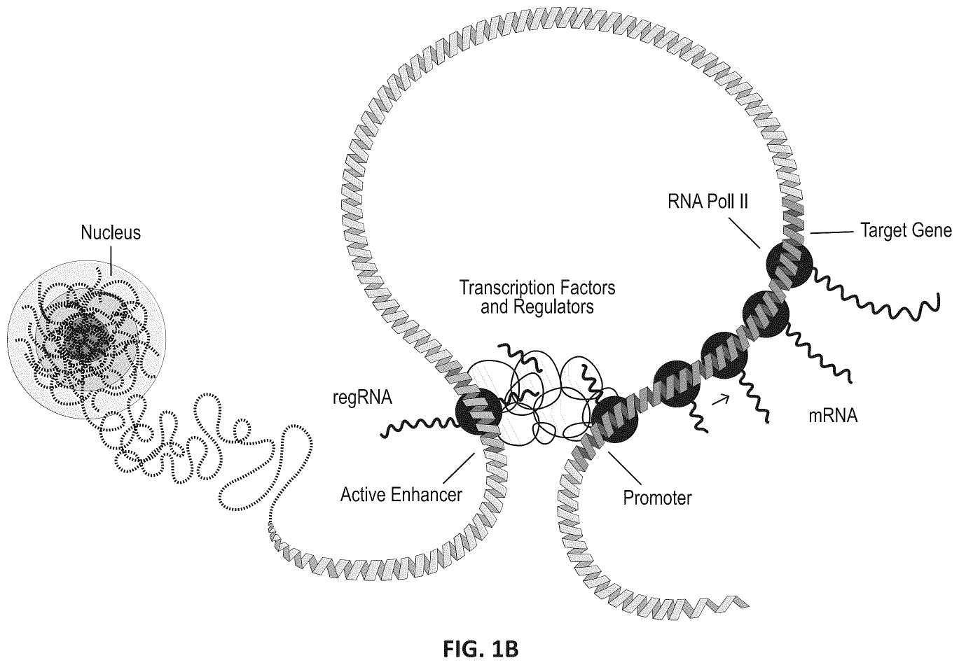

In some embodiments, the antisense oligonucleotide (ASO) disclosed herein hybridize with a regRNA (e.g., a regRNA or a paRNA) transcribed from a regulatory element of the GRN gene (also referred to herein as a “GRN regRNA”). It is understood that both eRNAs and paRNAs are regRNAs modulating (e.g., facilitating or upregulating) gene expression (FIG. 1). In some embodiments, the GRN regRNA is a murine GRN regRNA. In some embodiments, the GRN regRNA is a human GRN regRNA. In certain embodiments, the target GRN regRNA is an eRNA. In certain embodiments, the target GRN regRNA is a paRNA. eRNAs can be identified using methods known in the art, such as Assay for Transposase-Accessible Chromatin using sequencing (ATAC-seq), global run-on sequencing, precision run-on sequencing, cap analysis gene expression, and histone modification analysis (see, e.g., Sartorelli & Lauberth, Nat. Struct. Mol. Biol. (2020) 27:521-28; PCT Application Publication No. WO2013/177248). paRNAs are RNAs transcribed from promoters of target genes in the antisense direction (transcripts in the sense direction are mRNAs of the target genes). They can be identified by similar methods, taking into account of their specific location and orientation. The nucleotide sequences of exemplary regRNAs are provided in Table 1 below. Any of these GRN regRNAs are contemplated as a target GRN regRNA of an ASO disclosed herein.

TABLE 1
Exemplary regRNAs

| regRNA | Nucleotide Sequence |
|---|---|
| Human GRN | CTCCGTTATCTTCCACTAGGTGGCACCCCCCAACTCCGAGTGCCAAGGGGAGAGGA |
| (hGRN) | AATTCCCAAAAGGGGAGCCAACCTAGCTGCAAATGAGCAGGAAAAAGTGGCTGGG |
| paRNA 1 (−) | AGAGGCGGAAGCTAGCTCGTCCCACCCGCAACAAGAGGAAGTAAGGAGGAGCCA |
| strand | GACCAGGTTCAGTCACCACTCCCAAGTCCCTACTACCTTCGAGAAGCCAAGGTCTC |
| SEQ ID NO: 1 | AGGTCTCGTTCCCAGGCCCTCGGAGCTCCCAGCCCAGGGTCGCGCGCCCCTCCGGC |
| | TCCAGGCCGCCGCGGGAACCACCCACCACCACCAGGAGAGGGGAAGAAGCCAGCA |
| | CCTACCGACAGGGGTGGAGCTGGGTCAAGAATGGTGTGGTCCCTGCTTTGGGGGA |
| | ATGCTGGGGAGGTAGAAAGCCCCTTCTAACGGGGCGTCACTGCAATTACTGCTTCC |
| | TCTTTCCCATAAAACTCCCCCTAGTGTATCAGAACCCCCAAGGAGTTTCAGTAAGC |
| | GGTTCTTCTGTTGTCTCCGGCTGAGACTCCAGGGGAACCTCAAGCTCACATGGCCC |
| | TGGCGGGCCCCTGGGCAGGAGCAGGCGAGAGGTCTGCGCGGCCGCTCTCCTACCT |
| | GCGTCCGACTCCGCGGTCCTTGGGCAGCAGCAACCGGGTAGCGCTCAGACTACAG |
| | ACCCCAGCGCGATGACCCGCCTACCTCAGTTTCCATTGGCCAGGGATCAGGGCTAC |
| | CCGCTCCCATTGGCTACTTATCAATATAGAGGTGGGGCCAGCCTGGAATGCTGTGT |
| | TTCTTTTCTACTCAAATCACTCTCCTCCCTGCTTCCTCTCGCCCCACACCCCATTTCT |
| | AGGGATCATGTGATTGGAGAATCATGTGACGTCGGGACAGCCTCAGCAGGGCAGC |
| | TGGTGCTCGTCGCCTTCCTTAACCCATCCTGCAGGCGCCTGGGGGAGTTTGCTAGA |
| | GATGATAGCGCGTGTCTGGGGCATTGACAGTGCGAGGGAGTGTTATGAATTGGGA |
| | ATATTTGTGCTCGCCTTTCTATCCTTTTGTTTGTTTGTTCTGAGACGGAGTCTCACTC |
| | TGTCGCCAGGCTGGAGTGCAGTGGCACGATCTCGGCTCACTGCAACCTCTGCCTCC |
| | CGGGTTCAAGCGATTCTCCTGCCTCAGCCTCCCGAGTAGCTGGGACTACAGGCGCG |
| | CACCACCACGCCCAGCTAATTTTTGTATTTTTAGTAGAGACGGGGTTTCACCATGTT |
| | GGCCAGGATGGTCTCGATTTCTTGACCTCGTGATCCGCCCACCTCGGCCTCCCAAA |
| | GTGCTAAGATTACAGGCATGAGCCACCGCGCCCGGACTTGTGTGTTTGTTTTAGAG |
| | ACAGGGTCTCGCTCAGTCGCCCAAGCTGATCATAGCTCACTGCGGCCTGCAACTCC |
| | TGCTCCCAAAGCGATTCTCCTACCGCAGCCCCCCAAGGCGCTGGGATTACAAATGT |
| | GAGCTACTGAGCTGGCTCGCTCCTATCCTATCCTGCCCTGTCTTGTCCTGTCCTGTC |
| | CTGCCCTATTCTGCCCTATTCTCCAAACTCGGTTGACATGCTAGGCAGGCCCCAGA |
| | GTGAGTTGAAGGATGGTTTCCAGGACAAAGATAAACAGCCCCAAAGCCCTTTCCTC |
| | AAGGTGCTCAGCAGGACAGGGAGCAGGGATTGGTGGGAGACAGTCTCAGGCTGAA |
| | TCTAATTTAAGATACAAGGTTGTGCGCCAGTTGCCAGGTGTAGGGAGAGGAAGTA |
| | GATTGTCTCCAGGTGGGGAAGAAGAGATTTCATTCCACCTTAGGAGCATCTCCTTG |
| | AGCTGAGTCTGGGAAGATTTTAACTGGCAGGTGGAGGAGGAGGTGTTGGGAAGAG |
| | CCCTGGGAAACCAGATGGGAAGGGCAGGCTTGGCAGGGATTGAATGCCATGTTCA |
| | ATGCCAGTGGGCTTCAGGAGCTGCTGGGTGCCTGGCACGGGCTGGGCTGTGGTGGG |
| | GGCTGTCGAAAGAGAAGTGGCTTCCTAGTCTCCAGATCGGCCCTGCCTTCGAGGAA |
| | TTGATATGGGAGGGTGAGGAGAGAAAATGTACCACAGAACAGATTAGCCAGTGAC |
| | AAGAGTCAGCCTGCTTGGTGCCAAAGGGCCTGGGCCAGGCAGAGATGGATGATGG |
| | GAGATGGGCCAGGAGAGAGGAGAGGCCCAGGGAAAGCCTGATACTGGGCCGTGG |
| | AGGAACGGCAGTGTCTGCACAGGGGATGGAGACTGCTGCAGCCTGAGCAAGGAAC |
| | TGGAGCTGAGGATGAGGCAGGGGCAGGGGGATGGCTAACCAAGCTCGAGGCAGC |
| | AGAAGTCGGAGGAGGGAAAGGGTGCCAACCGAGCTGTGGAGTCGTGTGGCAGGG |
| | GTTCTAAGCCTGGAGCTAGAGCAGACCCAGACAGCGGATAAGACACCTGGCCTGC |
| | TGAGGACCTAGTACTAGTCTGTGCCCTGGTGACACATTTGTGCTTATTTTCCTTCCT |
| | GTGTATCTTGAGACTAGGGAGTATAGTTTATGCTTCTATTGAAACACACACACACA |
| | TGAAACTTTCATGAAATGTTAGGTGAATCGATCAATAACCTACTAATTACAACCAC |
| | CCAGAACCAGAAAAAAATACAGGTAGAAGAAAAAAAACCTAAACAGACTGAAAC |
| | AAATATCACAAAGTCTAATCGTCAAAGCCGTGATTCTTGAGCAGCGGCAATTTTGC |
| | CGGCCAGGGACATTTAGGAGTGTCTGGAGACAATCTGGGTTGTCACAACTCAGGGT |
| | GGAGGGACACTGGCATCTGGTAGGTAAGGGCCAGCAATCCCACTAAACATCACAC |
| | GACACACAGAACAATCCCCCACAACAAAGAAGTATCCTGCCCAAAAGGTCAATAG |
| | TGCTGAGACTCAACAACCTACTTCACAAATGTTTCTCTCTCTCTCTCTCGCTCTTTTT |
| | TTTTTTTTTTTTAACATCAGAAGGGTAAGGTGCCACGATAACGAGGTCTGTAATTCC |
| | CTACTCAGGAGGCCGAGGCAGGAGAATCGCTTGAACCCGGGATGCAGAGGTTGCA |
| | GTAAACCGAGATCGTGCCACTGCATTCCAGCCTGGACAACAGAGCGAGACTCCATC |
| | CACCCCCGCCAACACAAAAAAATTAAAGTGTACAACTCAATGGTTTTTAGTGTCTT |
| | AAGAGAGTTATGCAGCATCATTACAATCAGTTTAAGACCATTTTTATCACCACAGA |
| | AGGCAGCCTTATGTCCTTTAGCAGTCACCCTCCCTAACCCTAGGCA |
| hGRN eRNA | CGGAAGGCGGAGGTTGCGGTGAGCCGAGACTGCGCCATTGCACTCCAGCCTGGGC |
| 1 (+) strand | AACAAGAGCGAAACTCCGTCTGAAAAAGAAATAGATTACCCCGTTTTACAGAAAG |
| SEQ ID NO: 2 | TGAAACTGAGGCTCGACGTGAAGAGCCGGAGATTCGAACCGGCCGGGTGCTTTTCC |
| | CAGGGCTCCACACTGCCTCCCCGGGAGACAGGAAGGCTTAAGTCCAGCGCTGCCCT |
| | CTCCGATCCCGCTTGTCAGGGAGACACTTTATTTCCCGGGGCAGCCCCGTACCCCA |
| | GGCCCCACCACCCACTTCGCGTTTAGAACTTCTGTGCTATCTTGCTTATTTGTCCTA |
| | ACAGAGAAACCAGCACGCGCGGGCGGTGGCGGAGGGGGGCGGGCGGGCGGGTGG |
| | AGGCGGGGGGGGGGGGGCGGGGAGATGGGTACTCCTAGCCTGCGCCTTTAAGAAG |
| | GGTAGGCCTCGACTTTGACGTCTCTGCCTTCCCCGCCCTTCAGGCCCCCACTGGTCG |
| | CGTCCGGCGCTGGAGGAGCCCAGTCAGCCGGCGCCTGCCGGGTTAGCACGTGGAC |
| | TCCGAGGGGCCAACTATCAGCTTTCCCTGACAAAATGCCTTTGAGCTCCCCCACAG |
| | CTCTGAAACTCCAGCTTGGGAGCAGGGAGAGTGCAATCTGTGACCTGTAAAGGGG |
| | CGCTGGGCAAAAGGGCCCGAGAGAAGGCGTCCTTTCCATTCCCTTCCCATCTCAAG |
| | CTGAGTTCAGAAATGACACGAAATAATTTAATCAACTATCAGTCAAGGTCGGGCTC |
| | ACGCCTGTAATCCCAGCAGTTTGGGAGGCCGAGGTGGGCGGATCACCTGAGGACA |
| | GGAGTTCGAGACCAGCCTGACCAACATGACGAAGCCCCGTCTCTACTAAAAATAC |
| | AAAAATTAGCCAGCGGTGGTGGTGCGCACCTGTAGTCTCAGCTACTTGGGAGGCTG |
| | AGGCCAGAGAATCGCTTGAGCCCGCGAGGTCGAGGCTGCAGTGAGCCGAGATCAC |
| | ACCACTG |
| hGRN eRNA | CAGTGGTGTGATCTCGGCTCACTGCAGCCTCGACCTCGCGGGCTCAAGCGATTCTC |
| 2 (−) strand | TGGCCTCAGCCTCCCAAGTAGCTGAGACTACAGGTGCGCACCACCACCGCTGGCTA |
| SEQ ID NO: 3 | ATTTTTGTATTTTTAGTAGAGACGGGGCTTCGTCATGTTGGTCAGGCTGGTCTCGAA |
| | CTCCTGTCCTCAGGTGATCCGCCCACCTCGGCCTCCCAAACTGCTGGGATTACAGG |
| | CGTGAGCCCGACCTTGACTGATAGTTGATTAAATTATTTCGTGTCATTTCTGAACTC |
| | AGCTTGAGATGGGAAGGGAATGGAAAGGACGCCTTCTCTCGGGCCCTTTTGCCCAG |
| | CGCCCCTTTACAGGTCACAGATTGCACTCTCCCTGCTCCCAAGCTGGAGTTTCAGA |
| | GCTGTGGGGGAGCTCAAAGGCATTTTGTCAGGGAAAGCTGATAGTTGGCCCCTCGG |
| | AGTCCACGTGCTAACCCGGCAGGCGCCGGCTGACTGGGCTCCTCCAGCGCCGGACG |
| | CGACCAGTGGGGGCCTGAAGGGCGGGGAAGGCAGAGACGTCAAAGTCGAGGCCT |
| | ACCCTTCTTAAAGGCGCAGGCTAGGAGTACCCATCTCCCCGCCCGCCCCCCCCCGC |
| | CTCCACCCGCCCGCCCGCCCCCCTCCGCCACCGCCCGCGCGTGCTGGTTTCTCTGTT |
| | AGGACAAATAAGCAAGATAGCACAGAAGTTCTAAACGCGAAGTGGGTGGTGGGGC |
| | CTGGGGTACGGGGCTGCCCCGGGAAATAAAGTGTCTCCCTGACAAGCGGGATCGG |
| | AGAGGGCAGCGCTGGACTTAAGCCTTCCTGTCTCCCGGGGAGGCAGTGTGGAGCCC |
| | TGGGAAAAGCACCCGGCCGGTTCGAATCTCCGGCTCTTCACGTCGAGCCTCAGTTT |
| | CACTTTCTGTAAAACGGGGTAATCTATTTCTTTTTCAGACGGAGTTTCGCTCTTGTT |
| | GCCCAGGCTGGAGTGCAATGGCGCAGTCTCGGCTCACCGCAACCTCCGCCTTCCG |
| hGRN eRNA | ATATATATATATATATATAAAACAGCTGGGATCAGCCCAATTTGGGGAAGTCCTCC |
| 3 (+) strand | CTGTCTCCCCCTGCCTCTGTGGCCTGAGAAGGTGGGCTGGAAGTGTCTAGGCACCC |
| SEQ ID NO: 4 | AAGGCTTTATTGGGGGGCCCTGGGTGTGGTCTGAGGAGGTGGGCCCCTAGAGGGA |
| | GAGGGTCCTGGCTAAGACTTTCCAGCATGTCCCCCAGCCTGGGTCCCAGAGCTCTG |
| | CTGGGACAGCGTGGATTAATATGGAAGGTAGTTTTAGCCATAGCTATGAAAATTCT |
| | AAACGCCAAACCCTTGATCCAGCCAGGCCACTTCTAAGCGTTTCTCTAGTCATCTG |
| | CTCATACAAGTGTGAAATGATGGTACCAGCTTGGCCTTCTTCATTGCAGCCTTCTTT |
| | GTAACAGCAGAACATCCCGTGAAAGGGAACTGTTAAGTACGTGACCCTATCTCCAT |
| | GTGACAAATCCACACAGCCTTGAAAGGGCGGCATTCCCTGACCTATCCAGGTGGGG |
| | TGGGGGGTGAGGGCTGCAGATCTCTATATCATTATGTAAAACAATCTCAAAGACAG |
| | AGTTAAATTAAACAGCAGGTGCTGAGAGCGTGCTGGACACTCGTTCCAAACAGCA |
| | GCGTTACTGCCCCCTAGAGGACATTTTTTGAAATTTATGCGGCCACTGTCATTTCTG |
| | GTTGTCACAAGACTGGGGCTTCTGGCATTTGATAGATGGCAGGTCAGGGAAGCTAA |
| | ATGCCCTGTAAGGTGTAGGACAGTCTGACACAATAGGGAATTTCCAACAACAAGA |
| | ACACATCTCTCAATTTCCTTCTGTCTTTCTGGGCAATTACATACATGAGGAAATGTT |
| | TGCAATTCTCAGACCTGGAAAATGGTTCTGTTTTACCCATATACCCACACCTTTTTT |
| | TTTTTTTTTAGACAGGGTCTCTCACTCTGTCGCTGGTTGGAGTGCAGTAGCACGATC |
| | ACAGCTCACTACAGCCTCGACCTCCCCGGCTCAAGCGATCCTCCCACCTCAGCCTC |
| | CCGAGTAGCACACTCCTCTGCCTGACTAATTCGTTTGTATTTGTTGTAGAGACAGGG |
| | TTTCGCTATGCTGCCCAGGTTGTAACCATACTTCTTTTGAATACTTATATTCTGCTAT |
| | GCATTGAGTTTTCCAGGAATAAAACCACTCTGCAGTTGTATGGAAAATTGGGTTTT |
| | GTCTTCAAGAACTTTACCGAAGGTCATTTGCCATTTCATTTTTTGTTTGGTTTGTTTT |
| | GTTTTTTTGAGATG |
| hGRN eRNA | CATCTCAAAAAAACAAAACAAACCAAACAAAAAATGAAATGGCAAATGACCTTCG |
| 4 (−) strand | GTAAAGTTCTTGAAGACAAAACCCAATTTTCCATACAACTGCAGAGTGGTTTTATT |
| SEQ ID NO: 5 | CCTGGAAAACTCAATGCATAGCAGAATATAAGTATTCAAAAGAAGTATGGTTACA |
| | ACCTGGGCAGCATAGCGAAACCCTGTCTCTACAACAAATACAAACGAATTAGTCA |
| | GGCAGAGGAGTGTGCTACTCGGGAGGCTGAGGTGGGAGGATCGCTTGAGCCGGGG |
| | AGGTCGAGGCTGTAGTGAGCTGTGATCGTGCTACTGCACTCCAACCAGCGACAGAG |
| | TGAGAGACCCTGTCTAAAAAAAAAAAAAAAGGTGTGGGTATATGGGTAAAACAGA |
| | ACCATTTTCCAGGTCTGAGAATTGCAAACATTTCCTCATGTATGTAATTGCCCAGAA |
| | AGACAGAAGGAAATTGAGAGATGTGTTCTTGTTGTTGGAAATTCCCTATTGTGTCA |
| | GACTGTCCTACACCTTACAGGGCATTTAGCTTCCCTGACCTGCCATCTATCAAATGC |
| | CAGAAGCCCCAGTCTTGTGACAACCAGAAATGACAGTGGCCGCATAAATTTCAAA |
| | AAATGTCCTCTAGGGGGCAGTAACGCTGCTGTTTGGAACGAGTGTCCAGCACGCTC |
| | TCAGCACCTGCTGTTTAATTTAACTCTGTCTTTGAGATTGTTTTACATAATGATATA |
| | GAGATCTGCAGCCCTCACCCCCCACCCCACCTGGATAGGTCAGGGAATGCCGCCCT |
| | TTCAAGGCTGTGTGGATTTGTCACATGGAGATAGGGTCACGTACTTAACAGTTCCC |
| | TTTCACGGGATGTTCTGCTGTTACAAAGAAGGCTGCAATGAAGAAGGCCAAGCTGG |
| | TACCATCATTTCACACTTGTATGAGCAGATGACTAGAGAAACGCTTAGAAGTGGCC |
| | TGGCTGGATCAAGGGTTTGGCGTTTAGAATTTTCATAGCTATGGCTAAAACTACCTT |
| | CCATATTAATCCACGCTGTCCCAGCAGAGCTCTGGGACCCAGGCTGGGGGACATGC |
| | TGGAAAGTCTTAGCCAGGACCCTCTCCCTCTAGGGGCCCACCTCCTCAGACCACAC |
| | CCAGGGCCCCCCAATAAAGCCTTGGGTGCCTAGACACTTCCAGCCCACCTTCTCAG |
| | GCCACAGAGGCAGGGGGAGACAGGGAGGACTTCCCCAAATTGGGCTGATCCCAGC |
| | TGTTTTATATATATATATATATAT |
| hGRN eRNA | GGGCCCCACCCCTGTATCTGCACCCTGCCTCTGGTACCTGTGTGCGCAGGCAGCTT |
| 5 (−) strand | AGTGAGGAGGTCCGTGGTAGCGTTCTCCTTGGAGAGGCACTTACTCTGGATCAGGT |
| SEQ ID NO: 6 | CACACACAGTGTCTTGGGGGCAGCAGTGCAGGTGATCGGAGCAGCAGGTGGCCTG |
| | AGTGGGGACAGGGGTGGGTCTTTGTCACTTCCAGGCTCAGTAGCACACAGGGAGC |
| | AGCCAGGCTGATGGCTGGCACTGCCCCAATCCCCCACTAGGGGGTAGCATGAACCC |
| | TGCATCAGCCAGGCTCCCAGCAAGCAGCCAGCCAGCCCCATGCCACAGAGCCCCT |
| | GTAAGGTGCGTGTCAGGGCCAGGTGGCTGGGGGCACACAGCCAAGCTGGCTCCAG |
| | CCCCTCACTCACGTTGGGCATTGGGCAGCAGCCATACTTCCCACTGGGCAGCTCAC |
| | AGCAGGTAGAACCATCAGGGCACCGGGACCGTGCGTCCGGACACATGACCGAGCT |
| | GGACAAGGCCACTGGAAGAGGAGCAAACGTGAGGGGCAGCCAGGATGGAGGAAA |
| | CTTTTTCCTACACTTGGAGTCAATGAGGCCCCGCCCCAGCCCCTGGC |
| Mouse GRN | TTTAGCGATTCCACATTGTGTACATATATCAAAATATCATGTTGTACACCATAAATA |
| (mGRN) | CAGTTTTTTTTTAAGTATCCCACCGACTATTTTTAGTTTTTCTTTTTTAATCCAATGA |
| paRNA 1 (−) | CATAAGCACAGTTTCCTAAAATGGGAACTGACCCTCTCCTACAAAACATTTCATTA |
| strand | TGGTTATATCTTCATGACATGAAAACAACTAGTTGAAAAGTGATGACTTTTTTAGTC |
| SEQ ID NO: 7 | TATTACCTGACTGTTCAGTAAGAATTGGTTTGACTACTTTTTTCACCCCACATTGAA |
| | TTTGGTTCACTAGTTTAGTGGTCCCTCCCCCATGTCATCCCCACCCAGTATGTGTAT |
| | TATGTTGTATTTTGGGGAAGATACAAAGAGAAAAATTGCTTTGCTGTTAATTGAAT |
| | TTGGGCAAGCTACTTAATCTCTGCCTTCTCACTTCCTGCTATGAAATAGAGCTGATT |
| | ATAGAAAGATGGCTGTCTCATGCGGCTAAGAGAGTATAGCCATCTCATGGGTTAAA |
| | TGAGGTAACATGTAAAGCACCTGGCACAAAACCAGGACCTAAATAAACGTGTTTTT |
| | CCTAGCTCTGTGGCCACACTTACCTTTTTGGTTCCTCCTTTCTCTGCCCTCTCTGCCA |
| | TGAGACCTTTGCATGTTCTGTTCTCACTACCTGAAGAGCTCTTCCTTTCATCTTTGCC |
| | TTTTTCTGGCTACCAGTTACCCTTTCAGCCTTTAGATTGCTCAAGCTTGTTTCCTCAG |
| | GTCCAGTCCTAGACCTCTAGGCTTATAACTGGGATACCAGCCTTCAGAGGAGTTTG |
| | TGGTTTACTGGCTTATAGTAATGTAATGTGATGCTTCATCTCACTCTGCCTCCCCAA |
| | CTAGAAAGTCAGCTCCTTCAGGGGGAAGGCTGATTTTACTCACCACCACCTTCCCA |
| | GCACCCAGTGCTGTTTTTGTTGAAACAGTAAAGGATATGCGAATGCTATGGGAGGA |
| | GTAAACCAGTTTTTGAGTTTTTTGCTTGCTATACCAGCCTTAGTTGTGAGATGGAGC |
| | CCCTTGGTCAAAACAAATCACCACCTGCACTCTGGTCTTCTCACTTAACAGGGGAG |
| | AATTGCCACAGTTGTCTTCTGTGGTCCCGGGCAACTCTGTTCTTTGCCTCTTTGTTTC |
| | CATAAAGATGAAGCCCATCCAGAAAAGGTACTCATGCTGGCTGGGAGAGGTGGCA |
| | TGGGGAAGGGGATGGACAGAGCAATTTGGAAGATCAGAGAGGCCTCTGCCCCTAC |
| | GAAGGGCCATGCTTGCCACCAGTCTTCCTGCTGTGTGGTGCTCCCATCGCACAGGC |
| | TGCTTCTGGAGATGCTCGGGTTGTTCAGGCCCTGTGGCTTTTGGCAAGGCAGGAGC |
| | ATCTGTGCTTCTGCTGACATAAAAGAGGGTAATAAAAGTGACACAGGAAAGCGGT |
| | GTTGTGAGGCTTTAGTGATGGTGGAGCAGTGTTGCTGAAGTCCCTCAGGGAGTTTG |
| | GATTTGATTGCAGTGAGTCTGGCTACATCTGAAGCCTATAATAGTTTACAGTACAA |
| | GATGCTATGTATAAGATGTCAAACATTTGCTTGGTAGAATGCTATGATTGTCAGGA |
| | GTTTACTTTTGCACTGAAAATAGTCTTGTTGGTTTGGTGTAAGTAGAGTCACATTGC |
| | AGCACACACACAGGCATTCATGCTTTTATGTTTGTTAATTGTAAGGAAATGTTTTCT |
| | CAAGGCAAGAGTCAAAGGTTAGCAGAGTGCCAAAGACCTTCTCAAAGAGGAAAGT |
| | CTCATTTTAGAAACTTCATTTTGTACGCCAAAACTTGCCTTTCAGTAGTCTCATGTA |
| | ATGTTTTTGCTGACATGAGTTGAAAAGTGCATTCTGTTGTAAAGGAGGAAAAAAAC |
| | TGAAGAGGAACCGTAAGCGTGATTATGAAGAAGAGAAAAGAGCAGGAAGAAAGT |
| | AGACATCATCCCAGATTTAAATTTCATATTGTTAAAATCATTTGGTTGCAAGCAGTA |
| | GAAATTTTCTATAACTAACTTAGATAAACAAGGACTTGTTGGACACCTGCTGTCCA |
| | ACATTCACAAAACCTAAAGAAGAAAACTGAACTAGATCTAGTGTGGCTAGGGATT |
| | TCAGGGACAGGAATTAACAAATCTCCTGAGGAGGCTGCCATCAAAATGACTGTGTT |
| | AACTGTTGTCCATCCTGGTGTATTCTACTCACATTTCAGACTCCTGCGAGAGGCTGA |
| | TTGACCTGGTTTGAATCTCGTGCCTACCCACTCGTAGTCCCTCCAGACCCCAGGGG |
| | GTGAGCGAAGGGTGTTTGCCTGAAGGAAGAGTGGAACAGGAGCTGGGCAGGGAA |
| | AAAAGAACAGATGTTCATTCTACTGGCCACATAGCCACAGTCAGTGGTAATGATTT |
| | TCCCTGGCCAGACTTGGTGGCCAGTAGTTACTAGTCTCCTTTCTCTTCAGAAACATC |
| | TGCTCTTGTCAGAGTTATAGCCTATTCTTGATGAGCTGCAGCAAAAATGTACCACTT |
| | AAATTGGAGATGGATCATTTCCTGAGGATCTCAGAATATCTTTTTCAGTTAGCTTTA |
| | TAAAGTAATTGTCCAGATGGCCATTTAATTTGTGATATGGGATAAAGTAATCTAGG |
| | GGAAAAAACACTTCTTTAAAATGACTTTTTTTTTTTGGGGGGGCCGGGCACACTGG |
| | CTTACACCTGTAATCCCAGCACTTTGCGAGGCCAAGCTGGGTGGATCACCTGGGGT |
| | C |
| mGRN | AGGGGAGATGATGGTTAAAGGATACAGAGCCTCAGAGAAGAGGAAAACTTTCTTT |
| paRNA 2 (−) | TTTCTAGTTCTATTGCACAGCATAGTGAATATAGTTTATCGTGTATGGTACCTTCCA |
| strand | AAATCGCTGAGTAAAATTTCAATGTTCTCACCACAAAAATAAGTATTTGAGGTGAT |
| SEQ ID NO: 8 | GGATATGTTAATTAGCATGATTAAATTATTCTACATGTATTCATAAATCATAACATC |
| | ACTTTATACCTATAAATTTATACAGCTGTAATTTGTCAGTTTACAAAGATAAGTATG |
| | TGAGGTAATGCCTATATTTATTTAGCGATTCCACATTGTGTACATATATCAAAATAT |
| | CATGTTGTACACCATAAATACAGTT |
| mGRN eRNA | GGGAAAAGGATAGTCTCTTTAATGAATTATGTTTGGAAAACTGGTTATCCACATGC |
| 1 (−) strand | AGAAGAATGAAATAGCACCCTGATATCAGACCACATGATTTCCATATGGAAAAAT |
| SEQ ID NO: 9 | CAACTCAAAATGAGTTAAAGATTTAACATGAATCCTGAAGTTGTAAAACTACTAGA |
| | AAAAAATAGGAAAAACTACACAACATTGGTCTGGGCAATGATATTTTAGATTTGGC |
| | CCCAAAAGCACAGGCAACAAAAACAAAAATAGACAAGATTACATCAAACTGAAA |
| | AGCTTCTGCATAAAAAGGAAACAACAGAATGAAGAGACATCATACAGATTGGGAG |
| | AAAATATTTGTAAGCCATTTATTTGTTAAAGGGTTAGTATTAAAATATATTAAGAA |
| | CTCAACTCTATAGAAAGAAAATAAATAATCCAATTTAAAAATGGGCAAGGAACCT |
| | GAATAGACAGACATTTCTCAAAAGAAGACATACAGATAG |

The present disclosure describes ASOs that increase the amount or stability of the target GRN regRNA, to thereby increase expression of the GRN gene. These ASOs are different from the ASOs previously described which were designed to inhibit eRNAs (see, e.g., PCT Application Publication No. WO2013/177248 and PCT Application Publication No. WO2017/075406). Without wishing to be bound by theory, it is hypothesized that the ASOs' ability to upregulate GRN regRNAs is attributable to the selection of a target sequence in the regRNA and/or the chemical modifications of the ASOs.

### Sequences of ASOs

In certain embodiments, the ASO disclosed herein is complementary to a sequence in the GRN regRNA that is no more than 300, 250, 200, 150, 100, 50, 40, 30, 20, or 10 nucleotides from the 5′ or 3′ end of the GRN regRNA. In certain embodiments, the ASO disclosed herein is complementary to a sequence in the GRN regRNA that is no more than 300, 250, 200, 150, 100, 50, 40, 30, 20, or 10 nucleotides from the 5′ end of the GRN regRNA (i.e., the 5′ most nucleotide of the regRNA sequence forming a duplex with the ASO is no more than 300, 250, 200, 150, 100, 50, 40, 30, 20, or 10 nucleotides from the 5′ end of the GRN regRNA). In certain embodiments, the ASO disclosed herein is complementary to a sequence in the GRN regRNA that is no more than 300, 250, 200, 150, 100, 50, 40, 30, 20, or 10 nucleotides from the 3′ end of the GRN regRNA (i.e., the 3′ most nucleotide of the GRN regRNA sequence forming a duplex with the ASO is no more than 300, 250, 200, 150, 100, 50, 40, 30, 20, or 10 nucleotides from the 3′ end of the GRN regRNA).

In certain embodiments, the ASO is no more than 25, 30, 40, 50, 60, 70, 80, 90, or 100 nucleotides in length. In certain embodiments, the ASO is designed to lack a stable secondary structure formed within itself or between each other, thereby increasing the amount of the ASO in a single-stranded form ready to hybridize with the GRN regRNA. Methods to predict secondary structures are known in the art (see, e.g., Seetin and Mathews, Methods Mol. Biol. (2012) 905:99-122; Zhao et al., PLoS Comput. Biol. (2021) 17(8):e1009291) and web-based programs (e.g., RNAfold) are available to public users.

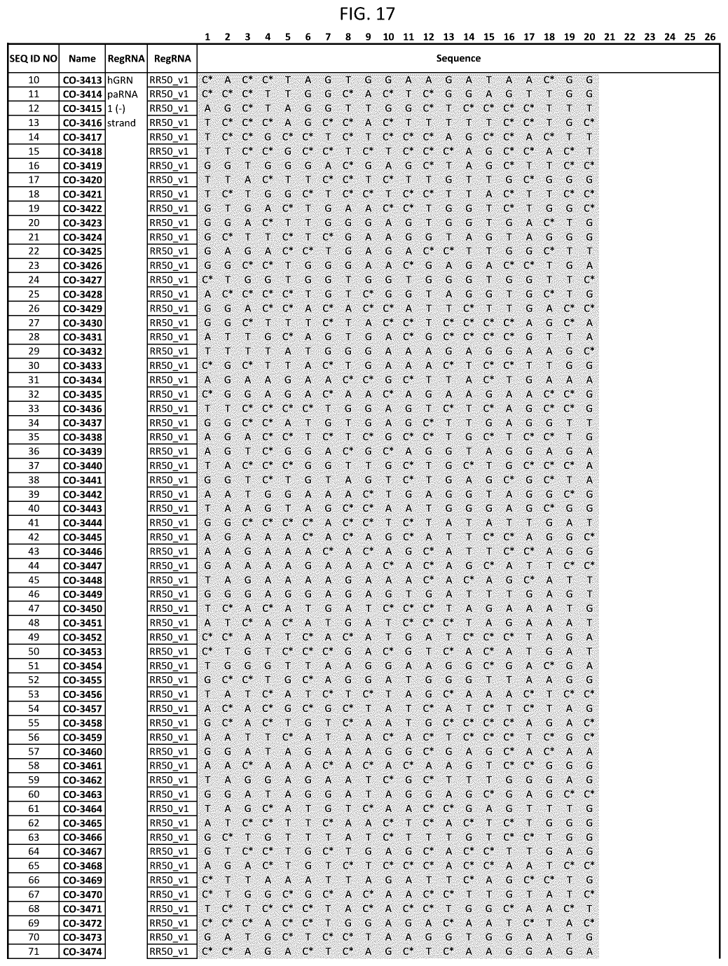

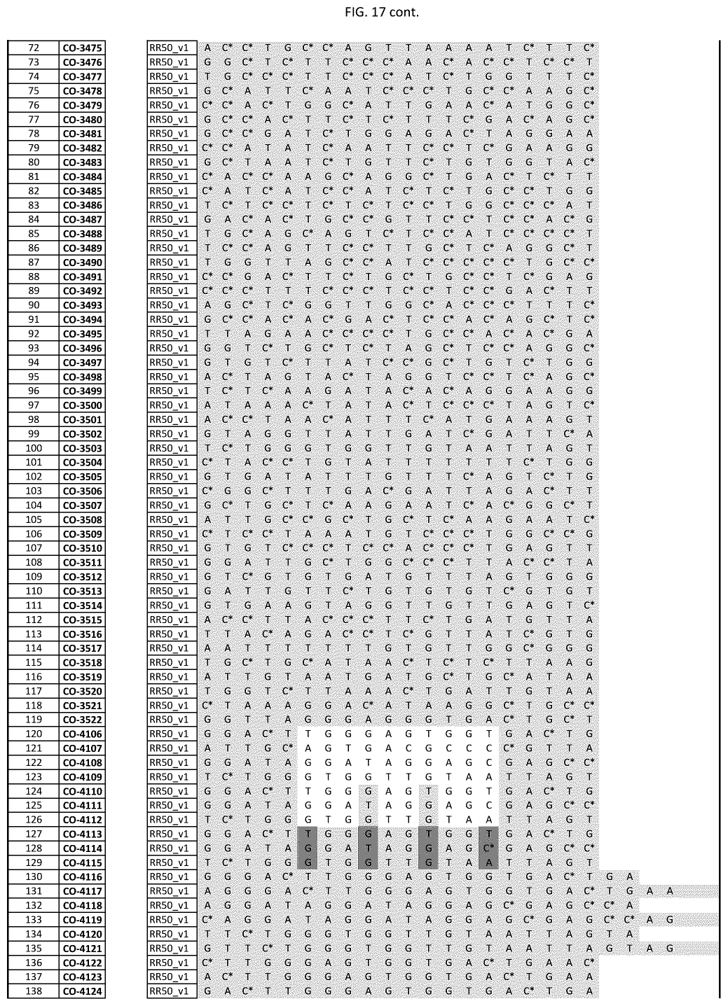

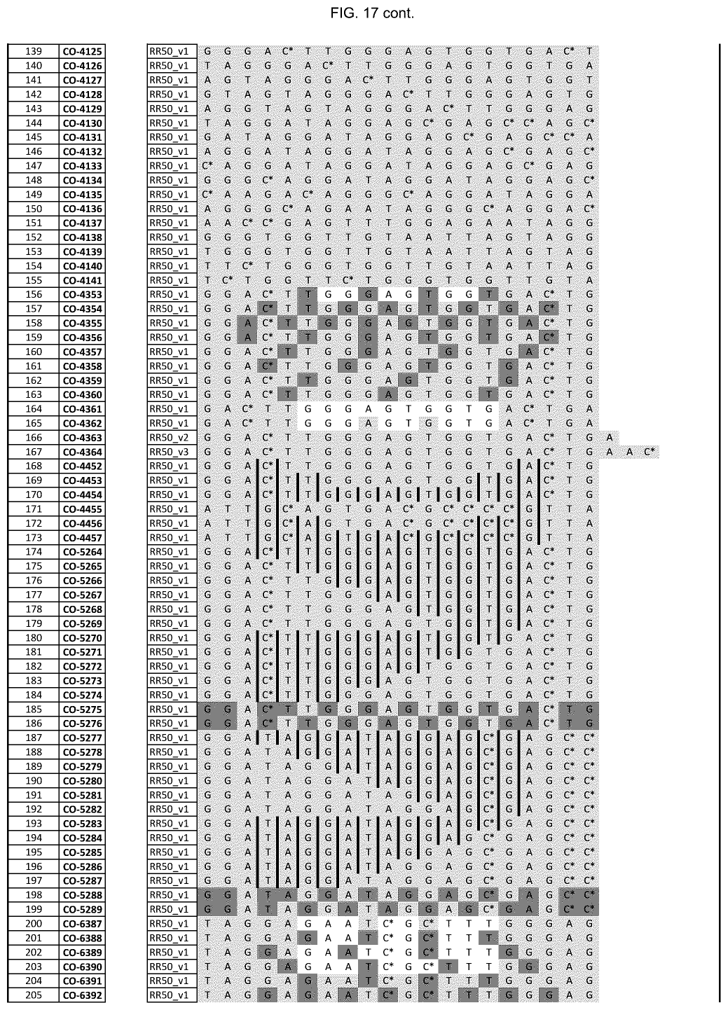

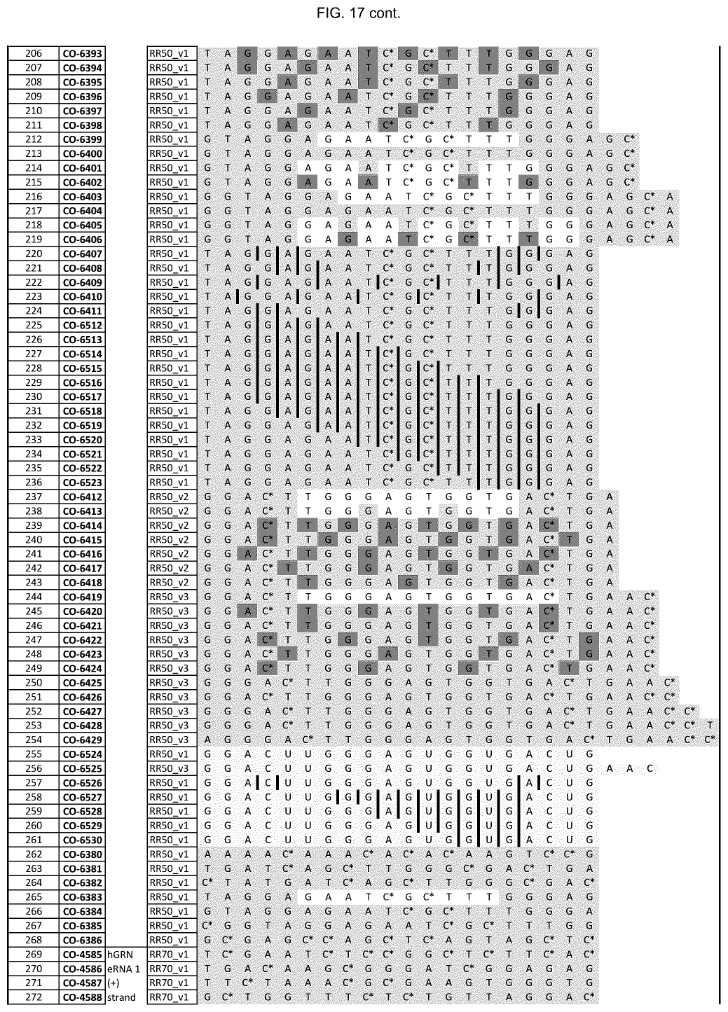

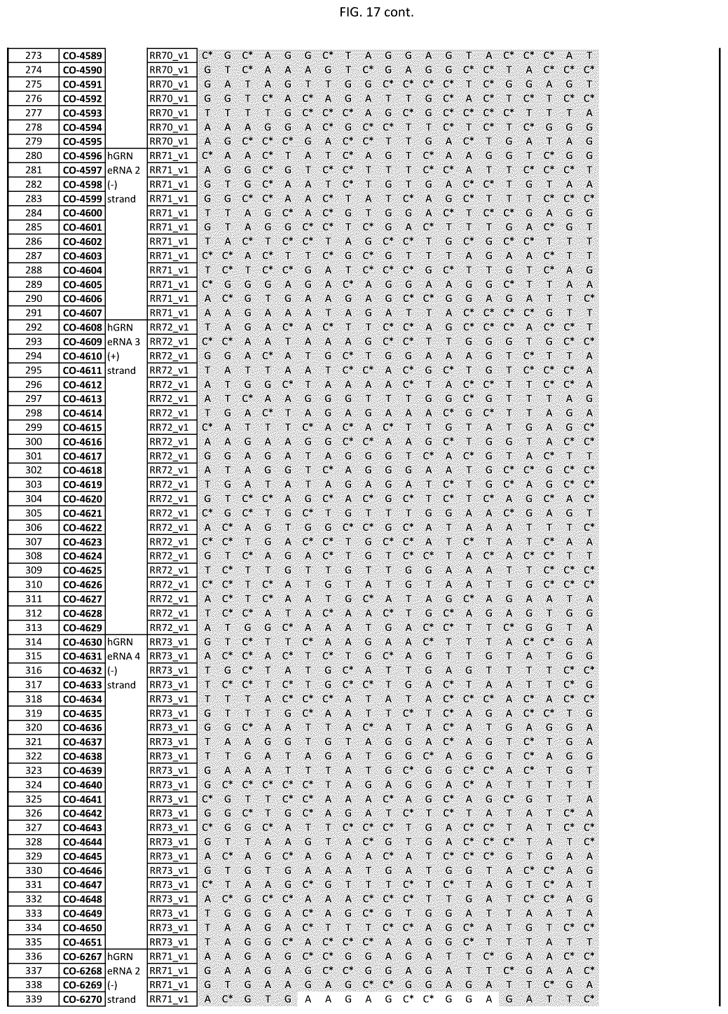

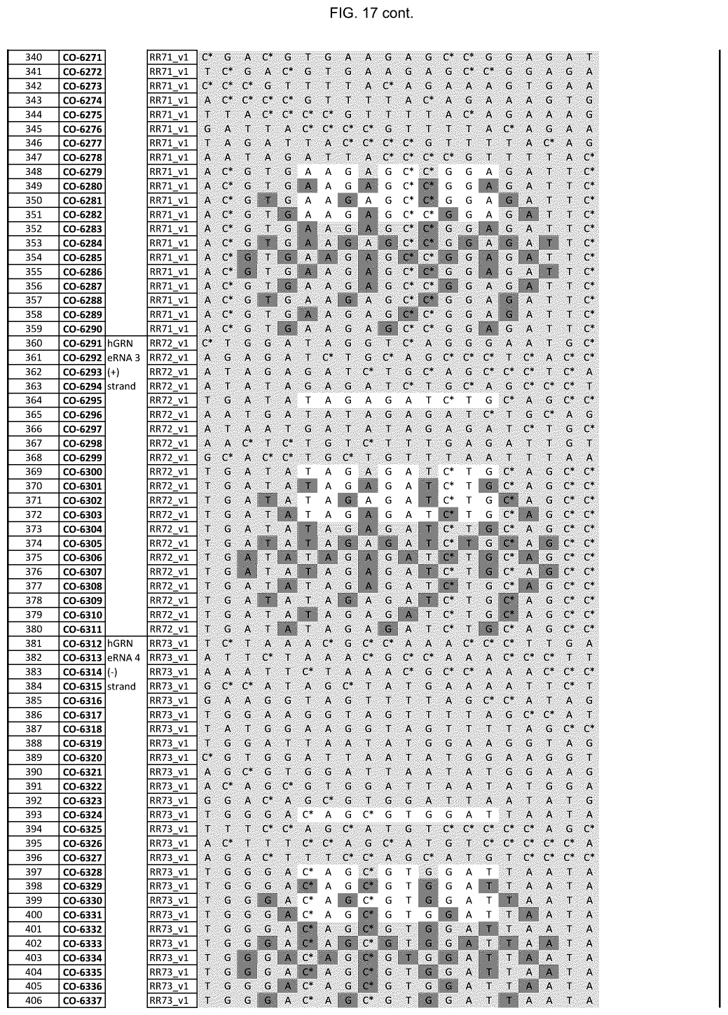

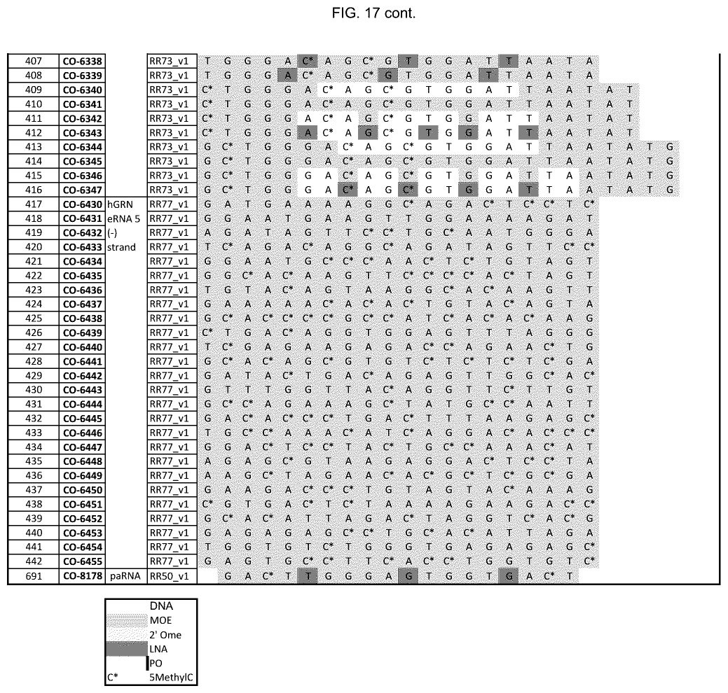

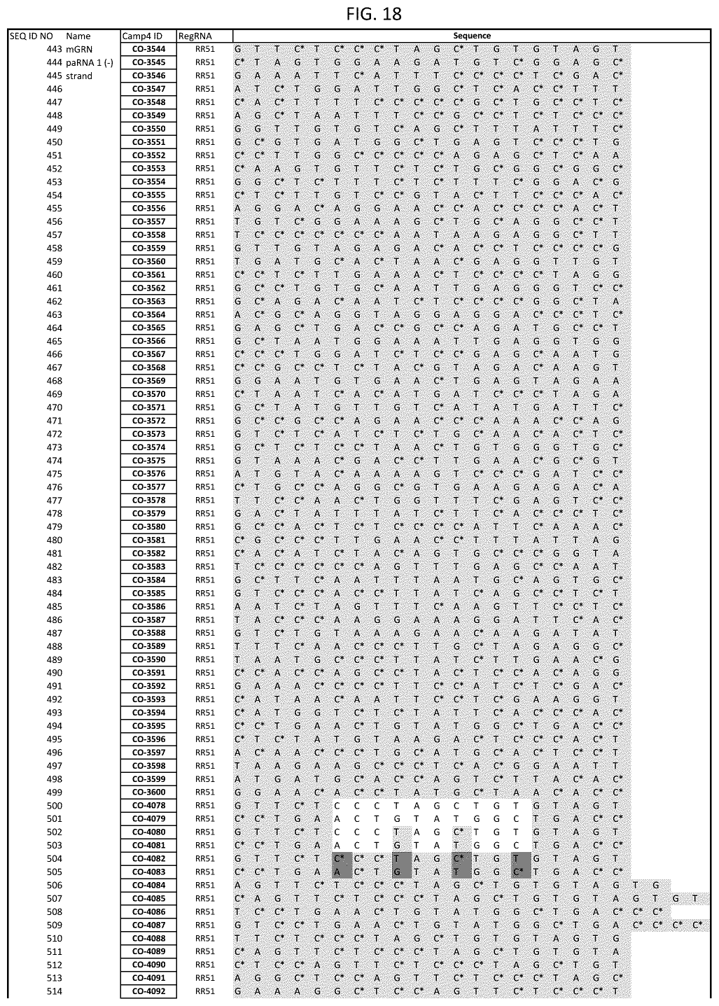

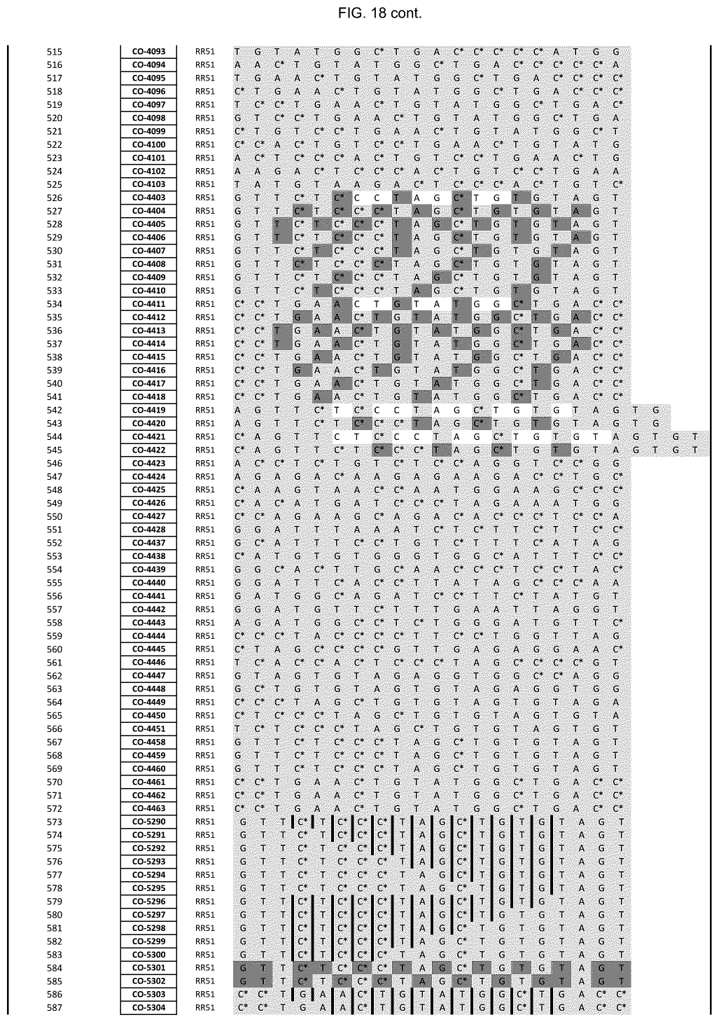

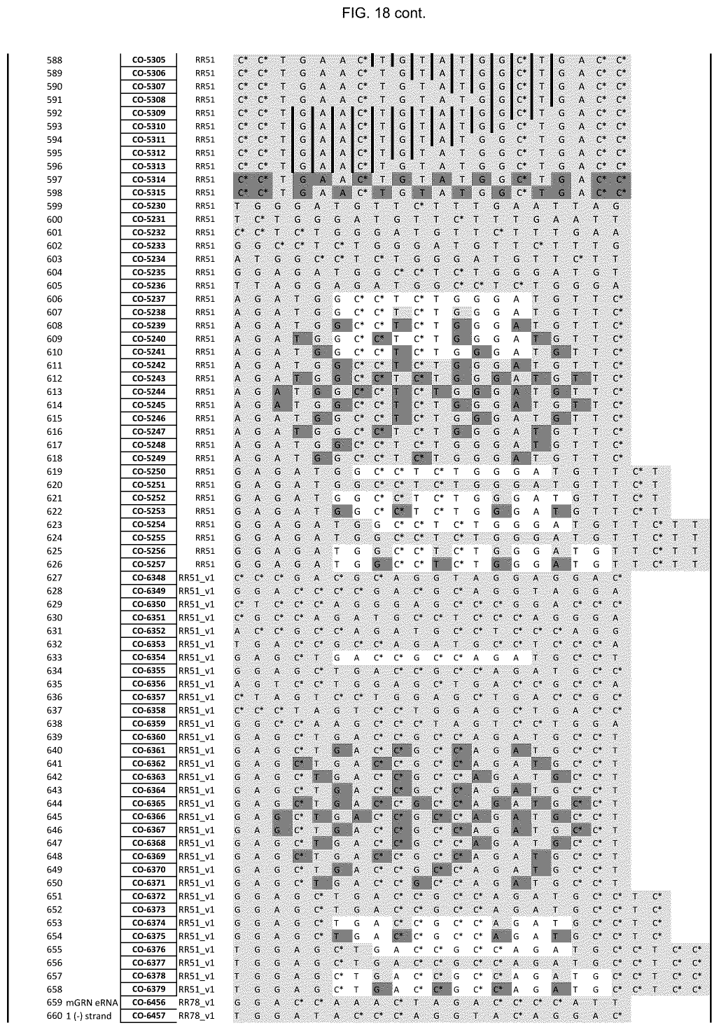

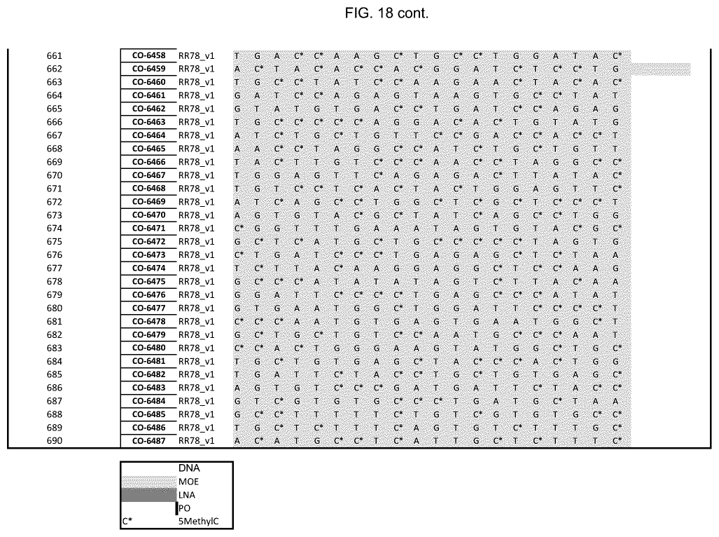

For example, ASOs have been designed to target a human GRN paRNA or GRN eRNA. The nucleotide sequences of some of these hGRN ASOs are provided in FIG. 17. Additional ASOs have been designed to target a mouse GRN paRNA or eRNA. The nucleotide sequences of some of these mGRN ASOs are provided in FIG. 18. In some embodiments, an ASO provided herein comprises a nucleotide sequence of any one of SEQ ID NOS: 1369-4738. In some embodiments, an ASO provided herein comprises a nucleotide sequence as provided in Table 19, below.

TABLE 19
Exemplary ASO Sequences.

| SEQ | | SEQ | | SEQ | |
|---|---|---|---|---|---|
| ID | | ID | | ID | |
| NO | ASO_sequence | NO | ASO_sequence | NO | ASO_sequence |
| 1369 | CCTAGTGGAAGATAACGGAG | 2493 | GGCTGAGGCAGGAGAATCGC | 3618 | TGGTTAGCCATCCCCCTGCC |
| 1370 | ACCTAGTGGAAGATAACGGA | 2494 | AGGCTGAGGCAGGAGAATCG | 3619 | TTGGTTAGCCATCCCCCTGC |
| 1371 | CACCTAGTGGAAGATAACGG | 2495 | GAGGCTGAGGCAGGAGAATC | 3620 | CTTGGTTAGCCATCCCCCTG |
| 1372 | CCACCTAGTGGAAGATAACG | 2496 | GGAGGCTGAGGCAGGAGAAT | 3621 | GCTTGGTTAGCCATCCCCCT |
| 1373 | GCCACCTAGTGGAAGATAAC | 2497 | GGGAGGCTGAGGCAGGAGAA | 3622 | AGCTTGGTTAGCCATCCCCC |
| 1374 | TGCCACCTAGTGGAAGATAA | 2498 | CGGGAGGCTGAGGCAGGAGA | 3623 | GAGCTTGGTTAGCCATCCCC |
| 1375 | GTGCCACCTAGTGGAAGATA | 2499 | TCGGGAGGCTGAGGCAGGAG | 3624 | CGAGCTTGGTTAGCCATCCC |
| 1376 | GGTGCCACCTAGTGGAAGAT | 2500 | CTCGGGAGGCTGAGGCAGGA | 3625 | TCGAGCTTGGTTAGCCATCC |
| 1377 | GGGTGCCACCTAGTGGAAGA | 2501 | ACTCGGGAGGCTGAGGCAGG | 3626 | CTCGAGCTTGGTTAGCCATC |
| 1378 | GGGGTGCCACCTAGTGGAAG | 2502 | TACTCGGGAGGCTGAGGCAG | 3627 | CCTCGAGCTTGGTTAGCCAT |
| 1379 | GGGGGTGCCACCTAGTGGAA | 2503 | CTACTCGGGAGGCTGAGGCA | 3628 | GCCTCGAGCTTGGTTAGCCA |
| 1380 | GGGGGGTGCCACCTAGTGGA | 2504 | GCTACTCGGGAGGCTGAGGC | 3629 | TGCCTCGAGCTTGGTTAGCC |
| 1381 | TGGGGGGTGCCACCTAGTGG | 2505 | AGCTACTCGGGAGGCTGAGG | 3630 | CTGCCTCGAGCTTGGTTAGC |
| 1382 | TTGGGGGGTGCCACCTAGTG | 2506 | CAGCTACTCGGGAGGCTGAG | 3631 | GCTGCCTCGAGCTTGGTTAG |
| 1383 | GTTGGGGGGTGCCACCTAGT | 2507 | CCAGCTACTCGGGAGGCTGA | 3632 | TGCTGCCTCGAGCTTGGTTA |
| 1384 | AGTTGGGGGGTGCCACCTAG | 2508 | CCCAGCTACTCGGGAGGCTG | 3633 | CTGCTGCCTCGAGCTTGGTT |
| 1385 | GAGTTGGGGGGTGCCACCTA | 2509 | TCCCAGCTACTCGGGAGGCT | 3634 | TCTGCTGCCTCGAGCTTGGT |
| 1386 | GGAGTTGGGGGGTGCCACCT | 2510 | GTCCCAGCTACTCGGGAGGC | 3635 | TTCTGCTGCCTCGAGCTTGG |
| 1387 | CGGAGTTGGGGGGTGCCACC | 2511 | AGTCCCAGCTACTCGGGAGG | 3636 | CTTCTGCTGCCTCGAGCTTG |
| 1388 | TCGGAGTTGGGGGGTGCCAC | 2512 | TAGTCCCAGCTACTCGGGAG | 3637 | ACTTCTGCTGCCTCGAGCTT |
| 1389 | CTCGGAGTTGGGGGGTGCCA | 2513 | GTAGTCCCAGCTACTCGGGA | 3638 | GACTTCTGCTGCCTCGAGCT |
| 1390 | ACTCGGAGTTGGGGGGTGCC | 2514 | TGTAGTCCCAGCTACTCGGG | 3639 | CGACTTCTGCTGCCTCGAGC |
| 1391 | CACTCGGAGTTGGGGGGTGC | 2515 | CTGTAGTCCCAGCTACTCGG | 3640 | CCGACTTCTGCTGCCTCGAG |
| 1392 | GCACTCGGAGTTGGGGGGTG | 2516 | CCTGTAGTCCCAGCTACTCG | 3641 | TCCGACTTCTGCTGCCTCGA |
| 1393 | GGCACTCGGAGTTGGGGGGT | 2517 | GCCTGTAGTCCCAGCTACTC | 3642 | CTCCGACTTCTGCTGCCTCG |
| 1394 | TGGCACTCGGAGTTGGGGGG | 2518 | CGCCTGTAGTCCCAGCTACT | 3643 | CCTCCGACTTCTGCTGCCTC |
| 1395 | TTGGCACTCGGAGTTGGGGG | 2519 | GCGCCTGTAGTCCCAGCTAC | 3644 | TCCTCCGACTTCTGCTGCCT |
| 1396 | CTTGGCACTCGGAGTTGGGG | 2520 | CGCGCCTGTAGTCCCAGCTA | 3645 | CTCCTCCGACTTCTGCTGCC |
| 1397 | CCTTGGCACTCGGAGTTGGG | 2521 | GCGCGCCTGTAGTCCCAGCT | 3646 | CCTCCTCCGACTTCTGCTGC |
| 1398 | CCCTTGGCACTCGGAGTTGG | 2522 | TGCGCGCCTGTAGTCCCAGC | 3647 | CCCTCCTCCGACTTCTGCTG |
| 1399 | CCCCTTGGCACTCGGAGTTG | 2523 | GTGCGCGCCTGTAGTCCCAG | 3648 | TCCCTCCTCCGACTTCTGCT |
| 1400 | TCCCCTTGGCACTCGGAGTT | 2524 | GGTGCGCGCCTGTAGTCCCA | 3649 | TTCCCTCCTCCGACTTCTGC |
| 1401 | CTCCCCTTGGCACTCGGAGT | 2525 | TGGTGCGCGCCTGTAGTCCC | 3650 | TTTCCCTCCTCCGACTTCTG |
| 1402 | TCTCCCCTTGGCACTCGGAG | 2526 | GTGGTGCGCGCCTGTAGTCC | 3651 | CTTTCCCTCCTCCGACTTCT |
| 1403 | CTCTCCCCTTGGCACTCGGA | 2527 | GGTGGTGCGCGCCTGTAGTC | 3652 | CCTTTCCCTCCTCCGACTTC |
| 1404 | CCTCTCCCCTTGGCACTCGG | 2528 | TGGTGGTGCGCGCCTGTAGT | 3653 | CCCTTTCCCTCCTCCGACTT |
| 1405 | TCCTCTCCCCTTGGCACTCG | 2529 | GTGGTGGTGCGCGCCTGTAG | 3654 | ACCCTTTCCCTCCTCCGACT |
| 1406 | TTCCTCTCCCCTTGGCACTC | 2530 | CGTGGTGGTGCGCGCCTGTA | 3655 | CACCCTTTCCCTCCTCCGAC |
| 1407 | TTTCCTCTCCCCTTGGCACT | 2531 | GCGTGGTGGTGCGCGCCTGT | 3656 | GCACCCTTTCCCTCCTCCGA |
| 1408 | ATTTCCTCTCCCCTTGGCAC | 2532 | GGCGTGGTGGTGCGCGCCTG | 3657 | GGCACCCTTTCCCTCCTCCG |
| 1409 | AATTTCCTCTCCCCTTGGCA | 2533 | GGGCGTGGTGGTGCGCGCCT | 3658 | TGGCACCCTTTCCCTCCTCC |
| 1410 | GAATTTCCTCTCCCCTTGGC | 2534 | TGGGCGTGGTGGTGCGCGCC | 3659 | TTGGCACCCTTTCCCTCCTC |
| 1411 | GGAATTTCCTCTCCCCTTGG | 2535 | CTGGGCGTGGTGGTGCGCGC | 3660 | GTTGGCACCCTTTCCCTCCT |
| 1412 | GGGAATTTCCTCTCCCCTTG | 2536 | GCTGGGCGTGGTGGTGCGCG | 3661 | GGTTGGCACCCTTTCCCTCC |
| 1413 | TGGGAATTTCCTCTCCCCTT | 2537 | AGCTGGGCGTGGTGGTGCGC | 3662 | CGGTTGGCACCCTTTCCCTC |
| 1414 | TTGGGAATTTCCTCTCCCCT | 2538 | TAGCTGGGCGTGGTGGTGCG | 3663 | TCGGTTGGCACCCTTTCCCT |
| 1415 | TTTGGGAATTTCCTCTCCCC | 2539 | TTAGCTGGGCGTGGTGGTGC | 3664 | CTCGGTTGGCACCCTTTCCC |
| 1416 | TTTTGGGAATTTCCTCTCCC | 2540 | ATTAGCTGGGCGTGGTGGTG | 3665 | GCTCGGTTGGCACCCTTTCC |
| 1417 | CTTTTGGGAATTTCCTCTCC | 2541 | AATTAGCTGGGCGTGGTGGT | 3666 | AGCTCGGTTGGCACCCTTTC |
| 1418 | CCTTTTGGGAATTTCCTCTC | 2542 | AAATTAGCTGGGCGTGGTGG | 3667 | CAGCTCGGTTGGCACCCTTT |
| 1419 | CCCTTTTGGGAATTTCCTCT | 2543 | AAAATTAGCTGGGCGTGGTG | 3668 | ACAGCTCGGTTGGCACCCTT |
| 1420 | CCCCTTTTGGGAATTTCCTC | 2544 | AAAAATTAGCTGGGCGTGGT | 3669 | CACAGCTCGGTTGGCACCCT |
| 1421 | TCCCCTTTTGGGAATTTCCT | 2545 | CAAAAATTAGCTGGGCGTGG | 3670 | CCACAGCTCGGTTGGCACCC |
| 1422 | CTCCCCTTTTGGGAATTTCC | 2546 | ACAAAAATTAGCTGGGCGTG | 3671 | TCCACAGCTCGGTTGGCACC |
| 1423 | GCTCCCCTTTTGGGAATTTC | 2547 | TACAAAAATTAGCTGGGCGT | 3672 | CTCCACAGCTCGGTTGGCAC |
| 1424 | GGCTCCCCTTTTGGGAATTT | 2548 | ATACAAAAATTAGCTGGGCG | 3673 | ACTCCACAGCTCGGTTGGCA |
| 1425 | TGGCTCCCCTTTTGGGAATT | 2549 | AATACAAAAATTAGCTGGGC | 3674 | GACTCCACAGCTCGGTTGGC |
| 1426 | TTGGCTCCCCTTTTGGGAAT | 2550 | AAATACAAAAATTAGCTGGG | 3675 | CGACTCCACAGCTCGGTTGG |
| 1427 | GTTGGCTCCCCTTTTGGGAA | 2551 | AAAATACAAAAATTAGCTGG | 3676 | ACGACTCCACAGCTCGGTTG |
| 1428 | GGTTGGCTCCCCTTTTGGGA | 2552 | AAAAATACAAAAATTAGCTG | 3677 | CACGACTCCACAGCTCGGTT |
| 1429 | AGGTTGGCTCCCCTTTTGGG | 2553 | TAAAAATACAAAAATTAGCT | 3678 | ACACGACTCCACAGCTCGGT |
| 1430 | TAGGTTGGCTCCCCTTTTGG | 2554 | CTAAAAATACAAAAATTAGC | 3679 | CACACGACTCCACAGCTCGG |
| 1431 | CTAGGTTGGCTCCCCTTTTG | 2555 | ACTAAAAATACAAAAATTAG | 3680 | CCACACGACTCCACAGCTCG |
| 1432 | GCTAGGTTGGCTCCCCTTTT | 2556 | TACTAAAAATACAAAAATTA | 3681 | GCCACACGACTCCACAGCTC |
| 1433 | AGCTAGGTTGGCTCCCCTTT | 2557 | CTACTAAAAATACAAAAATT | 3682 | TGCCACACGACTCCACAGCT |
| 1434 | CAGCTAGGTTGGCTCCCCTT | 2558 | TCTACTAAAAATACAAAAAT | 3683 | CTGCCACACGACTCCACAGC |
| 1435 | GCAGCTAGGTTGGCTCCCCT | 2559 | CTCTACTAAAAATACAAAAA | 3684 | CCTGCCACACGACTCCACAG |
| 1436 | TGCAGCTAGGTTGGCTCCCC | 2560 | TCTCTACTAAAAATACAAAA | 3685 | CCCTGCCACACGACTCCACA |
| 1437 | TTGCAGCTAGGTTGGCTCCC | 2561 | GTCTCTACTAAAAATACAAA | 3686 | CCCCTGCCACACGACTCCAC |
| 1438 | TTTGCAGCTAGGTTGGCTCC | 2562 | CGTCTCTACTAAAAATACAA | 3687 | ACCCCTGCCACACGACTCCA |
| 1439 | ATTTGCAGCTAGGTTGGCTC | 2563 | CCGTCTCTACTAAAAATACA | 3688 | AACCCCTGCCACACGACTCC |
| 1440 | CATTTGCAGCTAGGTTGGCT | 2564 | CCCGTCTCTACTAAAAATAC | 3689 | GAACCCCTGCCACACGACTC |
| 1441 | TCATTTGCAGCTAGGTTGGC | 2565 | CCCCGTCTCTACTAAAAATA | 3690 | AGAACCCCTGCCACACGACT |
| 1442 | CTCATTTGCAGCTAGGTTGG | 2566 | ACCCCGTCTCTACTAAAAAT | 3691 | TAGAACCCCTGCCACACGAC |
| 1443 | GCTCATTTGCAGCTAGGTTG | 2567 | AACCCCGTCTCTACTAAAAA | 3692 | TTAGAACCCCTGCCACACGA |
| 1444 | TGCTCATTTGCAGCTAGGTT | 2568 | AAACCCCGTCTCTACTAAAA | 3693 | CTTAGAACCCCTGCCACACG |
| 1445 | CTGCTCATTTGCAGCTAGGT | 2569 | GAAACCCCGTCTCTACTAAA | 3694 | GCTTAGAACCCCTGCCACAC |
| 1446 | CCTGCTCATTTGCAGCTAGG | 2570 | TGAAACCCCGTCTCTACTAA | 3695 | GGCTTAGAACCCCTGCCACA |
| 1447 | TCCTGCTCATTTGCAGCTAG | 2571 | GTGAAACCCCGTCTCTACTA | 3696 | AGGCTTAGAACCCCTGCCAC |
| 1448 | TTCCTGCTCATTTGCAGCTA | 2572 | GGTGAAACCCCGTCTCTACT | 3697 | CAGGCTTAGAACCCCTGCCA |
| 1449 | TTTCCTGCTCATTTGCAGCT | 2573 | TGGTGAAACCCCGTCTCTAC | 3698 | CCAGGCTTAGAACCCCTGCC |
| 1450 | TTTTCCTGCTCATTTGCAGC | 2574 | ATGGTGAAACCCCGTCTCTA | 3699 | TCCAGGCTTAGAACCCCTGC |
| 1451 | TTTTTCCTGCTCATTTGCAG | 2575 | CATGGTGAAACCCCGTCTCT | 3700 | CTCCAGGCTTAGAACCCCTG |
| 1452 | CTTTTTCCTGCTCATTTGCA | 2576 | ACATGGTGAAACCCCGTCTC | 3701 | GCTCCAGGCTTAGAACCCCT |
| 1453 | ACTTTTTCCTGCTCATTTGC | 2577 | AACATGGTGAAACCCCGTCT | 3702 | AGCTCCAGGCTTAGAACCCC |
| 1454 | CACTTTTTCCTGCTCATTTG | 2578 | CAACATGGTGAAACCCCGTC | 3703 | TAGCTCCAGGCTTAGAACCC |
| 1455 | CCACTTTTTCCTGCTCATTT | 2579 | CCAACATGGTGAAACCCCGT | 3704 | CTAGCTCCAGGCTTAGAACC |
| 1456 | GCCACTTTTTCCTGCTCATT | 2580 | GCCAACATGGTGAAACCCCG | 3705 | TCTAGCTCCAGGCTTAGAAC |
| 1457 | AGCCACTTTTTCCTGCTCAT | 2581 | GGCCAACATGGTGAAACCCC | 3706 | CTCTAGCTCCAGGCTTAGAA |
| 1458 | CAGCCACTTTTTCCTGCTCA | 2582 | TGGCCAACATGGTGAAACCC | 3707 | GCTCTAGCTCCAGGCTTAGA |
| 1459 | CCAGCCACTTTTTCCTGCTC | 2583 | CTGGCCAACATGGTGAAACC | 3708 | TGCTCTAGCTCCAGGCTTAG |
| 1460 | CCCAGCCACTTTTTCCTGCT | 2584 | CCTGGCCAACATGGTGAAAC | 3709 | CTGCTCTAGCTCCAGGCTTA |
| 1461 | TCCCAGCCACTTTTTCCTGC | 2585 | TCCTGGCCAACATGGTGAAA | 3710 | TCTGCTCTAGCTCCAGGCTT |
| 1462 | CTCCCAGCCACTTTTTCCTG | 2586 | ATCCTGGCCAACATGGTGAA | 3711 | GTCTGCTCTAGCTCCAGGCT |
| 1463 | TCTCCCAGCCACTTTTTCCT | 2587 | CATCCTGGCCAACATGGTGA | 3712 | GGTCTGCTCTAGCTCCAGGC |
| 1464 | CTCTCCCAGCCACTTTTTCC | 2588 | CCATCCTGGCCAACATGGTG | 3713 | GGGTCTGCTCTAGCTCCAGG |
| 1465 | CCTCTCCCAGCCACTTTTTC | 2589 | ACCATCCTGGCCAACATGGT | 3714 | TGGGTCTGCTCTAGCTCCAG |
| 1466 | GCCTCTCCCAGCCACTTTTT | 2590 | GACCATCCTGGCCAACATGG | 3715 | CTGGGTCTGCTCTAGCTCCA |
| 1467 | CGCCTCTCCCAGCCACTTTT | 2591 | AGACCATCCTGGCCAACATG | 3716 | TCTGGGTCTGCTCTAGCTCC |
| 1468 | CCGCCTCTCCCAGCCACTTT | 2592 | GAGACCATCCTGGCCAACAT | 3717 | GTCTGGGTCTGCTCTAGCTC |
| 1469 | TCCGCCTCTCCCAGCCACTT | 2593 | CGAGACCATCCTGGCCAACA | 3718 | TGTCTGGGTCTGCTCTAGCT |
| 1470 | TTCCGCCTCTCCCAGCCACT | 2594 | TCGAGACCATCCTGGCCAAC | 3719 | CTGTCTGGGTCTGCTCTAGC |
| 1471 | CTTCCGCCTCTCCCAGCCAC | 2595 | ATCGAGACCATCCTGGCCAA | 3720 | GCTGTCTGGGTCTGCTCTAG |
| 1472 | GCTTCCGCCTCTCCCAGCCA | 2596 | AATCGAGACCATCCTGGCCA | 3721 | CGCTGTCTGGGTCTGCTCTA |
| 1473 | AGCTTCCGCCTCTCCCAGCC | 2597 | AAATCGAGACCATCCTGGCC | 3722 | CCGCTGTCTGGGTCTGCTCT |
| 1474 | TAGCTTCCGCCTCTCCCAGC | 2598 | GAAATCGAGACCATCCTGGC | 3723 | TCCGCTGTCTGGGTCTGCTC |
| 1475 | CTAGCTTCCGCCTCTCCCAG | 2599 | AGAAATCGAGACCATCCTGG | 3724 | ATCCGCTGTCTGGGTCTGCT |
| 1476 | GCTAGCTTCCGCCTCTCCCA | 2600 | AAGAAATCGAGACCATCCTG | 3725 | TATCCGCTGTCTGGGTCTGC |
| 1477 | AGCTAGCTTCCGCCTCTCCC | 2601 | CAAGAAATCGAGACCATCCT | 3726 | TTATCCGCTGTCTGGGTCTG |
| 1478 | GAGCTAGCTTCCGCCTCTCC | 2602 | TCAAGAAATCGAGACCATCC | 3727 | CTTATCCGCTGTCTGGGTCT |
| 1479 | CGAGCTAGCTTCCGCCTCTC | 2603 | GTCAAGAAATCGAGACCATC | 3728 | TCTTATCCGCTGTCTGGGTC |
| 1480 | ACGAGCTAGCTTCCGCCTCT | 2604 | GGTCAAGAAATCGAGACCAT | 3729 | GTCTTATCCGCTGTCTGGGT |
| 1481 | GACGAGCTAGCTTCCGCCTC | 2605 | AGGTCAAGAAATCGAGACCA | 3730 | TGTCTTATCCGCTGTCTGGG |
| 1482 | GGACGAGCTAGCTTCCGCCT | 2606 | GAGGTCAAGAAATCGAGACC | 3731 | GTGTCTTATCCGCTGTCTGG |
| 1483 | GGGACGAGCTAGCTTCCGCC | 2607 | CGAGGTCAAGAAATCGAGAC | 3732 | GGTGTCTTATCCGCTGTCTG |
| 1484 | TGGGACGAGCTAGCTTCCGC | 2608 | ACGAGGTCAAGAAATCGAGA | 3733 | AGGTGTCTTATCCGCTGTCT |
| 1485 | GTGGGACGAGCTAGCTTCCG | 2609 | CACGAGGTCAAGAAATCGAG | 3734 | CAGGTGTCTTATCCGCTGTC |
| 1486 | GGTGGGACGAGCTAGCTTCC | 2610 | TCACGAGGTCAAGAAATCGA | 3735 | CCAGGTGTCTTATCCGCTGT |
| 1487 | GGGTGGGACGAGCTAGCTTC | 2611 | ATCACGAGGTCAAGAAATCG | 3736 | GCCAGGTGTCTTATCCGCTG |
| 1488 | CGGGTGGGACGAGCTAGCTT | 2612 | GATCACGAGGTCAAGAAATC | 3737 | GGCCAGGTGTCTTATCCGCT |
| 1489 | GCGGGTGGGACGAGCTAGCT | 2613 | GGATCACGAGGTCAAGAAAT | 3738 | AGGCCAGGTGTCTTATCCGC |
| 1490 | TGCGGGTGGGACGAGCTAGC | 2614 | CGGATCACGAGGTCAAGAAA | 3739 | CAGGCCAGGTGTCTTATCCG |
| 1491 | TTGCGGGTGGGACGAGCTAG | 2615 | GCGGATCACGAGGTCAAGAA | 3740 | GCAGGCCAGGTGTCTTATCC |
| 1492 | GTTGCGGGTGGGACGAGCTA | 2616 | GGCGGATCACGAGGTCAAGA | 3741 | AGCAGGCCAGGTGTCTTATC |
| 1493 | TGTTGCGGGTGGGACGAGCT | 2617 | GGGCGGATCACGAGGTCAAG | 3742 | CAGCAGGCCAGGTGTCTTAT |
| 1494 | TTGTTGCGGGTGGGACGAGC | 2618 | TGGGCGGATCACGAGGTCAA | 3743 | TCAGCAGGCCAGGTGTCTTA |
| 1495 | CTTGTTGCGGGTGGGACGAG | 2619 | GTGGGCGGATCACGAGGTCA | 3744 | CTCAGCAGGCCAGGTGTCTT |
| 1496 | TCTTGTTGCGGGTGGGACGA | 2620 | GGTGGGCGGATCACGAGGTC | 3745 | CCTCAGCAGGCCAGGTGTCT |
| 1497 | CTCTTGTTGCGGGTGGGACG | 2621 | AGGTGGGCGGATCACGAGGT | 3746 | TCCTCAGCAGGCCAGGTGTC |
| 1498 | CCTCTTGTTGCGGGTGGGAC | 2622 | GAGGTGGGCGGATCACGAGG | 3747 | GTCCTCAGCAGGCCAGGTGT |
| 1499 | TCCTCTTGTTGCGGGTGGGA | 2623 | CGAGGTGGGCGGATCACGAG | 3748 | GGTCCTCAGCAGGCCAGGTG |
| 1500 | TTCCTCTTGTTGCGGGTGGG | 2624 | CCGAGGTGGGCGGATCACGA | 3749 | AGGTCCTCAGCAGGCCAGGT |
| 1501 | CTTCCTCTTGTTGCGGGTGG | 2625 | GCCGAGGTGGGCGGATCACG | 3750 | TAGGTCCTCAGCAGGCCAGG |
| 1502 | ACTTCCTCTTGTTGCGGGTG | 2626 | GGCCGAGGTGGGCGGATCAC | 3751 | CTAGGTCCTCAGCAGGCCAG |
| 1503 | TACTTCCTCTTGTTGCGGGT | 2627 | AGGCCGAGGTGGGCGGATCA | 3752 | ACTAGGTCCTCAGCAGGCCA |
| 1504 | TTACTTCCTCTTGTTGCGGG | 2628 | GAGGCCGAGGTGGGCGGATC | 3753 | TACTAGGTCCTCAGCAGGCC |
| 1505 | CTTACTTCCTCTTGTTGCGG | 2629 | GGAGGCCGAGGTGGGCGGAT | 3754 | GTACTAGGTCCTCAGCAGGC |
| 1506 | CCTTACTTCCTCTTGTTGCG | 2630 | GGGAGGCCGAGGTGGGCGGA | 3755 | AGTACTAGGTCCTCAGCAGG |
| 1507 | TCCTTACTTCCTCTTGTTGC | 2631 | TGGGAGGCCGAGGTGGGCGG | 3756 | TAGTACTAGGTCCTCAGCAG |
| 1508 | CTCCTTACTTCCTCTTGTTG | 2632 | TTGGGAGGCCGAGGTGGGCG | 3757 | CTAGTACTAGGTCCTCAGCA |
| 1509 | CCTCCTTACTTCCTCTTGTT | 2633 | TTTGGGAGGCCGAGGTGGGC | 3758 | ACTAGTACTAGGTCCTCAGC |
| 1510 | TCCTCCTTACTTCCTCTTGT | 2634 | CTTTGGGAGGCCGAGGTGGG | 3759 | GACTAGTACTAGGTCCTCAG |
| 1511 | CTCCTCCTTACTTCCTCTTG | 2635 | ACTTTGGGAGGCCGAGGTGG | 3760 | AGACTAGTACTAGGTCCTCA |
| 1512 | GCTCCTCCTTACTTCCTCTT | 2636 | CACTTTGGGAGGCCGAGGTG | 3761 | CAGACTAGTACTAGGTCCTC |
| 1513 | GGCTCCTCCTTACTTCCTCT | 2637 | GCACTTTGGGAGGCCGAGGT | 3762 | ACAGACTAGTACTAGGTCCT |
| 1514 | TGGCTCCTCCTTACTTCCTC | 2638 | AGCACTTTGGGAGGCCGAGG | 3763 | CACAGACTAGTACTAGGTCC |
| 1515 | CTGGCTCCTCCTTACTTCCT | 2639 | TAGCACTTTGGGAGGCCGAG | 3764 | GCACAGACTAGTACTAGGTC |
| 1516 | TCTGGCTCCTCCTTACTTCC | 2640 | TTAGCACTTTGGGAGGCCGA | 3765 | GGCACAGACTAGTACTAGGT |
| 1517 | GTCTGGCTCCTCCTTACTTC | 2641 | CTTAGCACTTTGGGAGGCCG | 3766 | GGGCACAGACTAGTACTAGG |
| 1518 | GGTCTGGCTCCTCCTTACTT | 2642 | TCTTAGCACTTTGGGAGGCC | 3767 | AGGGCACAGACTAGTACTAG |
| 1519 | TGGTCTGGCTCCTCCTTACT | 2643 | ATCTTAGCACTTTGGGAGGC | 3768 | CAGGGCACAGACTAGTACTA |
| 1520 | CTGGTCTGGCTCCTCCTTAC | 2644 | AATCTTAGCACTTTGGGAGG | 3769 | CCAGGGCACAGACTAGTACT |
| 1521 | CCTGGTCTGGCTCCTCCTTA | 2645 | TAATCTTAGCACTTTGGGAG | 3770 | ACCAGGGCACAGACTAGTAC |
| 1522 | ACCTGGTCTGGCTCCTCCTT | 2646 | GTAATCTTAGCACTTTGGGA | 3771 | CACCAGGGCACAGACTAGTA |
| 1523 | AACCTGGTCTGGCTCCTCCT | 2647 | TGTAATCTTAGCACTTTGGG | 3772 | TCACCAGGGCACAGACTAGT |
| 1524 | GAACCTGGTCTGGCTCCTCC | 2648 | CTGTAATCTTAGCACTTTGG | 3773 | GTCACCAGGGCACAGACTAG |
| 1525 | TGAACCTGGTCTGGCTCCTC | 2649 | CCTGTAATCTTAGCACTTTG | 3774 | TGTCACCAGGGCACAGACTA |
| 1526 | CTGAACCTGGTCTGGCTCCT | 2650 | GCCTGTAATCTTAGCACTTT | 3775 | GTGTCACCAGGGCACAGACT |
| 1527 | ACTGAACCTGGTCTGGCTCC | 2651 | TGCCTGTAATCTTAGCACTT | 3776 | TGTGTCACCAGGGCACAGAC |
| 1528 | GACTGAACCTGGTCTGGCTC | 2652 | ATGCCTGTAATCTTAGCACT | 3777 | ATGTGTCACCAGGGCACAGA |
| 1529 | TGACTGAACCTGGTCTGGCT | 2653 | CATGCCTGTAATCTTAGCAC | 3778 | AATGTGTCACCAGGGCACAG |
| 1530 | GTGACTGAACCTGGTCTGGC | 2654 | TCATGCCTGTAATCTTAGCA | 3779 | AAATGTGTCACCAGGGCACA |
| 1531 | GGTGACTGAACCTGGTCTGG | 2655 | CTCATGCCTGTAATCTTAGC | 3780 | CAAATGTGTCACCAGGGCAC |
| 1532 | TGGTGACTGAACCTGGTCTG | 2656 | GCTCATGCCTGTAATCTTAG | 3781 | ACAAATGTGTCACCAGGGCA |
| 1533 | GTGGTGACTGAACCTGGTCT | 2657 | GGCTCATGCCTGTAATCTTA | 3782 | CACAAATGTGTCACCAGGGC |
| 1534 | AGTGGTGACTGAACCTGGTC | 2658 | TGGCTCATGCCTGTAATCTT | 3783 | GCACAAATGTGTCACCAGGG |
| 1535 | GAGTGGTGACTGAACCTGGT | 2659 | GTGGCTCATGCCTGTAATCT | 3784 | AGCACAAATGTGTCACCAGG |
| 1536 | GGAGTGGTGACTGAACCTGG | 2660 | GGTGGCTCATGCCTGTAATC | 3785 | AAGCACAAATGTGTCACCAG |
| 1537 | GGGAGTGGTGACTGAACCTG | 2661 | CGGTGGCTCATGCCTGTAAT | 3786 | TAAGCACAAATGTGTCACCA |
| 1538 | TGGGAGTGGTGACTGAACCT | 2662 | GCGGTGGCTCATGCCTGTAA | 3787 | ATAAGCACAAATGTGTCACC |
| 1539 | TTGGGAGTGGTGACTGAACC | 2663 | CGCGGTGGCTCATGCCTGTA | 3788 | AATAAGCACAAATGTGTCAC |
| 1540 | CTTGGGAGTGGTGACTGAAC | 2664 | GCGCGGTGGCTCATGCCTGT | 3789 | AAATAAGCACAAATGTGTCA |
| 1541 | ACTTGGGAGTGGTGACTGAA | 2665 | GGCGCGGTGGCTCATGCCTG | 3790 | AAAATAAGCACAAATGTGTC |
| 1542 | GACTTGGGAGTGGTGACTGA | 2666 | GGGCGCGGTGGCTCATGCCT | 3791 | GAAAATAAGCACAAATGTGT |
| 1543 | GGACTTGGGAGTGGTGACTG | 2667 | CGGGCGCGGTGGCTCATGCC | 3792 | GGAAAATAAGCACAAATGTG |
| 1544 | GGGACTTGGGAGTGGTGACT | 2668 | CCGGGCGCGGTGGCTCATGC | 3793 | AGGAAAATAAGCACAAATGT |
| 1545 | AGGGACTTGGGAGTGGTGAC | 2669 | TCCGGGCGCGGTGGCTCATG | 3794 | AAGGAAAATAAGCACAAATG |
| 1546 | TAGGGACTTGGGAGTGGTGA | 2670 | GTCCGGGCGCGGTGGCTCAT | 3795 | GAAGGAAAATAAGCACAAAT |
| 1547 | GTAGGGACTTGGGAGTGGTG | 2671 | AGTCCGGGCGCGGTGGCTCA | 3796 | GGAAGGAAAATAAGCACAAA |
| 1548 | AGTAGGGACTTGGGAGTGGT | 2672 | AAGTCCGGGCGCGGTGGCTC | 3797 | AGGAAGGAAAATAAGCACAA |
| 1549 | TAGTAGGGACTTGGGAGTGG | 2673 | CAAGTCCGGGCGCGGTGGCT | 3798 | CAGGAAGGAAAATAAGCACA |
| 1550 | GTAGTAGGGACTTGGGAGTG | 2674 | ACAAGTCCGGGCGCGGTGGC | 3799 | ACAGGAAGGAAAATAAGCAC |
| 1551 | GGTAGTAGGGACTTGGGAGT | 2675 | CACAAGTCCGGGCGCGGTGG | 3800 | CACAGGAAGGAAAATAAGCA |
| 1552 | AGGTAGTAGGGACTTGGGAG | 2676 | ACACAAGTCCGGGCGCGGTG | 3801 | ACACAGGAAGGAAAATAAGC |
| 1553 | AAGGTAGTAGGGACTTGGGA | 2677 | CACACAAGTCCGGGCGCGGT | 3802 | TACACAGGAAGGAAAATAAG |
| 1554 | GAAGGTAGTAGGGACTTGGG | 2678 | ACACACAAGTCCGGGCGCGG | 3803 | ATACACAGGAAGGAAAATAA |
| 1555 | CGAAGGTAGTAGGGACTTGG | 2679 | AACACACAAGTCCGGGCGCG | 3804 | GATACACAGGAAGGAAAATA |
| 1556 | TCGAAGGTAGTAGGGACTTG | 2680 | AAACACACAAGTCCGGGCGC | 3805 | AGATACACAGGAAGGAAAAT |
| 1557 | CTCGAAGGTAGTAGGGACTT | 2681 | CAAACACACAAGTCCGGGCG | 3806 | AAGATACACAGGAAGGAAAA |
| 1558 | TCTCGAAGGTAGTAGGGACT | 2682 | ACAAACACACAAGTCCGGGC | 3807 | CAAGATACACAGGAAGGAAA |
| 1559 | TTCTCGAAGGTAGTAGGGAC | 2683 | AACAAACACACAAGTCCGGG | 3808 | TCAAGATACACAGGAAGGAA |
| 1560 | CTTCTCGAAGGTAGTAGGGA | 2684 | AAACAAACACACAAGTCCGG | 3809 | CTCAAGATACACAGGAAGGA |
| 1561 | GCTTCTCGAAGGTAGTAGGG | 2685 | AAAACAAACACACAAGTCCG | 3810 | TCTCAAGATACACAGGAAGG |
| 1562 | GGCTTCTCGAAGGTAGTAGG | 2686 | TAAAACAAACACACAAGTCC | 3811 | GTCTCAAGATACACAGGAAG |
| 1563 | TGGCTTCTCGAAGGTAGTAG | 2687 | CTAAAACAAACACACAAGTC | 3812 | AGTCTCAAGATACACAGGAA |
| 1564 | TTGGCTTCTCGAAGGTAGTA | 2688 | TCTAAAACAAACACACAAGT | 3813 | TAGTCTCAAGATACACAGGA |
| 1565 | CTTGGCTTCTCGAAGGTAGT | 2689 | CTCTAAAACAAACACACAAG | 3814 | CTAGTCTCAAGATACACAGG |
| 1566 | CCTTGGCTTCTCGAAGGTAG | 2690 | TCTCTAAAACAAACACACAA | 3815 | CCTAGTCTCAAGATACACAG |
| 1567 | ACCTTGGCTTCTCGAAGGTA | 2691 | GTCTCTAAAACAAACACACA | 3816 | CCCTAGTCTCAAGATACACA |
| 1568 | GACCTTGGCTTCTCGAAGGT | 2692 | TGTCTCTAAAACAAACACAC | 3817 | TCCCTAGTCTCAAGATACAC |
| 1569 | AGACCTTGGCTTCTCGAAGG | 2693 | CTGTCTCTAAAACAAACACA | 3818 | CTCCCTAGTCTCAAGATACA |
| 1570 | GAGACCTTGGCTTCTCGAAG | 2694 | CCTGTCTCTAAAACAAACAC | 3819 | ACTCCCTAGTCTCAAGATAC |
| 1571 | TGAGACCTTGGCTTCTCGAA | 2695 | CCCTGTCTCTAAAACAAACA | 3820 | TACTCCCTAGTCTCAAGATA |
| 1572 | CTGAGACCTTGGCTTCTCGA | 2696 | ACCCTGTCTCTAAAACAAAC | 3821 | ATACTCCCTAGTCTCAAGAT |
| 1573 | CCTGAGACCTTGGCTTCTCG | 2697 | GACCCTGTCTCTAAAACAAA | 3822 | TATACTCCCTAGTCTCAAGA |
| 1574 | ACCTGAGACCTTGGCTTCTC | 2698 | AGACCCTGTCTCTAAAACAA | 3823 | CTATACTCCCTAGTCTCAAG |
| 1575 | GACCTGAGACCTTGGCTTCT | 2699 | GAGACCCTGTCTCTAAAACA | 3824 | ACTATACTCCCTAGTCTCAA |
| 1576 | AGACCTGAGACCTTGGCTTC | 2700 | CGAGACCCTGTCTCTAAAAC | 3825 | AACTATACTCCCTAGTCTCA |
| 1577 | GAGACCTGAGACCTTGGCTT | 2701 | GCGAGACCCTGTCTCTAAAA | 3826 | AAACTATACTCCCTAGTCTC |
| 1578 | CGAGACCTGAGACCTTGGCT | 2702 | AGCGAGACCCTGTCTCTAAA | 3827 | TAAACTATACTCCCTAGTCT |
| 1579 | ACGAGACCTGAGACCTTGGC | 2703 | GAGCGAGACCCTGTCTCTAA | 3828 | ATAAACTATACTCCCTAGTC |
| 1580 | AACGAGACCTGAGACCTTGG | 2704 | TGAGCGAGACCCTGTCTCTA | 3829 | CATAAACTATACTCCCTAGT |
| 1581 | GAACGAGACCTGAGACCTTG | 2705 | CTGAGCGAGACCCTGTCTCT | 3830 | GCATAAACTATACTCCCTAG |
| 1582 | GGAACGAGACCTGAGACCTT | 2706 | ACTGAGCGAGACCCTGTCTC | 3831 | AGCATAAACTATACTCCCTA |
| 1583 | GGGAACGAGACCTGAGACCT | 2707 | GACTGAGCGAGACCCTGTCT | 3832 | AAGCATAAACTATACTCCCT |
| 1584 | TGGGAACGAGACCTGAGACC | 2708 | CGACTGAGCGAGACCCTGTC | 3833 | GAAGCATAAACTATACTCCC |
| 1585 | CTGGGAACGAGACCTGAGAC | 2709 | GCGACTGAGCGAGACCCTGT | 3834 | AGAAGCATAAACTATACTCC |
| 1586 | CCTGGGAACGAGACCTGAGA | 2710 | GGCGACTGAGCGAGACCCTG | 3835 | TAGAAGCATAAACTATACTC |
| 1587 | GCCTGGGAACGAGACCTGAG | 2711 | GGGCGACTGAGCGAGACCCT | 3836 | ATAGAAGCATAAACTATACT |
| 1588 | GGCCTGGGAACGAGACCTGA | 2712 | TGGGCGACTGAGCGAGACCC | 3837 | AATAGAAGCATAAACTATAC |
| 1589 | GGGCCTGGGAACGAGACCTG | 2713 | TTGGGCGACTGAGCGAGACC | 3838 | CAATAGAAGCATAAACTATA |
| 1590 | AGGGCCTGGGAACGAGACCT | 2714 | CTTGGGCGACTGAGCGAGAC | 3839 | TCAATAGAAGCATAAACTAT |
| 1591 | GAGGGCCTGGGAACGAGACC | 2715 | GCTTGGGCGACTGAGCGAGA | 3840 | TTCAATAGAAGCATAAACTA |
| 1592 | CGAGGGCCTGGGAACGAGAC | 2716 | AGCTTGGGCGACTGAGCGAG | 3841 | TTTCAATAGAAGCATAAACT |
| 1593 | CCGAGGGCCTGGGAACGAGA | 2717 | CAGCTTGGGCGACTGAGCGA | 3842 | GTTTCAATAGAAGCATAAAC |
| 1594 | TCCGAGGGCCTGGGAACGAG | 2718 | TCAGCTTGGGCGACTGAGCG | 3843 | TGTTTCAATAGAAGCATAAA |
| 1595 | CTCCGAGGGCCTGGGAACGA | 2719 | ATCAGCTTGGGCGACTGAGC | 3844 | GTGTTTCAATAGAAGCATAA |
| 1596 | GCTCCGAGGGCCTGGGAACG | 2720 | GATCAGCTTGGGCGACTGAG | 3845 | TGTGTTTCAATAGAAGCATA |
| 1597 | AGCTCCGAGGGCCTGGGAAC | 2721 | TGATCAGCTTGGGCGACTGA | 3846 | GTGTGTTTCAATAGAAGCAT |
| 1598 | GAGCTCCGAGGGCCTGGGAA | 2722 | ATGATCAGCTTGGGCGACTG | 3847 | TGTGTGTTTCAATAGAAGCA |
| 1599 | GGAGCTCCGAGGGCCTGGGA | 2723 | TATGATCAGCTTGGGCGACT | 3848 | GTGTGTGTTTCAATAGAAGC |
| 1600 | GGGAGCTCCGAGGGCCTGGG | 2724 | CTATGATCAGCTTGGGCGAC | 3849 | TGTGTGTGTTTCAATAGAAG |
| 1601 | TGGGAGCTCCGAGGGCCTGG | 2725 | GCTATGATCAGCTTGGGCGA | 3850 | GTGTGTGTGTTTCAATAGAA |
| 1602 | CTGGGAGCTCCGAGGGCCTG | 2726 | AGCTATGATCAGCTTGGGCG | 3851 | TGTGTGTGTGTTTCAATAGA |
| 1603 | GCTGGGAGCTCCGAGGGCCT | 2727 | GAGCTATGATCAGCTTGGGC | 3852 | GTGTGTGTGTGTTTCAATAG |
| 1604 | GGCTGGGAGCTCCGAGGGCC | 2728 | TGAGCTATGATCAGCTTGGG | 3853 | TGTGTGTGTGTGTTTCAATA |
| 1605 | GGGCTGGGAGCTCCGAGGGC | 2729 | GTGAGCTATGATCAGCTTGG | 3854 | ATGTGTGTGTGTGTTTCAAT |
| 1606 | TGGGCTGGGAGCTCCGAGGG | 2730 | AGTGAGCTATGATCAGCTTG | 3855 | CATGTGTGTGTGTGTTTCAA |
| 1607 | CTGGGCTGGGAGCTCCGAGG | 2731 | CAGTGAGCTATGATCAGCTT | 3856 | TCATGTGTGTGTGTGTTTCA |
| 1608 | CCTGGGCTGGGAGCTCCGAG | 2732 | GCAGTGAGCTATGATCAGCT | 3857 | TTCATGTGTGTGTGTGTTTC |
| 1609 | CCCTGGGCTGGGAGCTCCGA | 2733 | CGCAGTGAGCTATGATCAGC | 3858 | TTTCATGTGTGTGTGTGTTT |
| 1610 | ACCCTGGGCTGGGAGCTCCG | 2734 | CCGCAGTGAGCTATGATCAG | 3859 | GTTTCATGTGTGTGTGTGTT |
| 1611 | GACCCTGGGCTGGGAGCTCC | 2735 | GCCGCAGTGAGCTATGATCA | 3860 | AGTTTCATGTGTGTGTGTGT |
| 1612 | CGACCCTGGGCTGGGAGCTC | 2736 | GGCCGCAGTGAGCTATGATC | 3861 | AAGTTTCATGTGTGTGTGTG |
| 1613 | GCGACCCTGGGCTGGGAGCT | 2737 | AGGCCGCAGTGAGCTATGAT | 3862 | AAAGTTTCATGTGTGTGTGT |
| 1614 | CGCGACCCTGGGCTGGGAGC | 2738 | CAGGCCGCAGTGAGCTATGA | 3863 | GAAAGTTTCATGTGTGTGTG |
| 1615 | GCGCGACCCTGGGCTGGGAG | 2739 | GCAGGCCGCAGTGAGCTATG | 3864 | TGAAAGTTTCATGTGTGTGT |
| 1616 | CGCGCGACCCTGGGCTGGGA | 2740 | TGCAGGCCGCAGTGAGCTAT | 3865 | ATGAAAGTTTCATGTGTGTG |
| 1617 | GCGCGCGACCCTGGGCTGGG | 2741 | TTGCAGGCCGCAGTGAGCTA | 3866 | CATGAAAGTTTCATGTGTGT |
| 1618 | GGCGCGCGACCCTGGGCTGG | 2742 | GTTGCAGGCCGCAGTGAGCT | 3867 | TCATGAAAGTTTCATGTGTG |
| 1619 | GGGCGCGCGACCCTGGGCTG | 2743 | AGTTGCAGGCCGCAGTGAGC | 3868 | TTCATGAAAGTTTCATGTGT |
| 1620 | GGGGCGCGCGACCCTGGGCT | 2744 | GAGTTGCAGGCCGCAGTGAG | 3869 | TTTCATGAAAGTTTCATGTG |
| 1621 | AGGGGCGCGCGACCCTGGGC | 2745 | GGAGTTGCAGGCCGCAGTGA | 3870 | ATTTCATGAAAGTTTCATGT |
| 1622 | GAGGGGCGCGCGACCCTGGG | 2746 | AGGAGTTGCAGGCCGCAGTG | 3871 | CATTTCATGAAAGTTTCATG |
| 1623 | GGAGGGGCGCGCGACCCTGG | 2747 | CAGGAGTTGCAGGCCGCAGT | 3872 | ACATTTCATGAAAGTTTCAT |
| 1624 | CGGAGGGGCGCGCGACCCTG | 2748 | GCAGGAGTTGCAGGCCGCAG | 3873 | AACATTTCATGAAAGTTTCA |
| 1625 | CCGGAGGGGCGCGCGACCCT | 2749 | AGCAGGAGTTGCAGGCCGCA | 3874 | TAACATTTCATGAAAGTTTC |
| 1626 | GCCGGAGGGGCGCGCGACCC | 2750 | GAGCAGGAGTTGCAGGCCGC | 3875 | CTAACATTTCATGAAAGTTT |
| 1627 | AGCCGGAGGGGCGCGCGACC | 2751 | GGAGCAGGAGTTGCAGGCCG | 3876 | CCTAACATTTCATGAAAGTT |
| 1628 | GAGCCGGAGGGGCGCGCGAC | 2752 | GGGAGCAGGAGTTGCAGGCC | 3877 | ACCTAACATTTCATGAAAGT |
| 1629 | GGAGCCGGAGGGGCGCGCGA | 2753 | TGGGAGCAGGAGTTGCAGGC | 3878 | CACCTAACATTTCATGAAAG |
| 1630 | TGGAGCCGGAGGGGCGCGCG | 2754 | TTGGGAGCAGGAGTTGCAGG | 3879 | TCACCTAACATTTCATGAAA |
| 1631 | CTGGAGCCGGAGGGGCGCGC | 2755 | TTTGGGAGCAGGAGTTGCAG | 3880 | TTCACCTAACATTTCATGAA |
| 1632 | CCTGGAGCCGGAGGGGCGCG | 2756 | CTTTGGGAGCAGGAGTTGCA | 3881 | ATTCACCTAACATTTCATGA |
| 1633 | GCCTGGAGCCGGAGGGGCGC | 2757 | GCTTTGGGAGCAGGAGTTGC | 3882 | GATTCACCTAACATTTCATG |
| 1634 | GGCCTGGAGCCGGAGGGGCG | 2758 | CGCTTTGGGAGCAGGAGTTG | 3883 | CGATTCACCTAACATTTCAT |
| 1635 | CGGCCTGGAGCCGGAGGGGC | 2759 | TCGCTTTGGGAGCAGGAGTT | 3884 | TCGATTCACCTAACATTTCA |
| 1636 | GCGGCCTGGAGCCGGAGGGG | 2760 | ATCGCTTTGGGAGCAGGAGT | 3885 | ATCGATTCACCTAACATTTC |
| 1637 | GGCGGCCTGGAGCCGGAGGG | 2761 | AATCGCTTTGGGAGCAGGAG | 3886 | GATCGATTCACCTAACATTT |
| 1638 | CGGCGGCCTGGAGCCGGAGG | 2762 | GAATCGCTTTGGGAGCAGGA | 3887 | TGATCGATTCACCTAACATT |
| 1639 | GCGGCGGCCTGGAGCCGGAG | 2763 | AGAATCGCTTTGGGAGCAGG | 3888 | TTGATCGATTCACCTAACAT |
| 1640 | CGCGGCGGCCTGGAGCCGGA | 2764 | GAGAATCGCTTTGGGAGCAG | 3889 | ATTGATCGATTCACCTAACA |
| 1641 | CCGCGGCGGCCTGGAGCCGG | 2765 | GGAGAATCGCTTTGGGAGCA | 3890 | TATTGATCGATTCACCTAAC |
| 1642 | CCCGCGGCGGCCTGGAGCCG | 2766 | AGGAGAATCGCTTTGGGAGC | 3891 | TTATTGATCGATTCACCTAA |
| 1643 | TCCCGCGGCGGCCTGGAGCC | 2767 | TAGGAGAATCGCTTTGGGAG | 3892 | GTTATTGATCGATTCACCTA |
| 1644 | TTCCCGCGGCGGCCTGGAGC | 2768 | GTAGGAGAATCGCTTTGGGA | 3893 | GGTTATTGATCGATTCACCT |
| 1645 | GTTCCCGCGGCGGCCTGGAG | 2769 | GGTAGGAGAATCGCTTTGGG | 3894 | AGGTTATTGATCGATTCACC |
| 1646 | GGTTCCCGCGGCGGCCTGGA | 2770 | CGGTAGGAGAATCGCTTTGG | 3895 | TAGGTTATTGATCGATTCAC |
| 1647 | TGGTTCCCGCGGCGGCCTGG | 2771 | GCGGTAGGAGAATCGCTTTG | 3896 | GTAGGTTATTGATCGATTCA |
| 1648 | GTGGTTCCCGCGGCGGCCTG | 2772 | TGCGGTAGGAGAATCGCTTT | 3897 | AGTAGGTTATTGATCGATTC |
| 1649 | GGTGGTTCCCGCGGCGGCCT | 2773 | CTGCGGTAGGAGAATCGCTT | 3898 | TAGTAGGTTATTGATCGATT |
| 1650 | GGGTGGTTCCCGCGGCGGCC | 2774 | GCTGCGGTAGGAGAATCGCT | 3899 | TTAGTAGGTTATTGATCGAT |
| 1651 | TGGGTGGTTCCCGCGGCGGC | 2775 | GGCTGCGGTAGGAGAATCGC | 3900 | ATTAGTAGGTTATTGATCGA |
| 1652 | GTGGGTGGTTCCCGCGGCGG | 2776 | GGGCTGCGGTAGGAGAATCG | 3901 | AATTAGTAGGTTATTGATCG |
| 1653 | GGTGGGTGGTTCCCGCGGCG | 2777 | GGGGCTGCGGTAGGAGAATC | 3902 | TAATTAGTAGGTTATTGATC |
| 1654 | TGGTGGGTGGTTCCCGCGGC | 2778 | GGGGGCTGCGGTAGGAGAAT | 3903 | GTAATTAGTAGGTTATTGAT |
| 1655 | GTGGTGGGTGGTTCCCGCGG | 2779 | GGGGGGCTGCGGTAGGAGAA | 3904 | TGTAATTAGTAGGTTATTGA |
| 1656 | GGTGGTGGGTGGTTCCCGCG | 2780 | TGGGGGGCTGCGGTAGGAGA | 3905 | TTGTAATTAGTAGGTTATTG |
| 1657 | TGGTGGTGGGTGGTTCCCGC | 2781 | TTGGGGGGCTGCGGTAGGAG | 3906 | GTTGTAATTAGTAGGTTATT |
| 1658 | GTGGTGGTGGGTGGTTCCCG | 2782 | CTTGGGGGGCTGCGGTAGGA | 3907 | GGTTGTAATTAGTAGGTTAT |
| 1659 | GGTGGTGGTGGGTGGTTCCC | 2783 | CCTTGGGGGGCTGCGGTAGG | 3908 | TGGTTGTAATTAGTAGGTTA |
| 1660 | TGGTGGTGGTGGGTGGTTCC | 2784 | GCCTTGGGGGGCTGCGGTAG | 3909 | GTGGTTGTAATTAGTAGGTT |
| 1661 | CTGGTGGTGGTGGGTGGTTC | 2785 | CGCCTTGGGGGGCTGCGGTA | 3910 | GGTGGTTGTAATTAGTAGGT |
| 1662 | CCTGGTGGTGGTGGGTGGTT | 2786 | GCGCCTTGGGGGGCTGCGGT | 3911 | GGGTGGTTGTAATTAGTAGG |
| 1663 | TCCTGGTGGTGGTGGGTGGT | 2787 | AGCGCCTTGGGGGGCTGCGG | 3912 | TGGGTGGTTGTAATTAGTAG |
| 1664 | CTCCTGGTGGTGGTGGGTGG | 2788 | CAGCGCCTTGGGGGGCTGCG | 3913 | CTGGGTGGTTGTAATTAGTA |
| 1665 | TCTCCTGGTGGTGGTGGGTG | 2789 | CCAGCGCCTTGGGGGGCTGC | 3914 | TCTGGGTGGTTGTAATTAGT |
| 1666 | CTCTCCTGGTGGTGGTGGGT | 2790 | CCCAGCGCCTTGGGGGGCTG | 3915 | TTCTGGGTGGTTGTAATTAG |
| 1667 | CCTCTCCTGGTGGTGGTGGG | 2791 | TCCCAGCGCCTTGGGGGGCT | 3916 | GTTCTGGGTGGTTGTAATTA |
| 1668 | CCCTCTCCTGGTGGTGGTGG | 2792 | ATCCCAGCGCCTTGGGGGGC | 3917 | GGTTCTGGGTGGTTGTAATT |
| 1669 | CCCCTCTCCTGGTGGTGGTG | 2793 | AATCCCAGCGCCTTGGGGGG | 3918 | TGGTTCTGGGTGGTTGTAAT |
| 1670 | TCCCCTCTCCTGGTGGTGGT | 2794 | TAATCCCAGCGCCTTGGGGG | 3919 | CTGGTTCTGGGTGGTTGTAA |
| 1671 | TTCCCCTCTCCTGGTGGTGG | 2795 | GTAATCCCAGCGCCTTGGGG | 3920 | TCTGGTTCTGGGTGGTTGTA |
| 1672 | CTTCCCCTCTCCTGGTGGTG | 2796 | TGTAATCCCAGCGCCTTGGG | 3921 | TTCTGGTTCTGGGTGGTTGT |
| 1673 | TCTTCCCCTCTCCTGGTGGT | 2797 | TTGTAATCCCAGCGCCTTGG | 3922 | TTTCTGGTTCTGGGTGGTTG |
| 1674 | TTCTTCCCCTCTCCTGGTGG | 2798 | TTTGTAATCCCAGCGCCTTG | 3923 | TTTTCTGGTTCTGGGTGGTT |
| 1675 | CTTCTTCCCCTCTCCTGGTG | 2799 | ATTTGTAATCCCAGCGCCTT | 3924 | TTTTTCTGGTTCTGGGTGGT |
| 1676 | GCTTCTTCCCCTCTCCTGGT | 2800 | CATTTGTAATCCCAGCGCCT | 3925 | TTTTTTCTGGTTCTGGGTGG |
| 1677 | GGCTTCTTCCCCTCTCCTGG | 2801 | ACATTTGTAATCCCAGCGCC | 3926 | TTTTTTTCTGGTTCTGGGTG |
| 1678 | TGGCTTCTTCCCCTCTCCTG | 2802 | CACATTTGTAATCCCAGCGC | 3927 | ATTTTTTTCTGGTTCTGGGT |
| 1679 | CTGGCTTCTTCCCCTCTCCT | 2803 | TCACATTTGTAATCCCAGCG | 3928 | TATTTTTTTCTGGTTCTGGG |
| 1680 | GCTGGCTTCTTCCCCTCTCC | 2804 | CTCACATTTGTAATCCCAGC | 3929 | GTATTTTTTTCTGGTTCTGG |
| 1681 | TGCTGGCTTCTTCCCCTCTC | 2805 | GCTCACATTTGTAATCCCAG | 3930 | TGTATTTTTTTCTGGTTCTG |
| 1682 | GTGCTGGCTTCTTCCCCTCT | 2806 | AGCTCACATTTGTAATCCCA | 3931 | CTGTATTTTTTTCTGGTTCT |
| 1683 | GGTGCTGGCTTCTTCCCCTC | 2807 | TAGCTCACATTTGTAATCCC | 3932 | CCTGTATTTTTTTCTGGTTC |
| 1684 | AGGTGCTGGCTTCTTCCCCT | 2808 | GTAGCTCACATTTGTAATCC | 3933 | ACCTGTATTTTTTTCTGGTT |
| 1685 | TAGGTGCTGGCTTCTTCCCC | 2809 | AGTAGCTCACATTTGTAATC | 3934 | TACCTGTATTTTTTTCTGGT |
| 1686 | GTAGGTGCTGGCTTCTTCCC | 2810 | CAGTAGCTCACATTTGTAAT | 3935 | CTACCTGTATTTTTTTCTGG |
| 1687 | GGTAGGTGCTGGCTTCTTCC | 2811 | TCAGTAGCTCACATTTGTAA | 3936 | TCTACCTGTATTTTTTTCTG |
| 1688 | CGGTAGGTGCTGGCTTCTTC | 2812 | CTCAGTAGCTCACATTTGTA | 3937 | TTCTACCTGTATTTTTTTCT |
| 1689 | TCGGTAGGTGCTGGCTTCTT | 2813 | GCTCAGTAGCTCACATTTGT | 3938 | CTTCTACCTGTATTTTTTTC |
| 1690 | GTCGGTAGGTGCTGGCTTCT | 2814 | AGCTCAGTAGCTCACATTTG | 3939 | TCTTCTACCTGTATTTTTTT |
| 1691 | TGTCGGTAGGTGCTGGCTTC | 2815 | CAGCTCAGTAGCTCACATTT | 3940 | TTCTTCTACCTGTATTTTTT |
| 1692 | CTGTCGGTAGGTGCTGGCTT | 2816 | CCAGCTCAGTAGCTCACATT | 3941 | TTTCTTCTACCTGTATTTTT |
| 1693 | CCTGTCGGTAGGTGCTGGCT | 2817 | GCCAGCTCAGTAGCTCACAT | 3942 | TTTTCTTCTACCTGTATTTT |
| 1694 | CCCTGTCGGTAGGTGCTGGC | 2818 | AGCCAGCTCAGTAGCTCACA | 3943 | TTTTTCTTCTACCTGTATTT |
| 1695 | CCCCTGTCGGTAGGTGCTGG | 2819 | GAGCCAGCTCAGTAGCTCAC | 3944 | TTTTTTCTTCTACCTGTATT |
| 1696 | ACCCCTGTCGGTAGGTGCTG | 2820 | CGAGCCAGCTCAGTAGCTCA | 3945 | TTTTTTTCTTCTACCTGTAT |
| 1697 | CACCCCTGTCGGTAGGTGCT | 2821 | GCGAGCCAGCTCAGTAGCTC | 3946 | TTTTTTTTCTTCTACCTGTA |
| 1698 | CCACCCCTGTCGGTAGGTGC | 2822 | AGCGAGCCAGCTCAGTAGCT | 3947 | GTTTTTTTTCTTCTACCTGT |
| 1699 | TCCACCCCTGTCGGTAGGTG | 2823 | GAGCGAGCCAGCTCAGTAGC | 3948 | GGTTTTTTTTCTTCTACCTG |
| 1700 | CTCCACCCCTGTCGGTAGGT | 2824 | GGAGCGAGCCAGCTCAGTAG | 3949 | AGGTTTTTTTTCTTCTACCT |
| 1701 | GCTCCACCCCTGTCGGTAGG | 2825 | AGGAGCGAGCCAGCTCAGTA | 3950 | TAGGTTTTTTTTCTTCTACC |
| 1702 | AGCTCCACCCCTGTCGGTAG | 2826 | TAGGAGCGAGCCAGCTCAGT | 3951 | TTAGGTTTTTTTTCTTCTAC |
| 1703 | CAGCTCCACCCCTGTCGGTA | 2827 | ATAGGAGCGAGCCAGCTCAG | 3952 | TTTAGGTTTTTTTTCTTCTA |
| 1704 | CCAGCTCCACCCCTGTCGGT | 2828 | GATAGGAGCGAGCCAGCTCA | 3953 | GTTTAGGTTTTTTTTCTTCT |
| 1705 | CCCAGCTCCACCCCTGTCGG | 2829 | GGATAGGAGCGAGCCAGCTC | 3954 | TGTTTAGGTTTTTTTTCTTC |
| 1706 | ACCCAGCTCCACCCCTGTCG | 2830 | AGGATAGGAGCGAGCCAGCT | 3955 | CTGTTTAGGTTTTTTTTCTT |
| 1707 | GACCCAGCTCCACCCCTGTC | 2831 | TAGGATAGGAGCGAGCCAGC | 3956 | TCTGTTTAGGTTTTTTTTCT |
| 1708 | TGACCCAGCTCCACCCCTGT | 2832 | ATAGGATAGGAGCGAGCCAG | 3957 | GTCTGTTTAGGTTTTTTTTC |
| 1709 | TTGACCCAGCTCCACCCCTG | 2833 | GATAGGATAGGAGCGAGCCA | 3958 | AGTCTGTTTAGGTTTTTTTT |
| 1710 | CTTGACCCAGCTCCACCCCT | 2834 | GGATAGGATAGGAGCGAGCC | 3959 | CAGTCTGTTTAGGTTTTTTT |
| 1711 | TCTTGACCCAGCTCCACCCC | 2835 | AGGATAGGATAGGAGCGAGC | 3960 | TCAGTCTGTTTAGGTTTTTT |
| 1712 | TTCTTGACCCAGCTCCACCC | 2836 | CAGGATAGGATAGGAGCGAG | 3961 | TTCAGTCTGTTTAGGTTTTT |
| 1713 | ATTCTTGACCCAGCTCCACC | 2837 | GCAGGATAGGATAGGAGCGA | 3962 | TTTCAGTCTGTTTAGGTTTT |
| 1714 | CATTCTTGACCCAGCTCCAC | 2838 | GGCAGGATAGGATAGGAGCG | 3963 | GTTTCAGTCTGTTTAGGTTT |
| 1715 | CCATTCTTGACCCAGCTCCA | 2839 | GGGCAGGATAGGATAGGAGC | 3964 | TGTTTCAGTCTGTTTAGGTT |
| 1716 | ACCATTCTTGACCCAGCTCC | 2840 | AGGGCAGGATAGGATAGGAG | 3965 | TTGTTTCAGTCTGTTTAGGT |
| 1717 | CACCATTCTTGACCCAGCTC | 2841 | CAGGGCAGGATAGGATAGGA | 3966 | TTTGTTTCAGTCTGTTTAGG |
| 1718 | ACACCATTCTTGACCCAGCT | 2842 | ACAGGGCAGGATAGGATAGG | 3967 | ATTTGTTTCAGTCTGTTTAG |
| 1719 | CACACCATTCTTGACCCAGC | 2843 | GACAGGGCAGGATAGGATAG | 3968 | TATTTGTTTCAGTCTGTTTA |
| 1720 | CCACACCATTCTTGACCCAG | 2844 | AGACAGGGCAGGATAGGATA | 3969 | ATATTTGTTTCAGTCTGTTT |
| 1721 | ACCACACCATTCTTGACCCA | 2845 | AAGACAGGGCAGGATAGGAT | 3970 | GATATTTGTTTCAGTCTGTT |
| 1722 | GACCACACCATTCTTGACCC | 2846 | CAAGACAGGGCAGGATAGGA | 3971 | TGATATTTGTTTCAGTCTGT |
| 1723 | GGACCACACCATTCTTGACC | 2847 | ACAAGACAGGGCAGGATAGG | 3972 | GTGATATTTGTTTCAGTCTG |
| 1724 | GGGACCACACCATTCTTGAC | 2848 | GACAAGACAGGGCAGGATAG | 3973 | TGTGATATTTGTTTCAGTCT |
| 1725 | AGGGACCACACCATTCTTGA | 2849 | GGACAAGACAGGGCAGGATA | 3974 | TTGTGATATTTGTTTCAGTC |
| 1726 | CAGGGACCACACCATTCTTG | 2850 | AGGACAAGACAGGGCAGGAT | 3975 | TTTGTGATATTTGTTTCAGT |
| 1727 | GCAGGGACCACACCATTCTT | 2851 | CAGGACAAGACAGGGCAGGA | 3976 | CTTTGTGATATTTGTTTCAG |
| 1728 | AGCAGGGACCACACCATTCT | 2852 | ACAGGACAAGACAGGGCAGG | 3977 | ACTTTGTGATATTTGTTTCA |
| 1729 | AAGCAGGGACCACACCATTC | 2853 | GACAGGACAAGACAGGGCAG | 3978 | GACTTTGTGATATTTGTTTC |
| 1730 | AAAGCAGGGACCACACCATT | 2854 | GGACAGGACAAGACAGGGCA | 3979 | AGACTTTGTGATATTTGTTT |
| 1731 | CAAAGCAGGGACCACACCAT | 2855 | AGGACAGGACAAGACAGGGC | 3980 | TAGACTTTGTGATATTTGTT |
| 1732 | CCAAAGCAGGGACCACACCA | 2856 | CAGGACAGGACAAGACAGGG | 3981 | TTAGACTTTGTGATATTTGT |
| 1733 | CCCAAAGCAGGGACCACACC | 2857 | ACAGGACAGGACAAGACAGG | 3982 | ATTAGACTTTGTGATATTTG |
| 1734 | CCCCAAAGCAGGGACCACAC | 2858 | GACAGGACAGGACAAGACAG | 3983 | GATTAGACTTTGTGATATTT |
| 1735 | CCCCCAAAGCAGGGACCACA | 2859 | GGACAGGACAGGACAAGACA | 3984 | CGATTAGACTTTGTGATATT |
| 1736 | TCCCCCAAAGCAGGGACCAC | 2860 | AGGACAGGACAGGACAAGAC | 3985 | ACGATTAGACTTTGTGATAT |
| 1737 | TTCCCCCAAAGCAGGGACCA | 2861 | CAGGACAGGACAGGACAAGA | 3986 | GACGATTAGACTTTGTGATA |
| 1738 | ATTCCCCCAAAGCAGGGACC | 2862 | GCAGGACAGGACAGGACAAG | 3987 | TGACGATTAGACTTTGTGAT |
| 1739 | CATTCCCCCAAAGCAGGGAC | 2863 | GGCAGGACAGGACAGGACAA | 3988 | TTGACGATTAGACTTTGTGA |
| 1740 | GCATTCCCCCAAAGCAGGGA | 2864 | GGGCAGGACAGGACAGGACA | 3989 | TTTGACGATTAGACTTTGTG |
| 1741 | AGCATTCCCCCAAAGCAGGG | 2865 | AGGGCAGGACAGGACAGGAC | 3990 | CTTTGACGATTAGACTTTGT |
| 1742 | CAGCATTCCCCCAAAGCAGG | 2866 | TAGGGCAGGACAGGACAGGA | 3991 | GCTTTGACGATTAGACTTTG |
| 1743 | CCAGCATTCCCCCAAAGCAG | 2867 | ATAGGGCAGGACAGGACAGG | 3992 | GGCTTTGACGATTAGACTTT |
| 1744 | CCCAGCATTCCCCCAAAGCA | 2868 | AATAGGGCAGGACAGGACAG | 3993 | CGGCTTTGACGATTAGACTT |
| 1745 | CCCCAGCATTCCCCCAAAGC | 2869 | GAATAGGGCAGGACAGGACA | 3994 | ACGGCTTTGACGATTAGACT |
| 1746 | TCCCCAGCATTCCCCCAAAG | 2870 | AGAATAGGGCAGGACAGGAC | 3995 | CACGGCTTTGACGATTAGAC |
| 1747 | CTCCCCAGCATTCCCCCAAA | 2871 | CAGAATAGGGCAGGACAGGA | 3996 | TCACGGCTTTGACGATTAGA |
| 1748 | CCTCCCCAGCATTCCCCCAA | 2872 | GCAGAATAGGGCAGGACAGG | 3997 | ATCACGGCTTTGACGATTAG |
| 1749 | ACCTCCCCAGCATTCCCCCA | 2873 | GGCAGAATAGGGCAGGACAG | 3998 | AATCACGGCTTTGACGATTA |
| 1750 | TACCTCCCCAGCATTCCCCC | 2874 | GGGCAGAATAGGGCAGGACA | 3999 | GAATCACGGCTTTGACGATT |
| 1751 | CTACCTCCCCAGCATTCCCC | 2875 | AGGGCAGAATAGGGCAGGAC | 4000 | AGAATCACGGCTTTGACGAT |
| 1752 | TCTACCTCCCCAGCATTCCC | 2876 | TAGGGCAGAATAGGGCAGGA | 4001 | AAGAATCACGGCTTTGACGA |
| 1753 | TTCTACCTCCCCAGCATTCC | 2877 | ATAGGGCAGAATAGGGCAGG | 4002 | CAAGAATCACGGCTTTGACG |
| 1754 | TTTCTACCTCCCCAGCATTC | 2878 | AATAGGGCAGAATAGGGCAG | 4003 | TCAAGAATCACGGCTTTGAC |
| 1755 | CTTTCTACCTCCCCAGCATT | 2879 | GAATAGGGCAGAATAGGGCA | 4004 | CTCAAGAATCACGGCTTTGA |
| 1756 | GCTTTCTACCTCCCCAGCAT | 2880 | AGAATAGGGCAGAATAGGGC | 4005 | GCTCAAGAATCACGGCTTTG |
| 1757 | GGCTTTCTACCTCCCCAGCA | 2881 | GAGAATAGGGCAGAATAGGG | 4006 | TGCTCAAGAATCACGGCTTT |
| 1758 | GGGCTTTCTACCTCCCCAGC | 2882 | GGAGAATAGGGCAGAATAGG | 4007 | CTGCTCAAGAATCACGGCTT |
| 1759 | GGGGCTTTCTACCTCCCCAG | 2883 | TGGAGAATAGGGCAGAATAG | 4008 | GCTGCTCAAGAATCACGGCT |
| 1760 | AGGGGCTTTCTACCTCCCCA | 2884 | TTGGAGAATAGGGCAGAATA | 4009 | CGCTGCTCAAGAATCACGGC |
| 1761 | AAGGGGCTTTCTACCTCCCC | 2885 | TTTGGAGAATAGGGCAGAAT | 4010 | CCGCTGCTCAAGAATCACGG |
| 1762 | GAAGGGGCTTTCTACCTCCC | 2886 | GTTTGGAGAATAGGGCAGAA | 4011 | GCCGCTGCTCAAGAATCACG |
| 1763 | AGAAGGGGCTTTCTACCTCC | 2887 | AGTTTGGAGAATAGGGCAGA | 4012 | TGCCGCTGCTCAAGAATCAC |
| 1764 | TAGAAGGGGCTTTCTACCTC | 2888 | GAGTTTGGAGAATAGGGCAG | 4013 | TTGCCGCTGCTCAAGAATCA |
| 1765 | TTAGAAGGGGCTTTCTACCT | 2889 | CGAGTTTGGAGAATAGGGCA | 4014 | ATTGCCGCTGCTCAAGAATC |
| 1766 | GTTAGAAGGGGCTTTCTACC | 2890 | CCGAGTTTGGAGAATAGGGC | 4015 | AATTGCCGCTGCTCAAGAAT |
| 1767 | CGTTAGAAGGGGCTTTCTAC | 2891 | ACCGAGTTTGGAGAATAGGG | 4016 | AAATTGCCGCTGCTCAAGAA |
| 1768 | CCGTTAGAAGGGGCTTTCTA | 2892 | AACCGAGTTTGGAGAATAGG | 4017 | AAAATTGCCGCTGCTCAAGA |
| 1769 | CCCGTTAGAAGGGGCTTTCT | 2893 | CAACCGAGTTTGGAGAATAG | 4018 | CAAAATTGCCGCTGCTCAAG |
| 1770 | CCCCGTTAGAAGGGGCTTTC | 2894 | TCAACCGAGTTTGGAGAATA | 4019 | GCAAAATTGCCGCTGCTCAA |
| 1771 | GCCCCGTTAGAAGGGGCTTT | 2895 | GTCAACCGAGTTTGGAGAAT | 4020 | GGCAAAATTGCCGCTGCTCA |
| 1772 | CGCCCCGTTAGAAGGGGCTT | 2896 | TGTCAACCGAGTTTGGAGAA | 4021 | CGGCAAAATTGCCGCTGCTC |
| 1773 | ACGCCCCGTTAGAAGGGGCT | 2897 | ATGTCAACCGAGTTTGGAGA | 4022 | CCGGCAAAATTGCCGCTGCT |
| 1774 | GACGCCCCGTTAGAAGGGGC | 2898 | CATGTCAACCGAGTTTGGAG | 4023 | GCCGGCAAAATTGCCGCTGC |
| 1775 | TGACGCCCCGTTAGAAGGGG | 2899 | GCATGTCAACCGAGTTTGGA | 4024 | GGCCGGCAAAATTGCCGCTG |
| 1776 | GTGACGCCCCGTTAGAAGGG | 2900 | AGCATGTCAACCGAGTTTGG | 4025 | TGGCCGGCAAAATTGCCGCT |
| 1777 | AGTGACGCCCCGTTAGAAGG | 2901 | TAGCATGTCAACCGAGTTTG | 4026 | CTGGCCGGCAAAATTGCCGC |
| 1778 | CAGTGACGCCCCGTTAGAAG | 2902 | CTAGCATGTCAACCGAGTTT | 4027 | CCTGGCCGGCAAAATTGCCG |
| 1779 | GCAGTGACGCCCCGTTAGAA | 2903 | CCTAGCATGTCAACCGAGTT | 4028 | CCCTGGCCGGCAAAATTGCC |
| 1780 | TGCAGTGACGCCCCGTTAGA | 2904 | GCCTAGCATGTCAACCGAGT | 4029 | TCCCTGGCCGGCAAAATTGC |
| 1781 | TTGCAGTGACGCCCCGTTAG | 2905 | TGCCTAGCATGTCAACCGAG | 4030 | GTCCCTGGCCGGCAAAATTG |
| 1782 | ATTGCAGTGACGCCCCGTTA | 2906 | CTGCCTAGCATGTCAACCGA | 4031 | TGTCCCTGGCCGGCAAAATT |
| 1783 | AATTGCAGTGACGCCCCGTT | 2907 | CCTGCCTAGCATGTCAACCG | 4032 | ATGTCCCTGGCCGGCAAAAT |
| 1784 | TAATTGCAGTGACGCCCCGT | 2908 | GCCTGCCTAGCATGTCAACC | 4033 | AATGTCCCTGGCCGGCAAAA |
| 1785 | GTAATTGCAGTGACGCCCCG | 2909 | GGCCTGCCTAGCATGTCAAC | 4034 | AAATGTCCCTGGCCGGCAAA |
| 1786 | AGTAATTGCAGTGACGCCCC | 2910 | GGGCCTGCCTAGCATGTCAA | 4035 | TAAATGTCCCTGGCCGGCAA |
| 1787 | CAGTAATTGCAGTGACGCCC | 2911 | GGGGCCTGCCTAGCATGTCA | 4036 | CTAAATGTCCCTGGCCGGCA |
| 1788 | GCAGTAATTGCAGTGACGCC | 2912 | TGGGGCCTGCCTAGCATGTC | 4037 | CCTAAATGTCCCTGGCCGGC |
| 1789 | AGCAGTAATTGCAGTGACGC | 2913 | CTGGGGCCTGCCTAGCATGT | 4038 | TCCTAAATGTCCCTGGCCGG |
| 1790 | AAGCAGTAATTGCAGTGACG | 2914 | TCTGGGGCCTGCCTAGCATG | 4039 | CTCCTAAATGTCCCTGGCCG |
| 1791 | GAAGCAGTAATTGCAGTGAC | 2915 | CTCTGGGGCCTGCCTAGCAT | 4040 | ACTCCTAAATGTCCCTGGCC |
| 1792 | GGAAGCAGTAATTGCAGTGA | 2916 | ACTCTGGGGCCTGCCTAGCA | 4041 | CACTCCTAAATGTCCCTGGC |
| 1793 | AGGAAGCAGTAATTGCAGTG | 2917 | CACTCTGGGGCCTGCCTAGC | 4042 | ACACTCCTAAATGTCCCTGG |
| 1794 | GAGGAAGCAGTAATTGCAGT | 2918 | TCACTCTGGGGCCTGCCTAG | 4043 | GACACTCCTAAATGTCCCTG |
| 1795 | AGAGGAAGCAGTAATTGCAG | 2919 | CTCACTCTGGGGCCTGCCTA | 4044 | AGACACTCCTAAATGTCCCT |
| 1796 | AAGAGGAAGCAGTAATTGCA | 2920 | ACTCACTCTGGGGCCTGCCT | 4045 | CAGACACTCCTAAATGTCCC |
| 1797 | AAAGAGGAAGCAGTAATTGC | 2921 | AACTCACTCTGGGGCCTGCC | 4046 | CCAGACACTCCTAAATGTCC |
| 1798 | GAAAGAGGAAGCAGTAATTG | 2922 | CAACTCACTCTGGGGCCTGC | 4047 | TCCAGACACTCCTAAATGTC |
| 1799 | GGAAAGAGGAAGCAGTAATT | 2923 | TCAACTCACTCTGGGGCCTG | 4048 | CTCCAGACACTCCTAAATGT |
| 1800 | GGGAAAGAGGAAGCAGTAAT | 2924 | TTCAACTCACTCTGGGGCCT | 4049 | TCTCCAGACACTCCTAAATG |
| 1801 | TGGGAAAGAGGAAGCAGTAA | 2925 | CTTCAACTCACTCTGGGGCC | 4050 | GTCTCCAGACACTCCTAAAT |
| 1802 | ATGGGAAAGAGGAAGCAGTA | 2926 | CCTTCAACTCACTCTGGGGC | 4051 | TGTCTCCAGACACTCCTAAA |
| 1803 | TATGGGAAAGAGGAAGCAGT | 2927 | TCCTTCAACTCACTCTGGGG | 4052 | TTGTCTCCAGACACTCCTAA |
| 1804 | TTATGGGAAAGAGGAAGCAG | 2928 | ATCCTTCAACTCACTCTGGG | 4053 | ATTGTCTCCAGACACTCCTA |
| 1805 | TTTATGGGAAAGAGGAAGCA | 2929 | CATCCTTCAACTCACTCTGG | 4054 | GATTGTCTCCAGACACTCCT |
| 1806 | TTTTATGGGAAAGAGGAAGC | 2930 | CCATCCTTCAACTCACTCTG | 4055 | AGATTGTCTCCAGACACTCC |
| 1807 | GTTTTATGGGAAAGAGGAAG | 2931 | ACCATCCTTCAACTCACTCT | 4056 | CAGATTGTCTCCAGACACTC |
| 1808 | AGTTTTATGGGAAAGAGGAA | 2932 | AACCATCCTTCAACTCACTC | 4057 | CCAGATTGTCTCCAGACACT |
| 1809 | GAGTTTTATGGGAAAGAGGA | 2933 | AAACCATCCTTCAACTCACT | 4058 | CCCAGATTGTCTCCAGACAC |
| 1810 | GGAGTTTTATGGGAAAGAGG | 2934 | GAAACCATCCTTCAACTCAC | 4059 | ACCCAGATTGTCTCCAGACA |
| 1811 | GGGAGTTTTATGGGAAAGAG | 2935 | GGAAACCATCCTTCAACTCA | 4060 | AACCCAGATTGTCTCCAGAC |
| 1812 | GGGGAGTTTTATGGGAAAGA | 2936 | TGGAAACCATCCTTCAACTC | 4061 | CAACCCAGATTGTCTCCAGA |
| 1813 | GGGGGAGTTTTATGGGAAAG | 2937 | CTGGAAACCATCCTTCAACT | 4062 | ACAACCCAGATTGTCTCCAG |
| 1814 | AGGGGGAGTTTTATGGGAAA | 2938 | CCTGGAAACCATCCTTCAAC | 4063 | GACAACCCAGATTGTCTCCA |
| 1815 | TAGGGGGAGTTTTATGGGAA | 2939 | TCCTGGAAACCATCCTTCAA | 4064 | TGACAACCCAGATTGTCTCC |
| 1816 | CTAGGGGGAGTTTTATGGGA | 2940 | GTCCTGGAAACCATCCTTCA | 4065 | GTGACAACCCAGATTGTCTC |
| 1817 | ACTAGGGGGAGTTTTATGGG | 2941 | TGTCCTGGAAACCATCCTTC | 4066 | TGTGACAACCCAGATTGTCT |
| 1818 | CACTAGGGGGAGTTTTATGG | 2942 | TTGTCCTGGAAACCATCCTT | 4067 | TTGTGACAACCCAGATTGTC |
| 1819 | ACACTAGGGGGAGTTTTATG | 2943 | TTTGTCCTGGAAACCATCCT | 4068 | GTTGTGACAACCCAGATTGT |
| 1820 | TACACTAGGGGGAGTTTTAT | 2944 | CTTTGTCCTGGAAACCATCC | 4069 | AGTTGTGACAACCCAGATTG |
| 1821 | ATACACTAGGGGGAGTTTTA | 2945 | TCTTTGTCCTGGAAACCATC | 4070 | GAGTTGTGACAACCCAGATT |
| 1822 | GATACACTAGGGGGAGTTTT | 2946 | ATCTTTGTCCTGGAAACCAT | 4071 | TGAGTTGTGACAACCCAGAT |
| 1823 | TGATACACTAGGGGGAGTTT | 2947 | TATCTTTGTCCTGGAAACCA | 4072 | CTGAGTTGTGACAACCCAGA |
| 1824 | CTGATACACTAGGGGGAGTT | 2948 | TTATCTTTGTCCTGGAAACC | 4073 | CCTGAGTTGTGACAACCCAG |
| 1825 | TCTGATACACTAGGGGGAGT | 2949 | TTTATCTTTGTCCTGGAAAC | 4074 | CCCTGAGTTGTGACAACCCA |
| 1826 | TTCTGATACACTAGGGGGAG | 2950 | GTTTATCTTTGTCCTGGAAA | 4075 | ACCCTGAGTTGTGACAACCC |
| 1827 | GTTCTGATACACTAGGGGGA | 2951 | TGTTTATCTTTGTCCTGGAA | 4076 | CACCCTGAGTTGTGACAACC |
| 1828 | GGTTCTGATACACTAGGGGG | 2952 | CTGTTTATCTTTGTCCTGGA | 4077 | CCACCCTGAGTTGTGACAAC |
| 1829 | GGGTTCTGATACACTAGGGG | 2953 | GCTGTTTATCTTTGTCCTGG | 4078 | TCCACCCTGAGTTGTGACAA |
| 1830 | GGGGTTCTGATACACTAGGG | 2954 | GGCTGTTTATCTTTGTCCTG | 4079 | CTCCACCCTGAGTTGTGACA |
| 1831 | GGGGGTTCTGATACACTAGG | 2955 | GGGCTGTTTATCTTTGTCCT | 4080 | CCTCCACCCTGAGTTGTGAC |
| 1832 | TGGGGGTTCTGATACACTAG | 2956 | GGGGCTGTTTATCTTTGTCC | 4081 | CCCTCCACCCTGAGTTGTGA |
| 1833 | TTGGGGGTTCTGATACACTA | 2957 | TGGGGCTGTTTATCTTTGTC | 4082 | TCCCTCCACCCTGAGTTGTG |
| 1834 | CTTGGGGGTTCTGATACACT | 2958 | TTGGGGCTGTTTATCTTTGT | 4083 | GTCCCTCCACCCTGAGTTGT |
| 1835 | CCTTGGGGGTTCTGATACAC | 2959 | TTTGGGGCTGTTTATCTTTG | 4084 | TGTCCCTCCACCCTGAGTTG |
| 1836 | TCCTTGGGGGTTCTGATACA | 2960 | CTTTGGGGCTGTTTATCTTT | 4085 | GTGTCCCTCCACCCTGAGTT |
| 1837 | CTCCTTGGGGGTTCTGATAC | 2961 | GCTTTGGGGCTGTTTATCTT | 4086 | AGTGTCCCTCCACCCTGAGT |
| 1838 | ACTCCTTGGGGGTTCTGATA | 2962 | GGCTTTGGGGCTGTTTATCT | 4087 | CAGTGTCCCTCCACCCTGAG |
| 1839 | AACTCCTTGGGGGTTCTGAT | 2963 | GGGCTTTGGGGCTGTTTATC | 4088 | CCAGTGTCCCTCCACCCTGA |
| 1840 | AAACTCCTTGGGGGTTCTGA | 2964 | AGGGCTTTGGGGCTGTTTAT | 4089 | GCCAGTGTCCCTCCACCCTG |
| 1841 | GAAACTCCTTGGGGGTTCTG | 2965 | AAGGGCTTTGGGGCTGTTTA | 4090 | TGCCAGTGTCCCTCCACCCT |
| 1842 | TGAAACTCCTTGGGGGTTCT | 2966 | AAAGGGCTTTGGGGCTGTTT | 4091 | ATGCCAGTGTCCCTCCACCC |
| 1843 | CTGAAACTCCTTGGGGGTTC | 2967 | GAAAGGGCTTTGGGGCTGTT | 4092 | GATGCCAGTGTCCCTCCACC |
| 1844 | ACTGAAACTCCTTGGGGGTT | 2968 | GGAAAGGGCTTTGGGGCTGT | 4093 | AGATGCCAGTGTCCCTCCAC |
| 1845 | TACTGAAACTCCTTGGGGGT | 2969 | AGGAAAGGGCTTTGGGGCTG | 4094 | CAGATGCCAGTGTCCCTCCA |
| 1846 | TTACTGAAACTCCTTGGGGG | 2970 | GAGGAAAGGGCTTTGGGGCT | 4095 | CCAGATGCCAGTGTCCCTCC |
| 1847 | CTTACTGAAACTCCTTGGGG | 2971 | TGAGGAAAGGGCTTTGGGGC | 4096 | ACCAGATGCCAGTGTCCCTC |
| 1848 | GCTTACTGAAACTCCTTGGG | 2972 | TTGAGGAAAGGGCTTTGGGG | 4097 | TACCAGATGCCAGTGTCCCT |
| 1849 | CGCTTACTGAAACTCCTTGG | 2973 | CTTGAGGAAAGGGCTTTGGG | 4098 | CTACCAGATGCCAGTGTCCC |
| 1850 | CCGCTTACTGAAACTCCTTG | 2974 | CCTTGAGGAAAGGGCTTTGG | 4099 | CCTACCAGATGCCAGTGTCC |
| 1851 | ACCGCTTACTGAAACTCCTT | 2975 | ACCTTGAGGAAAGGGCTTTG | 4100 | ACCTACCAGATGCCAGTGTC |
| 1852 | AACCGCTTACTGAAACTCCT | 2976 | CACCTTGAGGAAAGGGCTTT | 4101 | TACCTACCAGATGCCAGTGT |
| 1853 | GAACCGCTTACTGAAACTCC | 2977 | GCACCTTGAGGAAAGGGCTT | 4102 | TTACCTACCAGATGCCAGTG |
| 1854 | AGAACCGCTTACTGAAACTC | 2978 | AGCACCTTGAGGAAAGGGCT | 4103 | CTTACCTACCAGATGCCAGT |
| 1855 | AAGAACCGCTTACTGAAACT | 2979 | GAGCACCTTGAGGAAAGGGC | 4104 | CCTTACCTACCAGATGCCAG |
| 1856 | GAAGAACCGCTTACTGAAAC | 2980 | TGAGCACCTTGAGGAAAGGG | 4105 | CCCTTACCTACCAGATGCCA |
| 1857 | AGAAGAACCGCTTACTGAAA | 2981 | CTGAGCACCTTGAGGAAAGG | 4106 | GCCCTTACCTACCAGATGCC |
| 1858 | CAGAAGAACCGCTTACTGAA | 2982 | GCTGAGCACCTTGAGGAAAG | 4107 | GGCCCTTACCTACCAGATGC |
| 1859 | ACAGAAGAACCGCTTACTGA | 2983 | TGCTGAGCACCTTGAGGAAA | 4108 | TGGCCCTTACCTACCAGATG |
| 1860 | AACAGAAGAACCGCTTACTG | 2984 | CTGCTGAGCACCTTGAGGAA | 4109 | CTGGCCCTTACCTACCAGAT |
| 1861 | CAACAGAAGAACCGCTTACT | 2985 | CCTGCTGAGCACCTTGAGGA | 4110 | GCTGGCCCTTACCTACCAGA |
| 1862 | ACAACAGAAGAACCGCTTAC | 2986 | TCCTGCTGAGCACCTTGAGG | 4111 | TGCTGGCCCTTACCTACCAG |
| 1863 | GACAACAGAAGAACCGCTTA | 2987 | GTCCTGCTGAGCACCTTGAG | 4112 | TTGCTGGCCCTTACCTACCA |
| 1864 | AGACAACAGAAGAACCGCTT | 2988 | TGTCCTGCTGAGCACCTTGA | 4113 | ATTGCTGGCCCTTACCTACC |
| 1865 | GAGACAACAGAAGAACCGCT | 2989 | CTGTCCTGCTGAGCACCTTG | 4114 | GATTGCTGGCCCTTACCTAC |
| 1866 | GGAGACAACAGAAGAACCGC | 2990 | CCTGTCCTGCTGAGCACCTT | 4115 | GGATTGCTGGCCCTTACCTA |
| 1867 | CGGAGACAACAGAAGAACCG | 2991 | CCCTGTCCTGCTGAGCACCT | 4116 | GGGATTGCTGGCCCTTACCT |
| 1868 | CCGGAGACAACAGAAGAACC | 2992 | TCCCTGTCCTGCTGAGCACC | 4117 | TGGGATTGCTGGCCCTTACC |
| 1869 | GCCGGAGACAACAGAAGAAC | 2993 | CTCCCTGTCCTGCTGAGCAC | 4118 | GTGGGATTGCTGGCCCTTAC |
| 1870 | AGCCGGAGACAACAGAAGAA | 2994 | GCTCCCTGTCCTGCTGAGCA | 4119 | AGTGGGATTGCTGGCCCTTA |
| 1871 | CAGCCGGAGACAACAGAAGA | 2995 | TGCTCCCTGTCCTGCTGAGC | 4120 | TAGTGGGATTGCTGGCCCTT |
| 1872 | TCAGCCGGAGACAACAGAAG | 2996 | CTGCTCCCTGTCCTGCTGAG | 4121 | TTAGTGGGATTGCTGGCCCT |
| 1873 | CTCAGCCGGAGACAACAGAA | 2997 | CCTGCTCCCTGTCCTGCTGA | 4122 | TTTAGTGGGATTGCTGGCCC |
| 1874 | TCTCAGCCGGAGACAACAGA | 2998 | CCCTGCTCCCTGTCCTGCTG | 4123 | GTTTAGTGGGATTGCTGGCC |
| 1875 | GTCTCAGCCGGAGACAACAG | 2999 | TCCCTGCTCCCTGTCCTGCT | 4124 | TGTTTAGTGGGATTGCTGGC |
| 1876 | AGTCTCAGCCGGAGACAACA | 3000 | ATCCCTGCTCCCTGTCCTGC | 4125 | ATGTTTAGTGGGATTGCTGG |
| 1877 | GAGTCTCAGCCGGAGACAAC | 3001 | AATCCCTGCTCCCTGTCCTG | 4126 | GATGTTTAGTGGGATTGCTG |
| 1878 | GGAGTCTCAGCCGGAGACAA | 3002 | CAATCCCTGCTCCCTGTCCT | 4127 | TGATGTTTAGTGGGATTGCT |
| 1879 | TGGAGTCTCAGCCGGAGACA | 3003 | CCAATCCCTGCTCCCTGTCC | 4128 | GTGATGTTTAGTGGGATTGC |
| 1880 | CTGGAGTCTCAGCCGGAGAC | 3004 | ACCAATCCCTGCTCCCTGTC | 4129 | TGTGATGTTTAGTGGGATTG |
| 1881 | CCTGGAGTCTCAGCCGGAGA | 3005 | CACCAATCCCTGCTCCCTGT | 4130 | GTGTGATGTTTAGTGGGATT |
| 1882 | CCCTGGAGTCTCAGCCGGAG | 3006 | CCACCAATCCCTGCTCCCTG | 4131 | CGTGTGATGTTTAGTGGGAT |
| 1883 | CCCCTGGAGTCTCAGCCGGA | 3007 | CCCACCAATCCCTGCTCCCT | 4132 | TCGTGTGATGTTTAGTGGGA |
| 1884 | TCCCCTGGAGTCTCAGCCGG | 3008 | TCCCACCAATCCCTGCTCCC | 4133 | GTCGTGTGATGTTTAGTGGG |
| 1885 | TTCCCCTGGAGTCTCAGCCG | 3009 | CTCCCACCAATCCCTGCTCC | 4134 | TGTCGTGTGATGTTTAGTGG |
| 1886 | GTTCCCCTGGAGTCTCAGCC | 3010 | TCTCCCACCAATCCCTGCTC | 4135 | GTGTCGTGTGATGTTTAGTG |
| 1887 | GGTTCCCCTGGAGTCTCAGC | 3011 | GTCTCCCACCAATCCCTGCT | 4136 | TGTGTCGTGTGATGTTTAGT |
| 1888 | AGGTTCCCCTGGAGTCTCAG | 3012 | TGTCTCCCACCAATCCCTGC | 4137 | GTGTGTCGTGTGATGTTTAG |
| 1889 | GAGGTTCCCCTGGAGTCTCA | 3013 | CTGTCTCCCACCAATCCCTG | 4138 | TGTGTGTCGTGTGATGTTTA |
| 1890 | TGAGGTTCCCCTGGAGTCTC | 3014 | ACTGTCTCCCACCAATCCCT | 4139 | CTGTGTGTCGTGTGATGTTT |
| 1891 | TTGAGGTTCCCCTGGAGTCT | 3015 | GACTGTCTCCCACCAATCCC | 4140 | TCTGTGTGTCGTGTGATGTT |
| 1892 | CTTGAGGTTCCCCTGGAGTC | 3016 | AGACTGTCTCCCACCAATCC | 4141 | TTCTGTGTGTCGTGTGATGT |
| 1893 | GCTTGAGGTTCCCCTGGAGT | 3017 | GAGACTGTCTCCCACCAATC | 4142 | GTTCTGTGTGTCGTGTGATG |
| 1894 | AGCTTGAGGTTCCCCTGGAG | 3018 | TGAGACTGTCTCCCACCAAT | 4143 | TGTTCTGTGTGTCGTGTGAT |
| 1895 | GAGCTTGAGGTTCCCCTGGA | 3019 | CTGAGACTGTCTCCCACCAA | 4144 | TTGTTCTGTGTGTCGTGTGA |
| 1896 | TGAGCTTGAGGTTCCCCTGG | 3020 | CCTGAGACTGTCTCCCACCA | 4145 | ATTGTTCTGTGTGTCGTGTG |
| 1897 | GTGAGCTTGAGGTTCCCCTG | 3021 | GCCTGAGACTGTCTCCCACC | 4146 | GATTGTTCTGTGTGTCGTGT |
| 1898 | TGTGAGCTTGAGGTTCCCCT | 3022 | AGCCTGAGACTGTCTCCCAC | 4147 | GGATTGTTCTGTGTGTCGTG |
| 1899 | ATGTGAGCTTGAGGTTCCCC | 3023 | CAGCCTGAGACTGTCTCCCA | 4148 | GGGATTGTTCTGTGTGTCGT |
| 1900 | CATGTGAGCTTGAGGTTCCC | 3024 | TCAGCCTGAGACTGTCTCCC | 4149 | GGGGATTGTTCTGTGTGTCG |
| 1901 | CCATGTGAGCTTGAGGTTCC | 3025 | TTCAGCCTGAGACTGTCTCC | 4150 | GGGGGATTGTTCTGTGTGTC |
| 1902 | GCCATGTGAGCTTGAGGTTC | 3026 | ATTCAGCCTGAGACTGTCTC | 4151 | TGGGGGATTGTTCTGTGTGT |
| 1903 | GGCCATGTGAGCTTGAGGTT | 3027 | GATTCAGCCTGAGACTGTCT | 4152 | GTGGGGGATTGTTCTGTGTG |
| 1904 | GGGCCATGTGAGCTTGAGGT | 3028 | AGATTCAGCCTGAGACTGTC | 4153 | TGTGGGGGATTGTTCTGTGT |
| 1905 | AGGGCCATGTGAGCTTGAGG | 3029 | TAGATTCAGCCTGAGACTGT | 4154 | TTGTGGGGGATTGTTCTGTG |
| 1906 | CAGGGCCATGTGAGCTTGAG | 3030 | TTAGATTCAGCCTGAGACTG | 4155 | GTTGTGGGGGATTGTTCTGT |
| 1907 | CCAGGGCCATGTGAGCTTGA | 3031 | ATTAGATTCAGCCTGAGACT | 4156 | TGTTGTGGGGGATTGTTCTG |
| 1908 | GCCAGGGCCATGTGAGCTTG | 3032 | AATTAGATTCAGCCTGAGAC | 4157 | TTGTTGTGGGGGATTGTTCT |
| 1909 | CGCCAGGGCCATGTGAGCTT | 3033 | AAATTAGATTCAGCCTGAGA | 4158 | TTTGTTGTGGGGGATTGTTC |
| 1910 | CCGCCAGGGCCATGTGAGCT | 3034 | TAAATTAGATTCAGCCTGAG | 4159 | CTTTGTTGTGGGGGATTGTT |
| 1911 | CCCGCCAGGGCCATGTGAGC | 3035 | TTAAATTAGATTCAGCCTGA | 4160 | TCTTTGTTGTGGGGGATTGT |
| 1912 | GCCCGCCAGGGCCATGTGAG | 3036 | CTTAAATTAGATTCAGCCTG | 4161 | TTCTTTGTTGTGGGGGATTG |
| 1913 | GGCCCGCCAGGGCCATGTGA | 3037 | TCTTAAATTAGATTCAGCCT | 4162 | CTTCTTTGTTGTGGGGGATT |
| 1914 | GGGCCCGCCAGGGCCATGTG | 3038 | ATCTTAAATTAGATTCAGCC | 4163 | ACTTCTTTGTTGTGGGGGAT |
| 1915 | GGGGCCCGCCAGGGCCATGT | 3039 | TATCTTAAATTAGATTCAGC | 4164 | TACTTCTTTGTTGTGGGGGA |
| 1916 | AGGGGCCCGCCAGGGCCATG | 3040 | GTATCTTAAATTAGATTCAG | 4165 | ATACTTCTTTGTTGTGGGGG |
| 1917 | CAGGGGCCCGCCAGGGCCAT | 3041 | TGTATCTTAAATTAGATTCA | 4166 | GATACTTCTTTGTTGTGGGG |
| 1918 | CCAGGGGCCCGCCAGGGCCA | 3042 | TTGTATCTTAAATTAGATTC | 4167 | GGATACTTCTTTGTTGTGGG |
| 1919 | CCCAGGGGCCCGCCAGGGCC | 3043 | CTTGTATCTTAAATTAGATT | 4168 | AGGATACTTCTTTGTTGTGG |
| 1920 | GCCCAGGGGCCCGCCAGGGC | 3044 | CCTTGTATCTTAAATTAGAT | 4169 | CAGGATACTTCTTTGTTGTG |
| 1921 | TGCCCAGGGGCCCGCCAGGG | 3045 | ACCTTGTATCTTAAATTAGA | 4170 | GCAGGATACTTCTTTGTTGT |
| 1922 | CTGCCCAGGGGCCCGCCAGG | 3046 | AACCTTGTATCTTAAATTAG | 4171 | GGCAGGATACTTCTTTGTTG |
| 1923 | CCTGCCCAGGGGCCCGCCAG | 3047 | CAACCTTGTATCTTAAATTA | 4172 | GGGCAGGATACTTCTTTGTT |
| 1924 | TCCTGCCCAGGGGCCCGCCA | 3048 | ACAACCTTGTATCTTAAATT | 4173 | TGGGCAGGATACTTCTTTGT |
| 1925 | CTCCTGCCCAGGGGCCCGCC | 3049 | CACAACCTTGTATCTTAAAT | 4174 | TTGGGCAGGATACTTCTTTG |
| 1926 | GCTCCTGCCCAGGGGCCCGC | 3050 | GCACAACCTTGTATCTTAAA | 4175 | TTTGGGCAGGATACTTCTTT |
| 1927 | TGCTCCTGCCCAGGGGCCCG | 3051 | CGCACAACCTTGTATCTTAA | 4176 | TTTTGGGCAGGATACTTCTT |
| 1928 | CTGCTCCTGCCCAGGGGCCC | 3052 | GCGCACAACCTTGTATCTTA | 4177 | CTTTTGGGCAGGATACTTCT |
| 1929 | CCTGCTCCTGCCCAGGGGCC | 3053 | GGCGCACAACCTTGTATCTT | 4178 | CCTTTTGGGCAGGATACTTC |
| 1930 | GCCTGCTCCTGCCCAGGGGC | 3054 | TGGCGCACAACCTTGTATCT | 4179 | ACCTTTTGGGCAGGATACTT |
| 1931 | CGCCTGCTCCTGCCCAGGGG | 3055 | CTGGCGCACAACCTTGTATC | 4180 | GACCTTTTGGGCAGGATACT |
| 1932 | TCGCCTGCTCCTGCCCAGGG | 3056 | ACTGGCGCACAACCTTGTAT | 4181 | TGACCTTTTGGGCAGGATAC |
| 1933 | CTCGCCTGCTCCTGCCCAGG | 3057 | AACTGGCGCACAACCTTGTA | 4182 | TTGACCTTTTGGGCAGGATA |
| 1934 | TCTCGCCTGCTCCTGCCCAG | 3058 | CAACTGGCGCACAACCTTGT | 4183 | ATTGACCTTTTGGGCAGGAT |
| 1935 | CTCTCGCCTGCTCCTGCCCA | 3059 | GCAACTGGCGCACAACCTTG | 4184 | TATTGACCTTTTGGGCAGGA |
| 1936 | CCTCTCGCCTGCTCCTGCCC | 3060 | GGCAACTGGCGCACAACCTT | 4185 | CTATTGACCTTTTGGGCAGG |
| 1937 | ACCTCTCGCCTGCTCCTGCC | 3061 | TGGCAACTGGCGCACAACCT | 4186 | ACTATTGACCTTTTGGGCAG |
| 1938 | GACCTCTCGCCTGCTCCTGC | 3062 | CTGGCAACTGGCGCACAACC | 4187 | CACTATTGACCTTTTGGGCA |
| 1939 | AGACCTCTCGCCTGCTCCTG | 3063 | CCTGGCAACTGGCGCACAAC | 4188 | GCACTATTGACCTTTTGGGC |
| 1940 | CAGACCTCTCGCCTGCTCCT | 3064 | ACCTGGCAACTGGCGCACAA | 4189 | AGCACTATTGACCTTTTGGG |
| 1941 | GCAGACCTCTCGCCTGCTCC | 3065 | CACCTGGCAACTGGCGCACA | 4190 | CAGCACTATTGACCTTTTGG |
| 1942 | CGCAGACCTCTCGCCTGCTC | 3066 | ACACCTGGCAACTGGCGCAC | 4191 | TCAGCACTATTGACCTTTTG |
| 1943 | GCGCAGACCTCTCGCCTGCT | 3067 | TACACCTGGCAACTGGCGCA | 4192 | CTCAGCACTATTGACCTTTT |
| 1944 | CGCGCAGACCTCTCGCCTGC | 3068 | CTACACCTGGCAACTGGCGC | 4193 | TCTCAGCACTATTGACCTTT |
| 1945 | CCGCGCAGACCTCTCGCCTG | 3069 | CCTACACCTGGCAACTGGCG | 4194 | GTCTCAGCACTATTGACCTT |
| 1946 | GCCGCGCAGACCTCTCGCCT | 3070 | CCCTACACCTGGCAACTGGC | 4195 | AGTCTCAGCACTATTGACCT |
| 1947 | GGCCGCGCAGACCTCTCGCC | 3071 | TCCCTACACCTGGCAACTGG | 4196 | GAGTCTCAGCACTATTGACC |
| 1948 | CGGCCGCGCAGACCTCTCGC | 3072 | CTCCCTACACCTGGCAACTG | 4197 | TGAGTCTCAGCACTATTGAC |
| 1949 | GCGGCCGCGCAGACCTCTCG | 3073 | TCTCCCTACACCTGGCAACT | 4198 | TTGAGTCTCAGCACTATTGA |
| 1950 | AGCGGCCGCGCAGACCTCTC | 3074 | CTCTCCCTACACCTGGCAAC | 4199 | GTTGAGTCTCAGCACTATTG |
| 1951 | GAGCGGCCGCGCAGACCTCT | 3075 | CCTCTCCCTACACCTGGCAA | 4200 | TGTTGAGTCTCAGCACTATT |
| 1952 | AGAGCGGCCGCGCAGACCTC | 3076 | TCCTCTCCCTACACCTGGCA | 4201 | TTGTTGAGTCTCAGCACTAT |
| 1953 | GAGAGCGGCCGCGCAGACCT | 3077 | TTCCTCTCCCTACACCTGGC | 4202 | GTTGTTGAGTCTCAGCACTA |
| 1954 | GGAGAGCGGCCGCGCAGACC | 3078 | CTTCCTCTCCCTACACCTGG | 4203 | GGTTGTTGAGTCTCAGCACT |
| 1955 | AGGAGAGCGGCCGCGCAGAC | 3079 | ACTTCCTCTCCCTACACCTG | 4204 | AGGTTGTTGAGTCTCAGCAC |
| 1956 | TAGGAGAGCGGCCGCGCAGA | 3080 | TACTTCCTCTCCCTACACCT | 4205 | TAGGTTGTTGAGTCTCAGCA |
| 1957 | GTAGGAGAGCGGCCGCGCAG | 3081 | CTACTTCCTCTCCCTACACC | 4206 | GTAGGTTGTTGAGTCTCAGC |
| 1958 | GGTAGGAGAGCGGCCGCGCA | 3082 | TCTACTTCCTCTCCCTACAC | 4207 | AGTAGGTTGTTGAGTCTCAG |
| 1959 | AGGTAGGAGAGCGGCCGCGC | 3083 | ATCTACTTCCTCTCCCTACA | 4208 | AAGTAGGTTGTTGAGTCTCA |
| 1960 | CAGGTAGGAGAGCGGCCGCG | 3084 | AATCTACTTCCTCTCCCTAC | 4209 | GAAGTAGGTTGTTGAGTCTC |
| 1961 | GCAGGTAGGAGAGCGGCCGC | 3085 | CAATCTACTTCCTCTCCCTA | 4210 | TGAAGTAGGTTGTTGAGTCT |
| 1962 | CGCAGGTAGGAGAGCGGCCG | 3086 | ACAATCTACTTCCTCTCCCT | 4211 | GTGAAGTAGGTTGTTGAGTC |
| 1963 | ACGCAGGTAGGAGAGCGGCC | 3087 | GACAATCTACTTCCTCTCCC | 4212 | TGTGAAGTAGGTTGTTGAGT |
| 1964 | GACGCAGGTAGGAGAGCGGC | 3088 | AGACAATCTACTTCCTCTCC | 4213 | TTGTGAAGTAGGTTGTTGAG |
| 1965 | GGACGCAGGTAGGAGAGCGG | 3089 | GAGACAATCTACTTCCTCTC | 4214 | TTTGTGAAGTAGGTTGTTGA |
| 1966 | CGGACGCAGGTAGGAGAGCG | 3090 | GGAGACAATCTACTTCCTCT | 4215 | ATTTGTGAAGTAGGTTGTTG |
| 1967 | TCGGACGCAGGTAGGAGAGC | 3091 | TGGAGACAATCTACTTCCTC | 4216 | CATTTGTGAAGTAGGTTGTT |
| 1968 | GTCGGACGCAGGTAGGAGAG | 3092 | CTGGAGACAATCTACTTCCT | 4217 | ACATTTGTGAAGTAGGTTGT |
| 1969 | AGTCGGACGCAGGTAGGAGA | 3093 | CCTGGAGACAATCTACTTCC | 4218 | AACATTTGTGAAGTAGGTTG |
| 1970 | GAGTCGGACGCAGGTAGGAG | 3094 | ACCTGGAGACAATCTACTTC | 4219 | AAACATTTGTGAAGTAGGTT |
| 1971 | GGAGTCGGACGCAGGTAGGA | 3095 | CACCTGGAGACAATCTACTT | 4220 | GAAACATTTGTGAAGTAGGT |
| 1972 | CGGAGTCGGACGCAGGTAGG | 3096 | CCACCTGGAGACAATCTACT | 4221 | AGAAACATTTGTGAAGTAGG |
| 1973 | GCGGAGTCGGACGCAGGTAG | 3097 | CCCACCTGGAGACAATCTAC | 4222 | GAGAAACATTTGTGAAGTAG |
| 1974 | CGCGGAGTCGGACGCAGGTA | 3098 | CCCCACCTGGAGACAATCTA | 4223 | AGAGAAACATTTGTGAAGTA |
| 1975 | CCGCGGAGTCGGACGCAGGT | 3099 | TCCCCACCTGGAGACAATCT | 4224 | GAGAGAAACATTTGTGAAGT |
| 1976 | ACCGCGGAGTCGGACGCAGG | 3100 | TTCCCCACCTGGAGACAATC | 4225 | AGAGAGAAACATTTGTGAAG |
| 1977 | GACCGCGGAGTCGGACGCAG | 3101 | CTTCCCCACCTGGAGACAAT | 4226 | GAGAGAGAAACATTTGTGAA |
| 1978 | GGACCGCGGAGTCGGACGCA | 3102 | TCTTCCCCACCTGGAGACAA | 4227 | AGAGAGAGAAACATTTGTGA |
| 1979 | AGGACCGCGGAGTCGGACGC | 3103 | TTCTTCCCCACCTGGAGACA | 4228 | GAGAGAGAGAAACATTTGTG |
| 1980 | AAGGACCGCGGAGTCGGACG | 3104 | CTTCTTCCCCACCTGGAGAC | 4229 | AGAGAGAGAGAAACATTTGT |
| 1981 | CAAGGACCGCGGAGTCGGAC | 3105 | TCTTCTTCCCCACCTGGAGA | 4230 | GAGAGAGAGAGAAACATTTG |
| 1982 | CCAAGGACCGCGGAGTCGGA | 3106 | CTCTTCTTCCCCACCTGGAG | 4231 | AGAGAGAGAGAGAAACATTT |
| 1983 | CCCAAGGACCGCGGAGTCGG | 3107 | TCTCTTCTTCCCCACCTGGA | 4232 | GAGAGAGAGAGAGAAACATT |
| 1984 | GCCCAAGGACCGCGGAGTCG | 3108 | ATCTCTTCTTCCCCACCTGG | 4233 | AGAGAGAGAGAGAGAAACAT |
| 1985 | TGCCCAAGGACCGCGGAGTC | 3109 | AATCTCTTCTTCCCCACCTG | 4234 | GAGAGAGAGAGAGAGAAACA |
| 1986 | CTGCCCAAGGACCGCGGAGT | 3110 | AAATCTCTTCTTCCCCACCT | 4235 | CGAGAGAGAGAGAGAGAAAC |
| 1987 | GCTGCCCAAGGACCGCGGAG | 3111 | GAAATCTCTTCTTCCCCACC | 4236 | GCGAGAGAGAGAGAGAGAAA |
| 1988 | TGCTGCCCAAGGACCGCGGA | 3112 | TGAAATCTCTTCTTCCCCAC | 4237 | AGCGAGAGAGAGAGAGAGAA |
| 1989 | CTGCTGCCCAAGGACCGCGG | 3113 | ATGAAATCTCTTCTTCCCCA | 4238 | GAGCGAGAGAGAGAGAGAGA |
| 1990 | GCTGCTGCCCAAGGACCGCG | 3114 | AATGAAATCTCTTCTTCCCC | 4239 | AGAGCGAGAGAGAGAGAGAG |
| 1991 | TGCTGCTGCCCAAGGACCGC | 3115 | GAATGAAATCTCTTCTTCCC | 4240 | AAGAGCGAGAGAGAGAGAGA |
| 1992 | TTGCTGCTGCCCAAGGACCG | 3116 | GGAATGAAATCTCTTCTTCC | 4241 | AAAGAGCGAGAGAGAGAGAG |
| 1993 | GTTGCTGCTGCCCAAGGACC | 3117 | TGGAATGAAATCTCTTCTTC | 4242 | AAAAGAGCGAGAGAGAGAGA |
| 1994 | GGTTGCTGCTGCCCAAGGAC | 3118 | GTGGAATGAAATCTCTTCTT | 4243 | AAAAAGAGCGAGAGAGAGAG |
| 1995 | CGGTTGCTGCTGCCCAAGGA | 3119 | GGTGGAATGAAATCTCTTCT | 4244 | AAAAAAGAGCGAGAGAGAGA |
| 1996 | CCGGTTGCTGCTGCCCAAGG | 3120 | AGGTGGAATGAAATCTCTTC | 4245 | AAAAAAAGAGCGAGAGAGAG |
| 1997 | CCCGGTTGCTGCTGCCCAAG | 3121 | AAGGTGGAATGAAATCTCTT | 4246 | AAAAAAAAGAGCGAGAGAGA |
| 1998 | ACCCGGTTGCTGCTGCCCAA | 3122 | TAAGGTGGAATGAAATCTCT | 4247 | AAAAAAAAAGAGCGAGAGAG |
| 1999 | TACCCGGTTGCTGCTGCCCA | 3123 | CTAAGGTGGAATGAAATCTC | 4248 | AAAAAAAAAAGAGCGAGAGA |
| 2000 | CTACCCGGTTGCTGCTGCCC | 3124 | CCTAAGGTGGAATGAAATCT | 4249 | AAAAAAAAAAAGAGCGAGAG |
| 2001 | GCTACCCGGTTGCTGCTGCC | 3125 | TCCTAAGGTGGAATGAAATC | 4250 | AAAAAAAAAAAAGAGCGAGA |
| 2002 | CGCTACCCGGTTGCTGCTGC | 3126 | CTCCTAAGGTGGAATGAAAT | 4251 | AAAAAAAAAAAAAGAGCGAG |
| 2003 | GCGCTACCCGGTTGCTGCTG | 3127 | GCTCCTAAGGTGGAATGAAA | 4252 | AAAAAAAAAAAAAAGAGCGA |
| 2004 | AGCGCTACCCGGTTGCTGCT | 3128 | TGCTCCTAAGGTGGAATGAA | 4253 | AAAAAAAAAAAAAAAGAGCG |
| 2005 | GAGCGCTACCCGGTTGCTGC | 3129 | ATGCTCCTAAGGTGGAATGA | 4254 | AAAAAAAAAAAAAAAAGAGC |
| 2006 | TGAGCGCTACCCGGTTGCTG | 3130 | GATGCTCCTAAGGTGGAATG | 4255 | AAAAAAAAAAAAAAAAAGAG |
| 2007 | CTGAGCGCTACCCGGTTGCT | 3131 | AGATGCTCCTAAGGTGGAAT | 4256 | TAAAAAAAAAAAAAAAAAGA |
| 2008 | TCTGAGCGCTACCCGGTTGC | 3132 | GAGATGCTCCTAAGGTGGAA | 4257 | TTAAAAAAAAAAAAAAAAAG |
| 2009 | GTCTGAGCGCTACCCGGTTG | 3133 | GGAGATGCTCCTAAGGTGGA | 4258 | GTTAAAAAAAAAAAAAAAAA |
| 2010 | AGTCTGAGCGCTACCCGGTT | 3134 | AGGAGATGCTCCTAAGGTGG | 4259 | TGTTAAAAAAAAAAAAAAAA |
| 2011 | TAGTCTGAGCGCTACCCGGT | 3135 | AAGGAGATGCTCCTAAGGTG | 4260 | ATGTTAAAAAAAAAAAAAAA |
| 2012 | GTAGTCTGAGCGCTACCCGG | 3136 | CAAGGAGATGCTCCTAAGGT | 4261 | GATGTTAAAAAAAAAAAAAA |
| 2013 | TGTAGTCTGAGCGCTACCCG | 3137 | TCAAGGAGATGCTCCTAAGG | 4262 | TGATGTTAAAAAAAAAAAAA |
| 2014 | CTGTAGTCTGAGCGCTACCC | 3138 | CTCAAGGAGATGCTCCTAAG | 4263 | CTGATGTTAAAAAAAAAAAA |
| 2015 | TCTGTAGTCTGAGCGCTACC | 3139 | GCTCAAGGAGATGCTCCTAA | 4264 | TCTGATGTTAAAAAAAAAAA |
| 2016 | GTCTGTAGTCTGAGCGCTAC | 3140 | AGCTCAAGGAGATGCTCCTA | 4265 | TTCTGATGTTAAAAAAAAAA |
| 2017 | GGTCTGTAGTCTGAGCGCTA | 3141 | CAGCTCAAGGAGATGCTCCT | 4266 | CTTCTGATGTTAAAAAAAAA |
| 2018 | GGGTCTGTAGTCTGAGCGCT | 3142 | TCAGCTCAAGGAGATGCTCC | 4267 | CCTTCTGATGTTAAAAAAAA |
| 2019 | GGGGTCTGTAGTCTGAGCGC | 3143 | CTCAGCTCAAGGAGATGCTC | 4268 | CCCTTCTGATGTTAAAAAAA |
| 2020 | TGGGGTCTGTAGTCTGAGCG | 3144 | ACTCAGCTCAAGGAGATGCT | 4269 | ACCCTTCTGATGTTAAAAAA |
| 2021 | CTGGGGTCTGTAGTCTGAGC | 3145 | GACTCAGCTCAAGGAGATGC | 4270 | TACCCTTCTGATGTTAAAAA |
| 2022 | GCTGGGGTCTGTAGTCTGAG | 3146 | AGACTCAGCTCAAGGAGATG | 4271 | TTACCCTTCTGATGTTAAAA |
| 2023 | CGCTGGGGTCTGTAGTCTGA | 3147 | CAGACTCAGCTCAAGGAGAT | 4272 | CTTACCCTTCTGATGTTAAA |
| 2024 | GCGCTGGGGTCTGTAGTCTG | 3148 | CCAGACTCAGCTCAAGGAGA | 4273 | CCTTACCCTTCTGATGTTAA |
| 2025 | CGCGCTGGGGTCTGTAGTCT | 3149 | CCCAGACTCAGCTCAAGGAG | 4274 | ACCTTACCCTTCTGATGTTA |
| 2026 | TCGCGCTGGGGTCTGTAGTC | 3150 | TCCCAGACTCAGCTCAAGGA | 4275 | CACCTTACCCTTCTGATGTT |
| 2027 | ATCGCGCTGGGGTCTGTAGT | 3151 | TTCCCAGACTCAGCTCAAGG | 4276 | GCACCTTACCCTTCTGATGT |
| 2028 | CATCGCGCTGGGGTCTGTAG | 3152 | CTTCCCAGACTCAGCTCAAG | 4277 | GGCACCTTACCCTTCTGATG |
| 2029 | TCATCGCGCTGGGGTCTGTA | 3153 | TCTTCCCAGACTCAGCTCAA | 4278 | TGGCACCTTACCCTTCTGAT |
| 2030 | GTCATCGCGCTGGGGTCTGT | 3154 | ATCTTCCCAGACTCAGCTCA | 4279 | GTGGCACCTTACCCTTCTGA |
| 2031 | GGTCATCGCGCTGGGGTCTG | 3155 | AATCTTCCCAGACTCAGCTC | 4280 | CGTGGCACCTTACCCTTCTG |
| 2032 | GGGTCATCGCGCTGGGGTCT | 3156 | AAATCTTCCCAGACTCAGCT | 4281 | TCGTGGCACCTTACCCTTCT |
| 2033 | CGGGTCATCGCGCTGGGGTC | 3157 | AAAATCTTCCCAGACTCAGC | 4282 | ATCGTGGCACCTTACCCTTC |
| 2034 | GCGGGTCATCGCGCTGGGGT | 3158 | TAAAATCTTCCCAGACTCAG | 4283 | TATCGTGGCACCTTACCCTT |
| 2035 | GGCGGGTCATCGCGCTGGGG | 3159 | TTAAAATCTTCCCAGACTCA | 4284 | TTATCGTGGCACCTTACCCT |
| 2036 | AGGCGGGTCATCGCGCTGGG | 3160 | GTTAAAATCTTCCCAGACTC | 4285 | GTTATCGTGGCACCTTACCC |
| 2037 | TAGGCGGGTCATCGCGCTGG | 3161 | AGTTAAAATCTTCCCAGACT | 4286 | CGTTATCGTGGCACCTTACC |
| 2038 | GTAGGCGGGTCATCGCGCTG | 3162 | CAGTTAAAATCTTCCCAGAC | 4287 | TCGTTATCGTGGCACCTTAC |
| 2039 | GGTAGGCGGGTCATCGCGCT | 3163 | CCAGTTAAAATCTTCCCAGA | 4288 | CTCGTTATCGTGGCACCTTA |
| 2040 | AGGTAGGCGGGTCATCGCGC | 3164 | GCCAGTTAAAATCTTCCCAG | 4289 | CCTCGTTATCGTGGCACCTT |
| 2041 | GAGGTAGGCGGGTCATCGCG | 3165 | TGCCAGTTAAAATCTTCCCA | 4290 | ACCTCGTTATCGTGGCACCT |
| 2042 | TGAGGTAGGCGGGTCATCGC | 3166 | CTGCCAGTTAAAATCTTCCC | 4291 | GACCTCGTTATCGTGGCACC |
| 2043 | CTGAGGTAGGCGGGTCATCG | 3167 | CCTGCCAGTTAAAATCTTCC | 4292 | AGACCTCGTTATCGTGGCAC |
| 2044 | ACTGAGGTAGGCGGGTCATC | 3168 | ACCTGCCAGTTAAAATCTTC | 4293 | CAGACCTCGTTATCGTGGCA |
| 2045 | AACTGAGGTAGGCGGGTCAT | 3169 | CACCTGCCAGTTAAAATCTT | 4294 | ACAGACCTCGTTATCGTGGC |
| 2046 | AAACTGAGGTAGGCGGGTCA | 3170 | CCACCTGCCAGTTAAAATCT | 4295 | TACAGACCTCGTTATCGTGG |
| 2047 | GAAACTGAGGTAGGCGGGTC | 3171 | TCCACCTGCCAGTTAAAATC | 4296 | TTACAGACCTCGTTATCGTG |
| 2048 | GGAAACTGAGGTAGGCGGGT | 3172 | CTCCACCTGCCAGTTAAAAT | 4297 | ATTACAGACCTCGTTATCGT |
| 2049 | TGGAAACTGAGGTAGGCGGG | 3173 | CCTCCACCTGCCAGTTAAAA | 4298 | AATTACAGACCTCGTTATCG |
| 2050 | ATGGAAACTGAGGTAGGCGG | 3174 | TCCTCCACCTGCCAGTTAAA | 4299 | GAATTACAGACCTCGTTATC |
| 2051 | AATGGAAACTGAGGTAGGCG | 3175 | CTCCTCCACCTGCCAGTTAA | 4300 | GGAATTACAGACCTCGTTAT |
| 2052 | CAATGGAAACTGAGGTAGGC | 3176 | CCTCCTCCACCTGCCAGTTA | 4301 | GGGAATTACAGACCTCGTTA |
| 2053 | CCAATGGAAACTGAGGTAGG | 3177 | TCCTCCTCCACCTGCCAGTT | 4302 | AGGGAATTACAGACCTCGTT |
| 2054 | GCCAATGGAAACTGAGGTAG | 3178 | CTCCTCCTCCACCTGCCAGT | 4303 | TAGGGAATTACAGACCTCGT |
| 2055 | GGCCAATGGAAACTGAGGTA | 3179 | CCTCCTCCTCCACCTGCCAG | 4304 | GTAGGGAATTACAGACCTCG |
| 2056 | TGGCCAATGGAAACTGAGGT | 3180 | ACCTCCTCCTCCACCTGCCA | 4305 | AGTAGGGAATTACAGACCTC |
| 2057 | CTGGCCAATGGAAACTGAGG | 3181 | CACCTCCTCCTCCACCTGCC | 4306 | GAGTAGGGAATTACAGACCT |
| 2058 | CCTGGCCAATGGAAACTGAG | 3182 | ACACCTCCTCCTCCACCTGC | 4307 | TGAGTAGGGAATTACAGACC |
| 2059 | CCCTGGCCAATGGAAACTGA | 3183 | AACACCTCCTCCTCCACCTG | 4308 | CTGAGTAGGGAATTACAGAC |
| 2060 | TCCCTGGCCAATGGAAACTG | 3184 | CAACACCTCCTCCTCCACCT | 4309 | CCTGAGTAGGGAATTACAGA |
| 2061 | ATCCCTGGCCAATGGAAACT | 3185 | CCAACACCTCCTCCTCCACC | 4310 | TCCTGAGTAGGGAATTACAG |
| 2062 | GATCCCTGGCCAATGGAAAC | 3186 | CCCAACACCTCCTCCTCCAC | 4311 | CTCCTGAGTAGGGAATTACA |
| 2063 | TGATCCCTGGCCAATGGAAA | 3187 | TCCCAACACCTCCTCCTCCA | 4312 | CCTCCTGAGTAGGGAATTAC |
| 2064 | CTGATCCCTGGCCAATGGAA | 3188 | TTCCCAACACCTCCTCCTCC | 4313 | GCCTCCTGAGTAGGGAATTA |
| 2065 | CCTGATCCCTGGCCAATGGA | 3189 | CTTCCCAACACCTCCTCCTC | 4314 | GGCCTCCTGAGTAGGGAATT |
| 2066 | CCCTGATCCCTGGCCAATGG | 3190 | TCTTCCCAACACCTCCTCCT | 4315 | CGGCCTCCTGAGTAGGGAAT |
| 2067 | GCCCTGATCCCTGGCCAATG | 3191 | CTCTTCCCAACACCTCCTCC | 4316 | TCGGCCTCCTGAGTAGGGAA |
| 2068 | AGCCCTGATCCCTGGCCAAT | 3192 | GCTCTTCCCAACACCTCCTC | 4317 | CTCGGCCTCCTGAGTAGGGA |
| 2069 | TAGCCCTGATCCCTGGCCAA | 3193 | GGCTCTTCCCAACACCTCCT | 4318 | CCTCGGCCTCCTGAGTAGGG |
| 2070 | GTAGCCCTGATCCCTGGCCA | 3194 | GGGCTCTTCCCAACACCTCC | 4319 | GCCTCGGCCTCCTGAGTAGG |
| 2071 | GGTAGCCCTGATCCCTGGCC | 3195 | AGGGCTCTTCCCAACACCTC | 4320 | TGCCTCGGCCTCCTGAGTAG |
| 2072 | GGGTAGCCCTGATCCCTGGC | 3196 | CAGGGCTCTTCCCAACACCT | 4321 | CTGCCTCGGCCTCCTGAGTA |
| 2073 | CGGGTAGCCCTGATCCCTGG | 3197 | CCAGGGCTCTTCCCAACACC | 4322 | CCTGCCTCGGCCTCCTGAGT |
| 2074 | GCGGGTAGCCCTGATCCCTG | 3198 | CCCAGGGCTCTTCCCAACAC | 4323 | TCCTGCCTCGGCCTCCTGAG |
| 2075 | AGCGGGTAGCCCTGATCCCT | 3199 | TCCCAGGGCTCTTCCCAACA | 4324 | CTCCTGCCTCGGCCTCCTGA |
| 2076 | GAGCGGGTAGCCCTGATCCC | 3200 | TTCCCAGGGCTCTTCCCAAC | 4325 | TCTCCTGCCTCGGCCTCCTG |
| 2077 | GGAGCGGGTAGCCCTGATCC | 3201 | TTTCCCAGGGCTCTTCCCAA | 4326 | TTCTCCTGCCTCGGCCTCCT |
| 2078 | GGGAGCGGGTAGCCCTGATC | 3202 | GTTTCCCAGGGCTCTTCCCA | 4327 | ATTCTCCTGCCTCGGCCTCC |
| 2079 | TGGGAGCGGGTAGCCCTGAT | 3203 | GGTTTCCCAGGGCTCTTCCC | 4328 | GATTCTCCTGCCTCGGCCTC |
| 2080 | ATGGGAGCGGGTAGCCCTGA | 3204 | TGGTTTCCCAGGGCTCTTCC | 4329 | CGATTCTCCTGCCTCGGCCT |
| 2081 | AATGGGAGCGGGTAGCCCTG | 3205 | CTGGTTTCCCAGGGCTCTTC | 4330 | GCGATTCTCCTGCCTCGGCC |
| 2082 | CAATGGGAGCGGGTAGCCCT | 3206 | TCTGGTTTCCCAGGGCTCTT | 4331 | AGCGATTCTCCTGCCTCGGC |
| 2083 | CCAATGGGAGCGGGTAGCCC | 3207 | ATCTGGTTTCCCAGGGCTCT | 4332 | AAGCGATTCTCCTGCCTCGG |
| 2084 | GCCAATGGGAGCGGGTAGCC | 3208 | CATCTGGTTTCCCAGGGCTC | 4333 | CAAGCGATTCTCCTGCCTCG |
| 2085 | AGCCAATGGGAGCGGGTAGC | 3209 | CCATCTGGTTTCCCAGGGCT | 4334 | TCAAGCGATTCTCCTGCCTC |
| 2086 | TAGCCAATGGGAGCGGGTAG | 3210 | CCCATCTGGTTTCCCAGGGC | 4335 | TTCAAGCGATTCTCCTGCCT |
| 2087 | GTAGCCAATGGGAGCGGGTA | 3211 | TCCCATCTGGTTTCCCAGGG | 4336 | GTTCAAGCGATTCTCCTGCC |
| 2088 | AGTAGCCAATGGGAGCGGGT | 3212 | TTCCCATCTGGTTTCCCAGG | 4337 | GGTTCAAGCGATTCTCCTGC |
| 2089 | AAGTAGCCAATGGGAGCGGG | 3213 | CTTCCCATCTGGTTTCCCAG | 4338 | GGGTTCAAGCGATTCTCCTG |
| 2090 | TAAGTAGCCAATGGGAGCGG | 3214 | CCTTCCCATCTGGTTTCCCA | 4339 | CGGGTTCAAGCGATTCTCCT |
| 2091 | ATAAGTAGCCAATGGGAGCG | 3215 | CCCTTCCCATCTGGTTTCCC | 4340 | CCGGGTTCAAGCGATTCTCC |
| 2092 | GATAAGTAGCCAATGGGAGC | 3216 | GCCCTTCCCATCTGGTTTCC | 4341 | CCCGGGTTCAAGCGATTCTC |
| 2093 | TGATAAGTAGCCAATGGGAG | 3217 | TGCCCTTCCCATCTGGTTTC | 4342 | TCCCGGGTTCAAGCGATTCT |
| 2094 | TTGATAAGTAGCCAATGGGA | 3218 | CTGCCCTTCCCATCTGGTTT | 4343 | ATCCCGGGTTCAAGCGATTC |
| 2095 | ATTGATAAGTAGCCAATGGG | 3219 | CCTGCCCTTCCCATCTGGTT | 4344 | CATCCCGGGTTCAAGCGATT |
| 2096 | TATTGATAAGTAGCCAATGG | 3220 | GCCTGCCCTTCCCATCTGGT | 4345 | GCATCCCGGGTTCAAGCGAT |
| 2097 | ATATTGATAAGTAGCCAATG | 3221 | AGCCTGCCCTTCCCATCTGG | 4346 | TGCATCCCGGGTTCAAGCGA |
| 2098 | TATATTGATAAGTAGCCAAT | 3222 | AAGCCTGCCCTTCCCATCTG | 4347 | CTGCATCCCGGGTTCAAGCG |
| 2099 | CTATATTGATAAGTAGCCAA | 3223 | CAAGCCTGCCCTTCCCATCT | 4348 | TCTGCATCCCGGGTTCAAGC |
| 2100 | TCTATATTGATAAGTAGCCA | 3224 | CCAAGCCTGCCCTTCCCATC | 4349 | CTCTGCATCCCGGGTTCAAG |
| 2101 | CTCTATATTGATAAGTAGCC | 3225 | GCCAAGCCTGCCCTTCCCAT | 4350 | CCTCTGCATCCCGGGTTCAA |
| 2102 | CCTCTATATTGATAAGTAGC | 3226 | TGCCAAGCCTGCCCTTCCCA | 4351 | ACCTCTGCATCCCGGGTTCA |
| 2103 | ACCTCTATATTGATAAGTAG | 3227 | CTGCCAAGCCTGCCCTTCCC | 4352 | AACCTCTGCATCCCGGGTTC |
| 2104 | CACCTCTATATTGATAAGTA | 3228 | CCTGCCAAGCCTGCCCTTCC | 4353 | CAACCTCTGCATCCCGGGTT |
| 2105 | CCACCTCTATATTGATAAGT | 3229 | CCCTGCCAAGCCTGCCCTTC | 4354 | GCAACCTCTGCATCCCGGGT |
| 2106 | CCCACCTCTATATTGATAAG | 3230 | TCCCTGCCAAGCCTGCCCTT | 4355 | TGCAACCTCTGCATCCCGGG |
| 2107 | CCCCACCTCTATATTGATAA | 3231 | ATCCCTGCCAAGCCTGCCCT | 4356 | CTGCAACCTCTGCATCCCGG |
| 2108 | GCCCCACCTCTATATTGATA | 3232 | AATCCCTGCCAAGCCTGCCC | 4357 | ACTGCAACCTCTGCATCCCG |
| 2109 | GGCCCCACCTCTATATTGAT | 3233 | CAATCCCTGCCAAGCCTGCC | 4358 | TACTGCAACCTCTGCATCCC |
| 2110 | TGGCCCCACCTCTATATTGA | 3234 | TCAATCCCTGCCAAGCCTGC | 4359 | TTACTGCAACCTCTGCATCC |
| 2111 | CTGGCCCCACCTCTATATTG | 3235 | TTCAATCCCTGCCAAGCCTG | 4360 | TTTACTGCAACCTCTGCATC |
| 2112 | GCTGGCCCCACCTCTATATT | 3236 | ATTCAATCCCTGCCAAGCCT | 4361 | GTTTACTGCAACCTCTGCAT |
| 2113 | GGCTGGCCCCACCTCTATAT | 3237 | CATTCAATCCCTGCCAAGCC | 4362 | GGTTTACTGCAACCTCTGCA |
| 2114 | AGGCTGGCCCCACCTCTATA | 3238 | GCATTCAATCCCTGCCAAGC | 4363 | CGGTTTACTGCAACCTCTGC |
| 2115 | CAGGCTGGCCCCACCTCTAT | 3239 | GGCATTCAATCCCTGCCAAG | 4364 | TCGGTTTACTGCAACCTCTG |
| 2116 | CCAGGCTGGCCCCACCTCTA | 3240 | TGGCATTCAATCCCTGCCAA | 4365 | CTCGGTTTACTGCAACCTCT |
| 2117 | TCCAGGCTGGCCCCACCTCT | 3241 | ATGGCATTCAATCCCTGCCA | 4366 | TCTCGGTTTACTGCAACCTC |
| 2118 | TTCCAGGCTGGCCCCACCTC | 3242 | CATGGCATTCAATCCCTGCC | 4367 | ATCTCGGTTTACTGCAACCT |
| 2119 | ATTCCAGGCTGGCCCCACCT | 3243 | ACATGGCATTCAATCCCTGC | 4368 | GATCTCGGTTTACTGCAACC |
| 2120 | CATTCCAGGCTGGCCCCACC | 3244 | AACATGGCATTCAATCCCTG | 4369 | CGATCTCGGTTTACTGCAAC |
| 2121 | GCATTCCAGGCTGGCCCCAC | 3245 | GAACATGGCATTCAATCCCT | 4370 | ACGATCTCGGTTTACTGCAA |
| 2122 | AGCATTCCAGGCTGGCCCCA | 3246 | TGAACATGGCATTCAATCCC | 4371 | CACGATCTCGGTTTACTGCA |
| 2123 | CAGCATTCCAGGCTGGCCCC | 3247 | TTGAACATGGCATTCAATCC | 4372 | GCACGATCTCGGTTTACTGC |
| 2124 | ACAGCATTCCAGGCTGGCCC | 3248 | ATTGAACATGGCATTCAATC | 4373 | GGCACGATCTCGGTTTACTG |
| 2125 | CACAGCATTCCAGGCTGGCC | 3249 | CATTGAACATGGCATTCAAT | 4374 | TGGCACGATCTCGGTTTACT |
| 2126 | ACACAGCATTCCAGGCTGGC | 3250 | GCATTGAACATGGCATTCAA | 4375 | GTGGCACGATCTCGGTTTAC |
| 2127 | AACACAGCATTCCAGGCTGG | 3251 | GGCATTGAACATGGCATTCA | 4376 | AGTGGCACGATCTCGGTTTA |
| 2128 | AAACACAGCATTCCAGGCTG | 3252 | TGGCATTGAACATGGCATTC | 4377 | CAGTGGCACGATCTCGGTTT |
| 2129 | GAAACACAGCATTCCAGGCT | 3253 | CTGGCATTGAACATGGCATT | 4378 | GCAGTGGCACGATCTCGGTT |
| 2130 | AGAAACACAGCATTCCAGGC | 3254 | ACTGGCATTGAACATGGCAT | 4379 | TGCAGTGGCACGATCTCGGT |
| 2131 | AAGAAACACAGCATTCCAGG | 3255 | CACTGGCATTGAACATGGCA | 4380 | ATGCAGTGGCACGATCTCGG |
| 2132 | AAAGAAACACAGCATTCCAG | 3256 | CCACTGGCATTGAACATGGC | 4381 | AATGCAGTGGCACGATCTCG |
| 2133 | AAAAGAAACACAGCATTCCA | 3257 | CCCACTGGCATTGAACATGG | 4382 | GAATGCAGTGGCACGATCTC |
| 2134 | GAAAAGAAACACAGCATTCC | 3258 | GCCCACTGGCATTGAACATG | 4383 | GGAATGCAGTGGCACGATCT |
| 2135 | AGAAAAGAAACACAGCATTC | 3259 | AGCCCACTGGCATTGAACAT | 4384 | TGGAATGCAGTGGCACGATC |
| 2136 | TAGAAAAGAAACACAGCATT | 3260 | AAGCCCACTGGCATTGAACA | 4385 | CTGGAATGCAGTGGCACGAT |
| 2137 | GTAGAAAAGAAACACAGCAT | 3261 | GAAGCCCACTGGCATTGAAC | 4386 | GCTGGAATGCAGTGGCACGA |
| 2138 | AGTAGAAAAGAAACACAGCA | 3262 | TGAAGCCCACTGGCATTGAA | 4387 | GGCTGGAATGCAGTGGCACG |
| 2139 | GAGTAGAAAAGAAACACAGC | 3263 | CTGAAGCCCACTGGCATTGA | 4388 | AGGCTGGAATGCAGTGGCAC |
| 2140 | TGAGTAGAAAAGAAACACAG | 3264 | CCTGAAGCCCACTGGCATTG | 4389 | CAGGCTGGAATGCAGTGGCA |
| 2141 | TTGAGTAGAAAAGAAACACA | 3265 | TCCTGAAGCCCACTGGCATT | 4390 | CCAGGCTGGAATGCAGTGGC |
| 2142 | TTTGAGTAGAAAAGAAACAC | 3266 | CTCCTGAAGCCCACTGGCAT | 4391 | TCCAGGCTGGAATGCAGTGG |
| 2143 | ATTTGAGTAGAAAAGAAACA | 3267 | GCTCCTGAAGCCCACTGGCA | 4392 | GTCCAGGCTGGAATGCAGTG |
| 2144 | GATTTGAGTAGAAAAGAAAC | 3268 | AGCTCCTGAAGCCCACTGGC | 4393 | TGTCCAGGCTGGAATGCAGT |
| 2145 | TGATTTGAGTAGAAAAGAAA | 3269 | CAGCTCCTGAAGCCCACTGG | 4394 | TTGTCCAGGCTGGAATGCAG |
| 2146 | GTGATTTGAGTAGAAAAGAA | 3270 | GCAGCTCCTGAAGCCCACTG | 4395 | GTTGTCCAGGCTGGAATGCA |
| 2147 | AGTGATTTGAGTAGAAAAGA | 3271 | AGCAGCTCCTGAAGCCCACT | 4396 | TGTTGTCCAGGCTGGAATGC |
| 2148 | GAGTGATTTGAGTAGAAAAG | 3272 | CAGCAGCTCCTGAAGCCCAC | 4397 | CTGTTGTCCAGGCTGGAATG |
| 2149 | AGAGTGATTTGAGTAGAAAA | 3273 | CCAGCAGCTCCTGAAGCCCA | 4398 | TCTGTTGTCCAGGCTGGAAT |
| 2150 | GAGAGTGATTTGAGTAGAAA | 3274 | CCCAGCAGCTCCTGAAGCCC | 4399 | CTCTGTTGTCCAGGCTGGAA |
| 2151 | GGAGAGTGATTTGAGTAGAA | 3275 | ACCCAGCAGCTCCTGAAGCC | 4400 | GCTCTGTTGTCCAGGCTGGA |
| 2152 | AGGAGAGTGATTTGAGTAGA | 3276 | CACCCAGCAGCTCCTGAAGC | 4401 | CGCTCTGTTGTCCAGGCTGG |
| 2153 | GAGGAGAGTGATTTGAGTAG | 3277 | GCACCCAGCAGCTCCTGAAG | 4402 | TCGCTCTGTTGTCCAGGCTG |
| 2154 | GGAGGAGAGTGATTTGAGTA | 3278 | GGCACCCAGCAGCTCCTGAA | 4403 | CTCGCTCTGTTGTCCAGGCT |
| 2155 | GGGAGGAGAGTGATTTGAGT | 3279 | AGGCACCCAGCAGCTCCTGA | 4404 | TCTCGCTCTGTTGTCCAGGC |
| 2156 | AGGGAGGAGAGTGATTTGAG | 3280 | CAGGCACCCAGCAGCTCCTG | 4405 | GTCTCGCTCTGTTGTCCAGG |
| 2157 | CAGGGAGGAGAGTGATTTGA | 3281 | CCAGGCACCCAGCAGCTCCT | 4406 | AGTCTCGCTCTGTTGTCCAG |
| 2158 | GCAGGGAGGAGAGTGATTTG | 3282 | GCCAGGCACCCAGCAGCTCC | 4407 | GAGTCTCGCTCTGTTGTCCA |
| 2159 | AGCAGGGAGGAGAGTGATTT | 3283 | TGCCAGGCACCCAGCAGCTC | 4408 | GGAGTCTCGCTCTGTTGTCC |
| 2160 | AAGCAGGGAGGAGAGTGATT | 3284 | GTGCCAGGCACCCAGCAGCT | 4409 | TGGAGTCTCGCTCTGTTGTC |
| 2161 | GAAGCAGGGAGGAGAGTGAT | 3285 | CGTGCCAGGCACCCAGCAGC | 4410 | ATGGAGTCTCGCTCTGTTGT |
| 2162 | GGAAGCAGGGAGGAGAGTGA | 3286 | CCGTGCCAGGCACCCAGCAG | 4411 | GATGGAGTCTCGCTCTGTTG |
| 2163 | AGGAAGCAGGGAGGAGAGTG | 3287 | CCCGTGCCAGGCACCCAGCA | 4412 | GGATGGAGTCTCGCTCTGTT |
| 2164 | GAGGAAGCAGGGAGGAGAGT | 3288 | GCCCGTGCCAGGCACCCAGC | 4413 | TGGATGGAGTCTCGCTCTGT |
| 2165 | AGAGGAAGCAGGGAGGAGAG | 3289 | AGCCCGTGCCAGGCACCCAG | 4414 | GTGGATGGAGTCTCGCTCTG |
| 2166 | GAGAGGAAGCAGGGAGGAGA | 3290 | CAGCCCGTGCCAGGCACCCA | 4415 | GGTGGATGGAGTCTCGCTCT |
| 2167 | CGAGAGGAAGCAGGGAGGAG | 3291 | CCAGCCCGTGCCAGGCACCC | 4416 | GGGTGGATGGAGTCTCGCTC |
| 2168 | GCGAGAGGAAGCAGGGAGGA | 3292 | CCCAGCCCGTGCCAGGCACC | 4417 | GGGGTGGATGGAGTCTCGCT |
| 2169 | GGCGAGAGGAAGCAGGGAGG | 3293 | GCCCAGCCCGTGCCAGGCAC | 4418 | GGGGGTGGATGGAGTCTCGC |
| 2170 | GGGCGAGAGGAAGCAGGGAG | 3294 | AGCCCAGCCCGTGCCAGGCA | 4419 | CGGGGGTGGATGGAGTCTCG |
| 2171 | GGGGCGAGAGGAAGCAGGGA | 3295 | CAGCCCAGCCCGTGCCAGGC | 4420 | GCGGGGGTGGATGGAGTCTC |
| 2172 | TGGGGCGAGAGGAAGCAGGG | 3296 | ACAGCCCAGCCCGTGCCAGG | 4421 | GGCGGGGGTGGATGGAGTCT |
| 2173 | GTGGGGCGAGAGGAAGCAGG | 3297 | CACAGCCCAGCCCGTGCCAG | 4422 | TGGCGGGGGTGGATGGAGTC |
| 2174 | TGTGGGGCGAGAGGAAGCAG | 3298 | CCACAGCCCAGCCCGTGCCA | 4423 | TTGGCGGGGGTGGATGGAGT |
| 2175 | GTGTGGGGCGAGAGGAAGCA | 3299 | ACCACAGCCCAGCCCGTGCC | 4424 | GTTGGCGGGGGTGGATGGAG |
| 2176 | GGTGTGGGGCGAGAGGAAGC | 3300 | CACCACAGCCCAGCCCGTGC | 4425 | TGTTGGCGGGGGTGGATGGA |
| 2177 | GGGTGTGGGGCGAGAGGAAG | 3301 | CCACCACAGCCCAGCCCGTG | 4426 | GTGTTGGCGGGGGTGGATGG |
| 2178 | GGGGTGTGGGGCGAGAGGAA | 3302 | CCCACCACAGCCCAGCCCGT | 4427 | TGTGTTGGCGGGGGTGGATG |
| 2179 | TGGGGTGTGGGGCGAGAGGA | 3303 | CCCCACCACAGCCCAGCCCG | 4428 | TTGTGTTGGCGGGGGTGGAT |
| 2180 | ATGGGGTGTGGGGCGAGAGG | 3304 | CCCCCACCACAGCCCAGCCC | 4429 | TTTGTGTTGGCGGGGGTGGA |
| 2181 | AATGGGGTGTGGGGCGAGAG | 3305 | GCCCCCACCACAGCCCAGCC | 4430 | TTTTGTGTTGGCGGGGGTGG |
| 2182 | AAATGGGGTGTGGGGCGAGA | 3306 | AGCCCCCACCACAGCCCAGC | 4431 | TTTTTGTGTTGGCGGGGGTG |
| 2183 | GAAATGGGGTGTGGGGCGAG | 3307 | CAGCCCCCACCACAGCCCAG | 4432 | TTTTTTGTGTTGGCGGGGGT |
| 2184 | AGAAATGGGGTGTGGGGCGA | 3308 | ACAGCCCCCACCACAGCCCA | 4433 | TTTTTTTGTGTTGGCGGGGG |
| 2185 | TAGAAATGGGGTGTGGGGCG | 3309 | GACAGCCCCCACCACAGCCC | 4434 | ATTTTTTTGTGTTGGCGGGG |
| 2186 | CTAGAAATGGGGTGTGGGGC | 3310 | CGACAGCCCCCACCACAGCC | 4435 | AATTTTTTTGTGTTGGCGGG |
| 2187 | CCTAGAAATGGGGTGTGGGG | 3311 | TCGACAGCCCCCACCACAGC | 4436 | TAATTTTTTTGTGTTGGCGG |
| 2188 | CCCTAGAAATGGGGTGTGGG | 3312 | TTCGACAGCCCCCACCACAG | 4437 | TTAATTTTTTTGTGTTGGCG |
| 2189 | TCCCTAGAAATGGGGTGTGG | 3313 | TTTCGACAGCCCCCACCACA | 4438 | TTTAATTTTTTTGTGTTGGC |
| 2190 | ATCCCTAGAAATGGGGTGTG | 3314 | CTTTCGACAGCCCCCACCAC | 4439 | CTTTAATTTTTTTGTGTTGG |
| 2191 | GATCCCTAGAAATGGGGTGT | 3315 | TCTTTCGACAGCCCCCACCA | 4440 | ACTTTAATTTTTTTGTGTTG |
| 2192 | TGATCCCTAGAAATGGGGTG | 3316 | CTCTTTCGACAGCCCCCACC | 4441 | CACTTTAATTTTTTTGTGTT |
| 2193 | ATGATCCCTAGAAATGGGGT | 3317 | TCTCTTTCGACAGCCCCCAC | 4442 | ACACTTTAATTTTTTTGTGT |
| 2194 | CATGATCCCTAGAAATGGGG | 3318 | TTCTCTTTCGACAGCCCCCA | 4443 | TACACTTTAATTTTTTTGTG |
| 2195 | ACATGATCCCTAGAAATGGG | 3319 | CTTCTCTTTCGACAGCCCCC | 4444 | GTACACTTTAATTTTTTTGT |
| 2196 | CACATGATCCCTAGAAATGG | 3320 | ACTTCTCTTTCGACAGCCCC | 4445 | TGTACACTTTAATTTTTTTG |
| 2197 | TCACATGATCCCTAGAAATG | 3321 | CACTTCTCTTTCGACAGCCC | 4446 | TTGTACACTTTAATTTTTTT |
| 2198 | ATCACATGATCCCTAGAAAT | 3322 | CCACTTCTCTTTCGACAGCC | 4447 | GTTGTACACTTTAATTTTTT |
| 2199 | AATCACATGATCCCTAGAAA | 3323 | GCCACTTCTCTTTCGACAGC | 4448 | AGTTGTACACTTTAATTTTT |
| 2200 | CAATCACATGATCCCTAGAA | 3324 | AGCCACTTCTCTTTCGACAG | 4449 | GAGTTGTACACTTTAATTTT |
| 2201 | CCAATCACATGATCCCTAGA | 3325 | AAGCCACTTCTCTTTCGACA | 4450 | TGAGTTGTACACTTTAATTT |
| 2202 | TCCAATCACATGATCCCTAG | 3326 | GAAGCCACTTCTCTTTCGAC | 4451 | TTGAGTTGTACACTTTAATT |
| 2203 | CTCCAATCACATGATCCCTA | 3327 | GGAAGCCACTTCTCTTTCGA | 4452 | ATTGAGTTGTACACTTTAAT |
| 2204 | TCTCCAATCACATGATCCCT | 3328 | AGGAAGCCACTTCTCTTTCG | 4453 | CATTGAGTTGTACACTTTAA |
| 2205 | TTCTCCAATCACATGATCCC | 3329 | TAGGAAGCCACTTCTCTTTC | 4454 | CCATTGAGTTGTACACTTTA |
| 2206 | ATTCTCCAATCACATGATCC | 3330 | CTAGGAAGCCACTTCTCTTT | 4455 | ACCATTGAGTTGTACACTTT |
| 2207 | GATTCTCCAATCACATGATC | 3331 | ACTAGGAAGCCACTTCTCTT | 4456 | AACCATTGAGTTGTACACTT |
| 2208 | TGATTCTCCAATCACATGAT | 3332 | GACTAGGAAGCCACTTCTCT | 4457 | AAACCATTGAGTTGTACACT |
| 2209 | ATGATTCTCCAATCACATGA | 3333 | AGACTAGGAAGCCACTTCTC | 4458 | AAAACCATTGAGTTGTACAC |
| 2210 | CATGATTCTCCAATCACATG | 3334 | GAGACTAGGAAGCCACTTCT | 4459 | AAAAACCATTGAGTTGTACA |
| 2211 | ACATGATTCTCCAATCACAT | 3335 | GGAGACTAGGAAGCCACTTC | 4460 | TAAAAACCATTGAGTTGTAC |
| 2212 | CACATGATTCTCCAATCACA | 3336 | TGGAGACTAGGAAGCCACTT | 4461 | CTAAAAACCATTGAGTTGTA |
| 2213 | TCACATGATTCTCCAATCAC | 3337 | CTGGAGACTAGGAAGCCACT | 4462 | ACTAAAAACCATTGAGTTGT |
| 2214 | GTCACATGATTCTCCAATCA | 3338 | TCTGGAGACTAGGAAGCCAC | 4463 | CACTAAAAACCATTGAGTTG |
| 2215 | CGTCACATGATTCTCCAATC | 3339 | ATCTGGAGACTAGGAAGCCA | 4464 | ACACTAAAAACCATTGAGTT |
| 2216 | ACGTCACATGATTCTCCAAT | 3340 | GATCTGGAGACTAGGAAGCC | 4465 | GACACTAAAAACCATTGAGT |
| 2217 | GACGTCACATGATTCTCCAA | 3341 | CGATCTGGAGACTAGGAAGC | 4466 | AGACACTAAAAACCATTGAG |
| 2218 | CGACGTCACATGATTCTCCA | 3342 | CCGATCTGGAGACTAGGAAG | 4467 | AAGACACTAAAAACCATTGA |
| 2219 | CCGACGTCACATGATTCTCC | 3343 | GCCGATCTGGAGACTAGGAA | 4468 | TAAGACACTAAAAACCATTG |
| 2220 | CCCGACGTCACATGATTCTC | 3344 | GGCCGATCTGGAGACTAGGA | 4469 | TTAAGACACTAAAAACCATT |
| 2221 | TCCCGACGTCACATGATTCT | 3345 | GGGCCGATCTGGAGACTAGG | 4470 | CTTAAGACACTAAAAACCAT |
| 2222 | GTCCCGACGTCACATGATTC | 3346 | AGGGCCGATCTGGAGACTAG | 4471 | TCTTAAGACACTAAAAACCA |
| 2223 | TGTCCCGACGTCACATGATT | 3347 | CAGGGCCGATCTGGAGACTA | 4472 | CTCTTAAGACACTAAAAACC |
| 2224 | CTGTCCCGACGTCACATGAT | 3348 | GCAGGGCCGATCTGGAGACT | 4473 | TCTCTTAAGACACTAAAAAC |
| 2225 | GCTGTCCCGACGTCACATGA | 3349 | GGCAGGGCCGATCTGGAGAC | 4474 | CTCTCTTAAGACACTAAAAA |
| 2226 | GGCTGTCCCGACGTCACATG | 3350 | AGGCAGGGCCGATCTGGAGA | 4475 | ACTCTCTTAAGACACTAAAA |
| 2227 | AGGCTGTCCCGACGTCACAT | 3351 | AAGGCAGGGCCGATCTGGAG | 4476 | AACTCTCTTAAGACACTAAA |
| 2228 | GAGGCTGTCCCGACGTCACA | 3352 | GAAGGCAGGGCCGATCTGGA | 4477 | TAACTCTCTTAAGACACTAA |
| 2229 | TGAGGCTGTCCCGACGTCAC | 3353 | CGAAGGCAGGGCCGATCTGG | 4478 | ATAACTCTCTTAAGACACTA |
| 2230 | CTGAGGCTGTCCCGACGTCA | 3354 | TCGAAGGCAGGGCCGATCTG | 4479 | CATAACTCTCTTAAGACACT |
| 2231 | GCTGAGGCTGTCCCGACGTC | 3355 | CTCGAAGGCAGGGCCGATCT | 4480 | GCATAACTCTCTTAAGACAC |
| 2232 | TGCTGAGGCTGTCCCGACGT | 3356 | CCTCGAAGGCAGGGCCGATC | 4481 | TGCATAACTCTCTTAAGACA |
| 2233 | CTGCTGAGGCTGTCCCGACG | 3357 | TCCTCGAAGGCAGGGCCGAT | 4482 | CTGCATAACTCTCTTAAGAC |
| 2234 | CCTGCTGAGGCTGTCCCGAC | 3358 | TTCCTCGAAGGCAGGGCCGA | 4483 | GCTGCATAACTCTCTTAAGA |
| 2235 | CCCTGCTGAGGCTGTCCCGA | 3359 | ATTCCTCGAAGGCAGGGCCG | 4484 | TGCTGCATAACTCTCTTAAG |
| 2236 | GCCCTGCTGAGGCTGTCCCG | 3360 | AATTCCTCGAAGGCAGGGCC | 4485 | ATGCTGCATAACTCTCTTAA |
| 2237 | TGCCCTGCTGAGGCTGTCCC | 3361 | CAATTCCTCGAAGGCAGGGC | 4486 | GATGCTGCATAACTCTCTTA |
| 2238 | CTGCCCTGCTGAGGCTGTCC | 3362 | TCAATTCCTCGAAGGCAGGG | 4487 | TGATGCTGCATAACTCTCTT |
| 2239 | GCTGCCCTGCTGAGGCTGTC | 3363 | ATCAATTCCTCGAAGGCAGG | 4488 | ATGATGCTGCATAACTCTCT |
| 2240 | AGCTGCCCTGCTGAGGCTGT | 3364 | TATCAATTCCTCGAAGGCAG | 4489 | AATGATGCTGCATAACTCTC |
| 2241 | CAGCTGCCCTGCTGAGGCTG | 3365 | ATATCAATTCCTCGAAGGCA | 4490 | TAATGATGCTGCATAACTCT |
| 2242 | CCAGCTGCCCTGCTGAGGCT | 3366 | CATATCAATTCCTCGAAGGC | 4491 | GTAATGATGCTGCATAACTC |
| 2243 | ACCAGCTGCCCTGCTGAGGC | 3367 | CCATATCAATTCCTCGAAGG | 4492 | TGTAATGATGCTGCATAACT |
| 2244 | CACCAGCTGCCCTGCTGAGG | 3368 | CCCATATCAATTCCTCGAAG | 4493 | TTGTAATGATGCTGCATAAC |
| 2245 | GCACCAGCTGCCCTGCTGAG | 3369 | TCCCATATCAATTCCTCGAA | 4494 | ATTGTAATGATGCTGCATAA |
| 2246 | AGCACCAGCTGCCCTGCTGA | 3370 | CTCCCATATCAATTCCTCGA | 4495 | GATTGTAATGATGCTGCATA |
| 2247 | GAGCACCAGCTGCCCTGCTG | 3371 | CCTCCCATATCAATTCCTCG | 4496 | TGATTGTAATGATGCTGCAT |
| 2248 | CGAGCACCAGCTGCCCTGCT | 3372 | CCCTCCCATATCAATTCCTC | 4497 | CTGATTGTAATGATGCTGCA |
| 2249 | ACGAGCACCAGCTGCCCTGC | 3373 | ACCCTCCCATATCAATTCCT | 4498 | ACTGATTGTAATGATGCTGC |
| 2250 | GACGAGCACCAGCTGCCCTG | 3374 | CACCCTCCCATATCAATTCC | 4499 | AACTGATTGTAATGATGCTG |
| 2251 | CGACGAGCACCAGCTGCCCT | 3375 | TCACCCTCCCATATCAATTC | 4500 | AAACTGATTGTAATGATGCT |
| 2252 | GCGACGAGCACCAGCTGCCC | 3376 | CTCACCCTCCCATATCAATT | 4501 | TAAACTGATTGTAATGATGC |
| 2253 | GGCGACGAGCACCAGCTGCC | 3377 | CCTCACCCTCCCATATCAAT | 4502 | TTAAACTGATTGTAATGATG |
| 2254 | AGGCGACGAGCACCAGCTGC | 3378 | TCCTCACCCTCCCATATCAA | 4503 | CTTAAACTGATTGTAATGAT |
| 2255 | AAGGCGACGAGCACCAGCTG | 3379 | CTCCTCACCCTCCCATATCA | 4504 | TCTTAAACTGATTGTAATGA |
| 2256 | GAAGGCGACGAGCACCAGCT | 3380 | TCTCCTCACCCTCCCATATC | 4505 | GTCTTAAACTGATTGTAATG |
| 2257 | GGAAGGCGACGAGCACCAGC | 3381 | CTCTCCTCACCCTCCCATAT | 4506 | GGTCTTAAACTGATTGTAAT |
| 2258 | AGGAAGGCGACGAGCACCAG | 3382 | TCTCTCCTCACCCTCCCATA | 4507 | TGGTCTTAAACTGATTGTAA |
| 2259 | AAGGAAGGCGACGAGCACCA | 3383 | TTCTCTCCTCACCCTCCCAT | 4508 | ATGGTCTTAAACTGATTGTA |
| 2260 | TAAGGAAGGCGACGAGCACC | 3384 | TTTCTCTCCTCACCCTCCCA | 4509 | AATGGTCTTAAACTGATTGT |
| 2261 | TTAAGGAAGGCGACGAGCAC | 3385 | TTTTCTCTCCTCACCCTCCC | 4510 | AAATGGTCTTAAACTGATTG |
| 2262 | GTTAAGGAAGGCGACGAGCA | 3386 | ATTTTCTCTCCTCACCCTCC | 4511 | AAAATGGTCTTAAACTGATT |
| 2263 | GGTTAAGGAAGGCGACGAGC | 3387 | CATTTTCTCTCCTCACCCTC | 4512 | AAAAATGGTCTTAAACTGAT |
| 2264 | GGGTTAAGGAAGGCGACGAG | 3388 | ACATTTTCTCTCCTCACCCT | 4513 | TAAAAATGGTCTTAAACTGA |
| 2265 | TGGGTTAAGGAAGGCGACGA | 3389 | TACATTTTCTCTCCTCACCC | 4514 | ATAAAAATGGTCTTAAACTG |
| 2266 | ATGGGTTAAGGAAGGCGACG | 3390 | GTACATTTTCTCTCCTCACC | 4515 | GATAAAAATGGTCTTAAACT |
| 2267 | GATGGGTTAAGGAAGGCGAC | 3391 | GGTACATTTTCTCTCCTCAC | 4516 | TGATAAAAATGGTCTTAAAC |
| 2268 | GGATGGGTTAAGGAAGGCGA | 3392 | TGGTACATTTTCTCTCCTCA | 4517 | GTGATAAAAATGGTCTTAAA |
| 2269 | AGGATGGGTTAAGGAAGGCG | 3393 | GTGGTACATTTTCTCTCCTC | 4518 | GGTGATAAAAATGGTCTTAA |
| 2270 | CAGGATGGGTTAAGGAAGGC | 3394 | TGTGGTACATTTTCTCTCCT | 4519 | TGGTGATAAAAATGGTCTTA |
| 2271 | GCAGGATGGGTTAAGGAAGG | 3395 | CTGTGGTACATTTTCTCTCC | 4520 | GTGGTGATAAAAATGGTCTT |
| 2272 | TGCAGGATGGGTTAAGGAAG | 3396 | TCTGTGGTACATTTTCTCTC | 4521 | TGTGGTGATAAAAATGGTCT |
| 2273 | CTGCAGGATGGGTTAAGGAA | 3397 | TTCTGTGGTACATTTTCTCT | 4522 | CTGTGGTGATAAAAATGGTC |
| 2274 | CCTGCAGGATGGGTTAAGGA | 3398 | GTTCTGTGGTACATTTTCTC | 4523 | TCTGTGGTGATAAAAATGGT |
| 2275 | GCCTGCAGGATGGGTTAAGG | 3399 | TGTTCTGTGGTACATTTTCT | 4524 | TTCTGTGGTGATAAAAATGG |
| 2276 | CGCCTGCAGGATGGGTTAAG | 3400 | CTGTTCTGTGGTACATTTTC | 4525 | CTTCTGTGGTGATAAAAATG |
| 2277 | GCGCCTGCAGGATGGGTTAA | 3401 | TCTGTTCTGTGGTACATTTT | 4526 | CCTTCTGTGGTGATAAAAAT |
| 2278 | GGCGCCTGCAGGATGGGTTA | 3402 | ATCTGTTCTGTGGTACATTT | 4527 | GCCTTCTGTGGTGATAAAAA |
| 2279 | AGGCGCCTGCAGGATGGGTT | 3403 | AATCTGTTCTGTGGTACATT | 4528 | TGCCTTCTGTGGTGATAAAA |
| 2280 | CAGGCGCCTGCAGGATGGGT | 3404 | TAATCTGTTCTGTGGTACAT | 4529 | CTGCCTTCTGTGGTGATAAA |
| 2281 | CCAGGCGCCTGCAGGATGGG | 3405 | CTAATCTGTTCTGTGGTACA | 4530 | GCTGCCTTCTGTGGTGATAA |
| 2282 | CCCAGGCGCCTGCAGGATGG | 3406 | GCTAATCTGTTCTGTGGTAC | 4531 | GGCTGCCTTCTGTGGTGATA |
| 2283 | CCCCAGGCGCCTGCAGGATG | 3407 | GGCTAATCTGTTCTGTGGTA | 4532 | AGGCTGCCTTCTGTGGTGAT |
| 2284 | CCCCCAGGCGCCTGCAGGAT | 3408 | TGGCTAATCTGTTCTGTGGT | 4533 | AAGGCTGCCTTCTGTGGTGA |
| 2285 | TCCCCCAGGCGCCTGCAGGA | 3409 | CTGGCTAATCTGTTCTGTGG | 4534 | TAAGGCTGCCTTCTGTGGTG |
| 2286 | CTCCCCCAGGCGCCTGCAGG | 3410 | ACTGGCTAATCTGTTCTGTG | 4535 | ATAAGGCTGCCTTCTGTGGT |
| 2287 | ACTCCCCCAGGCGCCTGCAG | 3411 | CACTGGCTAATCTGTTCTGT | 4536 | CATAAGGCTGCCTTCTGTGG |
| 2288 | AACTCCCCCAGGCGCCTGCA | 3412 | TCACTGGCTAATCTGTTCTG | 4537 | ACATAAGGCTGCCTTCTGTG |
| 2289 | AAACTCCCCCAGGCGCCTGC | 3413 | GTCACTGGCTAATCTGTTCT | 4538 | GACATAAGGCTGCCTTCTGT |
| 2290 | CAAACTCCCCCAGGCGCCTG | 3414 | TGTCACTGGCTAATCTGTTC | 4539 | GGACATAAGGCTGCCTTCTG |
| 2291 | GCAAACTCCCCCAGGCGCCT | 3415 | TTGTCACTGGCTAATCTGTT | 4540 | AGGACATAAGGCTGCCTTCT |
| 2292 | AGCAAACTCCCCCAGGCGCC | 3416 | CTTGTCACTGGCTAATCTGT | 4541 | AAGGACATAAGGCTGCCTTC |
| 2293 | TAGCAAACTCCCCCAGGCGC | 3417 | TCTTGTCACTGGCTAATCTG | 4542 | AAAGGACATAAGGCTGCCTT |
| 2294 | CTAGCAAACTCCCCCAGGCG | 3418 | CTCTTGTCACTGGCTAATCT | 4543 | TAAAGGACATAAGGCTGCCT |
| 2295 | TCTAGCAAACTCCCCCAGGC | 3419 | ACTCTTGTCACTGGCTAATC | 4544 | CTAAAGGACATAAGGCTGCC |
| 2296 | CTCTAGCAAACTCCCCCAGG | 3420 | GACTCTTGTCACTGGCTAAT | 4545 | GCTAAAGGACATAAGGCTGC |
| 2297 | TCTCTAGCAAACTCCCCCAG | 3421 | TGACTCTTGTCACTGGCTAA | 4546 | TGCTAAAGGACATAAGGCTG |
| 2298 | ATCTCTAGCAAACTCCCCCA | 3422 | CTGACTCTTGTCACTGGCTA | 4547 | CTGCTAAAGGACATAAGGCT |
| 2299 | CATCTCTAGCAAACTCCCCC | 3423 | GCTGACTCTTGTCACTGGCT | 4548 | ACTGCTAAAGGACATAAGGC |
| 2300 | TCATCTCTAGCAAACTCCCC | 3424 | GGCTGACTCTTGTCACTGGC | 4549 | GACTGCTAAAGGACATAAGG |
| 2301 | ATCATCTCTAGCAAACTCCC | 3425 | AGGCTGACTCTTGTCACTGG | 4550 | TGACTGCTAAAGGACATAAG |
| 2302 | TATCATCTCTAGCAAACTCC | 3426 | CAGGCTGACTCTTGTCACTG | 4551 | GTGACTGCTAAAGGACATAA |
| 2303 | CTATCATCTCTAGCAAACTC | 3427 | GCAGGCTGACTCTTGTCACT | 4552 | GGTGACTGCTAAAGGACATA |
| 2304 | GCTATCATCTCTAGCAAACT | 3428 | AGCAGGCTGACTCTTGTCAC | 4553 | GGGTGACTGCTAAAGGACAT |
| 2305 | CGCTATCATCTCTAGCAAAC | 3429 | AAGCAGGCTGACTCTTGTCA | 4554 | AGGGTGACTGCTAAAGGACA |
| 2306 | GCGCTATCATCTCTAGCAAA | 3430 | CAAGCAGGCTGACTCTTGTC | 4555 | GAGGGTGACTGCTAAAGGAC |
| 2307 | CGCGCTATCATCTCTAGCAA | 3431 | CCAAGCAGGCTGACTCTTGT | 4556 | GGAGGGTGACTGCTAAAGGA |
| 2308 | ACGCGCTATCATCTCTAGCA | 3432 | ACCAAGCAGGCTGACTCTTG | 4557 | GGGAGGGTGACTGCTAAAGG |
| 2309 | CACGCGCTATCATCTCTAGC | 3433 | CACCAAGCAGGCTGACTCTT | 4558 | AGGGAGGGTGACTGCTAAAG |
| 2310 | ACACGCGCTATCATCTCTAG | 3434 | GCACCAAGCAGGCTGACTCT | 4559 | TAGGGAGGGTGACTGCTAAA |
| 2311 | GACACGCGCTATCATCTCTA | 3435 | GGCACCAAGCAGGCTGACTC | 4560 | TTAGGGAGGGTGACTGCTAA |
| 2312 | AGACACGCGCTATCATCTCT | 3436 | TGGCACCAAGCAGGCTGACT | 4561 | GTTAGGGAGGGTGACTGCTA |
| 2313 | CAGACACGCGCTATCATCTC | 3437 | TTGGCACCAAGCAGGCTGAC | 4562 | GGTTAGGGAGGGTGACTGCT |
| 2314 | CCAGACACGCGCTATCATCT | 3438 | TTTGGCACCAAGCAGGCTGA | 4563 | GGGTTAGGGAGGGTGACTGC |
| 2315 | CCCAGACACGCGCTATCATC | 3439 | CTTTGGCACCAAGCAGGCTG | 4564 | AGGGTTAGGGAGGGTGACTG |
| 2316 | CCCCAGACACGCGCTATCAT | 3440 | CCTTTGGCACCAAGCAGGCT | 4565 | TAGGGTTAGGGAGGGTGACT |
| 2317 | GCCCCAGACACGCGCTATCA | 3441 | CCCTTTGGCACCAAGCAGGC | 4566 | CTAGGGTTAGGGAGGGTGAC |
| 2318 | TGCCCCAGACACGCGCTATC | 3442 | GCCCTTTGGCACCAAGCAGG | 4567 | CCTAGGGTTAGGGAGGGTGA |
| 2319 | ATGCCCCAGACACGCGCTAT | 3443 | GGCCCTTTGGCACCAAGCAG | 4568 | GCCTAGGGTTAGGGAGGGTG |
| 2320 | AATGCCCCAGACACGCGCTA | 3444 | AGGCCCTTTGGCACCAAGCA | 4569 | TGCCTAGGGTTAGGGAGGGT |
| 2321 | CAATGCCCCAGACACGCGCT | 3445 | CAGGCCCTTTGGCACCAAGC | 4739 | TTTTTTTTTTGGACTTGGGAG |
| | | | | | TGGTGACTG |
| 2322 | TCAATGCCCCAGACACGCGC | 3446 | CCAGGCCCTTTGGCACCAAG | 4740 | TTTTTTTTTTGGACTTGGGAGT |
| | | | | | GGTGACTG |
| 2323 | GTCAATGCCCCAGACACGCG | 3447 | CCCAGGCCCTTTGGCACCAA | 4741 | GGGTGCCACCTAGTGGAAGA |
| 2324 | TGTCAATGCCCCAGACACGC | 3448 | GCCCAGGCCCTTTGGCACCA | 4742 | TGGGGGGTGCCACCTAGTGG |
| 2325 | CTGTCAATGCCCCAGACACG | 3449 | GGCCCAGGCCCTTTGGCACC | 4570 | TCGGAGTTGGGGGGTGCCAC |
| 2326 | ACTGTCAATGCCCCAGACAC | 3450 | TGGCCCAGGCCCTTTGGCAC | 4571 | GGCACTCGGAGTTGGGGGGT |
| 2327 | CACTGTCAATGCCCCAGACA | 3451 | CTGGCCCAGGCCCTTTGGCA | 4572 | TCCTCTCCCCTTGGCACTCG |
| 2328 | GCACTGTCAATGCCCCAGAC | 3452 | CCTGGCCCAGGCCCTTTGGC | 4573 | GAATTTCCTCTCCCCTTGGC |
| 2329 | CGCACTGTCAATGCCCCAGA | 3453 | GCCTGGCCCAGGCCCTTTGG | 4574 | TTTTGGGAATTTCCTCTCCC |
| 2330 | TCGCACTGTCAATGCCCCAG | 3454 | TGCCTGGCCCAGGCCCTTTG | 4575 | GCTCCCCTTTTGGGAATTTC |
| 2331 | CTCGCACTGTCAATGCCCCA | 3455 | CTGCCTGGCCCAGGCCCTTT | 4576 | TGAGACCTTGGCTTCTCGAA |
| 2332 | CCTCGCACTGTCAATGCCCC | 3456 | TCTGCCTGGCCCAGGCCCTT | 4577 | TGGGAACGAGACCTGAGACC |
| 2333 | CCCTCGCACTGTCAATGCCC | 3457 | CTCTGCCTGGCCCAGGCCCT | 4578 | CGAGGGCCTGGGAACGAGAC |
| 2334 | TCCCTCGCACTGTCAATGCC | 3458 | TCTCTGCCTGGCCCAGGCCC | 4579 | CTCTCCTGGTGGTGGTGGGT |
| 2335 | CTCCCTCGCACTGTCAATGC | 3459 | ATCTCTGCCTGGCCCAGGCC | 4580 | TCCCCTCTCCTGGTGGTGGT |
| 2336 | ACTCCCTCGCACTGTCAATG | 3460 | CATCTCTGCCTGGCCCAGGC | 4581 | TTCTTCCCCTCTCCTGGTGG |
| 2337 | CACTCCCTCGCACTGTCAAT | 3461 | CCATCTCTGCCTGGCCCAGG | 4582 | GCTGGCTTCTTCCCCTCTCC |
| 2338 | ACACTCCCTCGCACTGTCAA | 3462 | TCCATCTCTGCCTGGCCCAG | 4583 | CTGTCGGTAGGTGCTGGCTT |
| 2339 | AACACTCCCTCGCACTGTCA | 3463 | ATCCATCTCTGCCTGGCCCA | 4584 | AGCTCCACCCCTGTCGGTAG |
| 2340 | TAACACTCCCTCGCACTGTC | 3464 | CATCCATCTCTGCCTGGCCC | 4585 | ACCCAGCTCCACCCCTGTCG |
| 2341 | ATAACACTCCCTCGCACTGT | 3465 | TCATCCATCTCTGCCTGGCC | 4586 | CTTGACCCAGCTCCACCCCT |
| 2342 | CATAACACTCCCTCGCACTG | 3466 | ATCATCCATCTCTGCCTGGC | 4587 | GGAGTCTCAGCCGGAGACAA |
| 2343 | TCATAACACTCCCTCGCACT | 3467 | CATCATCCATCTCTGCCTGG | 4588 | GAGGTTCCCCTGGAGTCTCA |
| 2344 | TTCATAACACTCCCTCGCAC | 3468 | CCATCATCCATCTCTGCCTG | 4589 | GCTTGAGGTTCCCCTGGAGT |
| 2345 | ATTCATAACACTCCCTCGCA | 3469 | CCCATCATCCATCTCTGCCT | 4590 | GTGAGCTTGAGGTTCCCCTG |
| 2346 | AATTCATAACACTCCCTCGC | 3470 | TCCCATCATCCATCTCTGCC | 4591 | CGCCAGGGCCATGTGAGCTT |
| 2347 | CAATTCATAACACTCCCTCG | 3471 | CTCCCATCATCCATCTCTGC | 4592 | GGCCCGCCAGGGCCATGTGA |
| 2348 | CCAATTCATAACACTCCCTC | 3472 | TCTCCCATCATCCATCTCTG | 4593 | CAGGGGCCCGCCAGGGCCAT |
| 2349 | CCCAATTCATAACACTCCCT | 3473 | ATCTCCCATCATCCATCTCT | 4594 | TGCCCAGGGGCCCGCCAGGG |
| 2350 | TCCCAATTCATAACACTCCC | 3474 | CATCTCCCATCATCCATCTC | 4595 | TGCTCCTGCCCAGGGGCCCG |
| 2351 | TTCCCAATTCATAACACTCC | 3475 | CCATCTCCCATCATCCATCT | 4596 | GGATGGGTTAAGGAAGGCGA |
| 2352 | ATTCCCAATTCATAACACTC | 3476 | CCCATCTCCCATCATCCATC | 4597 | AGGCGCCTGCAGGATGGGTT |
| 2353 | TATTCCCAATTCATAACACT | 3477 | GCCCATCTCCCATCATCCAT | 4598 | CCCCAGGCGCCTGCAGGATG |
| 2354 | ATATTCCCAATTCATAACAC | 3478 | GGCCCATCTCCCATCATCCA | 4599 | AACTCCCCCAGGCGCCTGCA |
| 2355 | AATATTCCCAATTCATAACA | 3479 | TGGCCCATCTCCCATCATCC | 4600 | GCGCTATCATCTCTAGCAAA |
| 2356 | AAATATTCCCAATTCATAAC | 3480 | CTGGCCCATCTCCCATCATC | 4601 | TCTGCCTGGCCCAGGCCCTT |
| 2357 | CAAATATTCCCAATTCATAA | 3481 | CCTGGCCCATCTCCCATCAT | 4602 | ATCCATCTCTGCCTGGCCCA |
| 2358 | ACAAATATTCCCAATTCATA | 3482 | TCCTGGCCCATCTCCCATCA | 4603 | TCTCCCATCATCCATCTCTG |
| 2359 | CACAAATATTCCCAATTCAT | 3483 | CTCCTGGCCCATCTCCCATC | 4604 | CCCATCTCCCATCATCCATC |
| 2360 | GCACAAATATTCCCAATTCA | 3484 | TCTCCTGGCCCATCTCCCAT | 4605 | TCTCTCCTGGCCCATCTCCC |
| 2361 | AGCACAAATATTCCCAATTC | 3485 | CTCTCCTGGCCCATCTCCCA | 4606 | GGGCCTCTCCTCTCTCCTGG |
| 2362 | GAGCACAAATATTCCCAATT | 3486 | TCTCTCCTGGCCCATCTCCC | 4607 | TCCCTGGGCCTCTCCTCTCT |
| 2363 | CGAGCACAAATATTCCCAAT | 3487 | CTCTCTCCTGGCCCATCTCC | 4608 | GCTTTCCCTGGGCCTCTCCT |
| 2364 | GCGAGCACAAATATTCCCAA | 3488 | CCTCTCTCCTGGCCCATCTC | 4609 | GTGCAGACACTGCCGTTCCT |
| 2365 | GGCGAGCACAAATATTCCCA | 3489 | TCCTCTCTCCTGGCCCATCT | 4610 | CCCCTGTGCAGACACTGCCG |
| 2366 | AGGCGAGCACAAATATTCCC | 3490 | CTCCTCTCTCCTGGCCCATC | 4611 | CCATCCCCTGTGCAGACACT |
| 2367 | AAGGCGAGCACAAATATTCC | 3491 | TCTCCTCTCTCCTGGCCCAT | 4612 | AGTCTCCATCCCCTGTGCAG |
| 2368 | AAAGGCGAGCACAAATATTC | 3492 | CTCTCCTCTCTCCTGGCCCA | 4613 | CTTGCTCAGGCTGCAGCAGT |
| 2369 | GAAAGGCGAGCACAAATATT | 3493 | CCTCTCCTCTCTCCTGGCCC | 4614 | GTTCCTTGCTCAGGCTGCAG |
| 2370 | AGAAAGGCGAGCACAAATAT | 3494 | GCCTCTCCTCTCTCCTGGCC | 4615 | TCAGCTCCAGTTCCTTGCTC |
| 2371 | TAGAAAGGCGAGCACAAATA | 3495 | GGCCTCTCCTCTCTCCTGGC | 4616 | GGGTGCCACCTAGTGGAAGA |
| 2372 | ATAGAAAGGCGAGCACAAAT | 3496 | GGGCCTCTCCTCTCTCCTGG | 4617 | TGGGGGGTGCCACCTAGTGG |
| 2373 | GATAGAAAGGCGAGCACAAA | 3497 | TGGGCCTCTCCTCTCTCCTG | 4618 | TCGGAGTTGGGGGGTGCCAC |
| 2374 | GGATAGAAAGGCGAGCACAA | 3498 | CTGGGCCTCTCCTCTCTCCT | 4619 | GGCACTCGGAGTTGGGGGGT |
| 2375 | AGGATAGAAAGGCGAGCACA | 3499 | CCTGGGCCTCTCCTCTCTCC | 4620 | TCCTCTCCCCTTGGCACTCG |
| 2376 | AAGGATAGAAAGGCGAGCAC | 3500 | CCCTGGGCCTCTCCTCTCTC | 4621 | GAATTTCCTCTCCCCTTGGC |
| 2377 | AAAGGATAGAAAGGCGAGCA | 3501 | TCCCTGGGCCTCTCCTCTCT | 4622 | TTTTGGGAATTTCCTCTCCC |
| 2378 | AAAAGGATAGAAAGGCGAGC | 3502 | TTCCCTGGGCCTCTCCTCTC | 4623 | GCTCCCCTTTTGGGAATTTC |
| 2379 | CAAAAGGATAGAAAGGCGAG | 3503 | TTTCCCTGGGCCTCTCCTCT | 4624 | TGAGACCTTGGCTTCTCGAA |
| 2380 | ACAAAAGGATAGAAAGGCGA | 3504 | CTTTCCCTGGGCCTCTCCTC | 4625 | TGGGAACGAGACCTGAGACC |
| 2381 | AACAAAAGGATAGAAAGGCG | 3505 | GCTTTCCCTGGGCCTCTCCT | 4626 | CGAGGGCCTGGGAACGAGAC |
| 2382 | AAACAAAAGGATAGAAAGGC | 3506 | GGCTTTCCCTGGGCCTCTCC | 4627 | CTCTCCTGGTGGTGGTGGGT |
| 2383 | CAAACAAAAGGATAGAAAGG | 3507 | AGGCTTTCCCTGGGCCTCTC | 4628 | TCCCCTCTCCTGGTGGTGGT |
| 2384 | ACAAACAAAAGGATAGAAAG | 3508 | CAGGCTTTCCCTGGGCCTCT | 4629 | TTCTTCCCCTCTCCTGGTGG |
| 2385 | AACAAACAAAAGGATAGAAA | 3509 | TCAGGCTTTCCCTGGGCCTC | 4630 | GCTGGCTTCTTCCCCTCTCC |
| 2386 | AAACAAACAAAAGGATAGAA | 3510 | ATCAGGCTTTCCCTGGGCCT | 4631 | CTGTCGGTAGGTGCTGGCTT |
| 2387 | CAAACAAACAAAAGGATAGA | 3511 | TATCAGGCTTTCCCTGGGCC | 4632 | AGCTCCACCCCTGTCGGTAG |
| 2388 | ACAAACAAACAAAAGGATAG | 3512 | GTATCAGGCTTTCCCTGGGC | 4633 | ACCCAGCTCCACCCCTGTCG |
| 2389 | AACAAACAAACAAAAGGATA | 3513 | AGTATCAGGCTTTCCCTGGG | 4634 | CTTGACCCAGCTCCACCCCT |
| 2390 | GAACAAACAAACAAAAGGAT | 3514 | CAGTATCAGGCTTTCCCTGG | 4635 | GGAGTCTCAGCCGGAGACAA |
| 2391 | AGAACAAACAAACAAAAGGA | 3515 | CCAGTATCAGGCTTTCCCTG | 4636 | GAGGTTCCCCTGGAGTCTCA |
| 2392 | CAGAACAAACAAACAAAAGG | 3516 | CCCAGTATCAGGCTTTCCCT | 4637 | GCTTGAGGTTCCCCTGGAGT |
| 2393 | TCAGAACAAACAAACAAAAG | 3517 | GCCCAGTATCAGGCTTTCCC | 4638 | GTGAGCTTGAGGTTCCCCTG |
| 2394 | CTCAGAACAAACAAACAAAA | 3518 | GGCCCAGTATCAGGCTTTCC | 4639 | CGCCAGGGCCATGTGAGCTT |
| 2395 | TCTCAGAACAAACAAACAAA | 3519 | CGGCCCAGTATCAGGCTTTC | 4640 | GGCCCGCCAGGGCCATGTGA |
| 2396 | GTCTCAGAACAAACAAACAA | 3520 | ACGGCCCAGTATCAGGCTTT | 4641 | CAGGGGCCCGCCAGGGCCAT |
| 2397 | CGTCTCAGAACAAACAAACA | 3521 | CACGGCCCAGTATCAGGCTT | 4642 | TGCCCAGGGGCCCGCCAGGG |
| 2398 | CCGTCTCAGAACAAACAAAC | 3522 | CCACGGCCCAGTATCAGGCT | 4643 | TGCTCCTGCCCAGGGGCCCG |
| 2399 | TCCGTCTCAGAACAAACAAA | 3523 | TCCACGGCCCAGTATCAGGC | 4644 | GGATGGGTTAAGGAAGGCGA |
| 2400 | CTCCGTCTCAGAACAAACAA | 3524 | CTCCACGGCCCAGTATCAGG | 4645 | AGGCGCCTGCAGGATGGGTT |
| 2401 | ACTCCGTCTCAGAACAAACA | 3525 | CCTCCACGGCCCAGTATCAG | 4646 | CCCCAGGCGCCTGCAGGATG |
| 2402 | GACTCCGTCTCAGAACAAAC | 3526 | TCCTCCACGGCCCAGTATCA | 4647 | AACTCCCCCAGGCGCCTGCA |
| 2403 | AGACTCCGTCTCAGAACAAA | 3527 | TTCCTCCACGGCCCAGTATC | 4648 | GCGCTATCATCTCTAGCAAA |
| 2404 | GAGACTCCGTCTCAGAACAA | 3528 | GTTCCTCCACGGCCCAGTAT | 4649 | TCTGCCTGGCCCAGGCCCTT |
| 2405 | TGAGACTCCGTCTCAGAACA | 3529 | CGTTCCTCCACGGCCCAGTA | 4650 | ATCCATCTCTGCCTGGCCCA |
| 2406 | GTGAGACTCCGTCTCAGAAC | 3530 | CCGTTCCTCCACGGCCCAGT | 4651 | TCTCCCATCATCCATCTCTG |
| 2407 | AGTGAGACTCCGTCTCAGAA | 3531 | GCCGTTCCTCCACGGCCCAG | 4652 | CCCATCTCCCATCATCCATC |
| 2408 | GAGTGAGACTCCGTCTCAGA | 3532 | TGCCGTTCCTCCACGGCCCA | 4653 | TCTCTCCTGGCCCATCTCCC |
| 2409 | AGAGTGAGACTCCGTCTCAG | 3533 | CTGCCGTTCCTCCACGGCCC | 4654 | GGGCCTCTCCTCTCTCCTGG |
| 2410 | CAGAGTGAGACTCCGTCTCA | 3534 | ACTGCCGTTCCTCCACGGCC | 4655 | TCCCTGGGCCTCTCCTCTCT |
| 2411 | ACAGAGTGAGACTCCGTCTC | 3535 | CACTGCCGTTCCTCCACGGC | 4656 | GCTTTCCCTGGGCCTCTCCT |
| 2412 | GACAGAGTGAGACTCCGTCT | 3536 | ACACTGCCGTTCCTCCACGG | 4657 | GTGCAGACACTGCCGTTCCT |
| 2413 | CGACAGAGTGAGACTCCGTC | 3537 | GACACTGCCGTTCCTCCACG | 4658 | CCCCTGTGCAGACACTGCCG |
| 2414 | GCGACAGAGTGAGACTCCGT | 3538 | AGACACTGCCGTTCCTCCAC | 4659 | CCATCCCCTGTGCAGACACT |
| 2415 | GGCGACAGAGTGAGACTCCG | 3539 | CAGACACTGCCGTTCCTCCA | 4660 | AGTCTCCATCCCCTGTGCAG |
| 2416 | TGGCGACAGAGTGAGACTCC | 3540 | GCAGACACTGCCGTTCCTCC | 4661 | CTTGCTCAGGCTGCAGCAGT |
| 2417 | CTGGCGACAGAGTGAGACTC | 3541 | TGCAGACACTGCCGTTCCTC | 4662 | GTTCCTTGCTCAGGCTGCAG |
| 2418 | CCTGGCGACAGAGTGAGACT | 3542 | GTGCAGACACTGCCGTTCCT | 4663 | TCAGCTCCAGTTCCTTGCTC |
| 2419 | GCCTGGCGACAGAGTGAGAC | 3543 | TGTGCAGACACTGCCGTTCC | 4664 | GGTTGGCTCCCCTTTTGGGA |
| 2420 | AGCCTGGCGACAGAGTGAGA | 3544 | CTGTGCAGACACTGCCGTTC | 4665 | GCCACTTTTTCCTGCTCATT |
| 2421 | CAGCCTGGCGACAGAGTGAG | 3545 | CCTGTGCAGACACTGCCGTT | 4666 | CTCCGAGGGCCTGGGAACGA |
| 2422 | CCAGCCTGGCGACAGAGTGA | 3546 | CCCTGTGCAGACACTGCCGT | 4667 | AGCCGGAGGGGCGCGCGACC |
| 2423 | TCCAGCCTGGCGACAGAGTG | 3547 | CCCCTGTGCAGACACTGCCG | 4668 | TGGGTGGTTCCCGCGGCGGC |
| 2424 | CTCCAGCCTGGCGACAGAGT | 3548 | TCCCCTGTGCAGACACTGCC | 4669 | AAGCAGGGACCACACCATTC |
| 2425 | ACTCCAGCCTGGCGACAGAG | 3549 | ATCCCCTGTGCAGACACTGC | 4670 | GAAGGGGCTTTCTACCTCCC |
| 2426 | CACTCCAGCCTGGCGACAGA | 3550 | CATCCCCTGTGCAGACACTG | 4671 | AGTAATTGCAGTGACGCCCC |
| 2427 | GCACTCCAGCCTGGCGACAG | 3551 | CCATCCCCTGTGCAGACACT | 4672 | TCTGATACACTAGGGGGAGT |
| 2428 | TGCACTCCAGCCTGGCGACA | 3552 | TCCATCCCCTGTGCAGACAC | 4673 | GAACCGCTTACTGAAACTCC |
| 2429 | CTGCACTCCAGCCTGGCGAC | 3553 | CTCCATCCCCTGTGCAGACA | 4674 | GCCGCGCAGACCTCTCGCCT |
| 2430 | ACTGCACTCCAGCCTGGCGA | 3554 | TCTCCATCCCCTGTGCAGAC | 4675 | CAAGGACCGCGGAGTCGGAC |
| 2431 | CACTGCACTCCAGCCTGGCG | 3555 | GTCTCCATCCCCTGTGCAGA | 4676 | AGTCTGAGCGCTACCCGGTT |
| 2432 | CCACTGCACTCCAGCCTGGC | 3556 | AGTCTCCATCCCCTGTGCAG | 4677 | AGGTAGGCGGGTCATCGCGC |
| 2433 | GCCACTGCACTCCAGCCTGG | 3557 | CAGTCTCCATCCCCTGTGCA | 4678 | AGCGGGTAGCCCTGATCCCT |
| 2434 | TGCCACTGCACTCCAGCCTG | 3558 | GCAGTCTCCATCCCCTGTGC | 4679 | CCTCTATATTGATAAGTAGC |
| 2435 | GTGCCACTGCACTCCAGCCT | 3559 | AGCAGTCTCCATCCCCTGTG | 4680 | GAGTGATTTGAGTAGAAAAG |
| 2436 | CGTGCCACTGCACTCCAGCC | 3560 | CAGCAGTCTCCATCCCCTGT | 4681 | GATCCCTAGAAATGGGGTGT |
| 2437 | TCGTGCCACTGCACTCCAGC | 3561 | GCAGCAGTCTCCATCCCCTG | 4682 | CGACGTCACATGATTCTCCA |
| 2438 | ATCGTGCCACTGCACTCCAG | 3562 | TGCAGCAGTCTCCATCCCCT | 4683 | TAAGGAAGGCGACGAGCACC |
| 2439 | GATCGTGCCACTGCACTCCA | 3563 | CTGCAGCAGTCTCCATCCCC | 4684 | AATGCCCCAGACACGCGCTA |
| 2440 | AGATCGTGCCACTGCACTCC | 3564 | GCTGCAGCAGTCTCCATCCC | 4685 | AAAGGCGAGCACAAATATTC |
| 2441 | GAGATCGTGCCACTGCACTC | 3565 | GGCTGCAGCAGTCTCCATCC | 4686 | CAGAACAAACAAACAAAAGG |
| 2442 | CGAGATCGTGCCACTGCACT | 3566 | AGGCTGCAGCAGTCTCCATC | 4687 | AACACACAAGTCCGGGCGCG |
| 2443 | CCGAGATCGTGCCACTGCAC | 3567 | CAGGCTGCAGCAGTCTCCAT | 4688 | GTGAGCTATGATCAGCTTGG |
| 2444 | GCCGAGATCGTGCCACTGCA | 3568 | TCAGGCTGCAGCAGTCTCCA | 4689 | AGAATCGCTTTGGGAGCAGG |
| 2445 | AGCCGAGATCGTGCCACTGC | 3569 | CTCAGGCTGCAGCAGTCTCC | 4690 | CATTTGTAATCCCAGCGCCT |
| 2446 | GAGCCGAGATCGTGCCACTG | 3570 | GCTCAGGCTGCAGCAGTCTC | 4691 | AGGAGCGAGCCAGCTCAGTA |
| 2447 | TGAGCCGAGATCGTGCCACT | 3571 | TGCTCAGGCTGCAGCAGTCT | 4692 | GAATAGGGCAGGACAGGACA |
| 2448 | GTGAGCCGAGATCGTGCCAC | 3572 | TTGCTCAGGCTGCAGCAGTC | 4693 | ATGTCAACCGAGTTTGGAGA |
| 2449 | AGTGAGCCGAGATCGTGCCA | 3573 | CTTGCTCAGGCTGCAGCAGT | 4694 | TTCAACTCACTCTGGGGCCT |
| 2450 | CAGTGAGCCGAGATCGTGCC | 3574 | CCTTGCTCAGGCTGCAGCAG | 4695 | GGGCTTTGGGGCTGTTTATC |
| 2451 | GCAGTGAGCCGAGATCGTGC | 3575 | TCCTTGCTCAGGCTGCAGCA | 4696 | AATCCCTGCTCCCTGTCCTG |
| 2452 | TGCAGTGAGCCGAGATCGTG | 3576 | TTCCTTGCTCAGGCTGCAGC | 4697 | ATTAGATTCAGCCTGAGACT |
| 2453 | TTGCAGTGAGCCGAGATCGT | 3577 | GTTCCTTGCTCAGGCTGCAG | 4698 | TACACCTGGCAACTGGCGCA |
| 2454 | GTTGCAGTGAGCCGAGATCG | 3578 | AGTTCCTTGCTCAGGCTGCA | 4699 | CCTGGAGACAATCTACTTCC |
| 2455 | GGTTGCAGTGAGCCGAGATC | 3579 | CAGTTCCTTGCTCAGGCTGC | 4700 | GTGGAATGAAATCTCTTCTT |
| 2456 | AGGTTGCAGTGAGCCGAGAT | 3580 | CCAGTTCCTTGCTCAGGCTG | 4701 | TCAGCTCAAGGAGATGCTCC |
| 2457 | GAGGTTGCAGTGAGCCGAGA | 3581 | TCCAGTTCCTTGCTCAGGCT | 4702 | CTCCACCTGCCAGTTAAAAT |
| 2458 | AGAGGTTGCAGTGAGCCGAG | 3582 | CTCCAGTTCCTTGCTCAGGC | 4703 | CCATCTGGTTTCCCAGGGCT |
| 2459 | CAGAGGTTGCAGTGAGCCGA | 3583 | GCTCCAGTTCCTTGCTCAGG | 4704 | GGCATTGAACATGGCATTCA |
| 2460 | GCAGAGGTTGCAGTGAGCCG | 3584 | AGCTCCAGTTCCTTGCTCAG | 4705 | GGCACCCAGCAGCTCCTGAA |
| 2461 | GGCAGAGGTTGCAGTGAGCC | 3585 | CAGCTCCAGTTCCTTGCTCA | 4706 | CTTCTCTTTCGACAGCCCCC |
| 2462 | AGGCAGAGGTTGCAGTGAGC | 3586 | TCAGCTCCAGTTCCTTGCTC | 4707 | CAGGGCCGATCTGGAGACTA |
| 2463 | GAGGCAGAGGTTGCAGTGAG | 3587 | CTCAGCTCCAGTTCCTTGCT | 4708 | CCTCCCATATCAATTCCTCG |
| 2464 | GGAGGCAGAGGTTGCAGTGA | 3588 | CCTCAGCTCCAGTTCCTTGC | 4709 | TGTCACTGGCTAATCTGTTC |
| 2465 | GGGAGGCAGAGGTTGCAGTG | 3589 | TCCTCAGCTCCAGTTCCTTG | 4710 | TTTGGCACCAAGCAGGCTGA |
| 2466 | CGGGAGGCAGAGGTTGCAGT | 3590 | ATCCTCAGCTCCAGTTCCTT | 4711 | ACGGCCCAGTATCAGGCTTT |
| 2467 | CCGGGAGGCAGAGGTTGCAG | 3591 | CATCCTCAGCTCCAGTTCCT | 4712 | CCTCATCCTCAGCTCCAGTT |
| 2468 | CCCGGGAGGCAGAGGTTGCA | 3592 | TCATCCTCAGCTCCAGTTCC | 4713 | CGAGCTTGGTTAGCCATCCC |
| 2469 | ACCCGGGAGGCAGAGGTTGC | 3593 | CTCATCCTCAGCTCCAGTTC | 4714 | TCCCTCCTCCGACTTCTGCT |
| 2470 | AACCCGGGAGGCAGAGGTTG | 3594 | CCTCATCCTCAGCTCCAGTT | 4715 | ACGACTCCACAGCTCGGTTG |
| 2471 | GAACCCGGGAGGCAGAGGTT | 3595 | GCCTCATCCTCAGCTCCAGT | 4716 | GCTCTAGCTCCAGGCTTAGA |
| 2472 | TGAACCCGGGAGGCAGAGGT | 3596 | TGCCTCATCCTCAGCTCCAG | 4717 | GCCAGGTGTCTTATCCGCTG |
| 2473 | TTGAACCCGGGAGGCAGAGG | 3597 | CTGCCTCATCCTCAGCTCCA | 4718 | TCACCAGGGCACAGACTAGT |
| 2474 | CTTGAACCCGGGAGGCAGAG | 3598 | CCTGCCTCATCCTCAGCTCC | 4719 | ACAGGAAGGAAAATAAGCAC |
| 2475 | GCTTGAACCCGGGAGGCAGA | 3599 | CCCTGCCTCATCCTCAGCTC | 4720 | ACTATACTCCCTAGTCTCAA |
| 2476 | CGCTTGAACCCGGGAGGCAG | 3600 | CCCCTGCCTCATCCTCAGCT | 4721 | GTGTGTGTTTCAATAGAAGC |
| 2477 | TCGCTTGAACCCGGGAGGCA | 3601 | GCCCCTGCCTCATCCTCAGC | 4722 | TGATCGATTCACCTAACATT |
| 2478 | ATCGCTTGAACCCGGGAGGC | 3602 | TGCCCCTGCCTCATCCTCAG | 4723 | TTTTTCTGGTTCTGGGTGGT |
| 2479 | AATCGCTTGAACCCGGGAGG | 3603 | CTGCCCCTGCCTCATCCTCA | 4724 | TTGTTTCAGTCTGTTTAGGT |
| 2480 | GAATCGCTTGAACCCGGGAG | 3604 | CCTGCCCCTGCCTCATCCTC | 4725 | GAATCACGGCTTTGACGATT |
| 2481 | AGAATCGCTTGAACCCGGGA | 3605 | CCCTGCCCCTGCCTCATCCT | 4726 | GTCCCTGGCCGGCAAAATTG |
| 2482 | GAGAATCGCTTGAACCCGGG | 3606 | CCCCTGCCCCTGCCTCATCC | 4727 | GATTGTCTCCAGACACTCCT |
| 2483 | GGAGAATCGCTTGAACCCGG | 3607 | CCCCCTGCCCCTGCCTCATC | 4728 | ACCAGATGCCAGTGTCCCTC |
| 2484 | AGGAGAATCGCTTGAACCCG | 3608 | TCCCCCTGCCCCTGCCTCAT | 4729 | GTGATGTTTAGTGGGATTGC |
| 2485 | CAGGAGAATCGCTTGAACCC | 3609 | ATCCCCCTGCCCCTGCCTCA | 4730 | GTGGGGGATTGTTCTGTGTG |
| 2486 | GCAGGAGAATCGCTTGAACC | 3610 | CATCCCCCTGCCCCTGCCTC | 4731 | GACCTTTTGGGCAGGATACT |
| 2487 | GGCAGGAGAATCGCTTGAAC | 3611 | CCATCCCCCTGCCCCTGCCT | 4732 | AGTAGGTTGTTGAGTCTCAG |
| 2488 | AGGCAGGAGAATCGCTTGAA | 3612 | GCCATCCCCCTGCCCCTGCC | 4733 | ATCGTGGCACCTTACCCTTC |
| 2489 | GAGGCAGGAGAATCGCTTGA | 3613 | AGCCATCCCCCTGCCCCTGC | 4734 | GAGTAGGGAATTACAGACCT |
| 2490 | TGAGGCAGGAGAATCGCTTG | 3614 | TAGCCATCCCCCTGCCCCTG | 4735 | TTGTGTTGGCGGGGGTGGAT |
| 2491 | CTGAGGCAGGAGAATCGCTT | 3615 | TTAGCCATCCCCCTGCCCCT | 4736 | GCATAACTCTCTTAAGACAC |
| 2492 | GCTGAGGCAGGAGAATCGCT | 3616 | GTTAGCCATCCCCCTGCCCC | 4737 | GGCTGCCTTCTGTGGTGATA |
| | | 3617 | GGTTAGCCATCCCCCTGCCC | 4738 | CTAGGGTTAGGGAGGGTGAC |

### Hybridization and AG

The term “hybridizing” or “hybridizes” as used herein is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ, is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the free energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem, Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Aced Sci USA. 95: 1460-1465 using appropriately derived thermodynamic parameters described by Sugimoto et al., 1995, Biochemistry 34:11211-11216 and McTigue et al., 2004, Biochemistry 43:5388-5405. To have the possibility of modulating its intended nucleic acid target by hybridization, oligonucleotides of the present disclosure hybridize to a target nucleic acid with estimated ΔG° values below −10 kcal/mol for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal/mol, such as below −15 kcal/mol, such as below −20 kcal/mol and such as below −25 kcal/mol for oligonucleotides that are 8-30 nucleotides in length. In some embodiments the oligonucleotides hybridize to a target nucleic acid with an estimated ΔG° value of −10 to −60 kcal/mol, such as −12 to −40 kcal/mol, −15 to −30 kcal/mol, −16 to −27 kcal/mol, or −18 to −25 kcal/mol.

### Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 70% or greater complementarity. For example, a mismatch in a duplex region consisting of 19 base pairs results in 94.7% complementarity, rendering the duplex region substantially complementary. Duplex regions can be formed by two separate oligonucleotide strands, as well as by single oligonucleotide strands that can form hairpin structures comprising a duplex region.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of a GRN regRNA, such as an eRNA or paRNA. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides. Generally, the duplex structure is between 5 and 50 base pairs in length, e.g., between, 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 base pairs in length. Ranges and lengths intermediate to the above recited ranges and lengths are also contemplated to be part of the disclosure.

Similarly, the region of complementarity to the target sequence can be between 15 and 50 nucleotides in length, e.g., between 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 nucleotides in length. Ranges and lengths intermediate to the above recited ranges and lengths are also contemplated to be part of the disclosure.

### Chemical Modifications of ASOs

In certain embodiments, the ASO does not consist of only DNA. In certain embodiments, the ASO comprises at least one chemical modification relative to a natural nucleotide (e.g., ribonucleotide (e.g., 2′-deoxy-2′-ribonucleotide). Various chemical modifications can be included in the ASOs of the present disclosure. The modifications can include one or more modifications in a sugar group (e.g., ribose), one or more modifications in a phosphate group, one or more modifications in a nucleobase, one or more terminal modifications, or a combination thereof. In some embodiments, an exemplary ASO sequence targeting a regRNA as shown in FIG. 17, FIG. 18, or other sections of the instant disclosure, is chemically modified. Such modifications can be, but are not limited to, 2′-O-(2-methoxyethyl) (2′-MOE), locked nucleic acid (LNA), 5-methyl on the cytidine, constrained ethyl (cET), phosphorothioate (PS) linkage, and/or a phosphodiester (PO) linkage, or any combination thereof. Chemical modifications of RNA are known in the art and described in, for example, PCT Application Publication No. WO2013/177248, incorporated herein by reference. In certain embodiments, each cytidine in an ASO provided herein is modified by 5-methyl.

Various chemical modifications for use with ASOs of the present disclosure include, but are not limited to: 3′-terminal deoxy-thymine (dT) nucleotides, 2′-O-methyl modified nucleotides, 2′-fluoro modified nucleotides, 2′-deoxy-modified nucleotides, locked nucleotides, unlocked nucleotides, conformationally restricted nucleotides, constrained ethyl nucleotides, abasic nucleotides, 2′-amino-modified nucleotides, 2′-O-allyl-modified nucleotides, 2′-C-alkyl-modified nucleotides, 2′-hydroxyl-modified nucleotides, 2′-methoxyethyl modified nucleotides, 2′-O-alkyl-modified nucleotides, morpholino nucleotides, phosphoramidates, non-natural base comprising nucleotides, tetrahydropyran modified nucleotides, 1,5-anhydrohexitol modified nucleotides, cyclohexenyl modified nucleotides, nucleotides comprising a phosphorothioate group, nucleotides comprising a methylphosphonate group, nucleotides comprising a 5′-phosphate, and nucleotides comprising a 5′-phosphate mimic.

In certain embodiments, the ASO comprises an RNA polynucleotide chemically modified to be resistant to one or more nucleases (e.g., nuclear RNases (e.g., the exosome complex or RNaseH)). In some embodiments, all nucleotide bases are modified in the ASO. In certain embodiments, the chemical modifications comprises β-D-ribonucleotides, 2′-modified nucleotides (e.g., 2′-O-(2-methoxyethyl) (2′-MOE), 2′-O—CH₃, or 2′-fluoro-arabino (FANA)), bicyclic sugar modified nucleotides (e.g., having a constrained ethyl or locked nucleic acid (LNA)), and/or one or more modified internucleotide bonds (e.g., phosphorothioate internucleotide linkage). In certain embodiments, the chemical modification comprises 2′-MOE and a phosphorothioate internucleotide bond. In certain embodiments, at least 8, 9, 10, 11, 12, 13, 14, 15, 16, 17, 18, 19, 20, or more consecutive nucleotides of the ASO are modified by 2′-MOE. In certain embodiments, each nucleotide of the ASO is modified by 2′-MOE. In certain embodiments, at least 7, 8, 9, 10, 11, 12, 13, 14, 15, 16, 17, 18, 19, 20, or more consecutive internucleotide bonds of the ASO are phosphorothioate internucleotide bonds. In certain embodiments, each internucleotide bond of the ASO is a phosphorothioate internucleotide bond.

Internucleotide linkage modifications that can be used with the ASOs of the present disclosure include, but are not limited to, phosphorothioate “PS” (P(S)), phosphoramidate (P(NR₁R₂) such as dimethylaminophosphoramidate (P(N(CH₃)₂)), phosphonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂COO⁻)), alkylphosphonate (P(C₁₋₃alkyl) such as methylphosphonate —P(CH₃), boranophosphonate (P(BH₃)), and phosphorodithioate (P(S)₂).

In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PO bonds. In some embodiments, all internucleotide bonds of an ASO provided herein are PO internucleotide bonds. In some embodiments, an ASO provided herein does not comprise PO internucleotide bonds. In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS internucleotide bonds. In some embodiments, all internucleotide bonds of an ASO provided herein are PS bonds. In some embodiments, an ASO provided herein does not comprise PS internucleotide bonds.

In certain embodiments, the ASO comprises one or more chemical modifications at the 5′ end, the 3′ end, or both. Without wishing to be bound by theory, chemical modifications at one or both termini of a polynucleotide (e.g., polyribonucleotide) may stabilize the polynucleotide. In certain embodiments, the ASO comprises one or more chemical modifications in at least 1, 2, 3, 4, or 5 nucleotides at the 5′ end of the ASO. In certain embodiments, the ASO comprises one or more chemical modifications in at least 1, 2, 3, 4, or 5 nucleotides at the 3′ end of the ASO. In certain embodiments, the ASO comprises one or more chemical modifications in at least 1, 2, 3, 4, or 5 nucleotides at the 5′ end of the ASO and one or more chemical modifications in at least 1, 2, 3, 4, or 5 nucleotides at the 3′ end of the ASO.

The chemical structures can also be described in writing. In such cases, ‘M’ indicates MOE; ‘d’ indicates DNA, ‘L’ indicates LNA, “m” indicates 2′ OMethyl, ‘=’ indicates a phosphorothioate (PS) linkage, ‘-’ indicates a phosphodiester (PO) linkage; ‘5C’ indicates 5-MethylCytosine, ‘ag’ indicates GalNAc, ‘tg’ indicates Teg-GalNAc, ‘\hat{ }’ indicates FANA, “BioTeg” indicates Biotin; “Palm” indicates Palmitic acid; and “C18” indicates a Spacer 18 moiety.

To avoid ambiguity, this LNA has the formula:

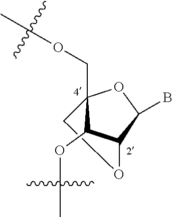

wherein B is the particular designated base.

Visual representations of exemplary ASOs with chemical modifications are provided in FIGS. 17 and 18. Additional exemplary ASOs with chemical modifications are provided in Tables 17 and 18. In some embodiments, an ASO provided herein comprises a nucleotide sequence as provided in Table 17, below. In some embodiments, an ASO provided herein comprises a nucleotide sequence as provided in Table 18, below. In some embodiments, the ASO comprises a nucleotide sequence and/or chemical modification of any one of the oligonucleotides provided in Tables 17 and 18, below. In some embodiments, the ASO comprises a nucleotide sequence and/or chemical modification of any one of SEQ ID NOs: 1-442, 691, 991-1368, or 4743-4915. In some embodiments, the ASO comprises a nucleotide sequence and/or chemical modification of any one of SEQ ID NOs: 443-690, 692-990, or 4916.

TABLE 17
Exemplary ASOs targeting hGRN regRNA with chemical modifications

| SEQ | | Chemistry and Sequence |
|---|---|---|
| ID | | MOE (M); DNA (d); LNA (L); 2′ OMethyl (m); PS (=); PO(−); 5-MethylCytosine (5C); Biotin (BioTEG); Palmitic acid |
| NO | Name | (Palm); Spacer 18 (C18) |
| 10 | CO-3413 | M5C = MA = M5C = M5C = MT = MA = MG = MT = MG = MG = MA = MA = MG = MA = MT = MA = MA = M5C = MG = MG |
| 11 | CO-3414 | M5C = M5C = M5C = MT = MT = MG = MG = M5C = MA = M5C = MT = M5C = MG = MG = MA = MG = MT = MT = MG = MG |
| 12 | CO-3415 | MA = MG = M5C = MT = MA = MG = MG = MT = MT = MG = MG = M5C = MT = M5C = M5C = M5C = M5C = MT = MT = MT |
| 13 | CO-3416 | MT = M5C = M5C = M5C = MA = MG = M5C = M5C = MA = M5C = MT = MT = MT = MT = MT = M5C = M5C = MT = MG = M5C |
| 14 | CO-3417 | MT = M5C = M5C = MG = M5C = M5C = MT = M5C = MT = M5C = M5C = M5C = MA = MG = M5C = M5C = MA = M5C = MT = MT |
| 15 | CO-3418 | MT = MT = M5C = M5C = MG = M5C = M5C = MT = M5C = MT = M5C = M5C = M5C = MA = MG = M5C = M5C = MA = M5C = MT |
| 16 | CO-3419 | MG = MG = MT = MG = MG = MG = MA = M5C = MG = MA = MG = M5C = MT = MA = MG = M5C = MT = MT = M5C = M5C |
| 17 | CO-3420 | MT = MT = MA = M5C = MT = MT = M5C = M5C = MT = M5C = MT = MT = MG = MT = MT = MG = M5C = MG = MG = MG |
| 18 | CO-3421 | MT = M5C = MT = MG = MG = M5C = MT = M5C = M5C = MT = M5C = M5C = MT = MT = MA = M5C = MT = MT = M5C = M5C |
| 19 | CO-3422 | MG = MT = MG = MA = M5C = MT = MG = MA = MA = M5C = M5C = MT = MG = MG = MT = M5C = MT = MG = MG = M5C |
| 20 | CO-3423 | MG = MG = MA = M5C = MT = MT = MG = MG = MG = MA = MG = MT = MG = MG = MT = MG = MA = M5C = MT = MG |
| 21 | CO-3424 | MG = M5C = MT = MT = M5C = MT = M5C = MG = MA = MA = MG = MG = MT = MA = MG = MT = MA = MG = MG = MG |
| 22 | CO-3425 | MG = MA = MG = MA = M5C = M5C = MT = MG = MA = MG = MA = M5C = M5C = MT = MT = MG = MG = M5C = MT = MT |
| 23 | CO-3426 | MG = MG = M5C = M5C = MT = MG = MG = MG = MA = MA = M5C = MG = MA = MG = MA = M5C = M5C = MT = MG = MA |
| 24 | CO-3427 | M5C = MT = MG = MG = MT = MG = MG = MT = MG = MG = MT = MG = MG = MG = MT = MG = MG = MT = MT = M5C |
| 25 | CO-3428 | MA = M5C = M5C = M5C = M5C = MT = MG = MT = M5C = MG = MG = MT = MA = MG = MG = MT = MG = M5C = MT = MG |
| 26 | CO-3429 | MG = MG = MA = M5C = M5C = MA = M5C = MA = M5C = M5C = MA = MT = MT = M5C = MT = MT = MG = MA = M5C = M5C |
| 27 | CO-3430 | MG = MG = M5C = MT = MT = MT = M5C = MT = MA = M5C = M5C = MT = M5C = M5C = M5C = M5C = MA = MG = M5C = MA |
| 28 | CO-3431 | MA = MT = MT = MG = M5C = MA = MG = MT = MG = MA = M5C = MG = M5C = M5C = M5C = M5C = MG = MT = MT = MA |
| 29 | CO-3432 | MT = MT = MT = MT = MA = MT = MG = MG = MG = MA = MA = MA = MG = MA = MG = MG = MA = MA = MG = M5C |
| 30 | CO-3433 | M5C = MG = M5C = MT = MT = MA = M5C = MT = MG = MA = MA = MA = M5C = MT = M5C = M5C = MT = MT = MG = MG |
| 31 | CO-3434 | MA = MG = MA = MA = MG = MA = MA = M5C = M5C = MG = M5C = MT = MT = MA = M5C = MT = MG = MA = MA = MA |
| 32 | CO-3435 | M5C = MG = MG = MA = MG = MA = M5C = MA = MA = M5C = MA = MG = MA = MA = MG = MA = MA = M5C = M5C = MG |
| 33 | CO-3436 | MT = MT = M5C = M5C = M5C = M5C = MT = MG = MG = MA = MG = MT = M5C = MT = M5C = MA = MG = M5C = M5C = MG |
| 34 | CO-3437 | MG = MG = M5C = M5C = MA = MT = MG = MT = MG = MA = MG = M5C = MT = MT = MG = MA = MG = MG = MT = MT |
| 35 | CO-3438 | MA = MG = MA = M5C = M5C = MT = M5C = MT = M5C = MG = M5C = M5C = MT = MG = M5C = MT = M5C = M5C = MT = MG |
| 36 | CO-3439 | MA = MG = MT = M5C = MG = MG = MA = M5C = MG = M5C = MA = MG = MG = MT = MA = MG = MG = MA = MG = MA |
| 37 | CO-3440 | MT = MA = M5C = M5C = M5C = MG = MG = MT = MT = MG = M5C = MT = MG = M5C = MT = MG = M5C = M5C = M5C = MA |
| 38 | CO-3441 | MG = MG = MT = M5C = MT = MG = MT = MA = MG = MT = M5C = MT = MG = MA = MG = M5C = MG = M5C = MT = MA |
| 39 | CO-3442 | MA = MA = MT = MG = MG = MA = MA = MA = M5C = MT = MG = MA = MG = MG = MT = MA = MG = MG = M5C = MG |
| 40 | CO-3443 | MT = MA = MA = MG = MT = MA = MG = M5C = M5C = MA = MA = MT = MG = MG = MG = MA = MG = M5C = MG = MG |
| 41 | CO-3444 | MG = MG = M5C = M5C = M5C = M5C = MA = M5C = M5C = MT = M5C = MT = MA = MT = MA = MT = MT = MG = MA = MT |
| 42 | CO-3445 | MA = MG = MA = MA = MA = M5C = MA = M5C = MA = MG = M5C = MA = MT = MT = M5C = M5C = MA = MG = MG = M5C |
| 43 | CO-3446 | MA = MA = MG = MA = MA = MA = M5C = MA = M5C = MA = MG = M5C = MA = MT = MT = M5C = M5C = MA = MG = MG |
| 44 | CO-3447 | MG = MA = MA = MA = MA = MG = MA = MA = MA = M5C = MA = M5C = MA = MG = M5C = MA = MT = MT = M5C = M5C |
| 45 | CO-3448 | MT = MA = MG = MA = MA = MA = MA = MG = MA = MA = MA = M5C = MA = M5C = MA = MG = M5C = MA = MT = MT |
| 46 | CO-3449 | MG = MG = MG = MA = MG = MG = MA = MG = MA = MG = MT = MG = MA = MT = MT = MT = MG = MA = MG = MT |
| 47 | CO-3450 | MT = M5C = MA = M5C = MA = MT = MG = MA = MT = M5C = M5C = M5C = MT = MA = MG = MA = MA = MA = MT = MG |
| 48 | CO-3451 | MA = MT = M5C = MA = M5C = MA = MT = MG = MA = MT = M5C = M5C = M5C = MT = MA = MG = MA = MA = MA = MT |
| 49 | CO-3452 | M5C = M5C = MA = MA = MT = M5C = MA = M5C = MA = MT = MG = MA = MT = M5C = M5C = M5C = MT = MA = MG = MA |
| 50 | CO-3453 | M5C = MT = MG = MT = M5C = M5C = M5C = MG = MA = M5C = MG = MT = M5C = MA = M5C = MA = MT = MG = MA = MT |
| 51 | CO-3454 | MT = MG = MG = MG = MT = MT = MA = MA = MG = MG = MA = MA = MG = MG = M5C = MG = MA = M5C = MG = MA |
| 52 | CO-3455 | MG = M5C = M5C = MT = MG = M5C = MA = MG = MG = MA = MT = MG = MG = MG = MT = MT = MA = MA = MG = MG |
| 53 | CO-3456 | MT = MA = MT = M5C = MA = MT = M5C = MT = M5C = MT = MA = MG = M5C = MA = MA = MA = M5C = MT = M5C = M5C |
| 54 | CO-3457 | MA = M5C = MA = M5C = MG = M5C = MG = M5C = MT = MA = MT = M5C = MA = MT = M5C = MT = M5C = MT = MA = MG |
| 55 | CO-3458 | MG = M5C = MA = M5C = MT = MG = MT = M5C = MA = MA = MT = MG = M5C = M5C = M5C = M5C = MA = MG = MA = M5C |
| 56 | CO-3459 | MA = MA = MT = MT = M5C = MA = MT = MA = MA = M5C = MA = M5C = MT = M5C = M5C = M5C = MT = M5C = MG = M5C |
| 57 | CO-3460 | MG = MG = MA = MT = MA = MG = MA = MA = MA = MG = MG = M5C = MG = MA = MG = M5C = MA = M5C = MA = MA |
| 58 | CO-3461 | MA = MA = M5C = MA = MA = MA = M5C = MA = M5C = MA = M5C = MA = MA = MG = MT = M5C = M5C = MG = MG = MG |
| 59 | CO-3462 | MT = MA = MG = MG = MA = MG = MA = MA = MT = M5C = MG = M5C = MT = MT = MT = MG = MG = MG = MA = MG |
| 60 | CO-3463 | MG = MG = MA = MT = MA = MG = MG = MA = MT = MA = MG = MG = MA = MG = M5C = MG = MA = MG = M5C = M5C |
| 61 | CO-3464 | MT = MA = MG = M5C = MA = MT = MG = MT = M5C = MA = MA = M5C = M5C = MG = MA = MG = MT = MT = MT = MG |
| 62 | CO-3465 | MA = MT = M5C = M5C = MT = MT = M5C = MA = MA = M5C = MT = M5C = MA = M5C = MT = M5C = MT = MG = MG = MG |
| 63 | CO-3466 | MG = M5C = MT = MG = MT = MT = MT = MA = MT = M5C = MT = MT = MT = MG = MT = M5C = M5C = MT = MG = MG |
| 64 | CO-3467 | MG = MT = M5C = M5C = MT = MG = M5C = MT = MG = MA = MG = M5C = MA = M5C = M5C = MT = MT = MG = MA = MG |
| 65 | CO-3468 | MA = MG = MA = M5C = MT = MG = MT = M5C = MT = M5C = M5C = M5C = MA = M5C = M5C = MA = MA = MT = M5C = M5C |
| 66 | CO-3469 | M5C = MT = MT = MA = MA = MA = MT = MT = MA = MG = MA = MT = MT = M5C = MA = MG = M5C = M5C = MT = MG |
| 67 | CO-3470 | M5C = MT = MG = MG = M5C = MG = M5C = MA = M5C = MA = MA = M5C = M5C = MT = MT = MG = MT = MA = MT = M5C |
| 68 | CO-3471 | MT = M5C = MT = M5C = M5C = M5C = MT = MA = M5C = MA = M5C = M5C = MT = MG = MG = M5C = MA = MA = M5C = MT |
| 69 | CO-3472 | M5C = M5C = M5C = MA = M5C = M5C = MT = MG = MG = MA = MG = MA = M5C = MA = MA = MT = M5C = MT = MA = M5C |
| 70 | CO-3473 | MG = MA = MT = MG = M5C = MT = M5C = M5C = MT = MA = MA = MG = MG = MT = MG = MG = MA = MA = MT = MG |
| 71 | CO-3474 | M5C = M5C = MA = MG = MA = M5C = MT = M5C = MA = MG = M5C = MT = M5C = MA = MA = MG = MG = MA = MG = MA |
| 72 | CO-3475 | MA = M5C = M5C = MT = MG = M5C = M5C = MA = MG = MT = MT = MA = MA = MA = MA = MT = M5C = MT = MT = M5C |
| 73 | CO-3476 | MG = MG = M5C = MT = M5C = MT = MT = M5C = M5C = M5C = MA = MA = M5C = MA = M5C = M5C = MT = M5C = M5C = MT |
| 74 | CO-3477 | MT = MG = M5C = M5C = M5C = MT = MT = M5C = M5C = M5C = MA = MT = M5C = MT = MG = MG = MT = MT = MT = M5C |
| 75 | CO-3478 | MG = M5C = MA = MT = MT = M5C = MA = MA = MT = M5C = M5C = M5C = MT = MG = M5C = M5C = MA = MA = MG = M5C |
| 76 | CO-3479 | M5C = M5C = MA = M5C = MT = MG = MG = M5C = MA = MT = MT = MG = MA = MA = M5C = MA = MT = MG = MG = M5C |
| 77 | CO-3480 | MG = M5C = M5C = MA = M5C = MT = MT = M5C = MT = M5C = MT = MT = MT = M5C = MG = MA = M5C = MA = MG = M5C |
| 78 | CO-3481 | MG = M5C = M5C = MG = MA = MT = M5C = MT = MG = MG = MA = MG = MA = M5C = MT = MA = MG = MG = MA = MA |
| 79 | CO-3482 | M5C = M5C = MA = MT = MA = MT = M5C = MA = MA = MT = MT = M5C = M5C = MT = M5C = MG = MA = MA = MG = MG |
| 80 | CO-3483 | MG = M5C = MT = MA = MA = MT = M5C = MT = MG = MT = MT = M5C = MT = MG = MT = MG = MG = MT = MA = M5C |
| 81 | CO-3484 | M5C = MA = M5C = M5C = MA = MA = MG = M5C = MA = MG = MG = M5C = MT = MG = MA = M5C = MT = M5C = MT = MT |
| 82 | CO-3485 | M5C = MA = MT = M5C = MA = MT = M5C = M5C = MA = MT = M5C = MT = M5C = MT = MG = M5C = M5C = MT = MG = MG |
| 83 | CO-3486 | MT = M5C = MT = M5C = M5C = MT = M5C = MT = M5C = MT = M5C = M5C = MT = MG = MG = M5C = M5C = M5C = MA = MT |
| 84 | CO-3487 | MG = MA = M5C = MA = M5C = MT = MG = M5C = M5C = MG = MT = MT = M5C = M5C = MT = M5C = M5C = MA = M5C = MG |
| 85 | CO-3488 | MT = MG = M5C = MA = MG = M5C = MA = MG = MT = M5C = MT = M5C = M5C = MA = MT = M5C = M5C = M5C = M5C = MT |
| 86 | CO-3489 | MT = M5C = M5C = MA = MG = MT = MT = M5C = M5C = MT = MT = MG = M5C = MT = M5C = MA = MG = MG = M5C = MT |
| 87 | CO-3490 | MT = MG = MG = MT = MT = MA = MG = M5C = M5C = MA = MT = M5C = M5C = M5C = M5C = M5C = MT = MG = M5C = M5C |
| 88 | CO-3491 | M5C = M5C = MG = MA = M5C = MT = MT = M5C = MT = MG = M5C = MT = MG = M5C = M5C = MT = M5C = MG = MA = MG |
| 89 | CO-3492 | M5C = M5C = M5C = MT = MT = MT = M5C = M5C = M5C = MT = M5C = M5C = MT = M5C = M5C = MG = MA = M5C = MT = MT |
| 90 | CO-3493 | MA = MG = M5C = MT = M5C = MG = MG = MT = MT = MG = MG = M5C = MA = M5C = M5C = M5C = MT = MT = MT = M5C |
| 91 | CO-3494 | MG = M5C = M5C = MA = M5C = MA = M5C = MG = MA = M5C = MT = M5C = M5C = MA = M5C = MA = MG = M5C = MT = M5C |
| 92 | CO-3495 | MT = MT = MA = MG = MA = MA = M5C = M5C = M5C = M5C = MT = MG = M5C = M5C = MA = M5C = MA = M5C = MG = MA |
| 93 | CO-3496 | MG = MG = MT = M5C = MT = MG = M5C = MT = M5C = MT = MA = MG = M5C = MT = M5C = M5C = MA = MG = MG = M5C |
| 94 | CO-3497 | MG = MT = MG = MT = M5C = MT = MT = MA = MT = M5C = M5C = MG = M5C = MT = MG = MT = M5C = MT = MG = MG |
| 95 | CO-3498 | MA = M5C = MT = MA = MG = MT = MA = M5C = MT = MA = MG = MG = MT = M5C = M5C = MT = M5C = MA = MG = M5C |
| 96 | CO-3499 | MT = M5C = MT = M5C = MA = MA = MG = MA = MT = MA = M5C = MA = M5C = MA = MG = MG = MA = MA = MG = MG |
| 97 | CO-3500 | MA = MT = MA = MA = MA = M5C = MT = MA = MT = MA = M5C = MT = M5C = M5C = M5C = MT = MA = MG = MT = M5C |
| 98 | CO-3501 | MA = M5C = M5C = MT = MA = MA = M5C = MA = MT = MT = MT = M5C = MA = MT = MG = MA = MA = MA = MG = MT |
| 99 | CO-3502 | MG = MT = MA = MG = MG = MT = MT = MA = MT = MT = MG = MA = MT = M5C = MG = MA = MT = MT = M5C = MA |
| 100 | CO-3503 | MT = M5C = MT = MG = MG = MG = MT = MG = MG = MT = MT = MG = MT = MA = MA = MT = MT = MA = MG = MT |
| 101 | CO-3504 | M5C = MT = MA = M5C = M5C = MT = MG = MT = MA = MT = MT = MT = MT = MT = MT = MT = M5C = MT = MG = MG |
| 102 | CO-3505 | MG = MT = MG = MA = MT = MA = MT = MT = MT = MG = MT = MT = MT = M5C = MA = MG = MT = M5C = MT = MG |
| 103 | CO-3506 | M5C = MG = MG = M5C = MT = MT = MT = MG = MA = M5C = MG = MA = MT = MT = MA = MG = MA = M5C = MT = MT |
| 104 | CO-3507 | MG = M5C = MT = MG = M5C = MT = M5C = MA = MA = MG = MA = MA = MT = M5C = MA = M5C = MG = MG = M5C = MT |
| 105 | CO-3508 | MA = MT = MT = MG = M5C = M5C = MG = M5C = MT = MG = M5C = MT = M5C = MA = MA = MG = MA = MA = MT = M5C |
| 106 | CO-3509 | M5C = MT = M5C = M5C = MT = MA = MA = MA = MT = MG = MT = M5C = M5C = M5C = MT = MG = MG = M5C = M5C = MG |
| 107 | CO-3510 | MG = MT = MG = MT = M5C = M5C = M5C = MT = M5C = M5C = MA = M5C = M5C = M5C = MT = MG = MA = MG = MT = MT |
| 108 | CO-3511 | MG = MG = MA = MT = MT = MG = M5C = MT = MG = MG = M5C = M5C = M5C = MT = MT = MA = M5C = M5C = MT = MA |
| 109 | CO-3512 | MG = MT = M5C = MG = MT = MG = MT = MG = MA = MT = MG = MT = MT = MT = MA = MG = MT = MG = MG = MG |
| 110 | CO-3513 | MG = MA = MT = MT = MG = MT = MT = M5C = MT = MG = MT = MG = MT = MG = MT = M5C = MG = MT = MG = MT |
| 111 | CO-3514 | MG = MT = MG = MA = MA = MG = MT = MA = MG = MG = MT = MT = MG = MT = MT = MG = MA = MG = MT = M5C |
| 112 | CO-3515 | MA = M5C = M5C = MT = MT = MA = M5C = M5C = M5C = MT = MT = M5C = MT = MG = MA = MT = MG = MT = MT = MA |
| 113 | CO-3516 | MT = MT = MA = M5C = MA = MG = MA = M5C = M5C = MT = M5C = MG = MT = MT = MA = MT = M5C = MG = MT = MG |
| 114 | CO-3517 | MA = MA = MT = MT = MT = MT = MT = MT = MT = MG = MT = MG = MT = MT = MG = MG = M5C = MG = MG = MG |
| 115 | CO-3518 | MT = MG = M5C = MT = MG = M5C = MA = MT = MA = MA = M5C = MT = M5C = MT = M5C = MT = MT = MA = MA = MG |
| 116 | CO-3519 | MA = MT = MT = MG = MT = MA = MA = MT = MG = MA = MT = MG = M5C = MT = MG = M5C = MA = MT = MA = MA |
| 117 | CO-3520 | MT = MG = MG = MT = M5C = MT = MT = MA = MA = MA = M5C = MT = MG = MA = MT = MT = MG = MT = MA = MA |
| 118 | CO-3521 | M5C = MT = MA = MA = MA = MG = MG = MA = M5C = MA = MT = MA = MA = MG = MG = M5C = MT = MG = M5C = M5C |
| 119 | CO-3522 | MG = MG = MT = MT = MA = MG = MG = MG = MA = MG = MG = MG = MT = MG = MA = M5C = MT = MG = M5C = MT |
| 120 | CO-4106 | MG = MG = MA = M5C = MT = dT = dG = dG = dG = dA = dG = dT = dG = dG = dT = MG = MA = M5C = MT = MG |
| 121 | CO-4107 | MA = MT = MT = MG = M5C = dA = dG = dT = dG = dA = dC = dG = dC = dC = dC = M5C = MG = MT = MT = MA |
| 122 | CO-4108 | MG = MG = MA = MT = MA = dG = dG = dA = dT = dA = dG = dG = dA = dG = dC = MG = MA = MG = M5C = M5C |
| 123 | CO-4109 | MT = M5C = MT = MG = MG = dG = dT = dG = dG = dT = dT = dG = dT = dA = dA = MT = MT = MA = MG = MT |
| 124 | CO-4110 | MG = MG = MA = M5C = MT = dT = dG = dG = MG = dA = dG = MT = dG = dG = dT = MG = MA = M5C = MT = MG |
| 125 | CO-4111 | MG = MG = MA = MT = MA = dG = dG = dA = MT = dA = dG = MG = dA = dG = dC = MG = MA = MG = M5C = M5C |
| 126 | CO-4112 | MT = M5C = MT = MG = MG = dG = dT = dG = MG = dT = dT = MG = dT = dA = dA = MT = MT = MA = MG = MT |
| 127 | CO-4113 | MG = MG = MA = M5C = MT = LT = MG = MG = LG = MA = MG = LT = MG = MG = LT = MG = MA = M5C = MT = MG |
| 128 | CO-4114 | MG = MG = MA = MT = MA = LG = MG = MA = LT = MA = MG = LG = MA = MG = L5C = MG = MA = MG = M5C = M5C |
| 129 | CO-4115 | MT = M5C = MT = MG = MG = LG = MT = MG = LG = MT = MT = LG = MT = MA = LA = MT = MT = MA = MG = MT |
| 130 | CO-4116 | MG = MG = MG = MA = M5C = MT = MT = MG = MG = MG = MA = MG = MT = MG = MG = MT = MG = MA = M5C = MT = MG = MA |
| 131 | CO-4117 | MA = MG = MG = MG = MA = M5C = MT = MT = MG = MG = MG = MA = MG = MT = MG = MG = MT = MG = MA = M5C = MT = MG = MA = MA |
| 132 | CO-4118 | MA = MG = MG = MA = MT = MA = MG = MG = MA = MT = MA = MG = MG = MA = MG = M5C = MG = MA = MG = M5C = M5C = MA |
| 133 | CO-4119 | M5C = MA = MG = MG = MA = MT = MA = MG = MG = MA = MT = MA = MG = MG = MA = MG = M5C = MG = MA = MG = M5C = M5C = MA = MG |
| 134 | CO-4120 | MT = MT = M5C = MT = MG = MG = MG = MT = MG = MG = MT = MT = MG = MT = MA = MA = MT = MT = MA = MG = MT = MA |
| 135 | CO-4121 | MG = MT = MT = M5C = MT = MG = MG = MG = MT = MG = MG = MT = MT = MG = MT = MA = MA = MT = MT = MA = MG = MT = MA = MG |
| 136 | CO-4122 | M5C = MT = MT = MG = MG = MG = MA = MG = MT = MG = MG = MT = MG = MA = M5C = MT = MG = MA = MA = M5C |
| 137 | CO-4123 | MA = M5C = MT = MT = MG = MG = MG = MA = MG = MT = MG = MG = MT = MG = MA = M5C = MT = MG = MA = MA |
| 138 | CO-4124 | MG = MA = M5C = MT = MT = MG = MG = MG = MA = MG = MT = MG = MG = MT = MG = MA = M5C = MT = MG = MA |
| 139 | CO-4125 | MG = MG = MG = MA = M5C = MT = MT = MG = MG = MG = MA = MG = MT = MG = MG = MT = MG = MA = M5C = MT |
| 140 | CO-4126 | MT = MA = MG = MG = MG = MA = M5C = MT = MT = MG = MG = MG = MA = MG = MT = MG = MG = MT = MG = MA |
| 141 | CO-4127 | MA = MG = MT = MA = MG = MG = MG = MA = M5C = MT = MT = MG = MG = MG = MA = MG = MT = MG = MG = MT |
| 142 | CO-4128 | MG = MT = MA = MG = MT = MA = MG = MG = MG = MA = M5C = MT = MT = MG = MG = MG = MA = MG = MT = MG |
| 143 | CO-4129 | MA = MG = MG = MT = MA = MG = MT = MA = MG = MG = MG = MA = M5C = MT = MT = MG = MG = MG = MA = MG |
| 144 | CO-4130 | MT = MA = MG = MG = MA = MT = MA = MG = MG = MA = MG = M5C = MG = MA = MG = M5C = M5C = MA = MG = M5C |
| 145 | CO-4131 | MG = MA = MT = MA = MG = MG = MA = MT = MA = MG = MG = MA = MG = M5C = MG = MA = MG = M5C = M5C = MA |
| 146 | CO-4132 | MA = MG = MG = MA = MT = MA = MG = MG = MA = MT = MA = MG = MG = MA = MG = M5C = MG = MA = MG = M5C |
| 147 | CO-4133 | M5C = MA = MG = MG = MA = MT = MA = MG = MG = MA = MT = MA = MG = MG = MA = MG = M5C = MG = MA = MG |
| 148 | CO-4134 | MG = MG = MG = M5C = MA = MG = MG = MA = MT = MA = MG = MG = MA = MT = MA = MG = MG = MA = MG = M5C |
| 149 | CO-4135 | M5C = MA = MA = MG = MA = M5C = MA = MG = MG = MG = M5C = MA = MG = MG = MA = MT = MA = MG = MG = MA |
| 150 | CO-4136 | MA = MG = MG = MG = M5C = MA = MG = MA = MA = MT = MA = MG = MG = MG = M5C = MA = MG = MG = MA = M5C |
| 151 | CO-4137 | MA = MA = M5C = M5C = MG = MA = MG = MT = MT = MT = MG = MG = MA = MG = MA = MA = MT = MA = MG = MG |
| 152 | CO-4138 | MG = MG = MG = MT = MG = MG = MT = MT = MG = MT = MA = MA = MT = MT = MA = MG = MT = MA = MG = MG |
| 153 | CO-4139 | MT = MG = MG = MG = MT = MG = MG = MT = MT = MG = MT = MA = MA = MT = MT = MA = MG = MT = MA = MG |
| 154 | CO-4140 | MT = MT = M5C = MT = MG = MG = MG = MT = MG = MG = MT = MT = MG = MT = MA = MA = MT = MT = MA = MG |
| 155 | CO-4141 | MT = M5C = MT = MG = MG = MT = MT = M5C = MT = MG = MG = MG = MT = MG = MG = MT = MT = MG = MT = MA |
| 156 | CO-4353 | MG = MG = MA = M5C = MT = LT = dG = dG = LG = dA = dG = LT = dG = dG = LT = MG = MA = M5C = MT = MG |
| 157 | CO-4354 | MG = MG = MA = L5C = MT = LT = MG = LG = MG = LA = MG = LT = MG = LG = MT = LG = MA = L5C = MT = MG |
| 158 | CO-4355 | MG = MG = LA = M5C = LT = MT = LG = MG = LG = MA = LG = MT = LG = MG = LT = MG = LA = M5C = MT = MG |
| 159 | CO-4356 | MG = MG = LA = M5C = MT = LT = MG = MG = LG = MA = MG = LT = MG = MG = LT = MG = MA = L5C = MT = MG |
| 160 | CO-4357 | MG = MG = MA = M5C = LT = MT = MG = MG = LG = MA = MG = MT = LG = MG = MT = MG = LA = M5C = MT = MG |
| 161 | CO-4358 | MG = MG = MA = L5C = MT = MT = MG = LG = MG = MA = MG = LT = MG = MG = MT = LG = MA = M5C = MT = MG |
| 162 | CO-4359 | MG = MG = MA = M5C = MT = LT = MG = MG = MG = MA = LG = MT = MG = MG = MT = LG = MA = M5C = MT = MG |
| 163 | CO-4360 | MG = MG = MA = M5C = LT = MT = MG = MG = MG = LA = MG = MT = MG = MG = LT = MG = MA = M5C = MT = MG |
| 164 | CO-4361 | MG = MA = M5C = MT = MT = dG = dG = dG = dA = dG = dT = dG = dG = dT = dG = MA = M5C = MT = MG = MA |
| 165 | CO-4362 | MG = MA = M5C = MT = MT = dG = dG = dG = MA = dG = dT = MG = dG = dT = dG = MA = M5C = MT = MG = MA |
| 166 | CO-4363 | MG = MG = MA = M5C = MT = MT = MG = MG = MG = MA = MG = MT = MG = MG = MT = MG = MA = M5C = MT = MG = MA |
| 167 | CO-4364 | MG = MG = MA = M5C = MT = MT = MG = MG = MG = MA = MG = MT = MG = MG = MT = MG = MA = M5C = MT = MG = MA = MA = M5C |
| 168 | CO-4452 | MG = MG = MA − M5C − MT = MT = MG = MG = MG = MA = MG = MT = MG = MG − MT = MG − MA − M5C = MT = MG |
| 169 | CO-4453 | MG = MG = MA − M5C − MT = MT = MG = MG = MG = MA = MG = MT = MG = MG = MT = MG − MA − M5C = MT = MG |
| 170 | CO-4454 | MG = MG = MA − M5C − MT − MT − MG − MG − MG − MA − MG − MT − MG − MG − MT − MG − MA − M5C = MT = MG |
| 171 | CO-4455 | MA = MT = MT − MG − M5C = MA = MG = MT = MG = MA = M5C = MG = M5C = M5C = M5C = M5C − MG − MT = MT = MA |
| 172 | CO-4456 | MA = MT = MT − MG − M5C − MA − MG = MT = MG = MA = M5C = MG = M5C = M5C − M5C − M5C − MG − MT = MT = MA |
| 173 | CO-4457 | MA = MT = MT − MG − M5C − MA − MG − MT − MG − MA − M5C − MG − M5C − M5C − M5C − M5C − MG − MT = MT = MA |
| 269 | CO-4585 | MT = M5C = MG = MA = MA = MT = M5C = MT = M5C = M5C = MG = MG = M5C = MT = M5C = MT = MT = M5C = MA = M5C |
| 270 | CO-4586 | MT = MG = MA = M5C = MA = MA = MG = M5C = MG = MG = MG = MA = MT = M5C = MG = MG = MA = MG = MA = MG |
| 271 | CO-4587 | MT = MT = M5C = MT = MA = MA = MA = M5C = MG = M5C = MG = MA = MA = MG = MT = MG = MG = MG = MT = MG |
| 272 | CO-4588 | MG = M5C = MT = MG = MG = MT = MT = MT = M5C = MT = M5C = MT = MG = MT = MT = MA = MG = MG = MA = M5C |
| 273 | CO-4589 | M5C = MG = M5C = MA = MG = MG = M5C = MT = MA = MG = MG = MA = MG = MT = MA = M5C = M5C = M5C = MA = MT |
| 274 | CO-4590 | MG = MT = M5C = MA = MA = MA = MG = MT = M5C = MG = MA = MG = MG = M5C = M5C = MT = MA = M5C = M5C = M5C |
| 275 | CO-4591 | MG = MA = MT = MA = MG = MT = MT = MG = MG = M5C = M5C = M5C = M5C = MT = M5C = MG = MG = MA = MG = MT |
| 276 | CO-4592 | MG = MG = MT = M5C = MA = M5C = MA = MG = MA = MT = MT = MG = M5C = MA = M5C = MT = M5C = MT = M5C = M5C |
| 277 | CO-4593 | MT = MT = MT = MT = MG = M5C = M5C = M5C = MA = MG = M5C = MG = M5C = M5C = M5C = M5C = MT = MT = MT = MA |
| 278 | CO-4594 | MA = MA = MA = MG = MG = MA = M5C = MG = M5C = M5C = MT = MT = M5C = MT = M5C = MT = M5C = MG = MG = MG |
| 279 | CO-4595 | MA = MG = M5C = M5C = M5C = MG = MA = M5C = M5C = MT = MT = MG = MA = M5C = MT = MG = MA = MT = MA = MG |
| 280 | CO-4596 | M5C = MA = MA = M5C = MT = MA = MT = M5C = MA = MG = MT = M5C = MA = MA = MG = MG = MT = M5C = MG = MG |
| 281 | CO-4597 | MA = MG = MG = M5C = MG = MT = M5C = M5C = MT = MT = MT = M5C = M5C = MA = MT = MT = M5C = M5C = M5C = MT |
| 282 | CO-4598 | MG = MT = MG = M5C = MA = MA = MT = M5C = MT = MG = MT = MG = MA = M5C = M5C = MT = MG = MT = MA = MA |
| 283 | CO-4599 | MG = MG = M5C = M5C = MA = MA = M5C = MT = MA = MT = M5C = MA = MG = M5C = MT = MT = MT = M5C = M5C = M5C |
| 284 | CO-4600 | MT = MT = MA = MG = M5C = MA = M5C = MG = MT = MG = MG = MA = M5C = MT = M5C = M5C = MG = MA = MG = MG |
| 285 | CO-4601 | MG = MT = MA = MG = MG = M5C = M5C = MT = M5C = MG = MA = M5C = MT = MT = MT = MG = MA = M5C = MG = MT |
| 286 | CO-4602 | MT = MA = M5C = MT = M5C = M5C = MT = MA = MG = M5C = M5C = MT = MG = M5C = MG = M5C = M5C = MT = MT = MT |
| 287 | CO-4603 | M5C = M5C = MA = M5C = MT = MT = M5C = MG = M5C = MG = MT = MT = MT = MA = MG = MA = MA = M5C = MT = MT |
| 288 | CO-4604 | MT = M5C = MT = M5C = M5C = MG = MA = MT = M5C = M5C = M5C = MG = M5C = MT = MT = MG = MT = M5C = MA = MG |
| 289 | CO-4605 | M5C = MG = MG = MG = MA = MG = MA = M5C = MA = MG = MG = MA = MA = MG = MG = M5C = MT = MT = MA = MA |
| 290 | CO-4606 | MA = M5C = MG = MT = MG = MA = MA = MG = MA = MG = M5C = M5C = MG = MG = MA = MG = MA = MT = MT = M5C |
| 291 | CO-4607 | MA = MA = MG = MA = MA = MA = MT = MA = MG = MA = MT = MT = MA = M5C = M5C = M5C = M5C = MG = MT = MT |
| 292 | CO-4608 | MT = MA = MG = MA = M5C = MA = M5C = MT = MT = M5C = M5C = MA = MG = M5C = M5C = M5C = MA = M5C = M5C = MT |
| 293 | CO-4609 | M5C = M5C = MA = MA = MT = MA = MA = MA = MG = M5C = M5C = MT = MT = MG = MG = MG = MT = MG = M5C = M5C |
| 294 | CO-4610 | MG = MG = MA = M5C = MA = MT = MG = M5C = MT = MG = MG = MA = MA = MA = MG = MT = M5C = MT = MT = MA |
| 295 | CO-4611 | MT = MA = MT = MT = MA = MA = MT = M5C = M5C = MA = M5C = MG = M5C = MT = MG = MT = M5C = M5C = M5C = MA |
| 296 | CO-4612 | MA = MT = MG = MG = M5C = MT = MA = MA = MA = MA = M5C = MT = MA = M5C = M5C = MT = MT = M5C = M5C = MA |
| 297 | CO-4613 | MA = MT = M5C = MA = MA = MG = MG = MG = MT = MT = MT = MG = MG = M5C = MG = MT = MT = MT = MA = MG |
| 298 | CO-4614 | MT = MG = MA = M5C = MT = MA = MG = MA = MG = MA = MA = MA = M5C = MG = M5C = MT = MT = MA = MG = MA |
| 299 | CO-4615 | M5C = MA = MT = MT = MT = M5C = MA = M5C = MA = M5C = MT = MT = MG = MT = MA = MT = MG = MA = MG = M5C |
| 300 | CO-4616 | MA = MA = MG = MA = MA = MG = MG = M5C = M5C = MA = MA = MG = M5C = MT = MG = MG = MT = MA = M5C = M5C |
| 301 | CO-4617 | MG = MG = MA = MG = MA = MT = MA = MG = MG = MG = MT = M5C = MA = M5C = MG = MT = MA = M5C = MT = MT |
| 302 | CO-4618 | MA = MT = MA = MG = MG = MT = M5C = MA = MG = MG = MG = MA = MA = MT = MG = M5C = M5C = MG = M5C = M5C |
| 303 | CO-4619 | MT = MG = MA = MT = MA = MT = MA = MG = MA = MG = MA = MT = M5C = MT = MG = M5C = MA = MG = M5C = M5C |
| 304 | CO-4620 | MG = MT = M5C = M5C = MA = MG = M5C = MA = M5C = MG = M5C = MT = M5C = MT = M5C = MA = MG = M5C = MA = M5C |
| 305 | CO-4621 | M5C = MG = M5C = MT = MG = M5C = MT = MG = MT = MT = MT = MG = MG = MA = MA = M5C = MG = MA = MG = MT |
| 306 | CO-4622 | MA = M5C = MA = MG = MT = MG = MG = M5C = M5C = MG = M5C = MA = MT = MA = MA = MA = MT = MT = MT = M5C |
| 307 | CO-4623 | M5C = M5C = MT = MG = MA = M5C = M5C = MT = MG = M5C = M5C = MA = MT = M5C = MT = MA = MT = M5C = MA = MA |
| 308 | CO-4624 | MG = MT = M5C = MA = MG = MA = M5C = MT = MG = MT = M5C = M5C = MT = MA = M5C = MA = M5C = M5C = MT = MT |
| 309 | CO-4625 | MT = M5C = MT = MT = MG = MT = MT = MG = MT = MT = MG = MG = MA = MA = MA = MT = MT = M5C = M5C = M5C |
| 310 | CO-4626 | M5C = M5C = MT = M5C = MA = MT = MG = MT = MA = MT = MG = MT = MA = MA = MT = MT = MG = M5C = M5C = M5C |
| 311 | CO-4627 | MA = M5C = MT = M5C = MA = MA = MT = MG = M5C = MA = MT = MA = MG = M5C = MA = MG = MA = MA = MT = MA |
| 312 | CO-4628 | MT = M5C = M5C = MA = MT = MA = M5C = MA = MA = M5C = MT = MG = M5C = MA = MG = MA = MG = MT = MG = MG |
| 313 | CO-4629 | MA = MT = MG = MG = M5C = MA = MA = MA = MT = MG = MA = M5C = M5C = MT = MT = M5C = MG = MG = MT = MA |
| 314 | CO-4630 | MG = MT = M5C = MT = MT = M5C = MA = MA = MG = MA = MA = M5C = MT = MT = MT = MA = M5C = M5C = MG = MA |
| 315 | CO-4631 | MA = M5C = M5C = MA = M5C = MT = M5C = MT = MG = M5C = MA = MG = MT = MT = MG = MT = MA = MT = MG = MG |
| 316 | CO-4632 | MT = MG = M5C = MT = MA = MT = MG = M5C = MA = MT = MT = MG = MA = MG = MT = MT = MT = MT = M5C = M5C |
| 317 | CO-4633 | MT = M5C = M5C = MT = M5C = MT = MG = M5C = M5C = MT = MG = MA = M5C = MT = MA = MA = MT = MT = M5C = MG |
| 318 | CO-4634 | MT = MT = MT = MA = M5C = M5C = M5C = MA = MT = MA = MT = MA = M5C = M5C = M5C = MA = M5C = MA = M5C = M5C |
| 319 | CO-4635 | MG = MT = MT = MT = MG = M5C = MA = MA = MT = MT = M5C = MT = M5C = MA = MG = MA = M5C = M5C = MT = MG |
| 320 | CO-4636 | MG = MG = M5C = MA = MA = MT = MT = MA = M5C = MA = MT = MA = M5C = MA = MT = MG = MA = MG = MG = MA |
| 321 | CO-4637 | MT = MA = MA = MG = MG = MT = MG = MT = MA = MG = MG = MA = M5C = MA = MG = MT = M5C = MT = MG = MA |
| 322 | CO-4638 | MT = MT = MG = MA = MT = MA = MG = MA = MT = MG = MG = M5C = MA = MG = MG = MT = M5C = MA = MG = MG |
| 323 | CO-4639 | MG = MA = MA = MA = MT = MT = MT = MA = MT = MG = M5C = MG = MG = M5C = M5C = MA = M5C = MT = MG = MT |
| 324 | CO-4640 | MG = M5C = M5C = M5C = M5C = M5C = MT = MA = MG = MA = MG = MG = MA = M5C = MA = MT = MT = MT = MT = MT |
| 325 | CO-4641 | M5C = MG = MT = MT = M5C = M5C = MA = MA = MA = M5C = MA = MG = M5C = MA = MG = M5C = MG = MT = MT = MA |
| 326 | CO-4642 | MG = MG = M5C = MT = MG = M5C = MA = MG = MA = MT = M5C = MT = M5C = MT = MA = MT = MA = MT = M5C = MA |
| 327 | CO-4643 | M5C = MG = MG = M5C = MA = MT = MT = M5C = M5C = M5C = MT = MG = MA = M5C = M5C = MT = MA = MT = M5C = M5C |
| 328 | CO-4644 | MG = MT = MT = MA = MA = MG = MT = MA = M5C = MG = MT = MG = MA = M5C = M5C = M5C = MT = MA = MT = M5C |
| 329 | CO-4645 | MA = M5C = MA = MG = M5C = MA = MG = MA = MA = M5C = MA = MT = M5C = M5C = M5C = MG = MT = MG = MA = MA |
| 330 | CO-4646 | MG = MT = MG = MT = MG = MA = MA = MA = MT = MG = MA = MT = MG = MG = MT = MA = M5C = M5C = MA = MG |
| 331 | CO-4647 | M5C = MT = MA = MA = MG = M5C = MG = MT = MT = MT = M5C = MT = M5C = MT = MA = MG = MT = M5C = MA = MT |
| 332 | CO-4648 | MA = M5C = MG = M5C = M5C = MA = MA = MA = M5C = M5C = M5C = MT = MT = MG = MA = MT = M5C = M5C = MA = MG |
| 333 | CO-4649 | MT = MG = MG = MG = MA = M5C = MA = MG = M5C = MG = MT = MG = MG = MA = MT = MT = MA = MA = MT = MA |
| 334 | CO-4650 | MT = MA = MA = MG = MA = M5C = MT = MT = MT = M5C = M5C = MA = MG = M5C = MA = MT = MG = MT = M5C = M5C |
| 335 | CO-4651 | MT = MA = MG = MG = M5C = MA = M5C = M5C = M5C = MA = MA = MG = MG = M5C = MT = MT = MT = MA = MT = MT |
| 174 | CO-5264 | MG = MG = MA − M5C − MT − MT − MG − MG − MG − MA − MG − MT − MG − MG − MT − MG − MA = M5C = MT = MG |
| 175 | CO-5265 | MG = MG = MA = M5C = MT − MT − MG − MG − MG − MA − MG − MT − MG − MG − MT − MG − MA = M5C = MT = MG |
| 176 | CO-5266 | MG = MG = MA = M5C = MT = MT = MG − MG − MG − MA − MG − MT − MG − MG − MT − MG − MA = M5C = MT = MG |
| 177 | CO-5267 | MG = MG = MA = M5C = MT = MT = MG = MG = MG − MA − MG − MT − MG − MG − MT − MG − MA = M5C = MT = MG |
| 178 | CO-5268 | MG = MG = MA = M5C = MT = MT = MG = MG = MG = MA = MG − MT − MG − MG − MT − MG − MA = M5C = MT = MG |
| 179 | CO-5269 | MG = MG = MA = M5C = MT = MT = MG = MG = MG = MA = MG = MT = MG − MG − MT − MG − MA = M5C = MT = MG |
| 180 | CO-5270 | MG = MG = MA − M5C − MT − MT − MG − MG − MG − MA − MG − MT − MG − MG − MT − MG = MA = M5C = MT = MG |
| 181 | CO-5271 | MG = MG = MA − M5C − MT − MT − MG − MG − MG − MA − MG − MT − MG − MG = MT = MG = MA = M5C = MT = MG |
| 182 | CO-5272 | MG = MG = MA − M5C − MT − MT − MG − MG − MG − MA − MG − MT = MG = MG = MT = MG = MA = M5C = MT = MG |
| 183 | CO-5273 | MG = MG = MA − M5C − MT − MT − MG − MG − MG − MA = MG = MT = MG = MG = MT = MG = MA = M5C = MT = MG |
| 184 | CO-5274 | MG = MG = MA − M5C − MT − MT − MG − MG = MG = MA = MG = MT = MG = MG = MT = MG = MA = M5C = MT = MG |
| 185 | CO-5275 | LG = LG = MA = L5C = LT = MT = LG = MG = LG = MA = LG = MT = LG = MG = LT = MG = LA = M5C = LT = LG |
| 186 | CO-5276 | LG = LG = MA = L5C = MT = LT = MG = LG = MG = LA = MG = LT = MG = LG = MT = LG = LA = M5C = LT = LG |
| 187 | CO-5277 | MG = MG = MA − MT − MA − MG − MG − MA − MT − MA − MG − MG − MA − MG − M5C − MG − MA = MG = M5C = M5C |
| 188 | CO-5278 | MG = MG = MA = MT = MA − MG − MG − MA − MT − MA − MG − MG − MA − MG − M5C − MG − MA = MG = M5C = M5C |
| 189 | CO-5279 | MG = MG = MA = MT = MA = MG = MG − MA − MT − MA − MG − MG − MA − MG − M5C − MG − MA = MG = M5C = M5C |
| 190 | CO-5280 | MG = MG = MA = MT = MA = MG = MG = MA = MT − MA − MG − MG − MA − MG − M5C − MG − MA = MG = M5C = M5C |
| 191 | CO-5281 | MG = MG = MA = MT = MA = MG = MG = MA = MT = MA = MG − MG − MA − MG − M5C − MG − MA = MG = M5C = M5C |
| 192 | CO-5282 | MG = MG = MA = MT = MA = MG = MG = MA = MT = MA = MG = MG = MA − MG − M5C − MG − MA = MG = M5C = M5C |
| 193 | CO-5283 | MG = MG = MA − MT − MA − MG − MG − MA − MT − MA − MG − MG − MA − MG − M5C − MG = MA = MG = M5C = M5C |
| 194 | CO-5284 | MG = MG = MA − MT − MA − MG − MG − MA − MT − MA − MG − MG − MA − MG = M5C = MG = MA = MG = M5C = M5C |
| 195 | CO-5285 | MG = MG = MA − MT − MA − MG − MG − MA − MT − MA − MG − MG = MA = MG = M5C = MG = MA = MG = M5C = M5C |
| 196 | CO-5286 | MG = MG = MA − MT − MA − MG − MG − MA − MT − MA = MG = MG = MA = MG = M5C = MG = MA = MG = M5C = M5C |
| 197 | CO-5287 | MG = MG = MA − MT − MA − MG − MG − MA = MT = MA = MG = MG = MA = MG = M5C = MG = MA = MG = M5C = M5C |
| 198 | CO-5288 | LG = LG = MA = LT = LA = MG = LG = MA = LT = MA = LG = MG = LA = MG = L5C = MG = LA = MG = L5C = L5C |
| 199 | CO-5289 | LG = LG = MA = LT = MA = LG = MG = LA = MT = LA = MG = LG = MA = LG = M5C = LG = LA = MG = L5C = L5C |
| 336 | CO-6267 | MA = MA = MG = MA = MG = M5C = M5C = MG = MG = MA = MG = MA = MT = MT = M5C = MG = MA = MA = M5C = M5C |
| 337 | CO-6268 | MG = MA = MA = MG = MA = MG = M5C = M5C = MG = MG = MA = MG = MA = MT = MT = M5C = MG = MA = MA = M5C |
| 338 | CO-6269 | MG = MT = MG = MA = MA = MG = MA = MG = M5C = M5C = MG = MG = MA = MG = MA = MT = MT = M5C = MG = MA |
| 339 | CO-6270 | MA = M5C = MG = MT = MG = dA = dA = dG = dA = dG = d5C = d5C = dG = dG = dA = MG = MA = MT = MT = M5C |
| 340 | CO-6271 | M5C = MG = MA = M5C = MG = MT = MG = MA = MA = MG = MA = MG = M5C = M5C = MG = MG = MA = MG = MA = MT |
| 341 | CO-6272 | MT = M5C = MG = MA = M5C = MG = MT = MG = MA = MA = MG = MA = MG = M5C = M5C = MG = MG = MA = MG = MA |
| 342 | CO-6273 | M5C = M5C = M5C = MG = MT = MT = MT = MT = MA = M5C = MA = MG = MA = MA = MA = MG = MT = MG = MA = MA |
| 343 | CO-6274 | MA = M5C = M5C = M5C = M5C = MG = MT = MT = MT = MT = MA = M5C = MA = MG = MA = MA = MA = MG = MT = MG |
| 344 | CO-6275 | MT = MT = MA = M5C = M5C = M5C = M5C = MG = MT = MT = MT = MT = MA = M5C = MA = MG = MA = MA = MA = MG |
| 345 | CO-6276 | MG = MA = MT = MT = MA = M5C = M5C = M5C = M5C = MG = MT = MT = MT = MT = MA = M5C = MA = MG = MA = MA |
| 346 | CO-6277 | MT = MA = MG = MA = MT = MT = MA = M5C = M5C = M5C = M5C = MG = MT = MT = MT = MT = MA = M5C = MA = MG |
| 347 | CO-6278 | MA = MA = MT = MA = MG = MA = MT = MT = MA = M5C = M5C = M5C = M5C = MG = MT = MT = MT = MT = MA = M5C |
| 348 | CO-6279 | MA = M5C = MG = MT = MG = dA = dA = dG = MA = dG = d5C = M5C = dG = dG = dA = MG = MA = MT = MT = M5C |
| 349 | CO-6280 | MA = M5C = MG = MT = MG = LA = dA = dG = LA = dG = d5C = L5C = dG = dG = LA = MG = MA = MT = MT = M5C |
| 350 | CO-6281 | MA = M5C = MG = LT = MG = dA = dA = LG = dA = dG = d5C = L5C = dG = dG = dA = LG = MA = MT = MT = M5C |
| 351 | CO-6282 | MA = M5C = MG = MT = LG = dA = dA = dG = LA = dG = d5C = d5C = LG = dG = dA = MG = LA = MT = MT = M5C |
| 352 | CO-6283 | MA = M5C = MG = MT = MG = LA = MA = MG = LA = MG = M5C = L5C = MG = MG = LA = MG = MA = MT = MT = M5C |
| 353 | CO-6284 | MA = M5C = MG = LT = MG = LA = MA = LG = MA = LG = M5C = L5C = MG = LG = MA = LG = MA = LT = MT = M5C |
| 354 | CO-6285 | MA = M5C = LG = MT = LG = MA = LA = MG = LA = MG = L5C = M5C = LG = MG = LA = MG = LA = MT = MT = M5C |
| 355 | CO-6286 | MA = M5C = LG = MT = MG = LA = MA = MG = LA = MG = M5C = L5C = MG = MG = LA = MG = MA = LT = MT = M5C |
| 356 | CO-6287 | MA = M5C = MG = MT = LG = MA = MA = MG = LA = MG = M5C = M5C = LG = MG = MA = MG = LA = MT = MT = M5C |
| 357 | CO-6288 | MA = M5C = MG = LT = MG = MA = MA = LG = MA = MG = M5C = L5C = MG = MG = MA = LG = MA = MT = MT = M5C |
| 358 | CO-6289 | MA = M5C = MG = MT = MG = LA = MA = MG = MA = MG = L5C = M5C = MG = MG = MA = LG = MA = MT = MT = M5C |
| 359 | CO-6290 | MA = M5C = MG = MT = LG = MA = MA = MG = MA = LG = M5C = M5C = MG = MG = LA = MG = MA = MT = MT = M5C |
| 360 | CO-6291 | M5C = MT = MG = MG = MA = MT = MA = MG = MG = MT = M5C = MA = MG = MG = MG = MA = MA = MT = MG = M5C |
| 361 | CO-6292 | MA = MG = MA = MG = MA = MT = M5C = MT = MG = M5C = MA = MG = M5C = M5C = M5C = MT = M5C = MA = M5C = M5C |
| 362 | CO-6293 | MA = MT = MA = MG = MA = MG = MA = MT = M5C = MT = MG = M5C = MA = MG = M5C = M5C = M5C = MT = M5C = MA |
| 363 | CO-6294 | MA = MT = MA = MT = MA = MG = MA = MG = MA = MT = M5C = MT = MG = M5C = MA = MG = M5C = M5C = M5C = MT |
| 364 | CO-6295 | MT = MG = MA = MT = MA = dT = dA = dG = dA = dG = dA = dT = d5C = dT = dG = M5C = MA = MG = M5C = M5C |
| 365 | CO-6296 | MA = MA = MT = MG = MA = MT = MA = MT = MA = MG = MA = MG = MA = MT = M5C = MT = MG = M5C = MA = MG |
| 366 | CO-6297 | MA = MT = MA = MA = MT = MG = MA = MT = MA = MT = MA = MG = MA = MG = MA = MT = M5C = MT = MG = M5C |
| 367 | CO-6298 | MA = MA = M5C = MT = M5C = MT = MG = MT = M5C = MT = MT = MT = MG = MA = MG = MA = MT = MT = MG = MT |
| 368 | CO-6299 | MG = M5C = MA = M5C = M5C = MT = MG = M5C = MT = MG = MT = MT = MT = MA = MA = MT = MT = MT = MA = MA |
| 369 | CO-6300 | MT = MG = MA = MT = MA = dT = dA = dG = MA = dG = dA = MT = d5C = dT = dG = M5C = MA = MG = M5C = M5C |
| 370 | CO-6301 | MT = MG = MA = MT = MA = LT = dA = dG = LA = dG = dA = LT = d5C = dT = LG = M5C = MA = MG = M5C = M5C |
| 371 | CO-6302 | MT = MG = MA = LT = MA = dT = dA = LG = dA = dG = dA = LT = d5C = dT = dG = L5C = MA = MG = M5C = M5C |
| 372 | CO-6303 | MT = MG = MA = MT = LA = dT = dA = dG = LA = dG = dA = dT = L5C = dT = dG = M5C = LA = MG = M5C = M5C |
| 373 | CO-6304 | MT = MG = MA = MT = MA = LT = MA = MG = LA = MG = MA = LT = M5C = MT = LG = M5C = MA = MG = M5C = M5C |
| 374 | CO-6305 | MT = MG = MA = LT = MA = LT = MA = LG = MA = LG = MA = LT = M5C = LT = MG = L5C = MA = LG = M5C = M5C |
| 375 | CO-6306 | MT = MG = LA = MT = LA = MT = LA = MG = LA = MG = LA = MT = L5C = MT = LG = M5C = LA = MG = M5C = M5C |
| 376 | CO-6307 | MT = MG = LA = MT = MA = LT = MA = MG = LA = MG = MA = LT = M5C = MT = LG = M5C = MA = LG = M5C = M5C |
| 377 | CO-6308 | MT = MG = MA = MT = LA = MT = MA = MG = LA = MG = MA = MT = L5C = MT = MG = M5C = LA = MG = M5C = M5C |
| 378 | CO-6309 | MT = MG = MA = LT = MA = MT = MA = LG = MA = MG = MA = LT = M5C = MT = MG = L5C = MA = MG = M5C = M5C |
| 379 | CO-6310 | MT = MG = MA = MT = MA = LT = MA = MG = MA = MG = LA = MT = M5C = MT = MG = L5C = MA = MG = M5C = M5C |
| 380 | CO-6311 | MT = MG = MA = MT = LA = MT = MA = MG = MA = LG = MA = MT = M5C = MT = LG = M5C = MA = MG = M5C = M5C |
| 381 | CO-6312 | MT = M5C = MT = MA = MA = MA = M5C = MG = M5C = M5C = MA = MA = MA = M5C = M5C = M5C = MT = MT = MG = MA |
| 382 | CO-6313 | MA = MT = MT = M5C = MT = MA = MA = MA = M5C = MG = M5C = M5C = MA = MA = MA = M5C = M5C = M5C = MT = MT |
| 383 | CO-6314 | MA = MA = MA = MT = MT = M5C = MT = MA = MA = MA = M5C = MG = M5C = M5C = MA = MA = MA = M5C = M5C = M5C |
| 384 | CO-6315 | MG = M5C = M5C = MA = MT = MA = MG = M5C = MT = MA = MT = MG = MA = MA = MA = MA = MT = MT = M5C = MT |
| 385 | CO-6316 | MG = MA = MA = MG = MG = MT = MA = MG = MT = MT = MT = MT = MA = MG = M5C = M5C = MA = MT = MA = MG |
| 386 | CO-6317 | MT = MG = MG = MA = MA = MG = MG = MT = MA = MG = MT = MT = MT = MT = MA = MG = M5C = M5C = MA = MT |
| 387 | CO-6318 | MT = MA = MT = MG = MG = MA = MA = MG = MG = MT = MA = MG = MT = MT = MT = MT = MA = MG = M5C = M5C |
| 388 | CO-6319 | MT = MG = MG = MA = MT = MT = MA = MA = MT = MA = MT = MG = MG = MA = MA = MG = MG = MT = MA = MG |
| 389 | CO-6320 | M5C = MG = MT = MG = MG = MA = MT = MT = MA = MA = MT = MA = MT = MG = MG = MA = MA = MG = MG = MT |
| 390 | CO-6321 | MA = MG = M5C = MG = MT = MG = MG = MA = MT = MT = MA = MA = MT = MA = MT = MG = MG = MA = MA = MG |
| 391 | CO-6322 | MA = M5C = MA = MG = M5C = MG = MT = MG = MG = MA = MT = MT = MA = MA = MT = MA = MT = MG = MG = MA |
| 392 | CO-6323 | MG = MG = MA = M5C = MA = MG = M5C = MG = MT = MG = MG = MA = MT = MT = MA = MA = MT = MA = MT = MG |
| 393 | CO-6324 | MT = MG = MG = MG = MA = d5C = dA = dG = d5C = dG = dT = dG = dG = dA = dT = MT = MA = MA = MT = MA |
| 394 | CO-6325 | MT = MT = MT = M5C = M5C = MA = MG = M5C = MA = MT = MG = MT = M5C = M5C = M5C = M5C = M5C = MA = MG = M5C |
| 395 | CO-6326 | MA = M5C = MT = MT = MT = M5C = M5C = MA = MG = M5C = MA = MT = MG = MT = M5C = M5C = M5C = M5C = M5C = MA |
| 396 | CO-6327 | MA = MG = MA = M5C = MT = MT = MT = M5C = M5C = MA = MG = M5C = MA = MT = MG = MT = M5C = M5C = M5C = M5C |
| 397 | CO-6328 | MT = MG = MG = MG = MA = d5C = dA = dG = M5C = dG = dT = MG = dG = dA = dT = MT = MA = MA = MT = MA |
| 398 | CO-6329 | MT = MG = MG = MG = MA = L5C = dA = dG = L5C = dG = dT = LG = dG = dA = LT = MT = MA = MA = MT = MA |
| 399 | CO-6330 | MT = MG = MG = LG = MA = d5C = dA = LG = d5C = dG = dT = LG = dG = dA = dT = LT = MA = MA = MT = MA |
| 400 | CO-6331 | MT = MG = MG = MG = LA = d5C = dA = dG = L5C = dG = dT = dG = LG = dA = dT = MT = LA = MA = MT = MA |
| 401 | CO-6332 | MT = MG = MG = MG = MA = L5C = MA = MG = L5C = MG = MT = LG = MG = MA = LT = MT = MA = MA = MT = MA |
| 402 | CO-6333 | MT = MG = MG = LG = MA = L5C = MA = LG = M5C = LG = MT = LG = MG = LA = MT = LT = MA = LA = MT = MA |
| 403 | CO-6334 | MT = MG = LG = MG = LA = M5C = LA = MG = L5C = MG = LT = MG = LG = MA = LT = MT = LA = MA = MT = MA |
| 404 | CO-6335 | MT = MG = LG = MG = MA = L5C = MA = MG = L5C = MG = MT = LG = MG = MA = LT = MT = MA = LA = MT = MA |
| 405 | CO-6336 | MT = MG = MG = MG = LA = M5C = MA = MG = L5C = MG = MT = MG = LG = MA = MT = MT = LA = MA = MT = MA |
| 406 | CO-6337 | MT = MG = MG = LG = MA = M5C = MA = LG = M5C = MG = MT = LG = MG = MA = MT = LT = MA = MA = MT = MA |
| 407 | CO-6338 | MT = MG = MG = MG = MA = L5C = MA = MG = M5C = MG = LT = MG = MG = MA = MT = LT = MA = MA = MT = MA |
| 408 | CO-6339 | MT = MG = MG = MG = LA = M5C = MA = MG = M5C = LG = MT = MG = MG = MA = LT = MT = MA = MA = MT = MA |
| 409 | CO-6340 | M5C = MT = MG = MG = MG = MA = d5C = dA = dG = d5C = dG = dT = dG = dG = dA = dT = MT = MA = MA = MT = MA = MT |
| 410 | CO-6341 | M5C = MT = MG = MG = MG = MA = M5C = MA = MG = M5C = MG = MT = MG = MG = MA = MT = MT = MA = MA = MT = MA = MT |
| 411 | CO-6342 | M5C = MT = MG = MG = MG = dA = d5C = dA = MG = d5C = dG = dT = dG = MG = dA = dT = dT = MA = MA = MT = MA = MT |
| 412 | CO-6343 | M5C = MT = MG = MG = MG = LA = d5C = dA = LG = d5C = dG = dT = dG = LG = dA = dT = LT = MA = MA = MT = MA = MT |
| 413 | CO-6344 | MG = M5C = MT = MG = MG = MG = MA = d5C = dA = dG = d5C = dG = dT = dG = dG = dA = dT = MT = MA = MA = MT = MA = MT = MG |
| 414 | CO-6345 | MG = M5C = MT = MG = MG = MG = MA = M5C = MA = MG = M5C = MG = MT = MG = MG = MA = MT = MT = MA = MA = MT = MA = MT = MG |
| 415 | CO-6346 | MG = M5C = MT = MG = MG = dG = dA = M5C = dA = dG = M5C = dG = dT = MG = dG = dA = MT = dT = dA = MA = MT = MA = MT = MG |
| 416 | CO-6347 | MG = M5C = MT = MG = MG = dG = dA = L5C = dA = dG = L5C = dG = dT = LG = dG = dA = LT = dT = dA = MA = MT = MA = MT = MG |
| 262 | CO-6380 | MA = MA = MA = MA = M5C = MA = MA = MA = M5C = MA = M5C = MA = M5C = MA = MA = MG = MT = M5C = M5C = MG |
| 263 | CO-6381 | MT = MG = MA = MT = M5C = MA = MG = M5C = MT = MT = MG = MG = MG = M5C = MG = MA = M5C = MT = MG = MA |
| 264 | CO-6382 | M5C = MT = MA = MT = MG = MA = MT = M5C = MA = MG = M5C = MT = MT = MG = MG = MG = M5C = MG = MA = M5C |
| 265 | CO-6383 | MT = MA = MG = MG = MA = dG = dA = dA = dT = d5C = dG = d5C = dT = dT = dT = MG = MG = MG = MA = MG |
| 266 | CO-6384 | MG = MT = MA = MG = MG = MA = MG = MA = MA = MT = M5C = MG = M5C = MT = MT = MT = MG = MG = MG = MA |
| 267 | CO-6385 | M5C = MG = MG = MT = MA = MG = MG = MA = MG = MA = MA = MT = M5C = MG = M5C = MT = MT = MT = MG = MG |
| 268 | CO-6386 | MG = M5C = MG = MA = MG = M5C = M5C = MA = MG = M5C = MT = M5C = MA = MG = MT = MA = MG = M5C = MT = M5C |
| 200 | CO-6387 | MT = MA = MG = MG = MA = dG = dA = dA = MT = d5C = dG = M5C = dT = dT = dT = MG = MG = MG = MA = MG |
| 201 | CO-6388 | MT = MA = MG = MG = MA = LG = dA = dA = LT = d5C = dG = L5C = dT = dT = LT = MG = MG = MG = MA = MG |
| 202 | CO-6389 | MT = MA = MG = LG = MA = dG = dA = LA = dT = d5C = dG = L5C = dT = dT = dT = LG = MG = MG = MA = MG |
| 203 | CO-6390 | MT = MA = MG = MG = LA = dG = dA = dA = LT = d5C = dG = d5C = LT = dT = dT = MG = LG = MG = MA = MG |
| 204 | CO-6391 | MT = MA = MG = MG = MA = LG = MA = MA = LT = M5C = MG = L5C = MT = MT = LT = MG = MG = MG = MA = MG |
| 205 | CO-6392 | MT = MA = MG = LG = MA = LG = MA = LA = MT = L5C = MG = L5C = MT = LT = MT = LG = MG = LG = MA = MG |
| 206 | CO-6393 | MT = MA = LG = MG = LA = MG = LA = MA = LT = M5C = LG = M5C = LT = MT = LT = MG = LG = MG = MA = MG |
| 207 | CO-6394 | MT = MA = LG = MG = MA = LG = MA = MA = LT = M5C = MG = L5C = MT = MT = LT = MG = MG = LG = MA = MG |
| 208 | CO-6395 | MT = MA = MG = MG = LA = MG = MA = MA = LT = M5C = MG = M5C = LT = MT = MT = MG = LG = MG = MA = MG |
| 209 | CO-6396 | MT = MA = MG = LG = MA = MG = MA = LA = MT = M5C = MG = L5C = MT = MT = MT = LG = MG = MG = MA = MG |
| 210 | CO-6397 | MT = MA = MG = MG = MA = LG = MA = MA = MT = M5C = LG = M5C = MT = MT = MT = LG = MG = MG = MA = MG |
| 211 | CO-6398 | MT = MA = MG = MG = LA = MG = MA = MA = MT = L5C = MG = M5C = MT = MT = LT = MG = MG = MG = MA = MG |
| 212 | CO-6399 | MG = MT = MA = MG = MG = MA = dG = dA = dA = dT = d5C = dG = d5C = dT = dT = dT = MG = MG = MG = MA = MG = M5C |
| 213 | CO-6400 | MG = MT = MA = MG = MG = MA = MG = MA = MA = MT = M5C = MG = M5C = MT = MT = MT = MG = MG = MG = MA = MG = M5C |
| 214 | CO-6401 | MG = MT = MA = MG = MG = dA = dG = dA = MA = dT = d5C = dG = d5C = MT = dT = dT = dG = MG = MG = MA = MG = M5C |
| 215 | CO-6402 | MG = MT = MA = MG = MG = LA = dG = dA = LA = dT = d5C = dG = d5C = LT = dT = dT = LG = MG = MG = MA = MG = M5C |
| 216 | CO-6403 | MG = MG = MT = MA = MG = MG = MA = dG = dA = dA = dT = d5C = dG = d5C = dT = dT = dT = MG = MG = MG = MA = MG = M5C = MA |
| 217 | CO-6404 | MG = MG = MT = MA = MG = MG = MA = MG = MA = MA = MT = M5C = MG = M5C = MT = MT = MT = MG = MG = MG = MA = MG = M5C = MA |
| 218 | CO-6405 | MG = MG = MT = MA = MG = dG = dA = MG = dA = dA = MT = d5C = dG = M5C = dT = dT = MT = dG = dG = MG = MA = MG = M5C = MA |
| 219 | CO-6406 | MG = MG = MT = MA = MG = dG = dA = LG = dA = dA = LT = d5C = dG = L5C = dT = dT = LT = dG = dG = MG = MA = MG = M5C = MA |
| 220 | CO-6407 | MT = MA = MG − MG − MA − MG = MA = MA = MT = M5C = MG = M5C = MT = MT = MT − MG − MG − MG = MA = MG |
| 221 | CO-6408 | MT = MA = MG = MG − MA − MG − MA = MA = MT = M5C = MG = M5C = MT = MT − MT − MG − MG = MG = MA = MG |
| 222 | CO-6409 | MT = MA = MG − MG = MA = MG − MA = MA = MT − M5C = MG = M5C − MT = MT = MT − MG = MG = MG − MA = MG |
| 223 | CO-6410 | MT = MA − MG = MG = MA − MG = MA = MA − MT = M5C = MG − M5C = MT = MT − MT = MG = MG − MG = MA = MG |
| 224 | CO-6411 | MT = MA = MG − MG − MA = MG = MA = MA = MT = M5C = MG = M5C = MT = MT = MT = MG − MG − MG = MA = MG |
| 991 | CO-6412 | MG = MG = MA = M5C = MT = dT = dG = dG = dG = dA = dG = dT = dG = dG = dT = dG = MA = M5C = MT = MG = MA |
| 992 | CO-6413 | MG = MG = MA = M5C = MT = dT = dG = dG = MG = dA = dG = dT = MG = dG = dT = dG = MA = M5C = MT = MG = MA |
| 993 | CO-6414 | MG = MG = MA = L5C = MT = LT = MG = LG = MG = LA = MG = LT = MG = LG = MT = LG = MA = L5C = MT = MG = MA |
| 994 | CO-6415 | MG = MG = MA = L5C = MT = MT = LG = MG = MG = LA = MG = MT = LG = MG = MT = LG = MA = M5C = LT = MG = MA |
| 995 | CO-6416 | MG = MG = LA = M5C = MT = LT = MG = MG = LG = MA = MG = LT = MG = MG = LT = MG = MA = L5C = MT = MG = MA |
| 996 | CO-6417 | MG = MG = MA = M5C = LT = MT = MG = MG = LG = MA = MG = MT = LG = MG = MT = MG = LA = M5C = MT = MG = MA |
| 997 | CO-6418 | MG = MG = MA = M5C = MT = LT = MG = MG = MG = MA = LG = MT = MG = MG = MT = LG = MA = M5C = MT = MG = MA |
| 998 | CO-6419 | MG = MG = MA = M5C = MT = dT = dG = dG = dG = dA = dG = dT = dG = dG = dT = dG = dA = d5C = MT = MG = MA = MA = M5C |
| 999 | CO-6420 | MG = MG = LA = M5C = MT = LT = MG = MG = LG = MA = MG = LT = MG = MG = LT = MG = MA = L5C = MT = MG = LA = MA = M5C |
| 1000 | CO-6421 | MG = MG = MA = M5C = MT = LT = MG = MG = MG = MA = MG = LT = MG = MG = MT = MG = MA = L5C = MT = MG = MA = MA = M5C |
| 1001 | CO-6422 | MG = MG = MA = L5C = MT = MT = MG = LG = MG = MA = MG = LT = MG = MG = MT = LG = MA = M5C = MT = LG = MA = MA = M5C |
| 1002 | CO-6423 | MG = MG = MA = M5C = LT = MT = MG = MG = MG = LA = MG = MT = MG = MG = LT = MG = MA = M5C = MT = LG = MA = MA = M5C |
| 1003 | CO-6424 | MG = MG = MA = L5C = MT = MT = MG = MG = LG = MA = MG = MT = MG = LG = MT = MG = MA = M5C = LT = MG = MA = MA = M5C |
| 1004 | CO-6425 | MG = MG = MG = MA = M5C = MT = MT = MG = MG = MG = MA = MG = MT = MG = MG = MT = MG = MA = M5C = MT = MG = MA = MA = M5C |
| 1005 | CO-6426 | MG = MG = MA = M5C = MT = MT = MG = MG = MG = MA = MG = MT = MG = MG = MT = MG = MA = M5C = MT = MG = MA = MA = M5C = M5C |
| 1006 | CO-6427 | MG = MG = MG = MA = M5C = MT = MT = MG = MG = MG = MA = MG = MT = MG = MG = MT = MG = MA = M5C = MT = MG = MA = MA = M5C = M5C |
| 1007 | CO-6428 | MG = MG = MG = MA = M5C = MT = MT = MG = MG = MG = MA = MG = MT = MG = MG = MT = MG = MA = M5C = MT = MG = MA = MA = M5C = M5C = MT |
| 1008 | CO-6429 | MA = MG = MG = MG = MA = M5C = MT = MT = MG = MG = MG = MA = MG = MT = MG = MG = MT = MG = MA = M5C = MT = MG = MA = MA = M5C = M5C |
| 4917 | CO-6430 | MT = MG = M5C = MG = M5C = MA = M5C = MA = M5C = MA = MG = MG = MT = MA = M5C = M5C = MA = MG = MA = MG |
| 4918 | CO-6431 | MA = M5C = MG = MG = MA = M5C = M5C = MT = M5C = M5C = MT = M5C = MA = M5C = MT = MA = MA = MG = M5C = MT |
| 4919 | CO-6432 | MG = MA = MG = MA = MA = M5C = MG = M5C = MT = MA = M5C = M5C = MA = M5C = MG = MG = MA = M5C = M5C = MT |
| 4920 | CO-6433 | MG = M5C = MA = M5C = MT = MG = M5C = MT = MG = M5C = M5C = M5C = M5C = M5C = MA = MA = MG = MA = M5C = MA |
| 4921 | CO-6434 | MT = M5C = M5C = MG = MA = MT = M5C = MA = M5C = M5C = MT = MG = M5C = MA = M5C = MT = MG = M5C = MT = MG |
| 4922 | CO-6435 | MA = MG = MG = M5C = M5C = MA = M5C = M5C = MT = MG = M5C = MT = MG = M5C = MT = M5C = M5C = MG = MA = MT |
| 4923 | CO-6436 | MT = MG = MT = M5C = M5C = M5C = M5C = MA = M5C = MT = M5C = MA = MG = MG = M5C = M5C = MA = M5C = M5C = MT |
| 4924 | CO-6437 | MG = MA = M5C = MA = MA = MA = MG = MA = M5C = M5C = M5C = MA = M5C = M5C = M5C = M5C = MT = MG = MT = M5C |
| 4925 | CO-6438 | M5C = MT = MA = M5C = MT = MG = MA = MG = M5C = M5C = MT = MG = MG = MA = MA = MG = MT = MG = MA = M5C |
| 4926 | CO-6439 | MG = M5C = MT = M5C = M5C = M5C = MT = MG = MT = MG = MT = MG = M5C = MT = MA = M5C = MT = MG = MA = MG |
| 4927 | CO-6440 | MG = MT = MT = M5C = MA = MT = MG = M5C = MT = MA = M5C = M5C = M5C = M5C = M5C = MT = MA = MG = MT = MG |
| 4928 | CO-6441 | MG = MG = M5C = MT = MG = MA = MT = MG = M5C = MA = MG = MG = MG = MT = MT = M5C = MA = MT = MG = M5C |
| 4929 | CO-6442 | M5C = MT = MG = MG = MG = MA = MG = M5C = M5C = MT = MG = MG = M5C = MT = MG = MA = MT = MG = M5C = MA |
| 4930 | CO-6443 | MG = M5C = M5C = M5C = MT = MG = MA = M5C = MA = M5C = MG = M5C = MA = M5C = M5C = MT = MT = MA = M5C = MA |
| 4931 | CO-6444 | MT = MG = M5C = M5C = M5C = MA = MA = MT = MG = M5C = M5C = M5C = MA = MA = M5C = MG = MT = MG = MA = MG |
| 4932 | CO-6445 | MG = MT = MG = MG = MG = MA = MA = MG = MT = MA = MT = MG = MG = M5C = MT = MG = M5C = MT = MG = M5C |
| 4933 | CO-6446 | MT = MG = M5C = MT = MG = MT = MG = MA = MG = M5C = MT = MG = M5C = M5C = M5C = MA = MG = MT = MG = MG |
| 4934 | CO-6447 | MA = MT = MG = MG = MT = MT = M5C = MT = MA = M5C = M5C = MT = MG = M5C = MT = MG = MT = MG = MA = MG |
| 4935 | CO-6448 | MT = M5C = M5C = M5C = MG = MG = MT = MG = M5C = M5C = M5C = MT = MG = MA = MT = MG = MG = MT = MT = M5C |
| 4936 | CO-6449 | MG = MT = M5C = M5C = MA = MG = M5C = MT = M5C = MG = MG = MT = M5C = MA = MT = MG = MT = MG = MT = M5C |
| 4937 | CO-6450 | M5C = MA = MG = MT = MG = MG = M5C = M5C = MT = MT = MG = MT = M5C = M5C = MA = MG = M5C = MT = M5C = MG |
| 4938 | CO-6451 | MA = M5C = MG = MT = MT = MT = MG = M5C = MT = M5C = M5C = MT = M5C = MT = MT = M5C = M5C = MA = MG = MT |
| 4939 | CO-6452 | MT = MG = MG = M5C = MT = MG = M5C = M5C = M5C = M5C = MT = M5C = MA = M5C = MG = MT = MT = MT = MG = M5C |
| 4940 | CO-6453 | MG = MA = MA = MA = MA = MA = MG = MT = MT = MT = M5C = M5C = MT = M5C = M5C = MA = MT = M5C = M5C = MT |
| 4941 | CO-6454 | M5C = M5C = MA = MA = MG = MT = MG = MT = MA = MG = MG = MA = MA = MA = MA = MA = MG = MT = MT = MT |
| 4942 | CO-6455 | M5C = MT = M5C = MA = MT = MT = MG = MA = M5C = MT = M5C = M5C = MA = MA = MG = MT = MG = MT = MA = MG |
| 1009 | CO-6512 | MT = MA = MG − MG − MA − MG − MA = MA = MT = M5C = MG = M5C − MT = MT = MT = MG = MG = MG = MA = MG |
| 1010 | CO-6513 | MT = MA = MG − MG − MA − MG − MA − MA − MT = M5C = MG = M5C = MT = MT = MT = MG = MG = MG = MA = MG |
| 1011 | CO-6514 | MT = MA = MG − MG − MA − MG − MA − MA − MT − M5C − MG = M5C = MT = MT = MT = MG = MG = MG = MA = MG |
| 1012 | CO-6515 | MT = MA = MG − MG − MA − MG − MA − MA − MT − M5C − MG − M5C − MT = MT = MT = MG = MG = MG = MA = MG |
| 1013 | CO-6516 | MT = MA = MG − MG − MA − MG − MA − MA − MT − M5C − MG − M5C − MT − MT − MT = MG = MG = MG = MA = MG |
| 1014 | CO-6517 | MT = MA = MG − MG − MA − MG − MA − MA − MT − M5C − MG − M5C − MT − MT − MT − MG − MG = MG = MA = MG |
| 1015 | CO-6518 | MT = MA = MG = MG − MA − MG − MA − MA − MT − M5C − MG − M5C − MT − MT − MT − MG − MG − MG = MA = MG |
| 1016 | CO-6519 | MT = MA = MG = MG = MA = MG − MA − MA − MT − M5C − MG − M5C − MT − MT − MT − MG − MG − MG = MA = MG |
| 1017 | CO-6520 | MT = MA = MG = MG = MA = MG = MA = MA − MT − M5C − MG − M5C − MT − MT − MT − MG − MG − MG = MA = MG |
| 225 | CO-6521 | MT = MA = MG = MG = MA = MG = MA = MA = MT = M5C − MG − M5C − MT − MT − MT − MG − MG − MG = MA = MG |
| 226 | CO-6522 | MT = MA = MG = MG = MA = MG = MA = MA = MT = M5C = MG = M5C − MT − MT − MT − MG − MG − MG = MA = MG |
| 227 | CO-6523 | MT = MA = MG = MG = MA = MG = MA = MA = MT = M5C = MG = M5C = MT = MT − MT − MG − MG − MG = MA = MG |
| 4943 | CO-6524 | mG = MG = MA = MC = MU = MU = MG = MG = MG = MA = MG = MU = MG = MG = MU = MG = MA = MC = MU = MG |
| 4944 | CO-6525 | mG = MG = MA = MC = MU = MU = MG = MG = MG = MA = MG = MU = MG = MG = MU = MG = MA = MC = MU = MG = MA = MA = MC |
| 4945 | CO-6526 | mG = MG = MA − MC − MU = MU = MG = MG = MG = MA = MG = MU = MG = MG = MU = MG − MA − MC = MU = MG |
| 4946 | CO-6527 | mG = MG = MA = MC = MU = MU = MG − MG − MG − MA − MG − MU − MG − MG − MU − MG − MA = MC = MU = MG |
| 4947 | CO-6528 | mG = MG = MA = MC = MU = MU = MG = MG = MG − MA − MG − MU − MG − MG − MU − MG − MA = MC = MU = MG |
| 4948 | CO-6529 | mG = MG = MA = MC = MU = MU = MG = MG = MG = MA = MG − MU − MG − MG − MU − MG − MA = MC = MU = MG |
| 4949 | CO-6530 | mG = MG = MA = MC = MU = MU = MG = MG = MG = MA = MG = MU = MG − MG − MU − MG − MA = MC = MU = MG |
| 1018 | CO-7262 | M5C = MT = M5C = M5C = MT = M5C = M5C = MT = MT = MA = M5C = MT = MT = M5C = M5C = MT = M5C = MT = MT = MG |
| 1019 | CO-7263 | MT = MG = MG = M5C = MT = M5C = M5C = MT = M5C = M5C = MT = MT = MA = M5C = MT = MT = M5C = M5C = MT = M5C |
| 1020 | CO-7264 | MG = MT = M5C = MT = MG = MG = M5C = MT = M5C = M5C = MT = M5C = M5C = MT = MT = MA = M5C = MT = MT = M5C |
| 1021 | CO-7265 | M5C = MT = MG = MG = MT = M5C = MT = MG = MG = M5C = MT = M5C = M5C = MT = M5C = M5C = MT = MT = MA = M5C |
| 1022 | CO-7266 | MA = MA = M5C = M5C = MT = MG = MG = MT = M5C = MT = MG = MG = M5C = MT = M5C = M5C = MT = M5C = M5C = MT |
| 1023 | CO-7267 | MG = MA = M5C = MT = MG = MA = MA = M5C = M5C = MT = MG = MG = MT = M5C = MT = MG = MG = M5C = MT = M5C |
| 1024 | CO-7268 | MT = MG = MG = MT = MG = MA = M5C = MT = MG = MA = MA = M5C = M5C = MT = MG = MG = MT = M5C = MT = MG |
| 1025 | CO-7269 | MG = MT = MG = MG = MT = MG = MA = M5C = MT = MG = MA = MA = M5C = M5C = MT = MG = MG = MT = M5C = MT |
| 1026 | CO-7270 | M5C = MT = M5C = MG = MA = MA = MG = MG = MT = MA = MG = MT = MA = MG = MG = MG = MA = M5C = MT = MT |
| 1027 | CO-7271 | MT = MT = M5C = MT = M5C = MG = MA = MA = MG = MG = MT = MA = MG = MT = MA = MG = MG = MG = MA = M5C |
| 1028 | CO-7272 | MT = MT = MG = MG = M5C = MT = MT = M5C = MT = M5C = MG = MA = MA = MG = MG = MT = MA = MG = MT = MA |
| 1029 | CO-7273 | MA = MA = M5C = MG = MA = MG = MA = M5C = M5C = MT = MG = MA = MG = MA = M5C = M5C = MT = MT = MG = MG |
| 1030 | CO-7274 | MG = MG = MA = M5C = MT = MT = MG = MG = MG = MA = MG = MT = MG = MG = MT = MG = MA = M5C = MT = M5C |
| 1031 | CO-7280 | MG = MG = MA = M5C = MT = MT = MG = MG = MG = MA = MG = MT = MG = MG = MT = MG = MA = M5C = MT = MA |
| 1032 | CO-7281 | MG = MG = MA = M5C = MT = MT = MG = MG = MG = MA = MG = MT = MG = MG = MT = MG = MA = M5C = MT = MT |
| 1033 | CO-7310 | MG = M5C = M5C = MT = MT = MG = MG = MG = MG = MG = MG = M5C = MT = MG = M5C = MG = MG = MT = MA = MG |
| 1034 | CO-7311 | M5C = MG = M5C = M5C = MT = MT = MG = MG = MG = MG = MG = MG = M5C = MT = MG = M5C = MG = MG = MT = MA |
| 1035 | CO-7312 | MG = M5C = MG = M5C = M5C = MT = MT = MG = MG = MG = MG = MG = MG = M5C = MT = MG = M5C = MG = MG = MT |
| 1036 | CO-7313 | MA = MG = M5C = MG = M5C = M5C = MT = MT = MG = MG = MG = MG = MG = MG = M5C = MT = MG = M5C = MG = MG |
| 1037 | CO-7314 | M5C = MA = MG = M5C = MG = M5C = M5C = MT = MT = MG = MG = MG = MG = MG = MG = M5C = MT = MG = M5C = MG |
| 1038 | CO-7315 | M5C = M5C = MA = MG = M5C = MG = M5C = M5C = MT = MT = MG = MG = MG = MG = MG = MG = M5C = MT = MG = M5C |
| 1039 | CO-7316 | M5C = MA = MG = M5C = MT = M5C = MA = MG = MT = MA = MG = M5C = MT = M5C = MA = M5C = MA = MT = MT = MT |
| 1040 | CO-7317 | M5C = M5C = MA = MG = M5C = MT = M5C = MA = MG = MT = MA = MG = M5C = MT = M5C = MA = M5C = MA = MT = MT |
| 1041 | CO-7694 | MT = MT = M5C = LT = M5C = LG = MA = LA = MG = LG = MT = LA = MG = LT = MA = LG = MG = LG = MA = M5C |
| 1042 | CO-7695 | MT = MT = L5C = MT = L5C = MG = LA = MA = LG = MG = LT = MA = LG = MT = LA = MG = LG = MG = MA = M5C |
| 1043 | CO-7696 | MT = MT = L5C = MT = M5C = LG = MA = MA = LG = MG = MT = LA = MG = MT = LA = MG = MG = LG = MA = M5C |
| 1044 | CO-7697 | MT = MT = M5C = MT = L5C = MG = MA = MA = LG = MG = MT = MA = LG = MT = MA = MG = LG = MG = MA = M5C |
| 1045 | CO-7698 | MT = MT = M5C = LT = M5C = MG = MA = LA = MG = MG = MT = LA = MG = MT = MA = LG = MG = MG = MA = M5C |
| 1046 | CO-7699 | MT = MT = M5C = MT = M5C = LG = MA = MA = MG = MG = LT = MA = MG = MT = MA = LG = MG = MG = MA = M5C |
| 1047 | CO-7700 | MT = MT = M5C = MT = L5C = MG = MA = MA = MG = LG = MT = MA = MG = MT = LA = MG = MG = MG = MA = M5C |
| 1048 | CO-7701 | MT = MT = M5C = MT − M5C − MG = MA = MA = MG = MG = MT = MA = MG = MT = MA − MG − MG = MG = MA = M5C |
| 1049 | CO-7702 | MT = MT = M5C − MT − M5C − MG = MA = MA = MG = MG = MT = MA = MG = MT = MA − MG − MG − MG = MA = M5C |
| 1050 | CO-7703 | MT = MT − M5C − MT − M5C − MG = MA = MA = MG = MG = MT = MA = MG = MT = MA − MG − MG − MG − MA = M5C |
| 1051 | CO-7704 | MT − MT − M5C − MT − M5C − MG = MA = MA = MG = MG = MT = MA = MG = MT = MA − MG − MG − MG − MA − M5C |
| 1052 | CO-7705 | MT = MT = M5C = MT = M5C − MG − MA − MA − MG − MG − MT − MA − MG − MT − MA − MG − MG = MG = MA = M5C |
| 1053 | CO-7706 | MT = MT = MG = LG = M5C = LT = MT = L5C = MT = L5C = MG = LA = MA = LG = MG = LT = MA = LG = MT = MA |
| 1054 | CO-7707 | MT = MT = LG = MG = L5C = MT = LT = M5C = LT = M5C = LG = MA = LA = MG = LG = MT = LA = MG = MT = MA |
| 1055 | CO-7708 | MT = MT = LG = MG = M5C = LT = MT = M5C = LT = M5C = MG = LA = MA = MG = LG = MT = MA = LG = MT = MA |
| 1056 | CO-7709 | MT = MT = MG = MG = L5C = MT = MT = M5C = LT = M5C = MG = MA = LA = MG = MG = MT = LA = MG = MT = MA |
| 1057 | CO-7710 | MT = MT = MG = LG = M5C = MT = MT = L5C = MT = M5C = MG = LA = MA = MG = MG = LT = MA = MG = MT = MA |
| 1058 | CO-7711 | MT = MT = MG = MG = M5C = LT = MT = M5C = MT = M5C = LG = MA = MA = MG = MG = LT = MA = MG = MT = MA |
| 1059 | CO-7712 | MT = MT = MG = MG = L5C = MT = MT = M5C = MT = L5C = MG = MA = MA = MG = LG = MT = MA = MG = MT = MA |
| 1060 | CO-7713 | MT = MT = MG = MG − M5C − MT = MT = M5C = MT = M5C = MG = MA = MA = MG = MG − MT − MA = MG = MT = MA |
| 1061 | CO-7714 | MT = MT = MG − MG − M5C − MT = MT = M5C = MT = M5C = MG = MA = MA = MG = MG − MT − MA − MG = MT = MA |
| 1062 | CO-7715 | MT = MT − MG − MG − M5C − MT = MT = M5C = MT = M5C = MG = MA = MA = MG = MG − MT − MA − MG − MT = MA |
| 1063 | CO-7716 | MT − MT − MG − MG − M5C − MT = MT = M5C = MT = M5C = MG = MA = MA = MG = MG − MT − MA − MG − MT − MA |
| 1064 | CO-7717 | MT = MT = MG = MG = M5C − MT − MT − M5C − MT − M5C − MG − MA − MA − MG − MG − MT − MA = MG = MT = MA |
| 1065 | CO-7730 | M5C = MT = MT = M5C = MT = M5C = MG = MA = MA = MG = MG = MT = MA = MG = MT = MA = MG = MG = MG = MA = M5C = MT |
| 1066 | CO-7731 | M5C = MT = MT = M5C = MT = L5C = MG = MA = LA = MG = MG = MT = MA = LG = MT = MA = LG = MG = MG = MA = M5C = MT |
| 1067 | CO-7732 | MG = M5C = MT = MT = M5C = MT = M5C = MG = MA = MA = MG = MG = MT = MA = MG = MT = MA = MG = MG = MG = MA = M5C = MT = MT |
| 1068 | CO-7733 | MG = M5C = MT = MT = L5C = MT = M5C = LG = MA = MA = LG = MG = MT = LA = MG = MT = LA = MG = MG = LG = MA = M5C = MT = MT |
| 1069 | CO-7734 | M5C = MT = MT = MG = MG = M5C = MT = MT = M5C = MT = M5C = MG = MA = MA = MG = MG = MT = MA = MG = MT = MA = MG |
| 1070 | CO-7735 | M5C = MT = MT = MG = MG = L5C = MT = MT = L5C = MT = M5C = MG = MA = LA = MG = MG = LT = MA = MG = MT = MA = MG |
| 1071 | CO-7736 | M5C = M5C = MT = MT = MG = MG = M5C = MT = MT = M5C = MT = M5C = MG = MA = MA = MG = MG = MT = MA = MG = MT = MA = MG = MG |
| 1072 | CO-7737 | M5C = M5C = MT = MT = LG = MG = M5C = LT = MT = M5C = LT = M5C = MG = LA = MA = MG = LG = MT = MA = LG = MT = MA = MG = MG |
| 1073 | CO-7760 | MG = MG = MA = M5C = MT = LT = MG = MG = LG = MA = MG = LT = MG = MG = LT = MG = MA = M5C = MT = MG − dT − dT − dT − dT − dT − |
| | | dT − dT − dT − dT − dT − .BioTEG |
| 1074 | CO-7762 | MG − MG − MA − M5C − MT − LT − MG − MG − LG − MA − MG − LT − MG − MG − LT − MG − MA − M5C − MT − MG − dT − dT − dT − dT − dT − dT − dT − dT − |
| | | dT − dT − .BioTEG |
| 1075 | CO-7764 | M5C = M5C = M5C = M5C = MT = dT = dG = dG = d5C = dA = d5C = dT = d5C = dG = dG = MA = MG = MT = MT = MG |
| 1076 | CO-7765 | MG = M5C = MA = MG = M5C = dT = dA = dG = dG = dT = dT = dG = dG = d5C = dT = M5C = M5C = M5C = M5C = MT |
| 1077 | CO-7766 | MT = MT = MG = M5C = MG = dG = dG = dT = dG = dG = dG = dA = d5C = dG = dA = MG = M5C = MT = MA = MG |
| 1078 | CO-7767 | MG = MG = MT = MG = MA = d5C = dT = dG = dA = dA = d5C = d5C = dT = dG = dG = MT = M5C = MT = MG = MG |
| 1079 | CO-7768 | M5C = MG = MA = MA = MG = dG = dT = dA = dG = dT = dA = dG = dG = dG = dA = M5C = MT = MT = MG = MG |
| 1080 | CO-7769 | MA = M5C = MG = MA = MG = dA = d5C = d5C = dT = dG = dA = dG = dA = d5C = d5C = MT = MT = MG = MG = M5C |
| 1081 | CO-7770 | MT = MA = MG = MG = MT = dG = d5C = dT = dG = dG = d5C = dT = dT = d5C = dT = MT = M5C = M5C = M5C = M5C |
| 1082 | CO-7771 | MA = M5C = M5C = MA = MT = dT = d5C = dT = dT = dG = dA = d5C = d5C = d5C = dA = MG = M5C = MT = M5C = M5C |
| 1083 | CO-7772 | MG = M5C = MA = MG = MT = dG = dA = d5C = dG = d5C = d5C = d5C = d5C = dG = dT = MT = MA = MG = MA = MA |
| 1084 | CO-7773 | MG = MA = MG = MA = M5C = dA = dA = d5C = dA = dG = dA = dA = dG = dA = dA = M5C = M5C = MG = M5C = MT |
| 1085 | CO-7774 | MG = M5C = M5C = MA = MT = dG = dT = dG = dA = dG = d5C = dT = dT = dG = dA = MG = MG = MT = MT = M5C |
| 1086 | CO-7775 | MG = MG = MA = MG = MT = d5C = dG = dG = dA = d5C = dG = d5C = dA = dG = dG = MT = MA = MG = MG = MA |
| 1087 | CO-7776 | MG = MG = MG = MT = M5C = dT = dG = dT = dA = dG = dT = d5C = dT = dG = dA = MG = M5C = MG = M5C = MT |
| 1088 | CO-7777 | MT = MG = MG = MA = MA = dA = d5C = dT = dG = dA = dG = dG = dT = dA = dG = MG = M5C = MG = MG = MG |
| 1089 | CO-7778 | MA = MG = M5C = M5C = MA = dA = dT = dG = dG = dG = dA = dG = d5C = dG = dG = MG = MT = MA = MG = M5C |
| 1090 | CO-7779 | MG = MG = M5C = MT = MG = dG = d5C = d5C = d5C = d5C = dA = d5C = d5C = dT = d5C = MT = MA = MT = MA = MT |
| 1091 | CO-7780 | MA = MG = MG = MA = MG = dA = dG = dT = dG = dA = dT = dT = dT = dG = dA = MG = MT = MA = MG = MA |
| 1092 | CO-7781 | MG = M5C = MT = MG = MT = d5C = d5C = d5C = dG = dA = d5C = dG = dT = d5C = dA = M5C = MA = MT = MG = MA |
| 1093 | CO-7782 | MG = MG = MG = MT = MT = dA = dA = dG = dG = dA = dA = dG = dG = d5C = dG = MA = M5C = MG = MA = MG |
| 1094 | CO-7783 | MT = MA = MG = M5C = MA = dA = dA = d5C = dT = d5C = d5C = d5C = d5C = d5C = dA = MG = MG = M5C = MG = M5C |
| 1095 | CO-7784 | MG = M5C = M5C = M5C = M5C = dA = dG = dA = d5C = dA = d5C = dG = d5C = dG = d5C = MT = MA = MT = M5C = MA |
| 1096 | CO-7785 | MG = MG = M5C = MA = MG = dG = dA = dT = dA = dG = dG = dA = dT = dA = dG = MG = MA = MG = M5C = MG |
| 1097 | CO-7786 | MT = MG = M5C = M5C = MT = dA = dG = d5C = dA = dT = dG = dT = d5C = dA = dA = M5C = M5C = MG = MA = MG |
| 1098 | CO-7787 | M5C = MA = MT = M5C = M5C = dT = dT = d5C = dA = dA = d5C = dT = d5C = dA = d5C = MT = M5C = MT = MG = MG |
| 1099 | CO-7788 | MG = MG = M5C = MT = MG = dT = dT = dT = dA = dT = d5C = dT = dT = dT = dG = MT = M5C = M5C = MT = MG |
| 1100 | CO-7789 | MT = MG = MT = M5C = M5C = dT = dG = d5C = dT = dG = dA = dG = d5C = dA = d5C = M5C = MT = MT = MG = MA |
| 1101 | CO-7790 | MG = MA = MT = MT = M5C = dA = dG = d5C = d5C = dT = dG = dA = dG = dA = d5C = MT = MG = MT = M5C = MT |
| 1102 | CO-7791 | MG = M5C = MA = MA = M5C = dT = dG = dG = d5C = dG = d5C = dA = d5C = dA = dA = M5C = M5C = MT = MT = MG |
| 1103 | CO-7792 | MG = MA = M5C = MA = MA = dT = d5C = dT = dA = d5C = dT = dT = d5C = d5C = dT = M5C = MT = M5C = M5C = M5C |
| 1104 | CO-7793 | MA = MA = MT = M5C = MT = dT = d5C = d5C = d5C = dA = dG = dA = d5C = dT = d5C = MA = MG = M5C = MT = M5C |
| 1105 | CO-7794 | MG = M5C = MT = M5C = MT = dT = d5C = d5C = d5C = dA = dA = d5C = dA = d5C = d5C = MT = M5C = M5C = MT = M5C |
| 1106 | CO-7795 | MG = M5C = M5C = MT = MG = d5C = d5C = d5C = dT = dT = d5C = d5C = d5C = dA = dT = M5C = MT = MG = MG = MT |
| 1107 | CO-7796 | MG = MG = MA = MG = MA = d5C = dT = dA = dG = dG = dA = dA = dG = d5C = d5C = MA = M5C = MT = MT = M5C |
| 1108 | CO-7797 | MA = MT = MA = MT = M5C = dA = dA = dT = dT = d5C = d5C = dT = d5C = dG = dA = MA = MG = MG = M5C = MA |
| 1109 | CO-7798 | MG = MG = M5C = MT = MA = dA = dT = d5C = dT = dG = dT = dT = d5C = dT = dG = MT = MG = MG = MT = MA |
| 1110 | CO-7799 | M5C = MT = MG = MG = M5C = d5C = d5C = dA = dT = d5C = dT = d5C = d5C = d5C = dA = MT = M5C = MA = MT = M5C |
| 1111 | CO-7800 | MG = MT = MT = M5C = M5C = dT = d5C = d5C = dA = d5C = dG = dG = d5C = d5C = d5C = MA = MG = MT = MA = MT |
| 1112 | CO-7801 | MG = M5C = MT = MG = M5C = dA = dG = d5C = dA = dG = dT = d5C = dT = d5C = d5C = MA = MT = M5C = M5C = M5C |
| 1113 | CO-7802 | MA = MT = M5C = M5C = MT = d5C = dA = dG = d5C = dT = d5C = d5C = dA = dG = dT = MT = M5C = M5C = MT = MT |
| 1114 | CO-7803 | M5C = MG = MA = M5C = MT = dT = d5C = dT = dG = d5C = dT = dG = d5C = d5C = dT = M5C = MG = MA = MG = M5C |
| 1115 | CO-7804 | MT = M5C = MG = MG = MT = dT = dG = dG = d5C = dA = d5C = d5C = d5C = dT = dT = MT = M5C = M5C = M5C = MT |
| 1116 | CO-7805 | MT = M5C = M5C = MA = MG = dG = d5C = dT = dT = dA = dG = dA = dA = d5C = d5C = M5C = M5C = MT = MG = M5C |
| 1117 | CO-7806 | MT = MG = MT = M5C = MT = dT = dA = dT = d5C = d5C = dG = d5C = dT = dG = dT = M5C = MT = MG = MG = MG |
| 1118 | CO-7807 | MG = MG = M5C = MA = M5C = dA = dG = dA = d5C = dT = dA = dG = dT = dA = d5C = MT = MA = MG = MG = MT |
| 1119 | CO-7808 | M5C = MT = MA = MG = MT = d5C = dT = d5C = dA = dA = dG = dA = dT = dA = d5C = MA = M5C = MA = MG = MG |
| 1120 | CO-7809 | MG = MT = MG = MG = MT = dT = dG = dT = dA = dA = dT = dT = dA = dG = dT = MA = MG = MG = MT = MT |
| 1121 | CO-7810 | M5C = MA = M5C = MG = MG = d5C = dT = dT = dT = dG = dA = d5C = dG = dA = dT = MT = MA = MG = MA = M5C |
| 1122 | CO-7811 | M5C = M5C = MT = MA = MA = dA = dT = dG = dT = d5C = d5C = d5C = dT = dG = dG = M5C = M5C = MG = MG = M5C |
| 1123 | CO-7812 | MG = MT = M5C = M5C = M5C = dT = d5C = d5C = dA = d5C = d5C = d5C = dT = dG = dA = MG = MT = MT = MG = MT |
| 1124 | CO-7813 | MG = MT = MT = M5C = MT = dG = dT = dG = dT = dG = dT = d5C = dG = dT = dG = MT = MG = MA = MT = MG |
| 1125 | CO-7814 | MG = MA = MG = MT = M5C = dT = d5C = dA = dG = d5C = dA = d5C = dT = dA = dT = MT = MG = MA = M5C = M5C |
| 1126 | CO-7815 | M5C = MG = MT = MT = MA = dT = d5C = dG = dT = dG = dG = d5C = dA = d5C = d5C = MT = MT = MA = M5C = M5C |
| 1127 | CO-7816 | MG = MA = MT = MG = M5C = dT = dG = d5C = dA = dT = dA = dA = d5C = dT = d5C = MT = M5C = MT = MT = MA |
| 1128 | CO-7817 | MG = MG = MA = M5C = MA = dT = dA = dA = dG = dG = d5C = dT = dG = d5C = d5C = MT = MT = M5C = MT = MG |
| 1129 | CO-7818 | MG = MT = MT = MA = MG = dG = dG = dA = dG = dG = dG = dT = dG = dA = d5C = MT = MG = M5C = MT = MA |
| 1130 | CO-7819 | M5C = M5C = M5C = M5C = MT = MT = MG = MG = M5C = MA = M5C = MT = M5C = MG = MG = MA = MG = MT = MT = MG |
| 1131 | CO-7820 | MG = M5C = MA = MG = M5C = MT = MA = MG = MG = MT = MT = MG = MG = M5C = MT = M5C = M5C = M5C = M5C = MT |
| 1132 | CO-7821 | MT = MT = MG = M5C = MG = MG = MG = MT = MG = MG = MG = MA = M5C = MG = MA = MG = M5C = MT = MA = MG |
| 1133 | CO-7822 | MG = MG = MT = MG = MA = M5C = MT = MG = MA = MA = M5C = M5C = MT = MG = MG = MT = M5C = MT = MG = MG |
| 1134 | CO-7823 | M5C = MG = MA = MA = MG = MG = MT = MA = MG = MT = MA = MG = MG = MG = MA = M5C = MT = MT = MG = MG |
| 1135 | CO-7824 | MA = M5C = MG = MA = MG = MA = M5C = M5C = MT = MG = MA = MG = MA = M5C = M5C = MT = MT = MG = MG = M5C |
| 1136 | CO-7825 | MT = MA = MG = MG = MT = MG = M5C = MT = MG = MG = M5C = MT = MT = M5C = MT = MT = M5C = M5C = M5C = M5C |
| 1137 | CO-7826 | MA = M5C = M5C = MA = MT = MT = M5C = MT = MT = MG = MA = M5C = M5C = M5C = MA = MG = M5C = MT = M5C = M5C |
| 1138 | CO-7827 | MG = M5C = MA = MG = MT = MG = MA = M5C = MG = M5C = M5C = M5C = M5C = MG = MT = MT = MA = MG = MA = MA |
| 1139 | CO-7828 | MG = MA = MG = MA = M5C = MA = MA = M5C = MA = MG = MA = MA = MG = MA = MA = M5C = M5C = MG = M5C = MT |
| 1140 | CO-7829 | MG = M5C = M5C = MA = MT = MG = MT = MG = MA = MG = M5C = MT = MT = MG = MA = MG = MG = MT = MT = M5C |
| 1141 | CO-7830 | MG = MG = MA = MG = MT = M5C = MG = MG = MA = M5C = MG = M5C = MA = MG = MG = MT = MA = MG = MG = MA |
| 1142 | CO-7831 | MG = MG = MG = MT = M5C = MT = MG = MT = MA = MG = MT = M5C = MT = MG = MA = MG = M5C = MG = M5C = MT |
| 1143 | CO-7832 | MT = MG = MG = MA = MA = MA = M5C = MT = MG = MA = MG = MG = MT = MA = MG = MG = M5C = MG = MG = MG |
| 1144 | CO-7833 | MA = MG = M5C = M5C = MA = MA = MT = MG = MG = MG = MA = MG = M5C = MG = MG = MG = MT = MA = MG = M5C |
| 1145 | CO-7834 | MG = MG = M5C = MT = MG = MG = M5C = M5C = M5C = M5C = MA = M5C = M5C = MT = M5C = MT = MA = MT = MA = MT |
| 1146 | CO-7835 | MA = MG = MG = MA = MG = MA = MG = MT = MG = MA = MT = MT = MT = MG = MA = MG = MT = MA = MG = MA |
| 1147 | CO-7836 | MG = M5C = MT = MG = MT = M5C = M5C = M5C = MG = MA = M5C = MG = MT = M5C = MA = M5C = MA = MT = MG = MA |
| 1148 | CO-7837 | MG = MG = MG = MT = MT = MA = MA = MG = MG = MA = MA = MG = MG = M5C = MG = MA = M5C = MG = MA = MG |
| 1149 | CO-7838 | MT = MA = MG = M5C = MA = MA = MA = M5C = MT = M5C = M5C = M5C = M5C = M5C = MA = MG = MG = M5C = MG = M5C |
| 1150 | CO-7839 | MG = M5C = M5C = M5C = M5C = MA = MG = MA = M5C = MA = M5C = MG = M5C = MG = M5C = MT = MA = MT = M5C = MA |
| 1151 | CO-7840 | MG = MG = M5C = MA = MG = MG = MA = MT = MA = MG = MG = MA = MT = MA = MG = MG = MA = MG = M5C = MG |
| 1152 | CO-7841 | MT = MG = M5C = M5C = MT = MA = MG = M5C = MA = MT = MG = MT = M5C = MA = MA = M5C = M5C = MG = MA = MG |
| 1153 | CO-7842 | M5C = MA = MT = M5C = M5C = MT = MT = M5C = MA = MA = M5C = MT = M5C = MA = M5C = MT = M5C = MT = MG = MG |
| 1154 | CO-7843 | MG = MG = M5C = MT = MG = MT = MT = MT = MA = MT = M5C = MT = MT = MT = MG = MT = M5C = M5C = MT = MG |
| 1155 | CO-7844 | MT = MG = MT = M5C = M5C = MT = MG = M5C = MT = MG = MA = MG = M5C = MA = M5C = M5C = MT = MT = MG = MA |
| 1156 | CO-7845 | MG = MA = MT = MT = M5C = MA = MG = M5C = M5C = MT = MG = MA = MG = MA = M5C = MT = MG = MT = M5C = MT |
| 1157 | CO-7846 | MG = M5C = MA = MA = M5C = MT = MG = MG = M5C = MG = M5C = MA = M5C = MA = MA = M5C = M5C = MT = MT = MG |
| 1158 | CO-7847 | MG = MA = M5C = MA = MA = MT = M5C = MT = MA = M5C = MT = MT = M5C = M5C = MT = M5C = MT = M5C = M5C = M5C |
| 1159 | CO-7848 | MA = MA = MT = M5C = MT = MT = M5C = M5C = M5C = MA = MG = MA = M5C = MT = M5C = MA = MG = M5C = MT = M5C |
| 1160 | CO-7849 | MG = M5C = MT = M5C = MT = MT = M5C = M5C = M5C = MA = MA = M5C = MA = M5C = M5C = MT = M5C = M5C = MT = M5C |
| 1161 | CO-7850 | MG = M5C = M5C = MT = MG = M5C = M5C = M5C = MT = MT = M5C = M5C = M5C = MA = MT = M5C = MT = MG = MG = MT |
| 1162 | CO-7851 | MG = MG = MA = MG = MA = M5C = MT = MA = MG = MG = MA = MA = MG = M5C = M5C = MA = M5C = MT = MT = M5C |
| 1163 | CO-7852 | MA = MT = MA = MT = M5C = MA = MA = MT = MT = M5C = M5C = MT = M5C = MG = MA = MA = MG = MG = M5C = MA |
| 1164 | CO-7853 | MG = MG = M5C = MT = MA = MA = MT = M5C = MT = MG = MT = MT = M5C = MT = MG = MT = MG = MG = MT = MA |
| 1165 | CO-7854 | M5C = MT = MG = MG = M5C = M5C = M5C = MA = MT = M5C = MT = M5C = M5C = M5C = MA = MT = M5C = MA = MT = M5C |
| 1166 | CO-7855 | MG = MT = MT = M5C = M5C = MT = M5C = M5C = MA = M5C = MG = MG = M5C = M5C = M5C = MA = MG = MT = MA = MT |
| 1167 | CO-7856 | MG = M5C = MT = MG = M5C = MA = MG = M5C = MA = MG = MT = M5C = MT = M5C = M5C = MA = MT = M5C = M5C = M5C |
| 1168 | CO-7857 | MA = MT = M5C = M5C = MT = M5C = MA = MG = M5C = MT = M5C = M5C = MA = MG = MT = MT = M5C = M5C = MT = MT |
| 1169 | CO-7858 | M5C = MG = MA = M5C = MT = MT = M5C = MT = MG = M5C = MT = MG = M5C = M5C = MT = M5C = MG = MA = MG = M5C |
| 1170 | CO-7859 | MT = M5C = MG = MG = MT = MT = MG = MG = M5C = MA = M5C = M5C = M5C = MT = MT = MT = M5C = M5C = M5C = MT |
| 1171 | CO-7860 | MT = M5C = M5C = MA = MG = MG = M5C = MT = MT = MA = MG = MA = MA = M5C = M5C = M5C = M5C = MT = MG = M5C |
| 1172 | CO-7861 | MT = MG = MT = M5C = MT = MT = MA = MT = M5C = M5C = MG = M5C = MT = MG = MT = M5C = MT = MG = MG = MG |
| 1173 | CO-7862 | MG = MG = M5C = MA = M5C = MA = MG = MA = M5C = MT = MA = MG = MT = MA = M5C = MT = MA = MG = MG = MT |
| 1174 | CO-7863 | M5C = MT = MA = MG = MT = M5C = MT = M5C = MA = MA = MG = MA = MT = MA = M5C = MA = M5C = MA = MG = MG |
| 1175 | CO-7864 | MG = MT = MG = MG = MT = MT = MG = MT = MA = MA = MT = MT = MA = MG = MT = MA = MG = MG = MT = MT |
| 1176 | CO-7865 | M5C = MA = M5C = MG = MG = M5C = MT = MT = MT = MG = MA = M5C = MG = MA = MT = MT = MA = MG = MA = M5C |
| 1177 | CO-7866 | M5C = M5C = MT = MA = MA = MA = MT = MG = MT = M5C = M5C = M5C = MT = MG = MG = M5C = M5C = MG = MG = M5C |
| 1178 | CO-7867 | MG = MT = M5C = M5C = M5C = MT = M5C = M5C = MA = M5C = M5C = M5C = MT = MG = MA = MG = MT = MT = MG = MT |
| 1179 | CO-7868 | MG = MT = MT = M5C = MT = MG = MT = MG = MT = MG = MT = M5C = MG = MT = MG = MT = MG = MA = MT = MG |
| 1180 | CO-7869 | MG = MA = MG = MT = M5C = MT = M5C = MA = MG = M5C = MA = M5C = MT = MA = MT = MT = MG = MA = M5C = M5C |
| 1181 | CO-7870 | M5C = MG = MT = MT = MA = MT = M5C = MG = MT = MG = MG = M5C = MA = M5C = M5C = MT = MT = MA = M5C = M5C |
| 1182 | CO-7871 | MG = MA = MT = MG = M5C = MT = MG = M5C = MA = MT = MA = MA = M5C = MT = M5C = MT = M5C = MT = MT = MA |
| 1183 | CO-7872 | MG = MG = MA = M5C = MA = MT = MA = MA = MG = MG = M5C = MT = MG = M5C = M5C = MT = MT = M5C = MT = MG |
| 1184 | CO-7873 | MG = MT = MT = MA = MG = MG = MG = MA = MG = MG = MG = MT = MG = MA = M5C = MT = MG = M5C = MT = MA |
| 1185 | CO-8046 | mG = MG = MA = M5C = MT = LT = MG = MG = LG = MA = MG = LT = MG = MG = LT = MG = MA = M5C = MT = MG |
| 1186 | CO-8170 | MG = MA = M5C = MT = MT = MG = MG = MG = MA = MG = MT = MG = MG = MT = MG = MA = M5C = MT = MG |
| 1187 | CO-8171 | MG = MA = M5C = MT = MT = MG = MG = MG = MA = MG = MT = MG = MG = MT = MG = MA = M5C = MT |
| 1188 | CO-8177 | MG = MA = M5C = MT = LT = MG = MG = MG = MA = LG = MT = MG = MG = MT = LG = MA = M5C = MT = MG |
| 691 | CO-8178 | MG = MA = M5C = MT = LT = MG = MG = MG = MA = LG = MT = MG = MG = MT = LG = MA = M5C = MT |
| 1189 | CO-8185 | MG = MG = MA = M5C = MT = LT = MG = MG = MG = MA = LG = MT = MG = MG = MT = LG = MA = M5C = MT = MG = MA = MA = M5C = M5C |
| 1190 | CO-8187 | MG = MG = MA = M5C = MT = MT = MG = MG = MG = MA = MG = MT = MG = MG = MT = MG = MA = M5C = MT = MG = MA = MA = M5C = M5C = MT |
| 1191 | CO-8188 | MG = MG = MA = M5C = MT = LT = MG = MG = MG = MA = LG = MT = MG = MG = MT = LG = MA = M5C = MT = MG = MA = MA = M5C = M5C = MT |
| 1192 | CO-8190 | MG = MG = MG = MA = M5C = MT = LT = MG = MG = MG = MA = LG = MT = MG = MG = MT = LG = MA = M5C = MT = MG = MA = MA = M5C = M5C = MT |
| 1193 | CO-8192 | MA = MG = MG = MG = MA = M5C = MT = MT = MG = MG = MG = MA = MG = MT = MG = MG = MT = MG = MA = M5C = MT = MG = MA = MA = M5C = M5C = MT |
| 1194 | CO-8193 | MA = MG = MG = MG = MA = M5C = MT = LT = MG = MG = MG = MA = LG = MT = MG = MG = MT = LG = MA = M5C = MT = MG = MA = MA = M5C = M5C = MT |
| 1195 | CO-8195 | MT = MA = MG = MG = MG = MA = M5C = MT = MT = MG = MG = MG = MA = MG = MT = MG = MG = MT = MG = MA = M5C = MT = MG = MA = MA = M5C = M5C = MT |
| 1196 | CO-8196 | MT = MA = MG = MG = MG = MA = M5C = MT = LT = MG = MG = MG = MA = LG = MT = MG = MG = MT = LG = MA = M5C = MT = MG = MA = MA = M5C = M5C = MT |
| 1197 | CO-8198 | MG = MT = MA = MG = MG = MG = MA = M5C = MT = MT = MG = MG = MG = MA = MG = MT = MG = MG = MT = MG = MA = M5C = MT = MG = MA = MA = M5C = M5C = MT |
| 1198 | CO-8199 | MG = MT = MA = MG = MG = MG = MA = M5C = MT = LT = MG = MG = MG = MA = LG = MT = MG = MG = MT = LG = MA = M5C = MT = MG = MA = MA = M5C = M5C = MT |
| 1199 | CO-8201 | MA = MG = MT = MA = MG = MG = MG = MA = M5C = MT = MT = MG = MG = MG = MA = MG = MT = MG = MG = MT = MG = MA = M5C = MT = MG = MA = MA = M5C = |
| | | M5C = MT |
| 1200 | CO-8202 | MA = MG = MT = MA = MG = MG = MG = MA = M5C = MT = LT = MG = MG = MG = MA = LG = MT = MG = MG = MT = LG = MA = M5C = MT = MG = MA = MA = M5C = |
| | | M5C = MT |
| 1201 | CO-8204 | MT = MA = MG = MT = MA = MG = MG = MG = MA = M5C = MT = MT = MG = MG = MG = MA = MG = MT = MG = MG = MT = MG = MA = M5C = MT = MG = MA = MA = M5C = |
| | | M5C = MT |
| 1202 | CO-8205 | MT = MA = MG = MT = MA = MG = MG = MG = MA = M5C = MT = LT = MG = MG = MG = MA = LG = MT = MG = MG = MT = LG = MA = M5C = MT = MG = MA = MA = M5C = |
| | | M5C = MT |
| 1203 | CO-8207 | MG = MG = MA = M5C = MT = MT = MG = MG = MG = MA = MG = MT = MG = MG = MT = MG = MA = M5C = MT = MG = MA = MA = M5C = M5C = MT = MG |
| 1204 | CO-8208 | MG = MG = MA = M5C = MT = LT = MG = MG = MG = MA = LG = MT = MG = MG = MT = LG = MA = M5C = MT = MG = MA = MA = M5C = M5C = MT = MG |
| 1205 | CO-8210 | MG = MG = MA = M5C = MT = MT = MG = MG = MG = MA = MG = MT = MG = MG = MT = MG = MA = M5C = MT = MG = MA = MA = M5C = M5C = MT = MG = MG |
| 1206 | CO-8211 | MG = MG = MA = M5C = MT = LT = MG = MG = MG = MA = LG = MT = MG = MG = MT = LG = MA = M5C = MT = MG = MA = MA = M5C = M5C = MT = MG = MG |
| 1207 | CO-8213 | MG = MG = MA = M5C = MT = MT = MG = MG = MG = MA = MG = MT = MG = MG = MT = MG = MA = M5C = MT = MG = MA = MA = M5C = M5C = MT = MG = MG = MT |
| 1208 | CO-8214 | MG = MG = MA = M5C = MT = LT = MG = MG = MG = MA = LG = MT = MG = MG = MT = LG = MA = M5C = MT = MG = MA = MA = M5C = M5C = MT = MG = MG = MT |
| 1209 | CO-8216 | MG = MG = MA = M5C = MT = MT = MG = MG = MG = MA = MG = MT = MG = MG = MT = MG = MA = M5C = MT = MG = MA = MA = M5C = M5C = MT = MG = MG = MT = M5C |
| 1210 | CO-8217 | MG = MG = MA = M5C = MT = LT = MG = MG = MG = MA = LG = MT = MG = MG = MT = LG = MA = M5C = MT = MG = MA = MA = M5C = M5C = MT = MG = MG = MT = M5C |
| 1211 | CO-8219 | MG = MG = MA = M5C = MT = MT = MG = MG = MG = MA = MG = MT = MG = MG = MT = MG = MA = M5C = MT = MG = MA = MA = M5C = M5C = MT = MG = MG = MT = |
| | | M5C = MT |
| 1212 | CO-8220 | MG = MG = MA = M5C = MT = LT = MG = MG = MG = MA = LG = MT = MG = MG = MT = LG = MA = M5C = MT = MG = MA = MA = M5C = M5C = MT = MG = MG = MT = |
| | | M5C = MT |
| 1213 | CO-8222 | MG = MG = MA = M5C = MT = MT = MG = MG = MG = MA = MG = MT = MG = MG = MT = MG = MA = M5C = MT = MG = MA = MA = M5C = M5C = MT = MG = MG = MT = |
| | | M5C = MT = M5C |
| 1214 | CO-8223 | MG = MG = MA = M5C = MT = LT = MG = MG = MG = MA = LG = MT = MG = MG = MT = LG = MA = M5C = MT = MG = MA = MA = M5C = M5C = MT = MG = MG = MT = |
| | | M5C = MT = M5C |
| 1215 | CO-8864 | MT = MA = MG = MG = MT = dG = d5C = MT = dG = dG = M5C = dT = dT = M5C = dT = MT = M5C = M5C = M5C = M5C |
| 1216 | CO-8865 | MT = MA = LG = MG = MT = dG = d5C = dT = dG = dG = d5C = dT = dT = d5C = dT = MT = M5C = L5C = M5C = M5C |
| 1217 | CO-8866 | LT = MA = MG = MG = MT = dG = d5C = dT = dG = dG = d5C = dT = dT = d5C = dT = MT = M5C = M5C = M5C = L5C |
| 1218 | CO-8867 | LT = MA = LG = MG = MT = dG = d5C = dT = dG = dG = d5C = dT = dT = d5C = dT = MT = M5C = L5C = M5C = L5C |
| 1219 | CO-8868 | MT = MA = MG − MG − MT = dG = d5C = dT = dG = dG = d5C = dT = dT = d5C = dT = MT − M5C − M5C = M5C = M5C |
| 1220 | CO-8869 | MT = MA − MG − MG − MT = dG = d5C = dT = dG = dG = d5C = dT = dT = d5C = dT = MT − M5C − M5C − M5C = M5C |
| 1221 | CO-8870 | MG = M5C = M5C = MA = MT = dG = dT = MG = dA = dG = M5C = dT = dT = MG = dA = MG = MG = MT = MT = M5C |
| 1222 | CO-8871 | MG = M5C = L5C = MA = MT = dG = dT = dG = dA = dG = d5C = dT = dT = dG = dA = MG = MG = LT = MT = M5C |
| 1223 | CO-8872 | LG = M5C = M5C = MA = MT = dG = dT = dG = dA = dG = d5C = dT = dT = dG = dA = MG = MG = MT = MT = L5C |
| 1224 | CO-8873 | LG = M5C = L5C = MA = MT = dG = dT = dG = dA = dG = d5C = dT = dT = dG = dA = MG = MG = LT = MT = L5C |
| 1225 | CO-8874 | MG = M5C = M5C − MA − MT = dG = dT = dG = dA = dG = d5C = dT = dT = dG = dA = MG − MG − MT = MT = M5C |
| 1226 | CO-8875 | MG = M5C − M5C − MA − MT = dG = dT = dG = dA = dG = d5C = dT = dT = dG = dA = MG − MG − MT − MT = M5C |
| 1227 | CO-8876 | MT = MA = MG = M5C = MA = dA = dA = M5C = dT = d5C = M5C = d5C = d5C = M5C = dA = MG = MG = M5C = MG = M5C |
| 1228 | CO-8877 | MT = MA = LG = M5C = MA = dA = dA = d5C = dT = d5C = d5C = d5C = d5C = d5C = dA = MG = MG = L5C = MG = M5C |
| 1229 | CO-8878 | LT = MA = MG = M5C = MA = dA = dA = d5C = dT = d5C = d5C = d5C = d5C = d5C = dA = MG = MG = M5C = MG = L5C |
| 1230 | CO-8879 | LT = MA = LG = M5C = MA = dA = dA = d5C = dT = d5C = d5C = d5C = d5C = d5C = dA = MG = MG = L5C = MG = L5C |
| 1231 | CO-8880 | MT = MA = MG − M5C − MA = dA = dA = d5C = dT = d5C = d5C = d5C = d5C = d5C = dA = MG − MG − M5C = MG = M5C |
| 1232 | CO-8881 | MT = MA − MG − M5C − MA = dA = dA = d5C = dT = d5C = d5C = d5C = d5C = d5C = dA = MG − MG − M5C − MG = M5C |
| 1233 | CO-8882 | M5C = MT = MG = MG = M5C = d5C = d5C = MA = dT = d5C = MT = d5C = d5C = M5C = dA = MT = M5C = MA = MT = M5C |
| 1234 | CO-8883 | M5C = MT = LG = MG = M5C = d5C = d5C = dA = dT = d5C = dT = d5C = d5C = d5C = dA = MT = M5C = LA = MT = M5C |
| 1235 | CO-8884 | L5C = MT = MG = MG = M5C = d5C = d5C = dA = dT = d5C = dT = d5C = d5C = d5C = dA = MT = M5C = MA = MT = L5C |
| 1236 | CO-8885 | L5C = MT = LG = MG = M5C = d5C = d5C = dA = dT = d5C = dT = d5C = d5C = d5C = dA = MT = M5C = LA = MT = L5C |
| 1237 | CO-8886 | M5C = MT = MG − MG − M5C = d5C = d5C = dA = dT = d5C = dT = d5C = d5C = d5C = dA = MT − M5C − MA = MT = M5C |
| 1238 | CO-8887 | M5C = MT − MG − MG − M5C = d5C = d5C = dA = dT = d5C = dT = d5C = d5C = d5C = dA = MT − M5C − MA − MT = M5C |
| 1239 | CO-8888 | MG = M5C = MT = MG = M5C = dA = dG = M5C = dA = dG = MT = d5C = dT = M5C = d5C = MA = MT = M5C = M5C = M5C |
| 1240 | CO-8889 | MG = M5C = LT = MG = M5C = dA = dG = d5C = dA = dG = dT = d5C = dT = d5C = d5C = MA = MT = L5C = M5C = M5C |
| 1241 | CO-8890 | LG = M5C = MT = MG = M5C = dA = dG = d5C = dA = dG = dT = d5C = dT = d5C = d5C = MA = MT = M5C = M5C = L5C |
| 1242 | CO-8891 | LG = M5C = LT = MG = M5C = dA = dG = d5C = dA = dG = dT = d5C = dT = d5C = d5C = MA = MT = L5C = M5C = L5C |
| 1243 | CO-8892 | MG = M5C = MT − MG − M5C = dA = dG = d5C = dA = dG = dT = d5C = dT = d5C = d5C = MA − MT − M5C = M5C = M5C |
| 1244 | CO-8893 | MG = M5C − MT − MG − M5C = dA = dG = d5C = dA = dG = dT = d5C = dT = d5C = d5C = MA − MT − M5C − M5C = M5C |
| 1245 | CO-8894 | MG = MT = MA = MG = MG = MT = dG = d5C = dT = dG = dG = d5C = dT = dT = d5C = dT = MT = M5C = M5C = M5C = M5C = MT |
| 1246 | CO-8895 | MG = MG = MT = MA = MG = MG = MT = dG = d5C = dT = dG = dG = d5C = dT = dT = d5C = dT = MT = M5C = M5C = M5C = M5C = MT = M5C |
| 1247 | CO-8896 | MG = MG = M5C = M5C = MA = MT = dG = dT = dG = dA = dG = d5C = dT = dT = dG = dA = MG = MG = MT = MT = M5C = M5C |
| 1248 | CO-8897 | MG = MG = MG = M5C = M5C = MA = MT = dG = dT = dG = dA = dG = d5C = dT = dT = dG = dA = MG = MG = MT = MT = M5C = M5C = M5C |
| 1249 | CO-8898 | M5C = MT = MA = MG = M5C = MA = dA = dA = d5C = dT = d5C = d5C = d5C = d5C = d5C = dA = MG = MG = M5C = MG = M5C = M5C |
| 1250 | CO-8899 | MT = M5C = MT = MA = MG = M5C = MA = dA = dA = d5C = dT = d5C = d5C = d5C = d5C = d5C = dA = MG = MG = M5C = MG = M5C = M5C = MT |
| 1251 | CO-8900 | M5C = M5C = MT = MG = MG = M5C = d5C = d5C = dA = dT = d5C = dT = d5C = d5C = d5C = dA = MT = M5C = MA = MT = M5C = M5C |
| 1252 | CO-8901 | MT = M5C = M5C = MT = MG = MG = M5C = d5C = d5C = dA = dT = d5C = dT = d5C = d5C = d5C = dA = MT = M5C = MA = MT = M5C = M5C = MA |
| 1253 | CO-8902 | MG = MG = M5C = MT = MG = M5C = dA = dG = d5C = dA = dG = dT = d5C = dT = d5C = d5C = MA = MT = M5C = M5C = M5C = M5C |
| 1254 | CO-8903 | MA = MG = MG = M5C = MT = MG = M5C = dA = dG = d5C = dA = dG = dT = d5C = dT = d5C = d5C = MA = MT = M5C = M5C = M5C = M5C = MT |
| 1255 | CO-8904 | M5C = M5C = M5C = M5C = MT = dT = dG = MG = d5C = dA = M5C = dT = d5C = MG = dG = MA = MG = MT = MT = MG |
| 1256 | CO-8905 | M5C = M5C = L5C = M5C = MT = LT = MG = MG = L5C = MA = M5C = LT = M5C = MG = LG = MA = MG = LT = MT = MG |
| 1257 | CO-8906 | M5C = M5C = M5C = M5C = LT = MT = MG = MG = L5C = MA = M5C = MT = L5C = MG = MG = MA = LG = MT = MT = MG |
| 1258 | CO-8907 | M5C = M5C = M5C = L5C = MT = MT = MG = LG = M5C = MA = M5C = LT = M5C = MG = MG = LA = MG = MT = MT = MG |
| 1259 | CO-8908 | M5C = L5C = M5C = M5C = LT = MT = MG = LG = M5C = MA = L5C = MT = M5C = LG = MG = MA = LG = MT = MT = LG |
| 1260 | CO-8909 | L5C = M5C = M5C = L5C = MT = MT = LG = MG = M5C = LA = M5C = MT = L5C = MG = MG = LA = MG = MT = LT = MG |
| 1261 | CO-8910 | MT = M5C = M5C = M5C = M5C = MT = dT = dG = dG = d5C = dA = d5C = dT = d5C = dG = dG = MA = MG = MT = MT = MG = MG |
| 1262 | CO-8911 | MT = M5C = M5C = M5C = M5C = MT = MT = MG = MG = M5C = MA = M5C = MT = M5C = MG = MG = MA = MG = MT = MT = MG = MG |
| 1263 | CO-8912 | M5C = MT = M5C = M5C = M5C = M5C = MT = dT = dG = dG = d5C = dA = d5C = dT = d5C = dG = dG = MA = MG = MT = MT = MG = MG = MG |
| 1264 | CO-8913 | M5C = MT = M5C = M5C = M5C = M5C = MT = MT = MG = MG = M5C = MA = M5C = MT = M5C = MG = MG = MA = MG = MT = MT = MG = MG = MG |
| 1265 | CO-10819 | [Palm] − MG = MG = MA = M5C = MT = LT = MG = MG = LG = MA = MG = LT = MG = MG = LT = MG = MA = M5C = MT = MG |
| 1266 | CO-10820 | [Palm] − MG = MG = MA = M5C = MT = LT = MG = MG = MG = MA = LG = MT = MG = MG = MT = LG = MA = M5C = MT = MG |
| 1267 | CO-10821 | [Palm] − MG = MG = MA = L5C = MT = MT = MG = MG = LG = MA = MG = MT = MG = LG = MT = MG = MA = M5C = LT = MG = MA = MA = M5C |
| 1268 | CO-10823 | [Palm] − MG = MG = MA − M5C − MT = MT = MG = MG = MG = MA = MG = MT = MG = MG = MT = MG − MA − M5C = MT = MG |
| 1269 | CO-10825 | MG = M5C = L5C = MA = MT = dG = dT = MG = dA = dG = M5C = dT = dT = MG = dA = MG = MG = LT = MT = M5C |
| 1270 | CO-10826 | LG = M5C = L5C = MA = MT = dG = dT = MG = dA = dG = M5C = dT = dT = MG = dA = MG = MG = LT = MT = L5C |
| 1271 | CO-10827 | MG = M5C = M5C − MA − MT = dG = dT = MG = dA = dG = M5C = dT = dT = MG = dA = MG − MG − MT = MT = M5C |
| 1272 | CO-10828 | MT = MA = LG = M5C = MA = dA = dA = M5C = dT = d5C = M5C = d5C = d5C = M5C = dA = MG = MG = L5C = MG = M5C |
| 1273 | CO-10829 | LT = MA = LG = M5C = MA = dA = dA = M5C = dT = d5C = M5C = d5C = d5C = M5C = dA = MG = MG = L5C = MG = L5C |
| 1274 | CO-10830 | MG = M5C = LT = MG = M5C = dA = dG = M5C = dA = dG = MT = d5C = dT = M5C = d5C = MA = MT = L5C = M5C = M5C |
| 1275 | CO-13669 | MG = d5C = d5C = dA = dT = dG = dT = dG = dA = dG = d5C = dT = dT = dG = dA = dG = dG = dT = dT = M5C |
| 1276 | CO-13670 | MG = M5C = d5C = dA = dT = dG = dT = dG = dA = dG = d5C = dT = dT = dG = dA = dG = dG = dT = MT = M5C |
| 1277 | CO-13671 | MG = M5C = M5C = dA = dT = dG = dT = dG = dA = dG = d5C = dT = dT = dG = dA = dG = dG = MT = MT = M5C |
| 1278 | CO-13672 | MG = M5C = M5C = MA = dT = dG = dT = dG = dA = dG = d5C = dT = dT = dG = dA = dG = MG = MT = MT = M5C |
| 1279 | CO-13673 | MG = M5C = M5C = MA = MT = MG = dT = dG = dA = dG = d5C = dT = dT = dG = MA = MG = MG = MT = MT = M5C |
| 1280 | CO-13674 | MG = M5C = M5C = MA = MT = MG = MT = dG = dA = dG = d5C = dT = dT = MG = MA = MG = MG = MT = MT = M5C |
| 1281 | CO-13675 | MG = M5C = M5C = MA = MT = MG = MT = MG = dA = dG = d5C = dT = MT = MG = MA = MG = MG = MT = MT = M5C |
| 1282 | CO-13676 | MG = M5C = M5C = MA = MT = MG = MT = MG = MA = dG = d5C = MT = MT = MG = MA = MG = MG = MT = MT = M5C |
| 1283 | CO-13677 | MG = d5C = d5C = dA = dT = dG = dT = dG = dA = dG = d5C = MT = MT = MG = MA = MG = MG = MT = MT = M5C |
| 1284 | CO-13678 | MG = M5C = d5C = dA = dT = dG = dT = dG = dA = dG = d5C = dT = MT = MG = MA = MG = MG = MT = MT = M5C |
| 1285 | CO-13679 | MG = M5C = M5C = dA = dT = dG = dT = dG = dA = dG = d5C = dT = dT = MG = MA = MG = MG = MT = MT = M5C |
| 1286 | CO-13680 | MG = M5C = M5C = MA = dT = dG = dT = dG = dA = dG = d5C = dT = dT = dG = MA = MG = MG = MT = MT = M5C |
| 1287 | CO-13681 | MG = M5C = M5C = MA = MT = MG = MT = dG = dA = dG = d5C = dT = dT = dG = dA = dG = dG = MT = MT = M5C |
| 1288 | CO-13682 | MG = M5C = M5C = MA = MT = MG = MT = MG = dA = dG = d5C = dT = dT = dG = dA = dG = dG = dT = MT = M5C |
| 1289 | CO-13683 | MG = M5C = M5C = MA = MT = MG = MT = MG = MA = dG = d5C = dT = dT = dG = dA = dG = dG = dT = dT = M5C |
| 1290 | CO-13684 | MG = M5C = M5C = MA = MT = MG = dT = dG = dA = dG = d5C = dT = dT = dG = dA = dG = MG = MT = MT = M5C |
| 1291 | CO-13685 | dG = d5C = d5C = dA = dT = dG = dT = dG = dA = dG = d5C = dT = dT = dG = dA = dG = dG = dT = dT = d5C |
| 1292 | CO-13686 | MG = d5C = M5C = dA = MT = dG = MT = dG = MA = dG = M5C = dT = MT = dG = MA = dG = MG = dT = MT = d5C |
| 1293 | CO-13687 | MG = M5C = M5C = MA = MT = MG = MT = dG = dA = dG = d5C = dT = MT = MG = MA = MG = MG = MT = MT = M5C |
| 1294 | CO-13688 | MG = M5C = M5C = MA = MT = MG = MT = MG = dA = dG = d5C = dT = dT = MG = MA = MG = MG = MT = MT = M5C |
| 1295 | CO-13689 | dG = d5C = d5C = dA = dT = dG = dT = dG = dA = dG = M5C = MT = MT = MG = MA = MG = MG = MT = MT = M5C |
| 1296 | CO-13690 | MG = M5C = M5C = MA = MT = MG = MT = MG = MA = MG = d5C = dT = dT = dG = dA = dG = dG = dT = dT = d5C |
| 1297 | CO-14208 | M5C = MT = M5C = M5C = M5C = dA = dG = d5C = d5C = dA = d5C = dT = dT = dT = dT = MT = M5C = M5C = MT = MG |
| 1298 | CO-14209 | M5C = MT = M5C = MT = M5C = d5C = d5C = dA = dG = d5C = d5C = dA = d5C = dT = dT = MT = MT = MT = M5C = M5C |
| 1299 | CO-14210 | M5C = MG = M5C = M5C = MT = d5C = dT = d5C = d5C = d5C = dA = dG = d5C = d5C = dA = M5C = MT = MT = MT = MT |
| 1300 | CO-14211 | MT = MA = MG = M5C = MT = dT = d5C = d5C = dG = d5C = d5C = dT = d5C = dT = d5C = M5C = M5C = MA = MG = M5C |
| 1301 | CO-14212 | MG = M5C = MT = MA = MG = d5C = dT = dT = d5C = d5C = dG = d5C = d5C = dT = d5C = MT = M5C = M5C = M5C = MA |
| 1302 | CO-14213 | MA = MG = M5C = MT = MA = dG = d5C = dT = dT = d5C = d5C = dG = d5C = d5C = dT = M5C = MT = M5C = M5C = M5C |
| 1303 | CO-14214 | M5C = MG = MA = MG = M5C = dT = dA = dG = d5C = dT = dT = d5C = d5C = dG = d5C = M5C = MT = M5C = MT = M5C |
| 1304 | CO-14215 | MG = MA = M5C = MG = MA = dG = d5C = dT = dA = dG = d5C = dT = dT = d5C = d5C = MG = M5C = M5C = MT = M5C |
| 1305 | CO-14216 | MG = MG = MA = M5C = MG = dA = dG = d5C = dT = dA = dG = d5C = dT = dT = d5C = M5C = MG = M5C = M5C = MT |
| 1306 | CO-14217 | MG = MT = MG = MG = MG = dA = d5C = dG = dA = dG = d5C = dT = dA = dG = d5C = MT = MT = M5C = M5C = MG |
| 1307 | CO-14218 | MG = MG = MG = MT = MG = dG = dG = dA = d5C = dG = dA = dG = d5C = dT = dA = MG = M5C = MT = MT = M5C |
| 1308 | CO-14219 | MG = M5C = MG = MG = MG = dT = dG = dG = dG = dA = d5C = dG = dA = dG = d5C = MT = MA = MG = M5C = MT |
| 1309 | CO-14220 | MG = MT = MT = MG = M5C = dG = dG = dG = dT = dG = dG = dG = dA = d5C = dG = MA = MG = M5C = MT = MA |
| 1310 | CO-14221 | MT = MT = MG = MT = MT = dG = d5C = dG = dG = dG = dT = dG = dG = dG = dA = M5C = MG = MA = MG = M5C |
| 1311 | CO-14222 | M5C = MT = M5C = MT = MT = dG = dT = dT = dG = d5C = dG = dG = dG = dT = dG = MG = MG = MA = M5C = MG |
| 1312 | CO-14223 | MT = M5C = M5C = MT = M5C = dT = dT = dG = dT = dT = dG = d5C = dG = dG = dG = MT = MG = MG = MG = MA |
| 1313 | CO-14224 | MA = M5C = MT = MT = M5C = d5C = dT = d5C = dT = dT = dG = dT = dT = dG = d5C = MG = MG = MG = MT = MG |
| 1314 | CO-14225 | M5C = MT = MT = MA = M5C = dT = dT = d5C = d5C = dT = d5C = dT = dT = dG = dT = MT = MG = M5C = MG = MG |
| 1315 | CO-14226 | MG = MG = M5C = MG = MA = dG = dA = dG = dG = dA = dA = dG = d5C = dA = dG = MG = MG = MA = MG = MG |
| 1316 | CO-14227 | M5C = M5C = MT = MA = MG = dA = dA = dA = dT = dG = dG = dG = dG = dT = dG = MT = MG = MG = MG = MG |
| 1317 | CO-14228 | MA = M5C = MA = MT = MG = dA = dT = d5C = d5C = d5C = dT = dA = dG = dA = dA = MA = MT = MG = MG = MG |
| 1318 | CO-14229 | M5C = MA = MA = MT = M5C = dA = d5C = dA = dT = dG = dA = dT = d5C = d5C = d5C = MT = MA = MG = MA = MA |
| 1319 | CO-14230 | MT = M5C = MT = M5C = M5C = dA = dA = dT = d5C = dA = d5C = dA = dT = dG = dA = MT = M5C = M5C = M5C = MT |
| 1320 | CO-14231 | MA = MT = MT = M5C = MT = d5C = d5C = dA = dA = dT = d5C = dA = d5C = dA = dT = MG = MA = MT = M5C = M5C |
| 1321 | CO-14232 | MT = MG = MA = MT = MT = d5C = dT = d5C = d5C = dA = dA = dT = d5C = dA = d5C = MA = MT = MG = MA = MT |
| 1322 | CO-14233 | M5C = MA = MT = MG = MA = dT = dT = d5C = dT = d5C = d5C = dA = dA = dT = d5C = MA = M5C = MA = MT = MG |
| 1323 | CO-14234 | M5C = MA = M5C = MA = MT = dG = dA = dT = dT = d5C = dT = d5C = d5C = dA = dA = MT = M5C = MA = M5C = MA |
| 1324 | CO-14235 | M5C = MT = M5C = M5C = M5C = MA = MG = M5C = M5C = MA = M5C = MT = MT = MT = MT = MT = M5C = M5C = MT = MG |
| 1325 | CO-14236 | M5C = MT = M5C = MT = M5C = M5C = M5C = MA = MG = M5C = M5C = MA = M5C = MT = MT = MT = MT = MT = M5C = M5C |
| 1326 | CO-14237 | M5C = MG = M5C = M5C = MT = M5C = MT = M5C = M5C = M5C = MA = MG = M5C = M5C = MA = M5C = MT = MT = MT = MT |
| 1327 | CO-14238 | MT = MA = MG = M5C = MT = MT = M5C = M5C = MG = M5C = M5C = MT = M5C = MT = M5C = M5C = M5C = MA = MG = M5C |
| 1328 | CO-14239 | MG = M5C = MT = MA = MG = M5C = MT = MT = M5C = M5C = MG = M5C = M5C = MT = M5C = MT = M5C = M5C = M5C = MA |
| 1329 | CO-14240 | MA = MG = M5C = MT = MA = MG = M5C = MT = MT = M5C = M5C = MG = M5C = M5C = MT = M5C = MT = M5C = M5C = M5C |
| 1330 | CO-14241 | M5C = MG = MA = MG = M5C = MT = MA = MG = M5C = MT = MT = M5C = M5C = MG = M5C = M5C = MT = M5C = MT = M5C |
| 1331 | CO-14242 | MG = MA = M5C = MG = MA = MG = M5C = MT = MA = MG = M5C = MT = MT = M5C = M5C = MG = M5C = M5C = MT = M5C |
| 1332 | CO-14243 | MG = MG = MA = M5C = MG = MA = MG = M5C = MT = MA = MG = M5C = MT = MT = M5C = M5C = MG = M5C = M5C = MT |
| 1333 | CO-14244 | MG = MT = MG = MG = MG = MA = M5C = MG = MA = MG = M5C = MT = MA = MG = M5C = MT = MT = M5C = M5C = MG |
| 1334 | CO-14245 | MG = MG = MG = MT = MG = MG = MG = MA = M5C = MG = MA = MG = M5C = MT = MA = MG = M5C = MT = MT = M5C |
| 1335 | CO-14246 | MG = M5C = MG = MG = MG = MT = MG = MG = MG = MA = M5C = MG = MA = MG = M5C = MT = MA = MG = M5C = MT |
| 1336 | CO-14247 | MG = MT = MT = MG = M5C = MG = MG = MG = MT = MG = MG = MG = MA = M5C = MG = MA = MG = M5C = MT = MA |
| 1337 | CO-14248 | MT = MT = MG = MT = MT = MG = M5C = MG = MG = MG = MT = MG = MG = MG = MA = M5C = MG = MA = MG = M5C |
| 1338 | CO-14249 | M5C = MT = M5C = MT = MT = MG = MT = MT = MG = M5C = MG = MG = MG = MT = MG = MG = MG = MA = M5C = MG |
| 1339 | CO-14250 | MT = M5C = M5C = MT = M5C = MT = MT = MG = MT = MT = MG = M5C = MG = MG = MG = MT = MG = MG = MG = MA |
| 1340 | CO-14251 | MA = M5C = MT = MT = M5C = M5C = MT = M5C = MT = MT = MG = MT = MT = MG = M5C = MG = MG = MG = MT = MG |
| 1341 | CO-14252 | M5C = MT = MT = MA = M5C = MT = MT = M5C = M5C = MT = M5C = MT = MT = MG = MT = MT = MG = M5C = MG = MG |
| 1342 | CO-14253 | MG = MG = M5C = MG = MA = MG = MA = MG = MG = MA = MA = MG = M5C = MA = MG = MG = MG = MA = MG = MG |
| 1343 | CO-14254 | M5C = M5C = MT = MA = MG = MA = MA = MA = MT = MG = MG = MG = MG = MT = MG = MT = MG = MG = MG = MG |
| 1344 | CO-14255 | MA = M5C = MA = MT = MG = MA = MT = M5C = M5C = M5C = MT = MA = MG = MA = MA = MA = MT = MG = MG = MG |
| 1345 | CO-14256 | M5C = MA = MA = MT = M5C = MA = M5C = MA = MT = MG = MA = MT = M5C = M5C = M5C = MT = MA = MG = MA = MA |
| 1346 | CO-14257 | MT = M5C = MT = M5C = M5C = MA = MA = MT = M5C = MA = M5C = MA = MT = MG = MA = MT = M5C = M5C = M5C = MT |
| 1347 | CO-14258 | MA = MT = MT = M5C = MT = M5C = M5C = MA = MA = MT = M5C = MA = M5C = MA = MT = MG = MA = MT = M5C = M5C |
| 1348 | CO-14259 | MT = MG = MA = MT = MT = M5C = MT = M5C = M5C = MA = MA = MT = M5C = MA = M5C = MA = MT = MG = MA = MT |
| 1349 | CO-14260 | M5C = MA = MT = MG = MA = MT = MT = M5C = MT = M5C = M5C = MA = MA = MT = M5C = MA = M5C = MA = MT = MG |
| 1350 | CO-14261 | M5C = MA = M5C = MA = MT = MG = MA = MT = MT = M5C = MT = M5C = M5C = MA = MA = MT = M5C = MA = M5C = MA |
| 1351 | CO-14677 | BioTEG = .[C18] = MG = MG = MA = M5C = MT = dT = dG = dG = dG = dA = dG = dT = dG = dG = dT = MG = MA = M5C = MT = MG |
| 1352 | CO-14678 | BioTEG = .[C18] = MG = MG = MA = M5C = MT = LT = MG = MG = LG = MA = MG = LT = MG = MG = LT = MG = MA = M5C = MT = MG |
| 1353 | CO-14679 | BioTEG = [C18] = LG = LG = LA = L5C = LT = dT = dG = dG = dG = dA = dG = dT = dG = dG = dT = LG = LA = L5C = LT = LG |
| 1354 | CO-14680 | BioTEG = .[C18] = MG = MG = MA = M5C = MT = MT = MG = MG = MG = MA = MG = MT = MG = MG = MT = MG = MA = M5C = MT = MG |
| 1355 | CO-14681 | BioTEG = .[C18] = MG = MG = MA = M5C = MT = MT = MG = MG = MG = MA − MG = MT = MG = MG = MT = MG = MA = M5C = MT = MG |
| 1356 | CO-14682 | BioTEG = .[C18] = MG = MG = MA = M5C = MT − MT = MG = MG = MG = MA − MG = MT = MG = MG = MT − MG = MA = M5C = MT = MG |
| 1357 | CO-14683 | BioTEG = .[C18] = MG = MG − MA = M5C − MT = MT − MG = MG − MG = MA − MG = MT − MG = MG − MT = MG − MA = M5C − MT = MG |
| 1358 | CO-14684 | BioTEG = .[C18] = MG − MG − MA − M5C − MT = MT − MG − MG − MG − MA = MG − MT − MG − MG − MT = MG − MA − M5C − MT − MG |
| 1359 | CO-14685 | BioTEG = .[C18] = MG − MG − MA − M5C − MT − MT − MG − MG − MG − MA = MG − MT − MG − MG − MT − MG − MA − M5C − MT − MG |
| 1360 | CO-14686 | BioTEG = .[C18] = MG = MG = MA = M5C = MT = dT = dG = dG = dG = dA − dG = dT = dG = dG = dT = MG = MA = M5C = MT = MG |
| 1361 | CO-14687 | BioTEG = .[C18] = MG = MG = MA = M5C = MT − dT = dG = dG = dG = dA − dG = dT = dG = dG = dT − MG = MA = M5C = MT = MG |
| 1362 | CO-14688 | BioTEG = .[C18] = MG = MG − MA = M5C − MT = dT − dG = dG − dG = dA − dG = dT − dG = dG − dT = MG − MA = M5C − MT = MG |
| 1363 | CO-14689 | BioTEG = .[C18] = MG − MG − MA − M5C − MT = dT − dG − dG − dG − dA = dG − dT − dG − dG − dT = MG − MA − M5C − MT − MG |
| 1364 | CO-14690 | BioTEG = .[C18] = MG − MG − MA − M5C − MT − dT − dG − dG − dG − dA = dG − dT − dG − dG − dT − MG − MA − M5C − MT − MG |
| 1365 | CO-14691 | BioTEG = .[C18] = dG = dG = dA = d5C = dT = dT = dG = dG = dG = dA = dG = dT = dG = dG = dT = dG = dA = d5C = dT = dG |
| 1366 | CO-14692 | BioTEG = .[C18] = dG − dG − dA − d5C − dT − dT − dG − dG − dG − dA − dG − dT − dG − dG − dT − dG − dA − d5C − dT − dG |
| 1367 | CO-14712 | BioTEG = .[C18] = MG = MG = MA = M5C = MT = LT = MG = MG = LG = MA = MG = LT = MG = MG = LT = MG = MA = M5C = MT |
| 1368 | CO-14713 | BioTEG = .[C18] = MG = MG = MA = M5C = MT = LT = MG = MG = LG = MA = MG = LT = MG = MG = LT = MG = MA = M5C |
| 4743 | CO-7761 | BioTEG − dT − dT − dT − dT − dT − dT − dT − dT − dT − dT − MG = MG = MA = M5C = MT = LT = MG = MG = LG = MA = MG = LT = MG = MG = LT = MG = MA = M5C = MT = MG |
| 4744 | CO-7763 | BioTEG − dT − dT − dT − dT − dT − dT − dT − dT − dT − dT − MG − MG − MA − M5C − MT − LT − MG − MG − LG − MA − MG − LT − MG − MG − LT − MG − MA − M5C − MT − MG |
| 4745 | CO-15889 | MG = MG = MG = MT = MG = d5C = d5C = dA = d5C = d5C = dT = dA = dG = dT = dG = MG = MA = MA = MG = MA |
| 4746 | CO-15890 | MT = MG = MG = MG = MG = dG = dG = dT = dG = d5C = d5C = dA = d5C = d5C = dT = MA = MG = MT = MG = MG |
| 4747 | CO-15891 | MT = M5C = MG = MG = MA = dG = dT = dT = dG = dG = dG = dG = dG = dG = dT = MG = M5C = M5C = MA = M5C |
| 4748 | CO-15892 | MG = MG = M5C = MA = M5C = dT = d5C = dG = dG = dA = dG = dT = dT = dG = dG = MG = MG = MG = MG = MT |
| 4749 | CO-15893 | MT = M5C = M5C = MT = M5C = dT = d5C = d5C = d5C = d5C = dT = dT = dG = dG = d5C = MA = M5C = MT = M5C = MG |
| 4750 | CO-15894 | MG = MA = MA = MT = MT = dT = d5C = d5C = dT = d5C = dT = d5C = d5C = d5C = d5C = MT = MT = MG = MG = M5C |
| 4751 | CO-15895 | MT = MT = MT = MT = MG = dG = dG = dA = dA = dT = dT = dT = d5C = d5C = dT = M5C = MT = M5C = M5C = M5C |
| 4752 | CO-15896 | MG = M5C = MT = M5C = M5C = d5C = d5C = dT = dT = dT = dT = dG = dG = dG = dA = MA = MT = MT = MT = M5C |
| 4753 | CO-15897 | MT = MG = MA = MG = MA = d5C = d5C = dT = dT = dG = dG = d5C = dT = dT = d5C = MT = M5C = MG = MA = MA |
| 4754 | CO-15898 | MT = MG = MG = MG = MA = dA = d5C = dG = dA = dG = dA = d5C = d5C = dT = dG = MA = MG = MA = M5C = M5C |
| 4755 | CO-15899 | M5C = MG = MA = MG = MG = dG = d5C = d5C = dT = dG = dG = dG = dA = dA = d5C = MG = MA = MG = MA = M5C |
| 4756 | CO-15900 | M5C = MT = M5C = MT = M5C = d5C = dT = dG = dG = dT = dG = dG = dT = dG = dG = MT = MG = MG = MG = MT |
| 4757 | CO-15901 | MT = M5C = M5C = M5C = M5C = dT = d5C = dT = d5C = d5C = dT = dG = dG = dT = dG = MG = MT = MG = MG = MT |
| 4758 | CO-15902 | MT = MT = M5C = MT = MT = d5C = d5C = d5C = d5C = dT = d5C = dT = d5C = d5C = dT = MG = MG = MT = MG = MG |
| 4759 | CO-15903 | MG = M5C = MT = MG = MG = d5C = dT = dT = d5C = dT = dT = d5C = d5C = d5C = d5C = MT = M5C = MT = M5C = M5C |
| 4760 | CO-15904 | M5C = MT = MG = MT = M5C = dG = dG = dT = dA = dG = dG = dT = dG = d5C = dT = MG = MG = M5C = MT = MT |
| 4761 | CO-15905 | MA = MG = M5C = MT = M5C = d5C = dA = d5C = d5C = d5C = d5C = dT = dG = dT = d5C = MG = MG = MT = MA = MG |
| 4762 | CO-15906 | MA = M5C = M5C = M5C = MA = dG = d5C = dT = d5C = d5C = dA = d5C = d5C = d5C = d5C = MT = MG = MT = M5C = MG |
| 4763 | CO-15907 | M5C = MT = MT = MG = MA = d5C = d5C = d5C = dA = dG = d5C = dT = d5C = d5C = dA = M5C = M5C = M5C = M5C = MT |
| 4764 | CO-15908 | MG = MG = MA = MG = MT = d5C = dT = d5C = dA = dG = d5C = d5C = dG = dG = dA = MG = MA = M5C = MA = MA |
| 4765 | CO-15909 | MG = MA = MG = MG = MT = dT = d5C = d5C = d5C = d5C = dT = dG = dG = dA = dG = MT = M5C = MT = M5C = MA |
| 4766 | CO-15910 | MG = M5C = MT = MT = MG = dA = dG = dG = dT = dT = d5C = d5C = d5C = d5C = dT = MG = MG = MA = MG = MT |
| 4767 | CO-15911 | MG = MT = MG = MA = MG = d5C = dT = dT = dG = dA = dG = dG = dT = dT = d5C = M5C = M5C = M5C = MT = MG |
| 4768 | CO-15912 | M5C = MG = M5C = M5C = MA = dG = dG = dG = d5C = d5C = dA = dT = dG = dT = dG = MA = MG = M5C = MT = MT |
| 4769 | CO-15913 | MG = MG = M5C = M5C = M5C = dG = d5C = d5C = dA = dG = dG = dG = d5C = d5C = dA = MT = MG = MT = MG = MA |
| 4770 | CO-15914 | M5C = MA = MG = MG = MG = dG = d5C = d5C = d5C = dG = d5C = d5C = dA = dG = dG = MG = M5C = M5C = MA = MT |
| 4771 | CO-15915 | MT = MG = M5C = M5C = M5C = dA = dG = dG = dG = dG = d5C = d5C = d5C = dG = d5C = M5C = MA = MG = MG = MG |
| 4772 | CO-15916 | MT = MG = M5C = MT = M5C = d5C = dT = dG = d5C = d5C = d5C = dA = dG = dG = dG = MG = M5C = M5C = M5C = MG |
| 4773 | CO-15917 | MG = MG = MA = MT = MG = dG = dG = dT = dT = dA = dA = dG = dG = dA = dA = MG = MG = M5C = MG = MA |
| 4774 | CO-15918 | MA = MG = MG = M5C = MG = d5C = d5C = dT = dG = d5C = dA = dG = dG = dA = dT = MG = MG = MG = MT = MT |
| 4775 | CO-15919 | M5C = M5C = M5C = M5C = MA = dG = dG = d5C = dG = d5C = d5C = dT = dG = d5C = dA = MG = MG = MA = MT = MG |
| 4776 | CO-15920 | MA = MA = M5C = MT = M5C = d5C = d5C = d5C = d5C = dA = dG = dG = d5C = dG = d5C = M5C = MT = MG = M5C = MA |
| 4777 | CO-15921 | MG = M5C = MG = M5C = MT = dA = dT = d5C = dA = dT = d5C = dT = d5C = dT = dA = MG = M5C = MA = MA = MA |
| 4778 | CO-15922 | MT = M5C = MT = MG = M5C = d5C = dT = dG = dG = d5C = d5C = d5C = dA = dG = dG = M5C = M5C = M5C = MT = MT |
| 4779 | CO-15923 | MA = MT = M5C = M5C = MA = dT = d5C = dT = d5C = dT = dG = d5C = d5C = dT = dG = MG = M5C = M5C = M5C = MA |
| 4780 | CO-15924 | MT = M5C = MT = M5C = M5C = d5C = dA = dT = d5C = dA = dT = d5C = d5C = dA = dT = M5C = MT = M5C = MT = MG |
| 4781 | CO-15925 | M5C = M5C = M5C = MA = MT = d5C = dT = d5C = d5C = d5C = dA = dT = d5C = dA = dT = M5C = M5C = MA = MT = M5C |
| 4782 | CO-15926 | MT = M5C = MT = M5C = MT = d5C = d5C = dT = dG = dG = d5C = d5C = d5C = dA = dT = M5C = MT = M5C = M5C = M5C |
| 4783 | CO-15927 | MG = MG = MG = M5C = M5C = dT = d5C = dT = d5C = d5C = dT = d5C = dT = d5C = dT = M5C = M5C = MT = MG = MG |
| 4784 | CO-15928 | MT = M5C = M5C = M5C = MT = dG = dG = dG = d5C = d5C = dT = d5C = dT = d5C = d5C = MT = M5C = MT = M5C = MT |
| 4785 | CO-15929 | MG = M5C = MT = MT = MT = d5C = d5C = d5C = dT = dG = dG = dG = d5C = d5C = dT = M5C = MT = M5C = M5C = MT |
| 4786 | CO-15930 | MG = MT = MG = M5C = MA = dG = dA = d5C = dA = d5C = dT = dG = d5C = d5C = dG = MT = MT = M5C = M5C = MT |
| 4787 | CO-15931 | M5C = M5C = M5C = M5C = MT = dG = dT = dG = d5C = dA = dG = dA = d5C = dA = d5C = MT = MG = M5C = M5C = MG |
| 4788 | CO-15932 | M5C = M5C = MA = MT = M5C = d5C = d5C = d5C = dT = dG = dT = dG = d5C = dA = dG = MA = M5C = MA = M5C = MT |
| 4789 | CO-15933 | MA = MG = MT = M5C = MT = d5C = d5C = dA = dT = d5C = d5C = d5C = d5C = dT = dG = MT = MG = M5C = MA = MG |
| 4790 | CO-15934 | M5C = MT = MT = MG = M5C = dT = d5C = dA = dG = dG = d5C = dT = dG = d5C = dA = MG = M5C = MA = MG = MT |
| 4791 | CO-15935 | MG = MT = MT = M5C = M5C = dT = dT = dG = d5C = dT = d5C = dA = dG = dG = d5C = MT = MG = M5C = MA = MG |
| 4792 | CO-15936 | MT = M5C = MA = MG = M5C = dT = d5C = d5C = dA = dG = dT = dT = d5C = d5C = dT = MT = MG = M5C = MT = M5C |
| 4793 | CO-15937 | MG = MG = MG = MT = MG = L5C = M5C = MA = L5C = M5C = MT = LA = MG = MT = LG = MG = MA = MA = MG = MA |
| 4794 | CO-15938 | MT = MG = MG = MG = MG = LG = MG = MT = LG = M5C = M5C = LA = M5C = M5C = LT = MA = MG = MT = MG = MG |
| 4795 | CO-15939 | MT = M5C = MG = MG = MA = LG = MT = MT = LG = MG = MG = LG = MG = MG = LT = MG = M5C = M5C = MA = M5C |
| 4796 | CO-15940 | MG = MG = M5C = MA = M5C = LT = M5C = MG = LG = MA = MG = LT = MT = MG = LG = MG = MG = MG = MG = MT |
| 4797 | CO-15941 | MT = M5C = M5C = MT = M5C = LT = M5C = M5C = L5C = M5C = MT = LT = MG = MG = L5C = MA = M5C = MT = M5C = MG |
| 4798 | CO-15942 | MG = MA = MA = MT = MT = LT = M5C = M5C = LT = M5C = MT = L5C = M5C = M5C = L5C = MT = MT = MG = MG = M5C |
| 4799 | CO-15943 | MT = MT = MT = MT = MG = LG = MG = MA = LA = MT = MT = LT = M5C = M5C = LT = M5C = MT = M5C = M5C = M5C |
| 4800 | CO-15944 | MG = M5C = MT = M5C = M5C = L5C = M5C = MT = LT = MT = MT = LG = MG = MG = LA = MA = MT = MT = MT = M5C |
| 4801 | CO-15945 | MT = MG = MA = MG = MA = L5C = M5C = MT = LT = MG = MG = L5C = MT = MT = L5C = MT = M5C = MG = MA = MA |
| 4802 | CO-15946 | MT = MG = MG = MG = MA = LA = M5C = MG = LA = MG = MA = L5C = M5C = MT = LG = MA = MG = MA = M5C = M5C |
| 4803 | CO-15947 | M5C = MG = MA = MG = MG = LG = M5C = M5C = LT = MG = MG = LG = MA = MA = L5C = MG = MA = MG = MA = M5C |
| 4804 | CO-15948 | M5C = MT = M5C = MT = M5C = L5C = MT = MG = LG = MT = MG = LG = MT = MG = LG = MT = MG = MG = MG = MT |
| 4805 | CO-15949 | MT = M5C = M5C = M5C = M5C = LT = M5C = MT = L5C = M5C = MT = LG = MG = MT = LG = MG = MT = MG = MG = MT |
| 4806 | CO-15950 | MT = MT = M5C = MT = MT = L5C = M5C = M5C = L5C = MT = M5C = LT = M5C = M5C = LT = MG = MG = MT = MG = MG |
| 4807 | CO-15951 | MG = M5C = MT = MG = MG = L5C = MT = MT = L5C = MT = MT = L5C = M5C = M5C = L5C = MT = M5C = MT = M5C = M5C |
| 4808 | CO-15952 | M5C = MT = MG = MT = M5C = LG = MG = MT = LA = MG = MG = LT = MG = M5C = LT = MG = MG = M5C = MT = MT |
| 4809 | CO-15953 | MA = MG = M5C = MT = M5C = L5C = MA = M5C = L5C = M5C = M5C = LT = MG = MT = L5C = MG = MG = MT = MA = MG |
| 4810 | CO-15954 | MA = M5C = M5C = M5C = MA = LG = M5C = MT = L5C = M5C = MA = L5C = M5C = M5C = L5C = MT = MG = MT = M5C = MG |
| 4811 | CO-15955 | M5C = MT = MT = MG = MA = L5C = M5C = M5C = LA = MG = M5C = LT = M5C = M5C = LA = M5C = M5C = M5C = M5C = MT |
| 4812 | CO-15956 | MG = MG = MA = MG = MT = L5C = MT = M5C = LA = MG = M5C = L5C = MG = MG = LA = MG = MA = M5C = MA = MA |
| 4813 | CO-15957 | MG = MA = MG = MG = MT = LT = M5C = M5C = L5C = M5C = MT = LG = MG = MA = LG = MT = M5C = MT = M5C = MA |
| 4814 | CO-15958 | MG = M5C = MT = MT = MG = LA = MG = MG = LT = MT = M5C = L5C = M5C = M5C = LT = MG = MG = MA = MG = MT |
| 4815 | CO-15959 | MG = MT = MG = MA = MG = L5C = MT = MT = LG = MA = MG = LG = MT = MT = L5C = M5C = M5C = M5C = MT = MG |
| 4816 | CO-15960 | M5C = MG = M5C = M5C = MA = LG = MG = MG = L5C = M5C = MA = LT = MG = MT = LG = MA = MG = M5C = MT = MT |
| 4817 | CO-15961 | MG = MG = M5C = M5C = M5C = LG = M5C = M5C = LA = MG = MG = LG = M5C = M5C = LA = MT = MG = MT = MG = MA |
| 4818 | CO-15962 | M5C = MA = MG = MG = MG = LG = M5C = M5C = L5C = MG = M5C = L5C = MA = MG = LG = MG = M5C = M5C = MA = MT |
| 4819 | CO-15963 | MT = MG = M5C = M5C = M5C = LA = MG = MG = LG = MG = M5C = L5C = M5C = MG = L5C = M5C = MA = MG = MG = MG |
| 4820 | CO-15964 | MT = MG = M5C = MT = M5C = L5C = MT = MG = L5C = M5C = M5C = LA = MG = MG = LG = MG = M5C = M5C = M5C = MG |
| 4821 | CO-15965 | MG = MG = MA = MT = MG = LG = MG = MT = LT = MA = MA = LG = MG = MA = LA = MG = MG = M5C = MG = MA |
| 4822 | CO-15966 | MA = MG = MG = M5C = MG = L5C = M5C = MT = LG = M5C = MA = LG = MG = MA = LT = MG = MG = MG = MT = MT |
| 4823 | CO-15967 | M5C = M5C = M5C = M5C = MA = LG = MG = M5C = LG = M5C = M5C = LT = MG = M5C = LA = MG = MG = MA = MT = MG |
| 4824 | CO-15968 | MA = MA = M5C = MT = M5C = L5C = M5C = M5C = L5C = MA = MG = LG = M5C = MG = L5C = M5C = MT = MG = M5C = MA |
| 4825 | CO-15969 | MG = M5C = MG = M5C = MT = LA = MT = M5C = LA = MT = M5C = LT = M5C = MT = LA = MG = M5C = MA = MA = MA |
| 4826 | CO-15970 | MT = M5C = MT = MG = M5C = L5C = MT = MG = LG = M5C = M5C = L5C = MA = MG = LG = M5C = M5C = M5C = MT = MT |
| 4827 | CO-15971 | MA = MT = M5C = M5C = MA = LT = M5C = MT = L5C = MT = MG = L5C = M5C = MT = LG = MG = M5C = M5C = M5C = MA |
| 4828 | CO-15972 | MT = M5C = MT = M5C = M5C = L5C = MA = MT = L5C = MA = MT = L5C = M5C = MA = LT = M5C = MT = M5C = MT = MG |
| 4829 | CO-15973 | M5C = M5C = M5C = MA = MT = L5C = MT = M5C = L5C = M5C = MA = LT = M5C = MA = LT = M5C = M5C = MA = MT = M5C |
| 4830 | CO-15974 | MT = M5C = MT = M5C = MT = L5C = M5C = MT = LG = MG = M5C = L5C = M5C = MA = LT = M5C = MT = M5C = M5C = M5C |
| 4831 | CO-15975 | MG = MG = MG = M5C = M5C = LT = M5C = MT = L5C = M5C = MT = L5C = MT = M5C = LT = M5C = M5C = MT = MG = MG |
| 4832 | CO-15976 | MT = M5C = M5C = M5C = MT = LG = MG = MG = L5C = M5C = MT = L5C = MT = M5C = L5C = MT = M5C = MT = M5C = MT |
| 4833 | CO-15977 | MG = M5C = MT = MT = MT = L5C = M5C = M5C = LT = MG = MG = LG = M5C = M5C = LT = M5C = MT = M5C = M5C = MT |
| 4834 | CO-15978 | MG = MT = MG = M5C = MA = LG = MA = M5C = LA = M5C = MT = LG = M5C = M5C = LG = MT = MT = M5C = M5C = MT |
| 4835 | CO-15979 | M5C = M5C = M5C = M5C = MT = LG = MT = MG = L5C = MA = MG = LA = M5C = MA = L5C = MT = MG = M5C = M5C = MG |
| 4836 | CO-15980 | M5C = M5C = MA = MT = M5C = L5C = M5C = M5C = LT = MG = MT = LG = M5C = MA = LG = MA = M5C = MA = M5C = MT |
| 4837 | CO-15981 | MA = MG = MT = M5C = MT = L5C = M5C = MA = LT = M5C = M5C = L5C = M5C = MT = LG = MT = MG = M5C = MA = MG |
| 4838 | CO-15982 | M5C = MT = MT = MG = M5C = LT = M5C = MA = LG = MG = M5C = LT = MG = M5C = LA = MG = M5C = MA = MG = MT |
| 4839 | CO-15983 | MG = MT = MT = M5C = M5C = LT = MT = MG = L5C = MT = M5C = LA = MG = MG = L5C = MT = MG = M5C = MA = MG |
| 4840 | CO-15984 | MT = M5C = MA = MG = M5C = LT = M5C = M5C = LA = MG = MT = LT = M5C = M5C = LT = MT = MG = M5C = MT = M5C |
| 4841 | CO-15985 | MG = MG = MT = MT = MG = LG = M5C = MT = L5C = M5C = M5C = L5C = MT = MT = LT = MT = MG = MG = MG = MA |
| 4842 | CO-15986 | MG = M5C = M5C = MA = M5C = LT = MT = MT = LT = MT = M5C = L5C = MT = MG = L5C = MT = M5C = MA = MT = MT |
| 4843 | CO-15987 | M5C = MT = M5C = M5C = MG = LA = MG = MG = LG = M5C = M5C = LT = MG = MG = LG = MA = MA = M5C = MG = MA |
| 4844 | CO-15988 | MA = MG = M5C = M5C = MG = LG = MA = MG = LG = MG = MG = L5C = MG = M5C = LG = M5C = MG = MA = M5C = M5C |
| 4845 | CO-15989 | MT = MG = MG = MG = MT = LG = MG = MT = LT = M5C = M5C = L5C = MG = M5C = LG = MG = M5C = MG = MG = M5C |
| 4846 | CO-15990 | MA = MA = MG = M5C = MA = LG = MG = MG = LA = M5C = M5C = LA = M5C = MA = L5C = M5C = MA = MT = MT = M5C |
| 4847 | CO-15991 | MG = MA = MA = MG = MG = LG = MG = M5C = LT = MT = MT = L5C = MT = MA = L5C = M5C = MT = M5C = M5C = M5C |
| 4848 | CO-15992 | MA = MG = MT = MA = MA = LT = MT = MG = L5C = MA = MG = LT = MG = MA = L5C = MG = M5C = M5C = M5C = M5C |
| 4849 | CO-15993 | MT = M5C = MT = MG = MA = LT = MA = M5C = LA = M5C = MT = LA = MG = MG = LG = MG = MG = MA = MG = MT |
| 4850 | CO-15994 | MG = MA = MA = M5C = M5C = LG = M5C = MT = LT = MA = M5C = LT = MG = MA = LA = MA = M5C = MT = M5C = M5C |
| 4851 | CO-15995 | MG = M5C = M5C = MG = M5C = LG = M5C = MA = LG = MA = M5C = L5C = MT = M5C = LT = M5C = MG = M5C = M5C = MT |
| 4852 | CO-15996 | M5C = MA = MA = MG = MG = LA = M5C = M5C = LG = M5C = MG = LG = MA = MG = LT = M5C = MG = MG = MA = M5C |
| 4853 | CO-15997 | MA = MG = MT = M5C = MT = LG = MA = MG = L5C = MG = M5C = LT = MA = M5C = L5C = M5C = MG = MG = MT = MT |
| 4854 | CO-15998 | MA = MG = MG = MT = MA = LG = MG = M5C = LG = MG = MG = LT = M5C = MA = LT = M5C = MG = M5C = MG = M5C |
| 4855 | CO-15999 | MA = MG = M5C = MG = MG = LG = MT = MA = LG = M5C = M5C = L5C = MT = MG = LA = MT = M5C = M5C = M5C = MT |
| 4856 | CO-16000 | M5C = M5C = MT = M5C = MT = LA = MT = MA = LT = MT = MG = LA = MT = MA = LA = MG = MT = MA = MG = M5C |
| 4857 | CO-16001 | MG = MA = MG = MT = MG = LA = MT = MT = LT = MG = MA = LG = MT = MA = LG = MA = MA = MA = MA = MG |
| 4858 | CO-16002 | MG = MA = MT = M5C = M5C = L5C = MT = MA = LG = MA = MA = LA = MT = MG = LG = MG = MG = MT = MG = MT |
| 4859 | CO-16003 | M5C = MG = MA = M5C = MG = LT = M5C = MA = L5C = MA = MT = LG = MA = MT = LT = M5C = MT = M5C = M5C = MA |
| 4860 | CO-16004 | MT = MA = MA = MG = MG = LA = MA = MG = LG = M5C = MG = LA = M5C = MG = LA = MG = M5C = MA = M5C = M5C |
| 4861 | CO-16005 | MA = MA = MT = MG = M5C = L5C = M5C = M5C = LA = MG = MA = L5C = MA = M5C = LG = M5C = MG = M5C = MT = MA |
| 4862 | CO-16006 | MA = MA = MA = MG = MG = L5C = MG = MA = LG = M5C = MA = L5C = MA = MA = LA = MT = MA = MT = MT = M5C |
| 4863 | CO-16007 | M5C = MA = MG = MA = MA = L5C = MA = MA = LA = M5C = MA = LA = MA = M5C = LA = MA = MA = MA = MG = MG |
| 4864 | CO-16008 | MA = MA = M5C = MA = M5C = LA = M5C = MA = LA = MG = MT = L5C = M5C = MG = LG = MG = M5C = MG = M5C = MG |
| 4865 | CO-16009 | MG = MT = MG = MA = MG = L5C = MT = MA = LT = MG = MA = LT = M5C = MA = LG = M5C = MT = MT = MG = MG |
| 4866 | CO-16010 | MA = MG = MA = MA = MT = L5C = MG = M5C = LT = MT = MT = LG = MG = MG = LA = MG = M5C = MA = MG = MG |
| 4867 | CO-16011 | M5C = MA = MT = MT = MT = LG = MT = MA = LA = MT = M5C = L5C = M5C = MA = LG = M5C = MG = M5C = M5C = MT |
| 4868 | CO-16012 | MA = MG = MG = MA = MG = L5C = MG = MA = LG = M5C = M5C = LA = MG = M5C = LT = M5C = MA = MG = MT = MA |
| 4869 | CO-16013 | MG = MA = MA = MT = MA = LG = MG = MG = L5C = MA = MG = LG = MA = M5C = LA = MG = MG = MA = M5C = MA |
| 4870 | CO-16014 | MA = MT = MG = MT = M5C = LA = MA = M5C = L5C = MG = MA = LG = MT = MT = LT = MG = MG = MA = MG = MA |
| 4871 | CO-16015 | MT = MT = M5C = MA = MA = L5C = MT = M5C = LA = M5C = MT = L5C = MT = MG = LG = MG = MG = M5C = M5C = MT |
| 4872 | CO-16016 | MG = MG = MG = M5C = MT = LT = MT = MG = LG = MG = MG = L5C = MT = MG = LT = MT = MT = MA = MT = M5C |
| 4873 | CO-16017 | MA = MA = MT = M5C = M5C = L5C = MT = MG = L5C = MT = M5C = L5C = M5C = MT = LG = MT = M5C = M5C = MT = MG |
| 4874 | CO-16018 | MA = MT = MT = MA = MG = LA = MT = MT = L5C = MA = MG = L5C = M5C = MT = LG = MA = MG = MA = M5C = MT |
| 4875 | CO-16019 | MT = MA = M5C = MA = M5C = L5C = MT = MG = LG = M5C = MA = LA = M5C = MT = LG = MG = M5C = MG = M5C = MA |
| 4876 | CO-16020 | M5C = M5C = MT = MG = MG = LA = MG = MA = L5C = MA = MA = LT = M5C = MT = LA = M5C = MT = MT = M5C = M5C |
| 4877 | CO-16021 | MG = MT = MG = MG = MA = LA = MT = MG = LA = MA = MA = LT = M5C = MT = L5C = MT = MT = M5C = MT = MT |
| 4878 | CO-16022 | MT = M5C = MA = MG = M5C = LT = M5C = MA = LA = MG = MG = LA = MG = MA = LT = MG = M5C = MT = M5C = M5C |
| 4879 | CO-16023 | M5C = MT = M5C = M5C = MA = L5C = M5C = MT = LG = M5C = M5C = LA = MG = MT = LT = MA = MA = MA = MA = MT |
| 4880 | CO-16024 | M5C = M5C = MA = MT = M5C = LT = MG = MG = LT = MT = MT = L5C = M5C = M5C = LA = MG = MG = MG = M5C = MT |
| 4881 | CO-16025 | MG = MG = M5C = MA = MT = LT = MG = MA = LA = M5C = MA = LT = MG = MG = L5C = MA = MT = MT = M5C = MA |
| 4882 | CO-16026 | MG = MG = M5C = MA = M5C = L5C = M5C = MA = LG = M5C = MA = LG = M5C = MT = L5C = M5C = MT = MG = MA = MA |
| 4883 | CO-16027 | M5C = MT = MT = M5C = MT = L5C = MT = MT = LT = M5C = MG = LA = M5C = MA = LG = M5C = M5C = M5C = M5C = M5C |
| 4884 | CO-16028 | M5C = MA = MG = MG = MG = L5C = M5C = MG = LA = MT = M5C = LT = MG = MG = LA = MG = MA = M5C = MT = MA |
| 4885 | CO-16029 | M5C = M5C = MT = M5C = M5C = L5C = MA = MT = LA = MT = M5C = LA = MA = MT = LT = M5C = M5C = MT = M5C = MG |
| 4886 | CO-16030 | MT = MG = MT = M5C = MA = L5C = MT = MG = LG = M5C = MT = LA = MA = MT = L5C = MT = MG = MT = MT = M5C |
| 4887 | CO-16031 | MT = MT = MT = MG = MG = L5C = MA = M5C = L5C = MA = MA = LG = M5C = MA = LG = MG = M5C = MT = MG = MA |
| 4888 | CO-16032 | MA = M5C = MG = MG = M5C = L5C = M5C = MA = LG = MT = MA = LT = M5C = MA = LG = MG = M5C = MT = MT = MT |
| 4889 | CO-16033 | M5C = M5C = MT = M5C = MA = LT = M5C = M5C = LT = M5C = MA = LG = M5C = MT = L5C = M5C = MA = MG = MT = MT |
| 4890 | CO-16034 | M5C = MG = MA = MG = M5C = LT = MT = MG = LG = MT = MT = LA = MG = M5C = L5C = MA = MT = M5C = M5C = M5C |
| 4891 | CO-16035 | MT = M5C = M5C = M5C = MT = L5C = M5C = MT = L5C = M5C = MG = LA = M5C = MT = LT = M5C = MT = MG = M5C = MT |
| 4892 | CO-16036 | MA = M5C = MG = MA = M5C = LT = M5C = M5C = LA = M5C = MA = LG = M5C = MT = L5C = MG = MG = MT = MT = MG |
| 4893 | CO-16037 | MG = M5C = MT = M5C = MT = LA = MG = M5C = LT = M5C = M5C = LA = MG = MG = L5C = MT = MT = MA = MG = MA |
| 4894 | CO-16038 | MG = M5C = M5C = MA = MG = LG = MT = MG = LT = M5C = MT = LT = MA = MT = L5C = M5C = MG = M5C = MT = MG |
| 4895 | CO-16039 | MT = M5C = MA = M5C = M5C = LA = MG = MG = LG = M5C = MA = L5C = MA = MG = LA = M5C = MT = MA = MG = MT |
| 4896 | CO-16040 | MA = M5C = MA = MG = MG = LA = MA = MG = LG = MA = MA = LA = MA = MT = LA = MA = MG = M5C = MA = M5C |
| 4897 | CO-16041 | MA = M5C = MT = MA = MT = LA = M5C = MT = L5C = M5C = M5C = LT = MA = MG = LT = M5C = MT = M5C = MA = MA |
| 4898 | CO-16042 | MG = MT = MG = MT = MG = LT = MG = MT = LT = MT = M5C = LA = MA = MT = LA = MG = MA = MA = MG = M5C |
| 4899 | CO-16043 | MT = MG = MA = MT = M5C = LG = MA = MT = LT = M5C = MA = L5C = M5C = MT = LA = MA = M5C = MA = MT = MT |
| 4900 | CO-16044 | MT = MT = MT = MT = MT = L5C = MT = MG = LG = MT = MT = L5C = MT = MG = LG = MG = MT = MG = MG = MT |
| 4901 | CO-16045 | MT = MT = MG = MT = MT = LT = M5C = MA = LG = MT = M5C = LT = MG = MT = LT = MT = MA = MG = MG = MT |
| 4902 | CO-16046 | MG = MA = MA = MT = M5C = LA = M5C = MG = LG = M5C = MT = LT = MT = MG = LA = M5C = MG = MA = MT = MT |
| 4903 | CO-16047 | MG = MT = M5C = M5C = M5C = LT = MG = MG = L5C = M5C = MG = LG = M5C = MA = LA = MA = MA = MT = MT = MG |
| 4904 | CO-16048 | MG = MA = MT = MT = MG = LT = M5C = MT = L5C = M5C = MA = LG = MA = M5C = LA = M5C = MT = M5C = M5C = MT |
| 4905 | CO-16049 | MA = M5C = M5C = MA = MG = LA = MT = MG = L5C = M5C = MA = LG = MT = MG = LT = M5C = M5C = M5C = MT = M5C |
| 4906 | CO-16050 | MG = MT = MG = MA = MT = LG = MT = MT = LT = MA = MG = LT = MG = MG = LG = MA = MT = MT = MG = M5C |
| 4907 | CO-16051 | MG = MT = MG = MG = MG = LG = MG = MA = LT = MT = MG = LT = MT = M5C = LT = MG = MT = MG = MT = MG |
| 4908 | CO-16052 | MG = MA = M5C = M5C = MT = LT = MT = MT = LG = MG = MG = L5C = MA = MG = LG = MA = MT = MA = M5C = MT |
| 4909 | CO-16053 | MA = MG = MT = MA = MG = LG = MT = MT = LG = MT = MT = LG = MA = MG = LT = M5C = MT = M5C = MA = MG |
| 4910 | CO-16054 | MA = MT = M5C = MG = MT = LG = MG = M5C = LA = M5C = M5C = LT = MT = MA = L5C = M5C = M5C = MT = MT = M5C |
| 4911 | CO-16055 | MG = MA = MG = MT = MA = LG = MG = MG = LA = MA = MT = LT = MA = M5C = LA = MG = MA = M5C = M5C = MT |
| 4912 | CO-16056 | MT = MT = MG = MT = MG = LT = MT = MG = LG = M5C = MG = LG = MG = MG = LG = MT = MG = MG = MA = MT |
| 4913 | CO-16057 | MG = M5C = MA = MT = MA = LA = M5C = MT = L5C = MT = M5C = LT = MT = MA = LA = MG = MA = M5C = MA = M5C |
| 4914 | CO-16058 | MG = MG = M5C = MT = MG = L5C = M5C = MT = LT = M5C = MT = LG = MT = MG = LG = MT = MG = MA = MT = MA |
| 4915 | CO-16059 | M5C = MT = MA = MG = MG = LG = MT = MT = LA = MG = MG = LG = MA = MG = LG = MG = MT = MG = MA = M5C |

TABLE 18
Exemplary ASOs targeting mGrn regRNA with chemical modifications

| | | Chemistry and Sequence |
|---|---|---|
| SEQ | | MOE (M); DNA (d); LNA (L); 2′ OMethyl (m); |
| ID | | PS (=); PO(−); 5-MethylCytosine (5C); Biotin (BioTEG); |
| NO | Name | Palmitic acid (Palm); Spacer 18 (C18) |
| 443 | CO-3544 | MG = MT = MT = M5C = MT = M5C = M5C = |
| | | M5C = MT = MA = MG = M5C = MT = MG = MT = MG = MT = MA = MG = MT |
| 444 | CO-3545 | M5C = MT = MA = MG = MT = MG = MG = MA = MA = MG = |
| | | MA = MT = MG = MT = M5C = MG = MG = MA = MG = M5C |
| 445 | CO-3546 | MG = MA = MA = MA = MT = MT = M5C = MA = MT = |
| | | MT = MT = M5C = M5C = M5C = M5C = MT = M5C = MG = MA = M5C |
| 446 | CO-3547 | MA = MT = M5C = MT = MG = MG = MA = MT = MT = MG = |
| | | MG = M5C = MT = M5C = MA = M5C = M5C = MT = MT = MT |
| 447 | CO-3548 | M5C = MA = M5C = MT = MT = MT = MT = M5C = M5C = |
| | | M5C = M5C = M5C = MG = M5C = MT = MG = M5C = M5C = MT = M5C |
| 448 | CO-3549 | MA = MG = M5C = MT = MA = MA = MT = MT = MT = |
| | | M5C = M5C = MG = M5C = M5C = MT = M5C = MT = M5C = M5C = M5C |
| 449 | CO-3550 | MG = MG = MT = MT = MG = MT = MG = MT = M5C = |
| | | MA = MG = M5C = MT = MT = MT = MA = MT = MT = MT = M5C |
| 450 | CO-3551 | MG = M5C = MG = MT = MG = MA = MT = MG = MG = |
| | | M5C = MT = MG = MA = MG = MT = M5C = M5C = M5C = MT = MG |
| 451 | CO-3552 | M5C = M5C = MT = MT = MG = MG = M5C = M5C = M5C = |
| | | M5C = M5C = MA = MG = MA = MG = M5C = MT = M5C = MA = MA |
| 452 | CO-3553 | M5C = MA = MA = MG = MT = MG = MT = MT = M5C = |
| | | MT = M5C = MT = MG = M5C = MG = MG = M5C = MG = MG = M5C |
| 453 | CO-3554 | MG = MG = M5C = MT = M5C = MT = MT = MT = M5C = |
| | | MT = M5C = MT = MT = MT = M5C = MG = MG = MA = M5C = MG |
| 454 | CO-3555 | M5C = MT = M5C = MT = MT = MG = MT = M5C = M5C = |
| | | MG = MT = MA = M5C = MT = MT = M5C = M5C = M5C = MA = M5C |
| 455 | CO-3556 | MA = MG = MG = MA = M5C = MA = MG = MG = MA = |
| | | MA = M5C = M5C = MA = M5C = M5C = M5C = M5C = MA = M5C = MT |
| 456 | CO-3557 | MT = MG = MT = M5C = MG = MG = MA = MA = MA = |
| | | MG = M5C = MT = MG = M5C = MA = MG = MG = M5C = M5C = MT |
| 457 | CO-3558 | MT = M5C = M5C = M5C = M5C = M5C = M5C = M5C = |
| | | MA = MA = MT = MA = MA = MG = MA = MG = MG = M5C = MT = MT |
| 458 | CO-3559 | MG = MT = MT = MG = MT = MA = MG = MA = MG = |
| | | MA = M5C = MA = M5C = M5C = MT = M5C = M5C = M5C = M5C = MG |
| 459 | CO-3560 | MT = MG = MA = MT = MG = M5C = MA = M5C = MT = |
| | | MA = MA = M5C = MG = MA = MG = MG = MT = MT = MG = MT |
| 460 | CO-3561 | M5C = M5C = MT = M5C = MT = MT = MG = MA = MA = |
| | | MA = M5C = MT = M5C = M5C = M5C = M5C = MT = MA = MG-MG |
| 461 | CO-3562 | MG = M5C = M5C = MT = MG = MT = MG = M5C = MA = |
| | | MA = MT = MT = MG = MA = MG-MG = MG = MT = M5C = M5C |
| 462 | CO-3563 | MG = M5C = MA = MG = MA = M5C = MA = MA = MT = |
| | | M5C = MT = M5C = M5C = M5C = M5C = MG = MG = M5C = MT = MA |
| 463 | CO-3564 | MA = M5C = MG = M5C = MA = MG = MG = MT = MA = |
| | | MG = MG = MA = MG = MG = MA = M5C = M5C = M5C = MT = M5C |
| 464 | CO-3565 | MG = MA = MG = M5C = MT = MG = MA = M5C = M5C = |
| | | MG = M5C = M5C = MA = MG = MA = MT = MG = M5C = M5C = MT |
| 465 | CO-3566 | MG = M5C = MT = MA = MA = MT = MG = MG = MA = |
| | | MA = MA = MT = MT = MG = MA = MG = MG = MT = MG = MG |
| 466 | CO-3567 | M5C = M5C = M5C = MT = MG = MG = MA = MT = M5C = |
| | | MT = M5C = M5C = MG = MA = MG = M5C = MA = MA = MT = MG |
| 467 | CO-3568 | M5C = M5C = MG = M5C = M5C = MT = M5C = MT = MA = |
| | | M5C = MG = MT = MA = MG = MA = M5C = MA = MA = MG = MT |
| 468 | CO-3569 | MG = MG = MA = MA = MT = MG = MT = MG = MA = |
| | | MA = M5C = MT = MG = MA = MG = MT = MA = MG = MA = MA |
| 469 | CO-3570 | M5C = MT = MA = MA = MT = M5C = MA = M5C = MA = |
| | | MT = MG = MA = MT = M5C = M5C = M5C = MT = MA = MG = MA |
| 470 | CO-3571 | MG = M5C = MT = MA = MT = MG = MT = MT = MG = |
| | | MT = M5C = MA = MT = MA = MT = MG = MA = MT = MT = M5C |
| 471 | CO-3572 | MG = M5C = M5C = MG = M5C = M5C = MA = MG = MA = |
| | | MA = M5C = M5C = M5C = MA = MA = MA = M5C = M5C = MA = MG |
| 472 | CO-3573 | MG = MT = M5C = MT = M5C = MA = MT = M5C = MT = |
| | | M5C = MT = MG = M5C = MA = MA = M5C = MA = M5C = MT = M5C |
| 473 | CO-3574 | MG = M5C = MT = M5C = MT = M5C = M5C = MT = MA = |
| | | MA = M5C = MT = MG = MT = MG = MG = MG = MT = MG = M5C |
| 474 | CO-3575 | MG = MT = MA = MA = MA = M5C = MG = MA = M5C = |
| | | M5C = MT = MT = MG = MA = MA = M5C = MG = M5C = MG = MT |
| 475 | CO-3576 | MA = MT = MG = MT = MA = M5C = MA = MA = MA = |
| | | MA = MG = MT = M5C = M5C = M5C = MG = MA = MT = M5C = M5C |
| 476 | CO-3577 | M5C = MT = MG = M5C = M5C = MA = MG = MG = M5C = |
| | | MG = MT = MG = MA = MA = MG = MA = MG = MA = M5C = MA |
| 477 | CO-3578 | MT = MT = M5C = M5C = MA = MA = M5C = MT = MG = |
| | | MG = MT = MT = MT = M5C = MG = MA = MG = MT = M5C = M5C |
| 478 | CO-3579 | MG = MA = M5C = MT = MA = MT = MT = MT = MA = |
| | | MT = M5C = MT = MT = M5C = MA = M5C = M5C = M5C = MT = M5C |
| 479 | CO-3580 | MG = M5C = M5C = MA = M5C = MT = M5C = MT = M5C = |
| | | M5C = M5C = M5C = MA = MT = MT = M5C = MA = MA = MA = M5C |
| 480 | CO-3581 | M5C = MG = M5C = M5C = M5C = MT = MT = MG = MA = |
| | | MA = M5C = M5C = MT = MT = MT = MA = MT = MT = MA = MG |
| 481 | CO-3582 | M5C = MA = M5C = MA = MT = M5C = MT = MA = M5C = |
| | | MA = MG = MT = MG = M5C = M5C = M5C = MG = MG = MT = MA |
| 482 | CO-3583 | MT = M5C = M5C = M5C = M5C = M5C = M5C = MA = |
| | | MG = MT = MT = MT = MG = MA = MG = M5C = M5C = MA = MA = MT |
| 483 | CO-3584 | MG = M5C = MT = MT = M5C = MA = MA = MT = MT = |
| | | MT = MA = MA = MT = MG = M5C = MA = MG = MT = MG = M5C |
| 484 | CO-3585 | MG = MT = M5C = M5C = M5C = MA = M5C = M5C = MT = |
| | | MT = MA = MT = M5C = MA = MG = M5C = M5C = MT = M5C = MT |
| 485 | CO-3586 | MA = MA = MT = M5C = MT = MA = MG = MT = MT = |
| | | MT = M5C = MA = MA = MG = MT = MT = M5C = M5C = MT = M5C |
| 486 | CO-3587 | MT = MA = M5C = M5C = M5C = MA = MA = MG = MG = |
| | | MA = MA = MA = MG = MG = MA = MT = MT = M5C = MA = M5C |
| 487 | CO-3588 | MG = MT = M5C = MT = MG = MT = MA = MA = MA = |
| | | MG = MA = MA = M5C = MA = MA = MG = MA = MT = MA = MT |
| 488 | CO-3589 | MT = MT = MT = M5C = MA = MA = M5C = M5C = M5C = |
| | | MT = MT = MG = M5C = MT = MA = MG = MA = MG = MG = M5C |
| 489 | CO-3590 | MT = MA = MA = MT = MG = M5C = M5C = M5C = MT = |
| | | MT = MA = MT = M5C = MT = MT = MG = MA = MA = M5C = MG |
| 490 | CO-3591 | M5C = M5C = MA = M5C = M5C = MA = MG = M5C = M5C = |
| | | MT = MA = M5C = MT = M5C = M5C = MA = M5C = MA = MG = MG |
| 491 | CO-3592 | MG = MA = MA = MA = M5C = M5C = M5C = M5C = MT = |
| | | MT = M5C = M5C = MA = MT = M5C = MT = M5C = MG = MA = M5C |
| 492 | CO-3593 | M5C = MA = MT = MA = MA = M5C = MA = MA = MT = |
| | | MT = M5C = M5C = MT = M5C = MG = MA = MA = MG = MG = MT |
| 493 | CO-3594 | M5C = MA = MT = MG = MG = MT = M5C = MT = M5C = |
| | | MT = MA = MT = MT = M5C = MA = M5C = M5C = M5C = MA = M5C |
| 494 | CO-3595 | M5C = M5C = MT = MG = MA = MA = M5C = MT = MG = |
| | | MT = MA = MT = MG = MG = M5C = MT = MG = MA = M5C = M5C |
| 495 | CO-3596 | M5C = MT = M5C = MT = MA = MT = MG = MT = MA = |
| | | MA = MG = MA = M5C = MT = M5C = M5C = M5C = MA = M5C = MT |
| 496 | CO-3597 | MA = M5C = MA = MA = M5C = M5C = M5C = MT = MG = |
| | | M5C = MA = MT = MG = M5C = MA = M5C = MT = M5C = M5C = MT |
| 497 | CO-3598 | MT = MA = MA = MG = MA = MA = MG = M5C = M5C = |
| | | M5C = MT = M5C = MA = M5C = MG = MG = MA = MA = MT = MT |
| 498 | CO-3599 | MA = MT = MG = MA = MT = MG = M5C = MA = M5C = |
| | | M5C = MA = MG = MT = M5C = MT = MT = MA = M5C = MA = M5C |
| 499 | CO-3600 | MG = MG = MA = MA = M5C = MA = M5C = M5C = MT = |
| | | MA = MT = MG = M5C = MT = MA = MA = M5C = MA = M5C = M5C |
| 500 | CO-4078 | MG = MT = MT = M5C = MT = dC = dC = dC = dT = |
| | | dA = dG = dC = dT = dG = dT = MG = MT = MA = MG = MT |
| 501 | CO-4079 | M5C = M5C = MT = MG = MA = dA = dC = dT = dG = |
| | | dT = dA = dT = dG = dG = dC = MT = MG = MA = M5C = M5C |
| 502 | CO-4080 | MG = MT = MT = M5C = MT = dC = dC = dC = MT = |
| | | dA = dG = M5C = dT = dG = dT = MG = MT = MA = MG = MT |
| 503 | CO-4081 | M5C = M5C = MT = MG = MA = dA = dC = dT = MG = |
| | | dT = dA = MT = dG = dG = dC = MT = MG = MA = M5C = M5C |
| 504 | CO-4082 | MG = MT = MT = M5C = MT = L5C = M5C = M5C = |
| | | LT = MA = MG = L5C = MT = MG = LT = MG = MT = MA = MG = MT |
| 505 | CO-4083 | M5C = M5C = MT = MG = MA = LA = M5C = MT = LG = |
| | | MT = MA = LT = MG = MG = L5C = MT = MG = MA = M5C = M5C |
| 506 | CO-4084 | MA = MG = MT = MT = M5C = MT = M5C = M5C = M5C = |
| | | MT = MA = MG = M5C = MT = MG = MT = MG = MT = MA = MG = MT = MG |
| 507 | CO-4085 | M5C = MA = MG = MT = MT = M5C = MT = M5C = M5C = |
| | | M5C = MT = MA = MG = M5C = MT = MG = MT = MG = MT = MA = MG = MT = |
| | | MG = MT |
| 508 | CO-4086 | MT = M5C = M5C = MT = MG = MA = MA = M5C = MT = |
| | | MG = MT = MA = MT = MG = MG = M5C = MT = MG = MA = M5C = M5C = M5C |
| 509 | CO-4087 | MG = MT = M5C = M5C = MT = MG = MA = MA = M5C = |
| | | MT = MG = MT = MA = MT = MG = MG = M5C = MT = MG = MA = M5C = M5C = |
| | | M5C = M5C |
| 510 | CO-4088 | MT = MT = M5C = MT = M5C = M5C = M5C = MT = MA = |
| | | MG = M5C = MT = MG = MT = MG = MT = MA = MG = MT = MG |
| 511 | CO-4089 | M5C = MA = MG = MT = MT = M5C = MT = M5C = M5C = |
| | | M5C = MT = MA = MG = M5C = MT = MG = MT = MG = MT = MA |
| 512 | CO-4090 | M5C = MT = M5C = M5C = MA = MG = MT = MT = M5C = |
| | | MT = M5C = M5C = M5C = MT = MA = MG = M5C = MT = MG = MT |
| 513 | CO-4091 | MA = MG = MG = M5C = MT = M5C = M5C = MA = MG = |
| | | MT = MT = M5C = MT = M5C = M5C = M5C = MT = MA = MG = M5C |
| 514 | CO-4092 | MG = MA = MA = MA = MG = MG = M5C = MT = M5C = |
| | | M5C = MA = MG = MT = MT = M5C = MT = M5C = M5C = M5C = MT |
| 515 | CO-4093 | MT = MG = MT = MA = MT = MG = MG = M5C = MT = |
| | | MG = MA = M5C = M5C = M5C = M5C = M5C = MA = MT = MG = MG |
| 516 | CO-4094 | MA = MA = M5C = MT = MG = MT = MA = MT = MG = |
| | | MG = M5C = MT = MG = MA = M5C = M5C = M5C = M5C = M5C = MA |
| 517 | CO-4095 | MT = MG = MA = MA = M5C = MT = MG = MT = MA = |
| | | MT = MG = MG = M5C = MT = MG = MA = M5C = M5C = M5C = M5C |
| 518 | CO-4096 | M5C = MT = MG = MA = MA = M5C = MT = MG = MT = |
| | | MA = MT = MG = MG = M5C = MT = MG = MA = M5C = M5C = M5C |
| 519 | CO-4097 | MT = M5C = M5C = MT = MG = MA = MA = M5C = MT = |
| | | MG = MT = MA = MT = MG = MG = M5C = MT = MG = MA = M5C |
| 520 | CO-4098 | MG = MT = M5C = M5C = MT = MG = MA = MA = M5C = |
| | | MT = MG = MT = MA = MT = MG = MG = M5C = MT = MG = MA |
| 521 | CO-4099 | M5C = MT = MG = MT = M5C = M5C = MT = MG = MA = |
| | | MA = M5C = MT = MG = MT = MA = MT = MG = MG = M5C = MT |
| 522 | CO-4100 | M5C = M5C = MA = M5C = MT = MG = MT = M5C = |
| | | M5C = MT = MG = MA = MA = M5C = MT = MG = MT = MA = MT = MG |
| 523 | CO-4101 | MA = M5C = MT = M5C = M5C = M5C = MA = M5C = |
| | | MT = MG = MT = M5C = M5C = MT = MG = MA = MA = M5C = MT = MG |
| 524 | CO-4102 | MA = MA = MG = MA = M5C = MT = M5C = M5C = |
| | | M5C = MA = M5C = MT = MG = MT = M5C = M5C = MT = MG = MA = MA |
| 525 | CO-4103 | MT = MA = MT = MG = MT = MA = MA = MG = MA = |
| | | M5C = MT = M5C = M5C = M5C = MA = M5C = MT = MG = MT = M5C |
| 526 | CO-4403 | MG = MT = MT = M5C = MT = L5C = dC = dC = LT = |
| | | dA = dG = L5C = dT = dG = LT = MG = MT = MA = MG = MT |
| 527 | CO-4404 | MG = MT = MT = L5C = MT = L5C = M5C = L5C = MT = |
| | | LA = MG = L5C = MT = LG = MT = LG = MT = LA = MG = MT |
| 528 | CO-4405 | MG = MT = LT = M5C = LT = M5C = L5C = M5C = LT = |
| | | MA = LG = M5C = LT = MG = LT = MG = LT = MA = MG = MT |
| 529 | CO-4406 | MG = MT = LT = M5C = MT = L5C = M5C = M5C = LT = |
| | | MA = MG = L5C = MT = MG = LT = MG = MT = LA = MG = MT |
| 530 | CO-4407 | MG = MT = MT = M5C = LT = M5C = M5C = M5C = |
| | | LT = MA = MG = M5C = LT = MG = MT = MG = LT = MA = MG = MT |
| 531 | CO-4408 | MG = MT = MT = L5C = MT = M5C = M5C = L5C = |
| | | MT = MA = MG = L5C = MT = MG = MT = LG = MT = MA = MG = MT |
| 532 | CO-4409 | MG = MT = MT = M5C = MT = L5C = M5C = M5C = |
| | | MT = MA = LG = M5C = MT = MG = MT = LG = MT = MA = MG = MT |
| 533 | CO-4410 | MG = MT = MT = M5C = LT = M5C = M5C = M5C = |
| | | MT = LA = MG = M5C = MT = MG = LT = MG = MT = MA = MG = MT |
| 534 | CO-4411 | M5C = M5C = MT = MG = MA = LA = dC = dT = LG = |
| | | dT = dA = LT = dG = dG = L5C = MT = MG = MA = M5C = M5C |
| 535 | CO-4412 | M5C = M5C = MT = LG = MA = LA = M5C = LT = MG = |
| | | LT = MA = LT = MG = LG = M5C = LT = MG = LA = M5C = M5C |
| 536 | CO-4413 | M5C = M5C = LT = MG = LA = MA = L5C = MT = LG = |
| | | MT = LA = MT = LG = MG = L5C = MT = LG = MA = M5C = M5C |
| 537 | CO-4414 | M5C = M5C = LT = MG = MA = LA = M5C = MT = LG = |
| | | MT = MA = LT = MG = MG = L5C = MT = MG = LA = M5C = M5C |
| 538 | CO-4415 | M5C = M5C = MT = MG = LA = MA = M5C = MT = LG = |
| | | MT = MA = MT = LG = MG = M5C = MT = LG = MA = M5C = M5C |
| 539 | CO-4416 | M5C = M5C = MT = LG = MA = MA = M5C = LT = MG = |
| | | MT = MA = LT = MG = MG = M5C = LT = MG = MA = M5C = M5C |
| 540 | CO-4417 | M5C = M5C = MT = MG = MA = LA = M5C = MT = MG = |
| | | MT = LA = MT = MG = MG = M5C = LT = MG = MA = M5C = M5C |
| 541 | CO-4418 | M5C = M5C = MT = MG = LA = MA = M5C = MT = MG = |
| | | LT = MA = MT = MG = MG = L5C = MT = MG = MA = M5C = M5C |
| 542 | CO-4419 | MA = MG = MT = MT = M5C = dT = M5C = dC = dC = |
| | | MT = dA = dG = M5C = dT = dG = MT = dG = MT = MA = MG = MT = MG |
| 543 | CO-4420 | MA = MG = MT = MT = M5C = MT = L5C = M5C = M5C = |
| | | LT = MA = MG = L5C = MT = MG = LT = MG = MT = MA = MG = MT = MG |
| 544 | CO-4421 | M5C = MA = MG = MT = MT = dC = dT = M5C = dC = dC = MT = |
| | | dA = dG = M5C = dT = dG = MT = dG = dT = MA = MG = MT = MG = MT |
| 545 | CO-4422 | M5C = MA = MG = MT = MT = M5C = MT = L5C = M5C = |
| | | M5C = LT = MA = MG = L5C = MT = MG = LT = MG = MT = MA = MG = MT = |
| | | MG = MT |
| 546 | CO-4423 | MA = M5C = M5C = MT = M5C = MT = MG = MT = M5C = |
| | | MT = M5C = M5C = MA = MG = MG = MT = M5C = M5C = MG = MG |
| 547 | CO-4424 | MA = MG = MA = MG = MA = M5C = MA = MA = MG = |
| | | MA = MG = MA = MA = MG = MA = M5C = M5C = MT = MG = M5C |
| 548 | CO-4425 | M5C = MA = MA = MG = MT = MA = MA = M5C = M5C = |
| | | MA = MA = MT = MG = MG = MA = MA = MG = M5C = M5C = M5C |
| 549 | CO-4426 | M5C = MA = M5C = MA = MT = MG = MA = MT = M5C = |
| | | M5C = M5C = MT = MA = MG = MA = MA = MA = MT = MG = MG |
| 550 | CO-4427 | M5C = M5C = MA = MG = MA = MA = MG = M5C = MA = |
| | | MG = MA = M5C = MA = M5C = M5C = M5C = MT = M5C = M5C = MA |
| 551 | CO-4428 | MG = MG = MA = MT = MT = MT = MA = MA = MA = |
| | | MT = M5C = MT = M5C = MT = MT = M5C = MT = MT = M5C = M5C |
| 552 | CO-4437 | MG = M5C = MA = MT = MT = MT = M5C = M5C = MT = |
| | | MG = MT = M5C = MT = MT = MT = M5C = MA = MT = MA = MG |
| 553 | CO-4438 | M5C = MA = MT = MG = MT = MG = MT = MG = MG = |
| | | MG = MT = MG = MG = M5C = MA = MT = MT = MT = M5C = M5C |
| 554 | CO-4439 | MG = MG = M5C = MA = M5C = MT = MT = MG = M5C = |
| | | MA = MA = M5C = M5C = M5C = MT = M5C = M5C = MT = MA = M5C |
| 555 | CO-4440 | MG = MG = MA = MT = MT = M5C = MA = M5C = M5C = |
| | | MT = MT = MA = MT = MA = MG = M5C = M5C = M5C = MA = MA |
| 556 | CO-4441 | MG = MA = MT = MG = MG = M5C = MA = MG = MA = |
| | | MT = M5C = M5C = MT = MT = M5C = MT = MA = MT = MG = MT |
| 557 | CO-4442 | MG = MG = MA = MT = MG = MT = MT = M5C = MT = |
| | | MT = MT = MG = MA = MA = MT = MT = MA = MG = MG = MT |
| 558 | CO-4443 | MA = MG = MA = MT = MG = MG = M5C = M5C = MT = |
| | | M5C = MT = MG = MG = MG = MA = MT = MG = MT = MT = M5C |
| 559 | CO-4444 | M5C = M5C = M5C = MT = MA = M5C = M5C = M5C = |
| | | M5C = MT = MT = M5C = M5C = MT = MG = MG = MT = MT = MA = MG |
| 560 | CO-4445 | M5C = MT = MA = MG = M5C = M5C = M5C = M5C = |
| | | MG = MT = MT = MG = MA = MG = MA = MG = MG = MA = MA = M5C |
| 561 | CO-4446 | MT = M5C = MA = M5C = M5C = MA = M5C = MT = |
| | | M5C = M5C = M5C = MT = MA = MG = M5C = M5C = M5C = M5C = MG = MT |
| 562 | CO-4447 | MG = MT = MA = MG = MT = MG = MT = MA = |
| | | MG = MA = MG = MG = MT = MG = MG = M5C = M5C = MA = MG = MG |
| 563 | CO-4448 | MG = M5C = MT = MG = MT = MG = MT = MA = |
| | | MG = MT = MG = MT = MA = MG = MA = MG = MG = MT = MG = MG |
| 564 | CO-4449 | M5C = M5C = M5C = MT = MA = MG = M5C = MT = |
| | | MG = MT = MG = MT = MA = MG = MT = MG = MT = MA = MG = MA |
| 565 | CO-4450 | M5C = MT = M5C = M5C = M5C = MT = MA = MG = |
| | | M5C = MT = MG = MT = MG = MT = MA = MG = MT = MG = MT = MA |
| 566 | CO-4451 | MT = M5C = MT = M5C = M5C = M5C = MT = MA = |
| | | MG = M5C = MT = MG = MT = MG = MT = MA = MG = MT = MG = MT |
| 567 | CO-4458 | MG = MT = MT-M5C-MT = M5C = M5C = M5C = |
| | | MT = MA = MG = M5C = MT = MG = MT = MG-MT-MA = MG = MT |
| 568 | CO-4459 | MG = MT = MT-M5C-MT-M5C-M5C = M5C = MT = |
| | | MA = MG = M5C = MT = MG-MT-MG-MT-MA = MG = MT |
| 569 | CO-4460 | MG = MT = MT-M5C-MT-M5C-M5C-M5C-MT-MA- |
| | | MG-M5C-MT-MG-MT-MG-MT-MA = MG = MT |
| 570 | CO-4461 | M5C = M5C = MT-MG-MA = MA = M5C = MT = MG = |
| | | MT = MA = MT = MG = MG = M5C = MT-MG-MA = M5C = M5C |
| 571 | CO-4462 | M5C = M5C = MT-MG-MA-MA-M5C = MT = MG = |
| | | MT = MA = MT = MG = MG-M5C-MT-MG-MA = M5C = M5C |
| 572 | CO-4463 | M5C = M5C = MT-MG-MA-MA-M5C-MT-MG-MT- |
| | | MA-MT-MG-MG-M5C-MT-MG-MA = M5C = M5C |
| 963 | CO-5230 | MT = MG = MG = MG = MA = MT = MG = MT = MT = |
| | | M5C = MT = MT = MT = MG = MA = MA = MT = MT = MA = MG |
| 964 | CO-5231 | MT = M5C = MT = MG = MG = MG = MA = MT = MG = |
| | | MT = MT = M5C = MT = MT = MT = MG = MA = MA = MT = MT |
| 965 | CO-5232 | M5C = M5C = MT = M5C = MT = MG = MG = MG = MA = |
| | | MT = MG = MT = MT = M5C = MT = MT = MT = MG = MA = MA |
| 966 | CO-5233 | MG = MG = M5C = M5C = MT = M5C = MT = MG = MG = |
| | | MG = MA = MT = MG = MT = MT = M5C = MT = MT = MT = MG |
| 967 | CO-5234 | MA = MT = MG = MG = M5C = M5C = MT = M5C = MT = |
| | | MG = MG = MG = MA = MT = MG-MT = MT = M5C = MT = MT |
| 968 | CO-5235 | MG = MG = MA = MG = MA = MT = MG = MG = M5C = |
| | | M5C = MT = M5C = MT = MG = MG = MG = MA = MT = MG = MT |
| 969 | CO-5236 | MT = MT = MA = MG-MG = MA = MG = MA = MT = MG = |
| | | MG = M5C = M5C = MT = M5C = MT = MG = MG = MG = MA |
| 970 | CO-5237 | MA = MG = MA = MT = MG = dG = d5C = d5C = dT = |
| | | d5C = dT = dG = dG = dG = dA = MT = MG = MT = MT = M5C |
| 971 | CO-5238 | MA = MG = MA = MT = MG = dG = d5C = d5C = MT = |
| | | d5C = dT = MG = dG = dG = dA = MT = MG = MT = MT = M5C |
| 972 | CO-5239 | MA = MG = MA = MT = MG = LG = d5C = d5C = LT = |
| | | d5C = dT = LG = dG = dG = LA = MT = MG = MT = MT = M5C |
| 973 | CO-5240 | MA = MG = MA = LT = MG = dG = d5C = L5C = dT = |
| | | d5C = dT = LG = dG = dG = dA = LT = MG = MT = MT = M5C |
| 974 | CO-5241 | MA = MG = MA = MT = LG = dG = d5C = d5C = LT = |
| | | d5C = dT = dG = LG = dG = dA = MT = LG = MT = MT = M5C |
| 975 | CO-5242 | MA = MG = MA = MT = MG = LG = M5C = M5C = LT = |
| | | M5C = MT = LG = MG = MG = LA = MT = MG = MT = MT = M5C |
| 976 | CO-5243 | MA = MG = MA = LT = MG = LG = M5C = L5C = MT = |
| | | L5C = MT = LG = MG = LG = MA = LT = MG = LT = MT = M5C |
| 977 | CO-5244 | MA = MG = LA = MT = LG = MG = L5C = M5C = LT = |
| | | M5C = LT = MG = LG = MG = LA = MT = LG = MT = MT = M5C |
| 978 | CO-5245 | MA = MG = LA = MT = MG = LG = M5C = M5C = LT = |
| | | M5C = MT = LG = MG = MG = LA = MT = MG = LT = MT = M5C |
| 979 | CO-5246 | MA = MG = MA = MT = LG = MG = M5C = M5C = LT = |
| | | M5C = MT = MG = LG = MG = MA = MT = LG = MT = MT = M5C |
| 980 | CO-5247 | MA = MG = MA = LT = MG = MG = M5C = L5C = MT = |
| | | M5C = MT = LG = MG = MG = MA = LT = MG = MT = MT = M5C |
| 981 | CO-5248 | MA = MG = MA = MT = MG = LG = M5C = M5C = MT = |
| | | M5C = LT = MG = MG = MG = MA = LT = MG = MT = MT = M5C |
| 982 | CO-5249 | MA = MG = MA = MT = LG = MG = M5C = M5C = MT = |
| | | L5C = MT = MG = MG = MG = LA = MT = MG = MT = MT = M5C |
| 983 | CO-5250 | MG = MA = MG = MA = MT = MG = dG = d5C = d5C = |
| | | dT = d5C = dT = dG = dG = dG = dA = MT = MG = MT = MT = M5C = MT |
| 984 | CO-5251 | MG = MA = MG = MA = MT = MG = MG = M5C = M5C = MT = |
| | | M5C = MT = MG = MG = MG = MA = MT = MG = MT = MT = M5C = MT |
| 985 | CO-5252 | MG = MA = MG = MA = MT = dG = dG = d5C = M5C = dT = |
| | | d5C = dT = dG = MG = dG = dA = dT = MG = MT = MT = M5C = MT |
| 986 | CO-5253 | MG = MA = MG = MA = MT = LG = dG = d5C = L5C = dT = |
| | | d5C = dT = dG = LG = dG = dA = LT = MG = MT = MT = M5C = MT |
| 987 | CO-5254 | MG = MG = MA = MG = MA = MT = MG = dG = d5C = d5C = |
| | | dT = d5C = dT = dG = dG = dG = dA = MT = MG = MT = MT = M5C = MT = MT |
| 988 | CO-5255 | MG = MG = MA = MG = MA = MT = MG = MG = M5C = M5C = |
| | | MT = M5C = MT = MG = MG = MG = MA = MT = MG = MT = MT = M5C = |
| | | MT = MT |
| 989 | CO-5256 | MG = MG = MA = MG = MA = dT = dG = MG = d5C = d5C = MT = |
| | | d5C = dT = MG = dG = dG = MA = dT = dG = MT = MT = M5C = MT = MT |
| 990 | CO-5257 | MG = MG = MA = MG = MA = dT = dG = LG = d5C = d5C = LT = |
| | | d5C = dT = LG = dG = dG = LA = dT = dG = MT = MT = M5C = MT = MT |
| 573 | CO-5290 | MG = MT = MT-M5C-MT-M5C-M5C-M5C-MT- |
| | | MA-MG-M5C-MT-MG-MT-MG-MT = MA = MG = MT |
| 574 | CO-5291 | MG = MT = MT = M5C = MT-M5C-M5C-M5C-MT- |
| | | MA-MG-M5C-MT-MG-MT-MG-MT = MA = MG = MT |
| 575 | CO-5292 | MG = MT = MT = M5C = MT = M5C = M5C-M5C- |
| | | MT-MA-MG-M5C-MT-MG-MT-MG-MT = MA = MG = MT |
| 576 | CO-5293 | MG = MT = MT = M5C = MT = M5C = M5C = M5C = |
| | | MT-MA-MG-M5C-MT-MG-MT-MG-MT = MA = MG = MT |
| 577 | CO-5294 | MG = MT = MT = M5C = MT = M5C = M5C = M5C = |
| | | MT = MA = MG-M5C-MT-MG-MT-MG-MT = MA = MG = MT |
| 578 | CO-5295 | MG = MT = MT = M5C = MT = M5C = M5C = M5C = |
| | | MT = MA = MG = M5C = MT-MG-MT-MG-MT = MA = MG = MT |
| 579 | CO-5296 | MG = MT = MT-M5C-MT-M5C-M5C-M5C-MT-MA-MG-M5C- |
| | | MT-MG-MT-MG = MT = MA = MG = MT |
| 580 | CO-5297 | MG = MT = MT-M5C-MT-M5C-M5C-M5C-MT-MA-MG- |
| | | M5C-MT-MG = MT = MG = MT = MA = MG = MT |
| 581 | CO-5298 | MG = MT = MT-M5C-MT-M5C-M5C-M5C-MT-MA-MG- |
| | | M5C = MT = MG = MT = MG = MT = MA = MG = MT |
| 582 | CO-5299 | MG = MT = MT-M5C-MT-M5C-M5C-M5C-MT-MA = MG = |
| | | M5C = MT = MG-MT = MG = MT = MA = MG = MT |
| 583 | CO-5300 | MG = MT = MT-M5C-MT-M5C-M5C-M5C = MT = MA = |
| | | MG = M5C = MT = MG = MT = MG = MT = MA = MG = MT |
| 584 | CO-5301 | LG = LT = MT = L5C = LT = M5C = L5C = M5C = LT = |
| | | MA = LG = M5C = LT = MG = LT = MG = LT = MA = LG = LT |
| 585 | CO-5302 | LG = LT = MT = L5C = MT = L5C = M5C = L5C = MT = |
| | | LA = MG = L5C = MT = LG = MT = LG = LT = MA = LG = LT |
| 586 | CO-5303 | M5C = M5C = MT-MG-MA-MA-M5C-MT-MG-MT- |
| | | MA-MT-MG-MG-M5C-MT-MG = MA = M5C = M5C |
| 587 | CO-5304 | M5C = M5C = MT = MG = MA-MA-M5C-MT-MG-MT- |
| | | MA-MT-MG-MG-M5C-MT-MG = MA = M5C = M5C |
| 588 | CO-5305 | M5C = M5C = MT = MG = MA = MA = M5C-MT- |
| | | MG-MT-MA-MT-MG-MG-M5C-MT-MG = MA = M5C = M5C |
| 589 | CO-5306 | M5C = M5C = MT = MG = MA = MA = M5C = MT = |
| | | MG-MT-MA-MT-MG-MG-M5C-MT-MG = MA = M5C = M5C |
| 590 | CO-5307 | M5C = M5C = MT = MG = MA = MA = M5C = MT = |
| | | MG = MT = MA-MT-MG-MG-M5C-MT-MG = MA = M5C = M5C |
| 591 | CO-5308 | M5C = M5C = MT = MG = MA = MA = M5C = MT = |
| | | MG = MT = MA = MT = MG-MG-M5C-MT-MG = MA = M5C = M5C |
| 592 | CO-5309 | M5C = M5C = MT-MG-MA-MA-M5C-MT-MG-MT- |
| | | MA-MT-MG-MG-M5C-MT = MG = MA = M5C = M5C |
| 593 | CO-5310 | M5C = M5C = MT-MG-MA-MA-M5C-MT-MG-MT-MA- |
| | | MT-MG-MG = M5C = MT = MG = MA = M5C = M5C |
| 594 | CO-5311 | M5C = M5C = MT-MG-MA-MA-M5C-MT-MG-MT-MA- |
| | | MT = MG = MG = M5C = MT = MG = MA = M5C = M5C |
| 595 | CO-5312 | M5C = M5C = MT-MG-MA-MA-M5C-MT-MG-MT = MA = |
| | | MT = MG = MG = M5C = MT = MG = MA = M5C = M5C |
| 596 | CO-5313 | M5C = M5C = MT-MG-MA-MA-M5C-MT = MG = MT = |
| | | MA = MT = MG = MG = M5C = MT = MG = MA = M5C = M5C |
| 597 | CO-5314 | L5C = L5C = MT = LG = LA = MA = L5C = MT = LG = |
| | | MT = LA = MT = LG = MG = L5C = MT = LG = MA = L5C = L5C |
| 598 | CO-5315 | L5C = L5C = MT = LG = MA = LA = M5C = LT = MG = |
| | | LT = MA = LT = MG = LG = M5C = LT = LG = MA = L5C = L5C |
| 627 | CO-6348 | M5C = M5C = M5C = MG = MA = M5C = MG = M5C = |
| | | MA = MG = MG = MT = MA = MG = MG = MA = MG = MG = MA = M5C |
| 628 | CO-6349 | MG = MG = MA = M5C = M5C = M5C = M5C = MG = MA = |
| | | M5C = MG = M5C = MA = MG = MG = MT = MA = MG = MG = MA |
| 629 | CO-6350 | M5C = MT = M5C = M5C = M5C = MA = MG = MG = MG = |
| | | MA = MG = M5C = M5C = M5C = MG = MG = MA = M5C = M5C = M5C |
| 630 | CO-6351 | M5C = MG = M5C = M5C = MA = MG = MA = MT = MG = |
| | | M5C = M5C = MT = M5C = M5C = M5C = MA = MG = MG = MG = MA |
| 631 | CO-6352 | MA = M5C = M5C = MG = M5C = M5C = MA = MG = MA = |
| | | MT = MG = M5C = M5C = MT = M5C = M5C = M5C = MA = MG = MG |
| 632 | CO-6353 | MT = MG = MA = M5C = M5C = MG = M5C = M5C = MA = |
| | | MG = MA = MT = MG = M5C = M5C = MT = M5C = M5C = M5C = MA |
| 633 | CO-6354 | MG = MA = MG = M5C = MT = dG = dA = d5C = d5C = dG = |
| | | d5C = d5C = dA = dG = dA = MT = MG = M5C = M5C = MT |
| 634 | CO-6355 | MG = MG = MA = MG = M5C = MT = MG = MA = M5C = |
| | | M5C = MG = M5C = M5C = MA = MG = MA = MT = MG = M5C = M5C |
| 635 | CO-6356 | MA = MG = MT = M5C = M5C = MT = MG = MG = MA = |
| | | MG = M5C = MT = MG = MA = M5C = M5C = MG = M5C = M5C = MA |
| 636 | CO-6357 | M5C = MT = MA = MG = MT = M5C = M5C = MT = MG = |
| | | MG = MA = MG = M5C = MT = MG = MA = M5C = M5C = MG = M5C |
| 637 | CO-6358 | M5C = M5C = M5C = MT = MA = MG = MT = M5C = M5C = |
| | | MT = MG = MG = MA = MG = M5C = MT = MG = MA = M5C = M5C |
| 638 | CO-6359 | MG = MG = M5C = M5C = MA = MA = MG = M5C = M5C = |
| | | M5C = MT = MA = MG = MT = M5C = M5C = MT = MG = MG = MA |
| 639 | CO-6360 | MG = MA = MG = M5C = MT = dG = dA = d5C = M5C = |
| | | dG = d5C = M5C = dA = dG = dA = MT = MG = M5C = M5C = MT |
| 640 | CO-6361 | MG = MA = MG = M5C = MT = LG = dA = d5C = L5C = |
| | | dG = d5C = L5C = dA = dG = LA = MT = MG = M5C = M5C = MT |
| 641 | CO-6362 | MG = MA = MG = L5C = MT = dG = dA = L5C = d5C = |
| | | dG = d5C = L5C = dA = dG = dA = LT = MG = M5C = M5C = MT |
| 642 | CO-6363 | MG = MA = MG = M5C = LT = dG = dA = d5C = L5C = |
| | | dG = d5C = d5C = LA = dG = dA = MT = LG = M5C = M5C = MT |
| 643 | CO-6364 | MG = MA = MG = M5C = MT = LG = MA = M5C = L5C = |
| | | MG = M5C = L5C = MA = MG = LA = MT = MG = M5C = M5C = MT |
| 644 | CO-6365 | MG = MA = MG = L5C = MT = LG = MA = L5C = M5C = |
| | | LG = M5C = L5C = MA = LG = MA = LT = MG = L5C = M5C = MT |
| 645 | CO-6366 | MG = MA = LG = M5C = LT = MG = LA = M5C = L5C = |
| | | MG = L5C = M5C = LA = MG = LA = MT = LG = M5C = M5C = MT |
| 646 | CO-6367 | MG = MA = LG = M5C = MT = LG = MA = M5C = L5C = |
| | | MG = M5C = L5C = MA = MG = LA = MT = MG = L5C = M5C = MT |
| 647 | CO-6368 | MG = MA = MG = M5C = LT = MG = MA = M5C = L5C = |
| | | MG = M5C = M5C = LA = MG = MA = MT = LG = M5C = M5C = MT |
| 648 | CO-6369 | MG = MA = MG = L5C = MT = MG = MA = L5C = M5C = |
| | | MG = M5C = L5C = MA = MG = MA = LT = MG = M5C = M5C = MT |
| 649 | CO-6370 | MG = MA = MG = M5C = MT = LG = MA = M5C = M5C = |
| | | MG = L5C = M5C = MA = MG = MA = LT = MG = M5C = M5C = MT |
| 650 | CO-6371 | MG = MA = MG = M5C = LT = MG = MA = M5C = M5C = |
| | | LG = M5C = M5C = MA = MG = LA = MT = MG = M5C = M5C = MT |
| 651 | CO-6372 | MG = MG = MA = MG = M5C = MT = dG = dA = d5C = |
| | | d5C = dG = d5C = d5C = dA = dG = dA = MT = MG = M5C = M5C = MT = M5C |
| 652 | CO-6373 | MG = MG = MA = MG = M5C = MT = MG = MA = M5C = |
| | | M5C = MG = M5C = M5C = MA = MG = MA = MT = MG = M5C = M5C = MT = |
| | | M5C |
| 653 | CO-6374 | MG = MG = MA = MG = M5C = dT = dG = dA = M5C = |
| | | d5C = dG = d5C = d5C = MA = dG = dA = dT = MG = |
| | | M5C = M5C = MT = M5C |
| 654 | CO-6375 | MG = MG = MA = MG = M5C = LT = dG = dA = L5C = |
| | | d5C = dG = d5C = d5C = LA = dG = dA = LT = MG = M5C = M5C = MT = M5C |
| 655 | CO-6376 | MT = MG = MG = MA = MG = M5C = MT = dG = dA = d5C = |
| | | d5C = dG = d5C = d5C = dA = dG = dA = MT = MG = M5C = M5C = MT = M5C = |
| | | M5C |
| 656 | CO-6377 | MT = MG = MG = MA = MG = M5C = MT = MG = MA = |
| | | M5C = M5C = MG = M5C = M5C = MA = MG = MA = MT = MG = M5C = M5C = |
| | | MT = M5C = M5C |
| 657 | CO-6378 | MT = MG = MG = MA = MG = d5C = dT = MG = dA = d5C = |
| | | M5C = dG = d5C = M5C = dA = dG = MA = dT = dG = M5C = M5C = MT = M5C = |
| | | M5C |
| 658 | CO-6379 | MT = MG = MG = MA = MG = d5C = dT = LG = dA = d5C = L5C = |
| | | dG = d5C = L5C = dA = dG = LA = dT = dG = M5C = M5C = MT = M5C = M5C |
| 659 | CO-6456 | MG = MG = MA = M5C = M5C = MA = MA = MA = M5C = |
| | | MT = MA = MG = MA = M5C = M5C = M5C = M5C = MA = MT = MT |
| 660 | CO-6457 | MT = MG = MG = MA = MT = MA = M5C = M5C = M5C = |
| | | MA = MG = MG = MT = MA = M5C = MA = MG = MG = MA = M5C |
| 661 | CO-6458 | MT = MG = MA = M5C = M5C = MA = MA = MG = M5C = |
| | | MT = MG = M5C = M5C = MT = MG = MG = MA = MT = MA = M5C |
| 662 | CO-6459 | MA = M5C = MT = MA = M5C = MA = M5C = M5C = MA = |
| | | M5C = MG = MG = MA = MT = M5C = MT = M5C = M5C = MT = MG |
| 663 | CO-6460 | MT = MG = M5C = M5C = MT = MA = MT = M5C = M5C = |
| | | MA = MA = MG = MA = MA = M5C = MT = MA = M5C = MA = M5C |
| 664 | CO-6461 | MG = MA = MT = M5C = M5C = MA = MG = MA = MG = |
| | | MT = MA = MA = MG = MT = MG = M5C = M5C = MT = MA = MT |
| 665 | CO-6462 | MG = MT = MA = MT = MG = MT = MG = MA = M5C = |
| | | M5C = MT = MG = MA = MT = M5C = M5C = MA = MG = MA = MG |
| 666 | CO-6463 | MT = MG = M5C = M5C = M5C = M5C = M5C = MA = MG = |
| | | MG = MA = M5C = MA = M5C = MT = MG = MT = MA = MT = MG |
| 667 | CO-6464 | MA = MT = M5C = MT = MG = M5C = MT = MG = MT = |
| | | MT = M5C = M5C = MG = MA = M5C = M5C = MA = M5C = M5C = MT |
| 668 | CO-6465 | MA = MA = M5C = M5C = MT = MA = MG = MG = M5C = |
| | | M5C = MA = MT = M5C = MT = MG = M5C = MT = MG = MT = MT |
| 669 | CO-6466 | MT = MA = M5C = MT = MT = MG = MT = M5C = M5C = |
| | | M5C = MA = MA = M5C = M5C = MT = MA = MG = MG = M5C = M5C |
| 670 | CO-6467 | MT = MG = MG = MA = MG = MT = MT = M5C = MA = |
| | | MG = MA = MG = MA = M5C = MT = MT = MA = MT = MA = M5C |
| 671 | CO-6468 | MT = MG = MT = M5C = M5C = MT = M5C = MA = M5C = |
| | | MT = MA = M5C = MT = MG = MG = MA = MG = MT = MT = M5C |
| 672 | CO-6469 | MA = MT = M5C = MA = MG = M5C = M5C = MT = MG = |
| | | MG = M5C = MT = M5C = MG = M5C = MT = M5C = M5C = M5C = MT |
| 673 | CO-6470 | MA = MG = MT = MG = MT = MA = M5C = MG = M5C = |
| | | MT = MA = MT = M5C = MA = MG = M5C = M5C = MT = MG = MG |
| 674 | CO-6471 | M5C = MG = MG = MT = MT = MT = MG = MA = MA = |
| | | MA = MT = MA = MG = MT = MG = MT = MA = M5C = MG = M5C |
| 675 | CO-6472 | MG = M5C = MT = M5C = MA = MT = MG = M5C = MT = |
| | | MG = M5C = M5C = M5C = M5C = M5C = MT = MA = MG = MT = MG |
| 676 | CO-6473 | M5C = MT = MG = MA = MT = M5C = M5C = M5C = MT = |
| | | MG = MA = MG = MA = MG = M5C = MT = M5C = MT = MA = MA |
| 677 | CO-6474 | MT = M5C = MT = MT = MA = M5C = MA = MA = MG = |
| | | MG = MA = MG = MG = M5C = MT = M5C = M5C = MA = MA = MG |
| 678 | CO-6475 | MG = M5C = M5C = M5C = MA = MT = MA = MT = MA = |
| | | MT = MA = MG = MT = M5C = MT = MT = MA = M5C = MA = MA |
| 679 | CO-6476 | MG = MG = MA = MT = MT = M5C = M5C = M5C = M5C = |
| | | MT = MG = MA = MG = M5C = M5C = M5C = MA = MT = MA = MT |
| 680 | CO-6477 | MG = MT = MG = MA = MA = MT = MG = MG = M5C = MT = |
| | | MG = MG = MA = MT = MT = M5C = M5C = M5C = M5C = MT |
| 681 | CO-6478 | M5C = M5C = M5C = MA = MA = MT = MG = MT = MG = |
| | | MA = MG = MT = MG = MA = MA = MT = MG = MG = M5C = MT |
| 682 | CO-6479 | MG = M5C = MT = MG = M5C = MT = MG = MT = M5C = |
| | | M5C = MA = MA = MT = MG = M5C = M5C = M5C = MA = MA = MT |
| 683 | CO-6480 | M5C = M5C = MA = M5C = MT = MG = MG = MG = MA = |
| | | MA = MG = MT = MA = MT = MG = MG = M5C = MT = MG = M5C |
| 684 | CO-6481 | MT = MG = M5C = MT = MG = MT = MG = MA = MG = |
| | | M5C = MT = MA = M5C = M5C = M5C = MA = M5C = MT = MG = MG |
| 685 | CO-6482 | MT = MG = MA = MT = MT = M5C = MT = MA = M5C = |
| | | M5C = MT = MG = M5C = MT = MG = MT = MG = MA = MG = M5C |
| 686 | CO-6483 | MA = MG = MT = MG = MT = M5C = M5C = M5C = MG = |
| | | MA = MT = MG = MA = MT = MT = M5C = MT = MA = M5C = M5C |
| 687 | CO-6484 | MG = MT = M5C = MG = MT = MG = MT = MG = M5C = |
| | | M5C = M5C = MT = MG = MA = MT = MG = M5C = MT = MA = MA |
| 688 | CO-6485 | MG = M5C = M5C = MT = MT = MT = MT = MT = M5C = |
| | | MT = MG = MT = M5C = MG = MT = MG = MT = MG = M5C = M5C |
| 689 | CO-6486 | MT = MG = M5C = MT = M5C = MT = MT = MT = M5C = |
| | | MA = MG = MT = MG = MT = M5C = MT = MT = MT = MG = M5C |
| 690 | CO-6487 | MA = M5C = MA = MT = MG = M5C = M5C = MT = M5C = |
| | | MA = MT = MT = MG = M5C = MT = M5C = MT = MT = MT = M5C |
| 4916 | CO-6531 | mG = MU = MU = MC = MU = MC = MC = MC = MU = |
| | | MA = MG = MC = MU = MG = MU = MG = MU = MA = MG = MU |
| 692 | CO-6532 | mG = MU = MU-MC-MU-MC-MC-MC-MU-MA-MG-MC = |
| | | MU = MG = MU = MG = MU = MA = MG = MU |
| 693 | CO-6533 | mG = MU = MU-MC-MU-MC-MC-MC-MU-MA = MG = |
| | | MC = MU = MG = MU = MG = MU = MA = MG = MU |
| 694 | CO-6545 | MA = M5C = M5C = MA = MG = LG = MG = M5C = LG = |
| | | MT = MG = LA = MT = MG = LG = M5C = MT = MG = MA = MG |
| 695 | CO-6546 | MA = MG = MG = MA = M5C = L5C = MA = MG = LG = |
| | | MG = M5C = LG = MT = MG = LA = MT = MG = MG = M5C = MT |
| 696 | CO-6547 | MG = MG = MA = M5C = M5C = LA = MG = MG = LG = |
| | | M5C = MG = LT = MG = MA = LT = MG = MG = M5C = MT = MG |
| 697 | CO-6548 | MG = MA = M5C = M5C = MA = LG = MG = MG = L5C = |
| | | MG = MT = LG = MA = MT = LG = MG = M5C = MT = MG = MA |
| 698 | CO-7388 | MG = MG = M5C = M5C = MA = dA = dG = d5C = d5C = |
| | | d5C = dT = dA = dG = dT = d5C = M5C = MT = MG = MG = MA |
| 699 | CO-7389 | MG = MG = M5C = M5C = MA = dA = dG = d5C = M5C = |
| | | d5C = dT = MA = dG = dT = d5C = M5C = MT = MG = MG = MA |
| 700 | CO-7390 | MG = MG = M5C = M5C = MA = LA = dG = d5C = L5C = |
| | | d5C = dT = LA = dG = dT = L5C = M5C = MT = MG = MG = MA |
| 701 | CO-7391 | MG = MG = M5C = L5C = MA = dA = dG = L5C = d5C = |
| | | d5C = dT = LA = dG = dT = d5C = L5C = MT = MG = MG = MA |
| 702 | CO-7392 | MG = MG = M5C = M5C = LA = dA = dG = d5C = L5C = |
| | | d5C = dT = dA = LG = dT = d5C = M5C = LT = MG = MG = MA |
| 703 | CO-7393 | MG = MG = M5C = M5C = MA = LA = MG = M5C = L5C = |
| | | M5C = MT = LA = MG = MT = L5C = M5C = MT = MG = MG = MA |
| 704 | CO-7394 | MG = MG = M5C = L5C = MA = LA = MG = L5C = M5C = |
| | | L5C = MT = LA = MG = LT = M5C = L5C = MT = LG = MG = MA |
| 705 | CO-7395 | MG = MG = L5C = M5C = LA = MA = LG = M5C = L5C = |
| | | M5C = LT = MA = LG = MT = L5C = M5C = LT = MG = MG = MA |
| 706 | CO-7396 | MG = MG = L5C = M5C = MA = LA = MG = M5C = L5C = |
| | | M5C = MT = LA = MG = MT = L5C = M5C = MT = LG = MG = MA |
| 707 | CO-7397 | MG = MG = M5C = M5C = LA = MA = MG = M5C = L5C = |
| | | M5C = MT = MA = LG = MT = M5C = M5C = LT = MG = MG = MA |
| 708 | CO-7398 | MG = MG = M5C = L5C = MA = MA = MG = L5C = M5C = |
| | | M5C = MT = LA = MG = MT = M5C = L5C = MT = MG = MG = MA |
| 709 | CO-7399 | MG = MG = M5C = M5C = MA = LA = MG = M5C = M5C = |
| | | M5C = LT = MA = MG = MT = M5C = L5C = MT = MG = MG = MA |
| 710 | CO-7400 | MG = MG = M5C = M5C = LA = MA = MG = M5C = M5C = |
| | | L5C = MT = MA = MG = MT = L5C = M5C = MT = MG = MG = MA |
| 711 | CO-7401 | M5C = MG = MG = M5C = M5C = MA = dA = dG = d5C = |
| | | d5C = d5C = dT = dA = dG = dT = d5C = M5C = MT = MG = MG = MA = MG |
| 712 | CO-7402 | M5C = MG = MG = M5C = M5C = MA = MA = MG = M5C = |
| | | M5C = M5C = MT = MA = MG = MT = M5C = M5C = MT = MG = MG = MA = |
| | | MG |
| 713 | CO-7403 | M5C = MG = MG = M5C = M5C = dA = dA = dG = M5C = |
| | | d5C = d5C = dT = dA = MG = dT = d5C = d5C = MT = MG = MG = MA = MG |
| 714 | CO-7404 | M5C = MG = MG = M5C = M5C = LA = dA = dG = L5C = |
| | | d5C = d5C = dT = dA = LG = dT = d5C = L5C = MT = MG = MG = MA = MG |
| 715 | CO-7405 | M5C = MG = MG = M5C = M5C = LA = MA = MG = L5C = |
| | | M5C = M5C = MT = MA = LG = MT = M5C = L5C = MT = MG = MG = |
| | | MA = MG |
| 716 | CO-7406 | MG = M5C = MG = MG = M5C = M5C = MA = dA = dG = |
| | | d5C = d5C = d5C = dT = dA = dG = dT = d5C = M5C = MT = MG = MG = |
| | | MA = MG = M5C |
| 717 | CO-7407 | MG = M5C = MG = MG = M5C = M5C = MA = MA = MG = |
| | | M5C = M5C = M5C = MT = MA = MG = MT = M5C = M5C = MT = MG = MG = |
| | | MA = MG = M5C |
| 718 | CO-7408 | MG = M5C = MG = MG = M5C = d5C = dA = MA = dG = d5C = |
| | | M5C = d5C = dT = MA = dG = dT = M5C = d5C = dT = MG = MG = MA = MG = |
| | | M5C |
| 719 | CO-7409 | MG = M5C = MG = MG = M5C = d5C = dA = LA = dG = d5C = |
| | | L5C = d5C = dT = LA = dG = dT = L5C = d5C = dT = MG = MG = MA = |
| | | MG = M5C |
| 720 | CO-7410 | MG = M5C = MG = MG = M5C = M5C = MA = LA = MG = |
| | | M5C = L5C = M5C = MT = LA = MG = MT = L5C = M5C = MT = MG = |
| | | MG = MA = MG = M5C |
| 721 | CO-7411 | MG = MG = MA = MG = MA = MT = MG = LG = M5C = |
| | | M5C = LT = M5C = MT = LG = MG = MG = LA = MT = MG = MT = MT = |
| | | M5C = MT = MT |
| 722 | CO-7412 | MG = MG = MA = MG-MA-MT = MG = LG = M5C = M5C = |
| | | LT = M5C = MT = LG = MG = MG = LA = MT = MG-MT- |
| | | MT = M5C = MT = MT |
| 723 | CO-7413 | MG = MG = MA-MG-MA-MT = MG = LG = M5C = M5C = |
| | | LT = M5C = MT = LG = MG = MG = LA = MT = MG-MT-MT- |
| | | M5C = MT = MT |
| 724 | CO-7414 | MG = MG-MA-MG-MA-MT = MG = LG = M5C = M5C = |
| | | LT = M5C = MT = LG = MG = MG = LA = MT = MG-MT-MT-M5C- |
| | | MT = MT |
| 725 | CO-7415 | MG = MG = MA = MG = MA = MT = MG-LG-M5C-M5C-LT- |
| | | M5C-MT-LG-MG-MG-LA-MT-MG = MT = MT = M5C = MT = MT |
| 726 | CO-7416 | MG = MG = MA = MG = MA = LT = MG = MG = L5C = |
| | | M5C = MT = L5C = MT = MG = LG-MG-MA = LT = MG = MT = MT = |
| | | M5C = MT = MT |
| 727 | CO-7417 | MG = MG = MA = MG-MA-LT = MG = MG = L5C = |
| | | M5C = MT = L5C = MT = MG = LG = MG = MA = LT = MG-MT- |
| | | MT = M5C = MT = MT |
| 728 | CO-7418 | MG = MG = MA-MG-MA-LT = MG = MG = L5C = M5C = |
| | | MT = L5C = MT = MG = LG = MG = MA = LT = MG-MT-MT- |
| | | M5C = MT = MT |
| 729 | CO-7419 | MG = MG-MA-MG-MA-LT = MG = MG = L5C = M5C = |
| | | MT = L5C = MT = MG = LG = MG = MA = LT = MG-MT-MT-M5C- |
| | | MT = MT |
| 730 | CO-7420 | MG = MG = MA = MG = MA = LT = MG-MG-L5C-M5C-MT- |
| | | L5C-MT-MG-LG-MG-MA-LT-MG = MT = MT = M5C = MT = MT |
| 731 | CO-7421 | MG = MG = MA = MG = MA = MT = LG = MG = M5C = |
| | | L5C = MT = M5C = LT = MG = MG = LG = MA = MT = LG = MT = |
| | | MT = M5C = MT = MT |
| 732 | CO-7422 | MG = MG = MA = MG-MA-MT = LG = MG = M5C = |
| | | L5C = MT = M5C = LT = MG = MG = LG = MA = MT = LG-MT- |
| | | MT = M5C = MT = MT |
| 733 | CO-7423 | MG = MG = MA-MG-MA-MT = LG = MG = M5C = L5C = |
| | | MT = M5C = LT = MG = MG = LG = MA = MT = LG-MT-MT- |
| | | M5C = MT = MT |
| 734 | CO-7424 | MG = MG-MA-MG-MA-MT = LG = MG = M5C = L5C = |
| | | MT = M5C = LT = MG = MG = LG = MA = MT = LG-MT-MT-M5C- |
| | | MT = MT |
| 735 | CO-7425 | MG = MG = MA = MG = MA = MT = LG-MG-M5C-L5C-MT- |
| | | M5C-LT-MG-MG-LG-MA-MT-LG = MT = MT = M5C = MT = MT |
| 736 | CO-8051 | MT = MG = MG = M5C = MA = d5C = dT = dT = dG = |
| | | d5C = dA = dA = d5C = d5C = d5C = MT = M5C = M5C = MT = MA |
| 737 | CO-8052 | MG = MG = M5C = MA = MG = dA = dT = d5C = d5C = |
| | | dT = dT = d5C = dT = dA = dT = MG = MT = MA = MG = M5C |
| 738 | CO-8053 | MG = MG = MG = MA = MT = dG = dT = dT = d5C = |
| | | dT = dT = dT = dG = dA = dA = MT = MT = MA = MG = MG |
| 739 | CO-8054 | M5C = M5C = MG = MT = MT = dG = dA = dG = dA = |
| | | dG = dG = dA = dA = d5C = dT = MG = MG = MG = MT = M5C |
| 740 | CO-8055 | MG = MT = MG = MT = MA = dG = dT = dG = dT = |
| | | dA = dG = dA = dG = dG = dT = MG = MG = M5C = M5C = MA |
| 741 | CO-8056 | MA = MA = MG = MG = M5C = dT = d5C = d5C = dA = |
| | | dG = dT = dT = d5C = dT = d5C = M5C = M5C = MT = MA = MG |
| 742 | CO-8057 | MT = MA = MG = MT = MG = dG = dA = dA = dG = |
| | | dA = dT = dG = dT = d5C = dG = MG = MA = MG = M5C = MT |
| 743 | CO-8058 | M5C = MA = MT = MT = MT = d5C = d5C = d5C = d5C = |
| | | dT = d5C = dG = dA = d5C = dA = M5C = MT = M5C = MA = MG |
| 744 | CO-8059 | M5C = MA = MT = M5C = MT = dG = dG = dA = dT = |
| | | dT = dG = dG = d5C = dT = d5C = MA = M5C = M5C = MT = MT |
| 745 | CO-8060 | MA = M5C = MT = MT = MT = dT = d5C = d5C = d5C = |
| | | d5C = d5C = dG = d5C = dT = dG = M5C = M5C = MT = M5C = MT |
| 746 | CO-8061 | MG = MG = M5C = MG = MT = dG = dA = dT = dG = |
| | | dG = d5C = dT = dG = dA = dG = MT = M5C = M5C = M5C = MT |
| 747 | CO-8062 | M5C = MT = M5C = MT = MT = dT = d5C = dG = dG = |
| | | dA = d5C = dG = d5C = dT = dG = M5C = MA = MA = MG = MT |
| 748 | CO-8063 | MG = MT = M5C = MT = M5C = d5C = dA = dG = dG = |
| | | dT = d5C = d5C = dG = dG = d5C = MT = M5C = MT = MT = MT |
| 749 | CO-8064 | MG = M5C = MT = M5C = M5C = dT = d5C = dT = d5C = |
| | | dT = dT = dG = dT = d5C = d5C = MG = MT = MA = M5C = MT |
| 750 | CO-8065 | MT = MT = M5C = MT = M5C = dT = dT = dG = d5C = |
| | | d5C = d5C = dA = dG = d5C = dA = MT = MG = MT = M5C = MG |
| 751 | CO-8066 | MG = MT = MA = MG = MA = dG = dA = d5C = dA = |
| | | d5C = d5C = dT = d5C = d5C = d5C = M5C = MG = M5C = MA = M5C |
| 752 | CO-8067 | M5C = MT = MG = MA = MT = dG = d5C = dA = d5C = |
| | | dT = dA = dA = d5C = dG = dA = MG = MG = MT = MT = MG |
| 753 | CO-8068 | MA = M5C = MA = MA = MT = d5C = dT = d5C = d5C = |
| | | d5C = d5C = dG = dG = d5C = dT = MA = M5C = MT = MG = MT |
| 754 | CO-8069 | MG = MA = MA = MG = M5C = d5C = d5C = dT = dG = |
| | | dG = dA = dT = d5C = dT = d5C = M5C = MG = MA = MG = M5C |
| 755 | CO-8070 | M5C = M5C = M5C = MG = M5C = d5C = dT = d5C = dT = |
| | | dA = d5C = dG = dT = dA = dG = MA = M5C = MA = MA = MG |
| 756 | CO-8071 | MG = MG = MA = MG = MG = dA = dA = dT = dG = dT = |
| | | dG = dA = dA = d5C = dT = MG = MA = MG = MT = MA |
| 757 | CO-8072 | M5C = MT = MG = M5C = MA = dA = d5C = dA = d5C = |
| | | dT = d5C = dT = dG = d5C = d5C = MG = M5C = M5C = MA = MG |
| 758 | CO-8073 | M5C = M5C = MT = M5C = M5C = dA = dG = dT = dA = |
| | | dA = dA = d5C = dG = dA = d5C = M5C = MT = MT = MG = MA |
| 759 | CO-8074 | MA = MA = MG = MT = M5C = d5C = d5C = dG = dA = |
| | | dT = d5C = d5C = dA = dG = dA = MA = MG = M5C = MA = MG |
| 760 | CO-8075 | MT = M5C = MT = MG = M5C = d5C = dA = dG = dG = |
| | | d5C = dG = dT = dG = dA = dA = MG = MA = MG = MA = M5C |
| 761 | CO-8076 | MA = MT = M5C = MT = MT = d5C = d5C = dA = dA = |
| | | d5C = dT = dG = dG = dT = dT = MT = M5C = MG = MA = MG |
| 762 | CO-8077 | MT = MA = MG = MT = M5C = dT = dA = dA = dG = |
| | | d5C = d5C = dA = d5C = dT = d5C = MT = M5C = M5C = M5C = M5C |
| 763 | CO-8078 | MA = M5C = MA = MT = M5C = dT = dA = d5C = dA = |
| | | dG = dT = dG = d5C = d5C = d5C = MG = MG = MT = MA = M5C |
| 764 | CO-8079 | MT = M5C = MT = MT = M5C = d5C = d5C = d5C = d5C = |
| | | d5C = dA = dG = dT = dT = dT = MG = MA = MG = M5C = M5C |
| 765 | CO-8080 | MG = M5C = MA = MG = MT = dG = d5C = dT = dA = |
| | | dT = d5C = dA = dA = dT = dA = MT = MG = MG = MA = MT |
| 766 | CO-8081 | MT = MG = MT = M5C = M5C = d5C = dA = d5C = d5C = |
| | | dT = dT = dA = dT = d5C = dA = MG = M5C = M5C = MT = M5C |
| 767 | CO-8082 | M5C = M5C = M5C = MT = MT = dG = d5C = dT = dA = |
| | | dG = dA = dG = dG = d5C = dT = MG = MT = M5C = MT = MG |
| 768 | CO-8083 | MG = M5C = M5C = M5C = MT = dT = dA = dT = d5C = |
| | | dT = dT = dG = dA = dA = d5C = MG = MT = MG = MG = M5C |
| 769 | CO-8084 | MT = M5C = M5C = MA = MT = d5C = dT = d5C = dG = |
| | | dA = d5C = dA = dG = d5C = d5C = MA = M5C = M5C = MA = MG |
| 770 | CO-8085 | M5C = M5C = MA = MT = MG = dG = dT = d5C = dT = |
| | | d5C = dT = dA = dT = dT = d5C = MA = M5C = M5C = M5C = MA |
| 771 | CO-8086 | MA = M5C = MT = MG = MT = d5C = d5C = dT = dG = |
| | | dA = dA = d5C = dT = dG = dT = MA = MT = MG = MG = M5C |
| 772 | CO-8087 | M5C = MG = MG = MA = MA = dT = dT = d5C = d5C = |
| | | dA = d5C = dA = dA = d5C = d5C = M5C = MT = MG = M5C = MA |
| 773 | CO-8088 | MG = MA = MT = MG = M5C = dA = d5C = d5C = dA = |
| | | dG = dT = d5C = dT = dT = dA = M5C = MA = M5C = MA = M5C |
| 774 | CO-8089 | MG = MG = MG = MA = M5C = dA = d5C = d5C = dT = |
| | | dG = dT = dA = dT = dG = dA = MA = MT = MG = MT = M5C |
| 775 | CO-8090 | MG = MG = M5C = MT = MG = dT = dG = dT = d5C = |
| | | dT = d5C = dA = d5C = dT = dA = MG = MA = MA = MG = M5C |
| 776 | CO-8091 | MG = MT = MT = M5C = MA = dG = d5C = dA = dG = |
| | | d5C = dA = d5C = d5C = d5C = d5C = MT = MT = M5C = M5C = MT |
| 777 | CO-8092 | MG = MA = MG = MG = MG = dT = dG = dA = dG = |
| | | d5C = dT = dG = d5C = dA = dA = MT = MG = MT = MT = MG |
| 778 | CO-8093 | MG = M5C = MT = MG = MA = dA = dG = d5C = dT = |
| | | d5C = dG = dT = d5C = dT = dG = MG = MA = MA = M5C = MA |
| 779 | CO-8094 | MG = MT = MT = M5C = MT = dG = dT = dG = dT = |
| | | d5C = dA = dG = d5C = dT = dA = MT = M5C = MT = MA = MT |
| 780 | CO-8095 | MT = MT = MG = M5C = MA = dA = dT = d5C = dG = |
| | | dT = dG = dG = d5C = dG = d5C = MT = MT = MG = MG = MG |
| 781 | CO-8096 | M5C = MT = MA = MG = MG = dT = d5C = dT = dA = |
| | | dA = dT = d5C = d5C = d5C = dA = MG = M5C = MA = MG = M5C |
| 782 | CO-8097 | M5C = MA = MG = MT = MT = dG = dG = dT = dA = |
| | | dG = d5C = dA = d5C = dT = dT = MG = MT = MA = MG = M5C |
| 783 | CO-8098 | MA = MT = MT = MT = M5C = d5C = d5C = d5C = dT = |
| | | dT = dG = dA = dG = dG = d5C = MA = MG = MT = MG = M5C |
| 784 | CO-8099 | MA = MG = MT = M5C = MG = dT = dG = dG = dT = |
| | | dT = dT = d5C = dT = dG = dG = MG = MA = MG = MT = M5C |
| 785 | CO-8100 | MG = MG = MG = MT = MG = d5C = dG = d5C = dT = |
| | | dA = dA = dT = dG = d5C = dT = MA = MT = MG = MG = MT |
| 786 | CO-8101 | MG = M5C = M5C = MA = MT = dT = dA = dA = dG = |
| | | d5C = dT = dG = dT = dA = dG = M5C = M5C = M5C = M5C = MA |
| 787 | CO-8102 | M5C = M5C = MA = MG = MA = dA = d5C = d5C = dT = |
| | | d5C = dT = d5C = dG = dT = dG = MT = MT = MA = MG = MA |
| 788 | CO-8103 | MG = MT = MG = MA = MA = dA = dT = dA = dT = |
| | | dG = d5C = dG = d5C = dT = d5C = M5C = M5C = MT = M5C = M5C |
| 789 | CO-8104 | MG = MG = MT = MG = MG = dT = dG = dG = dT = |
| | | dA = dT = dG = dT = dG = dG = M5C = MT = MT = MT = MG |
| 790 | CO-8105 | M5C = MA = MA = MG = MA = dT = dA = dA = dA = |
| | | d5C = dA = dA = dG = dG = dT = MA = MG = MG = MG = MT |
| 791 | CO-8106 | MT = MG = MG = M5C = MA = M5C = MT = MT = MG = |
| | | M5C = MA = MA = M5C = M5C = M5C = MT = M5C = M5C = MT = MA |
| 792 | CO-8107 | MG = MG = M5C = MA = MG = MA = MT = M5C = M5C = |
| | | MT = MT = M5C = MT = MA = MT = MG = MT = MA = MG = M5C |
| 793 | CO-8108 | MG = MG = MG = MA = MT = MG = MT = MT = M5C = |
| | | MT = MT = MT = MG = MA = MA = MT = MT = MA = MG = MG |
| 794 | CO-8109 | M5C = M5C = MG = MT = MT = MG = MA = MG = MA = |
| | | MG = MG = MA = MA = M5C = MT = MG = MG = MG = MT = M5C |
| 795 | CO-8110 | MG = MT = MG = MT = MA = MG = MT = MG = MT = |
| | | MA = MG = MA = MG = MG = MT = MG = MG = M5C = M5C = MA |
| 796 | CO-8111 | MA = MA = MG = MG = M5C = MT = M5C = M5C = MA = |
| | | MG = MT = MT = M5C = MT = M5C = M5C = M5C = MT = MA = MG |
| 797 | CO-8112 | MT = MA = MG = MT = MG = MG = MA = MA = MG = MA = |
| | | MT = MG = MT = M5C = MG = MG = MA = MG = M5C = MT |
| 798 | CO-8113 | M5C = MA = MT = MT = MT = M5C = M5C = M5C = M5C = |
| | | MT = M5C = MG = MA = M5C = MA = M5C = MT = M5C = MA = MG |
| 799 | CO-8114 | M5C = MA = MT = M5C = MT = MG = MG = MA = MT = |
| | | MT = MG = MG = M5C = MT = M5C = MA = M5C = M5C = MT = MT |
| 800 | CO-8115 | MA = M5C = MT = MT = MT = MT = M5C = M5C = M5C = |
| | | M5C = M5C = MG = M5C = MT = MG = M5C = M5C = MT = M5C = MT |
| 801 | CO-8116 | MG = MG = M5C = MG = MT = MG = MA = MT = MG = |
| | | MG = M5C = MT = MG = MA = MG = MT = M5C = M5C = M5C = MT |
| 802 | CO-8117 | M5C = MT = M5C = MT = MT = MT = M5C = MG = MG = |
| | | MA = M5C = MG = M5C = MT = MG = M5C = MA = MA = MG = MT |
| 803 | CO-8118 | MG = MT = M5C = MT = M5C = M5C = MA = MG = MG = |
| | | MT = M5C = M5C = MG = MG = M5C = MT = M5C = MT = MT = MT |
| 804 | CO-8119 | MG = M5C = MT = M5C = M5C = MT = M5C = MT = M5C = |
| | | MT = MT = MG = MT = M5C = M5C = MG = MT = MA = M5C = MT |
| 805 | CO-8120 | MT = MT = M5C = MT = M5C = MT = MT = MG = M5C = |
| | | M5C = M5C = MA = MG = M5C = MA = MT = MG = MT = M5C = MG |
| 806 | CO-8121 | MG = MT = MA = MG = MA = MG = MA = M5C = MA = |
| | | M5C = M5C = MT = M5C = M5C = M5C = M5C = MG = M5C = MA = M5C |
| 807 | CO-8122 | M5C = MT = MG = MA = MT = MG = M5C = MA = M5C = |
| | | MT = MA = MA = M5C = MG = MA = MG = MG = MT = MT = MG |
| 808 | CO-8123 | MA = M5C = MA = MA = MT = M5C = MT = M5C = M5C = |
| | | M5C = M5C = MG = MG = M5C = MT = MA = M5C = MT = MG = MT |
| 809 | CO-8124 | MG = MA = MA = MG = M5C = M5C = M5C = MT = MG = |
| | | MG = MA = MT = M5C = MT = M5C = M5C = MG = MA = MG = M5C |
| 810 | CO-8125 | M5C = M5C = M5C = MG = M5C = M5C = MT = M5C = MT = |
| | | MA = M5C = MG = MT = MA = MG = MA = M5C = MA = MA = MG |
| 811 | CO-8126 | MG = MG = MA = MG = MG = MA = MA = MT = MG = |
| | | MT = MG = MA = MA = M5C = MT = MG = MA = MG = MT = MA |
| 812 | CO-8127 | M5C = MT = MG = M5C = MA = MA = M5C = MA = M5C = |
| | | MT = M5C = MT = MG = M5C = M5C = MG = M5C = M5C = MA = MG |
| 813 | CO-8128 | M5C = M5C = MT = M5C = M5C = MA = MG = MT = MA = |
| | | MA = MA = M5C = MG = MA = M5C = M5C = MT = MT = MG = MA |
| 814 | CO-8129 | MA = MA = MG = MT = M5C = M5C = M5C = MG = MA = |
| | | MT = M5C = M5C = MA = MG = MA = MA = MG = M5C = MA = MG |
| 815 | CO-8130 | MT = M5C = MT = MG = M5C = M5C = MA = MG = MG = |
| | | M5C = MG = MT = MG = MA = MA = MG = MA = MG = MA = M5C |
| 816 | CO-8131 | MA = MT = M5C = MT = MT = M5C = M5C = MA = MA = |
| | | M5C = MT = MG = MG = MT = MT = MT = M5C = MG = MA = MG |
| 817 | CO-8132 | MT = MA = MG = MT = M5C = MT = MA = MA = MG = |
| | | M5C = M5C = MA = M5C = MT = M5C = MT = M5C = M5C = M5C = M5C |
| 818 | CO-8133 | MA = M5C = MA = MT = M5C = MT = MA = M5C = MA = |
| | | MG = MT = MG = M5C = M5C = M5C = MG = MG = MT = MA = M5C |
| 819 | CO-8134 | MT = M5C = MT = MT = M5C = M5C = M5C = M5C = M5C = |
| | | M5C = MA = MG = MT = MT = MT = MG = MA = MG = M5C = M5C |
| 820 | CO-8135 | MG = M5C = MA = MG = MT = MG = M5C = MT = MA = |
| | | MT = M5C = MA = MA = MT = MA = MT = MG = MG = MA = MT |
| 821 | CO-8136 | MT = MG = MT = M5C = M5C = M5C = MA = M5C = M5C = |
| | | MT = MT = MA = MT = M5C = MA = MG = M5C = M5C = MT = M5C |
| 822 | CO-8137 | M5C = M5C = M5C = MT = MT = MG = M5C = MT = MA = |
| | | MG = MA = MG = MG = M5C = MT = MG = MT = M5C = MT = MG |
| 823 | CO-8138 | MG = M5C = M5C = M5C = MT = MT = MA = MT = M5C = |
| | | MT = MT = MG = MA = MA = M5C = MG = MT = MG = MG = M5C |
| 824 | CO-8139 | MT = M5C = M5C = MA = MT = M5C = MT = M5C = MG = |
| | | MA = M5C = MA = MG = M5C = M5C = MA = M5C = M5C = MA = MG |
| 825 | CO-8140 | M5C = M5C = MA = MT = MG = MG = MT = M5C = MT = |
| | | M5C = MT = MA = MT = MT = M5C = MA = M5C = M5C = M5C = MA |
| 826 | CO-8141 | MA = M5C = MT = MG = MT = M5C = M5C = MT = MG = |
| | | MA = MA = M5C = MT = MG = MT = MA = MT = MG = MG = M5C |
| 827 | CO-8142 | M5C = MG = MG = MA = MA = MT = MT = M5C = M5C = |
| | | MA = M5C = MA = MA = M5C = M5C = M5C = MT = MG = M5C = MA |
| 828 | CO-8143 | MG = MA = MT = MG = M5C = MA = M5C = M5C = MA = |
| | | MG = MT = M5C = MT = MT = MA = M5C = MA = M5C = MA = M5C |
| 829 | CO-8144 | MG = MG = MG = MA = M5C = MA = M5C = M5C = MT = |
| | | MG = MT = MA = MT = MG = MA = MA = MT = MG = MT = M5C |
| 830 | CO-8145 | MG = MG = M5C = MT = MG = MT = MG = MT = M5C = |
| | | MT = M5C = MA = M5C = MT = MA = MG = MA = MA = MG = M5C |
| 831 | CO-8146 | MG = MT = MT = M5C = MA = MG = M5C = MA = MG = |
| | | M5C = MA = M5C = M5C = M5C = M5C = MT = MT = M5C = M5C = MT |
| 832 | CO-8147 | MG = MA = MG = MG = MG = MT = MG = MA = MG = |
| | | M5C = MT = MG = M5C = MA = MA = MT = MG = MT = MT = MG |
| 833 | CO-8148 | MG = M5C = MT = MG = MA = MA = MG = M5C = MT = |
| | | M5C = MG = MT = M5C = MT = MG = MG = MA = MA = M5C = MA |
| 834 | CO-8149 | MG = MT = MT = M5C = MT = MG = MT = MG = MT = |
| | | M5C = MA = MG = M5C = MT = MA = MT = M5C = MT = MA = MT |
| 835 | CO-8150 | MT = MT = MG = M5C = MA = MA = MT = M5C = MG = |
| | | MT = MG = MG = M5C = MG = M5C = MT = MT = MG = MG = MG |
| 836 | CO-8151 | M5C = MT = MA = MG = MG = MT = M5C = MT = MA = |
| | | MA = MT = M5C = M5C = M5C = MA = MG = M5C = MA = MG = M5C |
| 837 | CO-8152 | M5C = MA = MG = MT = MT = MG = MG = MT = MA = |
| | | MG = M5C = MA = M5C = MT = MT = MG = MT = MA = MG = M5C |
| 838 | CO-8153 | MA = MT = MT = MT = M5C = M5C = M5C = M5C = MT = |
| | | MT = MG = MA = MG = MG = M5C = MA = MG = MT = MG = M5C |
| 839 | CO-8154 | MA = MG = MT = M5C = MG = MT = MG = MG = MT = |
| | | MT = MT = M5C = MT = MG = MG = MG = MA = MG = MT = M5C |
| 840 | CO-8155 | MG = MG = MG = MT = MG = M5C = MG = M5C = MT = |
| | | MA = MA = MT = MG = M5C = MT = MA = MT = MG = MG = MT |
| 841 | CO-8156 | MG = M5C = M5C = MA = MT = MT = MA = MA = MG = |
| | | M5C = MT = MG = MT = MA = MG = M5C = M5C = M5C = M5C = MA |
| 842 | CO-8157 | M5C = M5C = MA = MG = MA = MA = M5C = M5C = MT = |
| | | M5C = MT = M5C = MG = MT = MG = MT = MT = MA = MG = MA |
| 843 | CO-8158 | MG = MT = MG = MA = MA = MA = MT = MA = MT = |
| | | MG = M5C = MG = M5C = MT = M5C = M5C = M5C = MT = M5C = M5C |
| 844 | CO-8159 | MG = MG = MT = MG = MG = MT = MG = MG = MT = |
| | | MA = MT = MG = MT = MG = MG = M5C = MT = MT = MT = MG |
| 845 | CO-8160 | M5C = MA = MA = MG = MA = MT = MA = MA = MA = |
| | | M5C = MA = MA = MG = MG = MT = MA = MG = MG = MG = MT |
| 846 | CO-10673 | MG = MT = M5C = MT = M5C = d5C = dA = MG = dG = |
| | | dT = M5C = d5C = dG = MG = d5C = MT = M5C = MT = MT = MT |
| 847 | CO-10674 | MG = MT = L5C = MT = M5C = d5C = dA = dG = dG = |
| | | dT = d5C = d5C = dG = dG = d5C = MT = M5C = LT = MT = MT |
| 848 | CO-10675 | MG = MT = L5C = MT = L5C = d5C = dA = dG = dG = |
| | | dT = d5C = d5C = dG = dG = d5C = LT = M5C = LT = MT = MT |
| 849 | CO-10676 | MG = LT = M5C = LT = M5C = d5C = dA = dG = dG = |
| | | dT = d5C = d5C = dG = dG = d5C = MT = L5C = MT = LT = MT |
| 850 | CO-10677 | MG = LT = M5C = LT = M5C = d5C = dA = MG = |
| | | dG = dT = M5C = d5C = dG = MG = d5C = MT = L5C = MT = LT = MT |
| 851 | CO-10678 | LG = MT = L5C = MT = M5C = d5C = dA = dG = |
| | | dG = dT = d5C = d5C = dG = dG = d5C = MT = M5C = LT = MT = LT |
| 852 | CO-10679 | MG = MT = M5C-MT-M5C = d5C = dA = dG = dG = |
| | | dT = d5C = d5C = dG = dG = d5C = MT-M5C-MT = MT = MT |
| 853 | CO-10680 | MG = MT-M5C-MT-M5C = d5C = dA = dG = dG = |
| | | dT = d5C = d5C = dG = dG = d5C = MT-M5C-MT-MT = MT |
| 854 | CO-10681 | MT = MG = MT = M5C = MT = M5C = d5C = dA = dG = |
| | | dG = dT = d5C = d5C = dG = dG = d5C = MT = M5C = MT = MT = MT = M5C |
| 855 | CO-10683 | M5C = M5C = MG = MG = M5C = dT = d5C = dT = |
| | | dT = dT = d5C = dT = d5C = dT = dT = MT = M5C = MG = MG = MA |
| 856 | CO-10684 | MG = MT = M5C = M5C = MG = dG = d5C = dT = |
| | | d5C = dT = dT = dT = d5C = dT = d5C = MT = MT = MT = M5C = MG |
| 857 | CO-10685 | MA = MG = MG = MT = M5C = d5C = dG = dG = d5C = |
| | | dT = d5C = dT = dT = dT = d5C = MT = M5C = MT = MT = MT |
| 858 | CO-10686 | M5C = M5C = MA = MG = MG = dT = d5C = d5C = dG = |
| | | dG = d5C = dT = d5C = dT = dT = MT = M5C = MT = M5C = MT |
| 859 | CO-10687 | M5C = MT = M5C = M5C = MA = dG = dG = dT = d5C = |
| | | d5C = dG = dG = d5C = dT = d5C = MT = MT = MT = M5C = MT |
| 860 | CO-10688 | MT = MG = MT = M5C = MT = d5C = d5C = dA = dG = |
| | | dG = dT = d5C = d5C = dG = dG = M5C = MT = M5C = MT = MT |
| 861 | CO-10689 | MG = MT = MA = MG = MA = dG = dA = M5C = dA = |
| | | d5C = M5C = dT = d5C = M5C = d5C = M5C = MG = M5C = MA = M5C |
| 862 | CO-10690 | MG = MT = LA = MG = MA = dG = dA = d5C = dA = |
| | | d5C = d5C = dT = d5C = d5C = d5C = M5C = MG = L5C = MA = M5C |
| 863 | CO-10691 | MG = MT = LA = MG = LA = dG = dA = d5C = dA = |
| | | d5C = d5C = dT = d5C = d5C = d5C = L5C = MG = L5C = MA = M5C |
| 864 | CO-10692 | MG = LT = MA = LG = MA = dG = dA = d5C = dA = |
| | | d5C = d5C = dT = d5C = d5C = d5C = M5C = LG = M5C = LA = M5C |
| 865 | CO-10693 | MG = LT = MA = LG = MA = dG = dA = M5C = dA = |
| | | d5C = M5C = dT = d5C = M5C = d5C = M5C = LG = M5C = LA = M5C |
| 866 | CO-10694 | LG = MT = LA = MG = MA = dG = dA = d5C = dA = |
| | | d5C = d5C = dT = d5C = d5C = d5C = M5C = MG = L5C = MA = L5C |
| 867 | CO-10695 | MG = MT = MA-MG-MA = dG = dA = d5C = dA = |
| | | d5C = d5C = dT = d5C = d5C = d5C = M5C-MG-M5C = MA = M5C |
| 868 | CO-10696 | MG = MT-MA-MG-MA = dG = dA = d5C = dA = d5C = |
| | | d5C = dT = d5C = d5C = d5C = M5C-MG-M5C-MA = M5C |
| 869 | CO-10697 | MT = MG = MT = MA = MG = MA = dG = dA = d5C = |
| | | dA = d5C = d5C = dT = d5C = d5C = d5C = M5C = MG = M5C = MA = M5C = M5C |
| 870 | CO-10699 | MT = MA = MG = MA = MG = dA = d5C = dA = |
| | | d5C = d5C = dT = d5C = d5C = d5C = d5C = MG = M5C = MA = M5C = M5C |
| 871 | CO-10700 | MT = MT = MG = MT = MA = dG = dA = dG = dA = |
| | | d5C = dA = d5C = d5C = dT = d5C = M5C = M5C = M5C = MG = M5C |
| 872 | CO-10701 | MT = MT = MG = MT = MT = dG = dT = dA = dG = |
| | | dA = dG = dA = d5C = dA = d5C = M5C = MT = M5C = M5C = M5C |
| 873 | CO-10702 | MA = MG = MT = MT = MG = dT = dT = dG = dT = |
| | | dA = dG = dA = dG = dA = d5C = MA = M5C = M5C = MT = M5C |
| 874 | CO-10703 | MG = M5C = MA = MG = MT = dT = dG = dT = dT = |
| | | dG = dT = dA = dG = dA = dG = MA = M5C = MA = M5C = M5C |
| 875 | CO-10704 | MA = M5C = MA = MT = M5C = dT = dA = M5C = dA = |
| | | dG = MT = dG = d5C = M5C = d5C = MG = MG = MT = MA = M5C |
| 876 | CO-10705 | MA = M5C = LA = MT = M5C = dT = dA = d5C = dA = |
| | | dG = dT = dG = d5C = d5C = d5C = MG = MG = LT = MA = M5C |
| 877 | CO-10706 | MA = M5C = LA = MT = L5C = dT = dA = d5C = dA = |
| | | dG = dT = dG = d5C = d5C = d5C = LG = MG = LT = MA = M5C |
| 878 | CO-10707 | MA = L5C = MA = LT = M5C = dT = dA = d5C = dA = |
| | | dG = dT = dG = d5C = d5C = d5C = MG = LG = MT = LA = M5C |
| 879 | CO-10708 | MA = L5C = MA = LT = M5C = dT = dA = M5C = dA = |
| | | dG = MT = dG = d5C = M5C = d5C = MG = LG = MT = LA = M5C |
| 880 | CO-10709 | LA = M5C = LA = MT = M5C = dT = dA = d5C = dA = |
| | | dG = dT = dG = d5C = d5C = d5C = MG = MG = LT = MA = L5C |
| 881 | CO-10710 | MA = M5C = MA-MT-M5C = dT = dA = d5C = dA = |
| | | dG = dT = dG = d5C = d5C = d5C = MG-MG-MT = MA = M5C |
| 882 | CO-10711 | MA = M5C-MA-MT-M5C = dT = dA = d5C = dA = |
| | | dG = dT = dG = d5C = d5C = d5C = MG-MG-MT-MA = M5C |
| 883 | CO-10712 | M5C = MA = M5C = MA = MT = M5C = dT = dA = d5C = |
| | | dA = dG = dT = dG = d5C = d5C = d5C = MG = MG = MT = MA = M5C = MT |
| 884 | CO-10714 | M5C = MT = MA = M5C = MA = dG = dT = dG = |
| | | d5C = d5C = d5C = dG = dG = dT = dA = M5C = MT = M5C = MG = M5C |
| 885 | CO-10715 | MA = MT = M5C = MT = MA = d5C = dA = dG = |
| | | dT = dG = d5C = d5C = d5C = dG = dG = MT = MA = M5C = MT = M5C |
| 886 | CO-10716 | MA = M5C = MA = M5C = MA = dT = d5C = dT = |
| | | dA = d5C = dA = dG = dT = dG = d5C = M5C = M5C = MG = MG = MT |
| 887 | CO-10717 | MT = MA = M5C = MA = M5C = dA = d5C = dA = |
| | | dT = d5C = dT = dA = d5C = dA = dG = MT = MG = M5C = M5C = M5C |
| 888 | CO-10718 | M5C = M5C = MT = MA = M5C = dA = d5C = dA = |
| | | d5C = dA = dT = d5C = dT = dA = d5C = MA = MG = MT = MG = M5C |
| 889 | CO-10719 | MT = M5C = M5C = M5C = MT = dA = d5C = dA = |
| | | d5C = dA = d5C = dA = dT = d5C = dT = MA = M5C = MA = MG = MT |
| 890 | CO-10720 | MA = M5C = MT = MG = MT = d5C = d5C = MT = |
| | | dG = dA = MA = d5C = dT = MG = dT = MA = MT = MG = MG = M5C |
| 891 | CO-10721 | MA = M5C = LT = MG = MT = d5C = d5C = dT = |
| | | dG = dA = dA = d5C = dT = dG = dT = MA = MT = LG = MG = M5C |
| 892 | CO-10722 | MA = M5C = LT = MG = LT = d5C = d5C = dT = |
| | | dG = dA = dA = d5C = dT = dG = dT = LA = MT = LG = MG = M5C |
| 893 | CO-10723 | MA = L5C = MT = LG = MT = d5C = d5C = dT = dG = |
| | | dA = dA = d5C = dT = dG = dT = MA = LT = MG = LG = M5C |
| 894 | CO-10724 | MA = L5C = MT = LG = MT = d5C = d5C = MT = dG = |
| | | dA = MA = d5C = dT = MG = dT = MA = LT = MG = LG = M5C |
| 895 | CO-10725 | LA = M5C = LT = MG = MT = d5C = d5C = dT = dG = |
| | | dA = dA = d5C = dT = dG = dT = MA = MT = LG = MG = L5C |
| 896 | CO-10726 | MA = M5C = MT-MG-MT = d5C = d5C = dT = dG = |
| | | dA = dA = d5C = dT = dG = dT = MA-MT-MG = MG = M5C |
| 897 | CO-10727 | MA = M5C-MT-MG-MT = d5C = d5C = dT = dG = |
| | | dA = dA = d5C = dT = dG = dT = MA-MT-MG-MG = M5C |
| 898 | CO-10728 | M5C = MA = M5C = MT = MG = MT = d5C = d5C = |
| | | dT = dG = dA = dA = d5C = dT = dG = dT = MA = MT = MG = MG = M5C = MT |
| 899 | CO-10730 | MT = MG = MT = M5C = M5C = dT = dG = dA = dA = |
| | | d5C = dT = dG = dT = dA = dT = MG = MG = M5C = MT = MG |
| 900 | CO-10731 | M5C = MA = M5C = MT = MG = dT = d5C = d5C = |
| | | dT = dG = dA = dA = d5C = dT = dG = MT = MA = MT = MG = MG |
| 901 | CO-10732 | MT = M5C = M5C = M5C = MA = d5C = dT = dG = |
| | | dT = d5C = d5C = dT = dG = dA = dA = M5C = MT = MG = MT = MA |
| 902 | CO-10733 | M5C = MT = M5C = M5C = M5C = dA = d5C = dT = |
| | | dG = dT = d5C = d5C = dT = dG = dA = MA = M5C = MT = MG = MT |
| 903 | CO-10734 | MA = MG = MA = M5C = MT = d5C = d5C = d5C = dA = |
| | | d5C = dT = dG = dT = d5C = d5C = MT = MG = MA = MA = M5C |
| 904 | CO-10735 | MG = MT = MA = MA = MG = dA = d5C = dT = d5C = |
| | | d5C = d5C = dA = d5C = dT = dG = MT = M5C = M5C = MT = MG |
| 905 | CO-10736 | MG = MT = MG = MA = MA = dA = dT = MA = dT = |
| | | dG = M5C = dG = d5C = MT = d5C = M5C = M5C = MT = M5C = M5C |
| 906 | CO-10737 | MG = MT = LG = MA = MA = dA = dT = dA = dT = |
| | | dG = d5C = dG = d5C = dT = d5C = M5C = M5C = LT = M5C = M5C |
| 907 | CO-10738 | MG = MT = LG = MA = LA = dA = dT = dA = dT = |
| | | dG = d5C = dG = d5C = dT = d5C = L5C = M5C = LT = M5C = M5C |
| 908 | CO-10739 | MG = LT = MG = LA = MA = dA = dT = dA = dT = |
| | | dG = d5C = dG = d5C = dT = d5C = M5C = L5C = MT = L5C = M5C |
| 909 | CO-10740 | MG = LT = MG = LA = MA = dA = dT = MA = dT = |
| | | dG = M5C = dG = d5C = MT = d5C = M5C = L5C = MT = L5C = M5C |
| 910 | CO-10741 | LG = MT = LG = MA = MA = dA = dT = dA = dT = |
| | | dG = d5C = dG = d5C = dT = d5C = M5C = M5C = LT = M5C = L5C |
| 911 | CO-10742 | MG = MT = MG-MA-MA = dA = dT = dA = dT = dG = |
| | | d5C = dG = d5C = dT = d5C = M5C-M5C-MT = M5C = M5C |
| 912 | CO-10743 | MG = MT-MG-MA-MA = dA = dT = dA = dT = dG = |
| | | d5C = dG = d5C = dT = d5C = M5C-M5C-MT-M5C = M5C |
| 913 | CO-10744 | MT = MG = MT = MG = MA = MA = dA = dT = dA = |
| | | dT = dG = d5C = dG = d5C = dT = d5C = M5C = M5C = MT = M5C = M5C = MT |
| 914 | CO-10746 | MT = MA = MT = MG = M5C = dG = d5C = dT = d5C = |
| | | d5C = d5C = dT = d5C = d5C = dT = MT = M5C = MA = MG = MA |
| 915 | CO-10747 | MA = MA = MT = MA = MT = dG = d5C = dG = d5C = |
| | | dT = d5C = d5C = d5C = dT = d5C = M5C = MT = MT = M5C = MA |
| 916 | CO-10748 | MG = MA = MA = MA = MT = dA = dT = dG = d5C = |
| | | dG = d5C = dT = d5C = d5C = d5C = MT = M5C = M5C = MT = MT |
| 917 | CO-10749 | M5C = MT = MG = MT = MG = dA = dA = dA = dT = |
| | | dA = dT = dG = d5C = dG = d5C = MT = M5C = M5C = M5C = MT |
| 918 | CO-10750 | MT = MT = M5C = MT = MG = dT = dG = dA = dA = |
| | | dA = dT = dA = dT = dG = d5C = MG = M5C = MT = M5C = M5C |
| 919 | CO-10751 | MT = MG = MT = MT = M5C = dT = dG = dT = dG = |
| | | dA = dA = dA = dT = dA = dT = MG = M5C = MG = M5C = MT |
| 920 | CO-10752 | MG = MT = MT = MG = MT = dT = d5C = dT = dG = |
| | | dT = dG = dA = dA = dA = dT = MA = MT = MG = M5C = MG |
| 921 | CO-10753 | MT = MA = LG = MT = MG = LG = MA = MA = LG = |
| | | MA = MT = LG = MT = M5C = LG = MG = MA = LG = M5C = MT |
| 922 | CO-10754 | MT = MA = MG = MT = LG = MG = MA = MA = LG = |
| | | MA = MT = MG = LT = M5C = MG = MG = LA = MG = M5C = MT |
| 923 | CO-10755 | MT = MA = MG = LT = MG = MG = MA = LA = MG = |
| | | MA = MT = LG = MT = M5C = MG = LG = MA = MG = M5C = MT |
| 924 | CO-10756 | MT = LA = MG = MT = LG = MG = MA = LA = MG = |
| | | MA = LT = MG = MT = L5C = MG = MG = LA = MG = M5C = LT |
| 925 | CO-10757 | LT = MA = MG = LT = MG = MG = LA = MA = MG = |
| | | LA = MT = MG = LT = M5C = MG = LG = MA = MG = L5C = MT |
| 926 | CO-10758 | MT = MA = MG = MT = MG = dG = dA = MA = dG = |
| | | dA = MT = dG = dT = M5C = dG = MG = MA = MG = M5C = MT |
| 927 | CO-10759 | MT = MA = MG-MT-MG = MG = MA = MA = MG = |
| | | MA = MT = MG = MT = M5C = MG = MG-MA-MG = M5C = MT |
| 928 | CO-10760 | MT = MA-MG-MT-MG = MG = MA = MA = MG = MA = |
| | | MT = MG = MT = M5C = MG = MG-MA-MG-M5C = MT |
| 929 | CO-10761 | M5C = MT = MA = MG = MT = MG = MG = MA = MA = MG = |
| | | MA = MT = MG = MT = M5C = MG = MG = MA = MG = M5C = MT = MA |
| 930 | CO-10762 | MG = MG = MA = MA = MG = MA = MT = MG = MT = |
| | | M5C = MG = MG = MA = MG = M5C = MT = MA = MG = MG = MG |
| 931 | CO-10763 | MG = MT = MG = MG = MA = MA = MG = MA = MT = |
| | | MG = MT = M5C = MG = MG = MA = MG = M5C = MT = MA = MG |
| 932 | CO-10764 | M5C = M5C = MT = MA = MG = MT = MG = MG = MA = |
| | | MA = MG = MA = MT = MG = MT = M5C = MG = MG = MA = MG |
| 933 | CO-10765 | M5C = MA = M5C = M5C = MT = MA = MG = MT = MG = |
| | | MG = MA = MA = MG = MA = MT = MG = MT = M5C = MG = MG |
| 934 | CO-10766 | MG = M5C = LT = M5C = M5C = LT = M5C = MT = L5C = |
| | | MT = MT = LG = MT = M5C = L5C = MG = MT = LA = M5C = MT |
| 935 | CO-10767 | MG = M5C = MT = M5C = L5C = MT = M5C = MT = L5C = |
| | | MT = MT = MG = LT = M5C = M5C = MG = LT = MA = M5C = MT |
| 936 | CO-10768 | MG = M5C = MT = L5C = M5C = MT = M5C = LT = M5C = |
| | | MT = MT = LG = MT = M5C = M5C = LG = MT = MA = M5C = MT |
| 937 | CO-10769 | MG = L5C = MT = M5C = L5C = MT = M5C = LT = M5C = |
| | | MT = LT = MG = MT = L5C = M5C = MG = LT = MA = M5C = LT |
| 938 | CO-10770 | LG = M5C = MT = L5C = M5C = MT = L5C = MT = M5C = |
| | | LT = MT = MG = LT = M5C = M5C = LG = MT = MA = L5C = MT |
| 939 | CO-10771 | MG = M5C = MT = M5C = M5C = dT = d5C = MT = d5C = |
| | | dT = MT = dG = dT = M5C = d5C = MG = MT = MA = M5C = MT |
| 940 | CO-10772 | MG = M5C = MT-M5C-M5C = MT = M5C = MT = M5C = |
| | | MT = MT = MG = MT = M5C = M5C = MG-MT-MA = M5C = MT |
| 941 | CO-10773 | MG = M5C-MT-M5C-M5C = MT = M5C = MT = M5C = |
| | | MT = MT = MG = MT = M5C = M5C = MG-MT-MA-M5C = MT |
| 942 | CO-10774 | MA = MG = M5C = MT = M5C = M5C = MT = M5C = MT = |
| | | M5C = MT = MT = MG = MT = M5C = M5C = MG = MT = MA = |
| | | M5C = MT = MT |
| 943 | CO-10775 | M5C = MT = M5C = MT = M5C = MT = MT = MG = MT = |
| | | M5C = M5C = MG = MT = MA = M5C = MT = MT = M5C = M5C = M5C |
| 944 | CO-10776 | MT = M5C = M5C = MT = M5C = MT = M5C = MT = MT = |
| | | MG = MT = M5C = M5C = MG = MT = MA = M5C = MT = MT = M5C |
| 945 | CO-10777 | M5C = MA = MG = M5C = MT = M5C = M5C = MT = M5C = |
| | | MT = M5C = MT = MT = MG = MT = M5C = M5C = MG = MT = MA |
| 946 | CO-10778 | MG = M5C = M5C = MA = MG = M5C = MT = M5C = M5C = |
| | | MT = M5C = MT = M5C = MT = MT = MG = MT = M5C = M5C = MG |
| 947 | CO-10779 | MA = MG = MG = MA = MA = M5C = M5C = MA = M5C = |
| | | M5C = M5C = M5C = MA = M5C = MT = M5C = MT = MT = MG = MG |
| 948 | CO-10780 | M5C = M5C = L5C = MG = M5C = L5C = MT = M5C = LT = |
| | | MA = M5C = LG = MT = MA = LG = MA = M5C = LA = MA = MG |
| 949 | CO-10781 | M5C = M5C = M5C = MG = L5C = M5C = MT = M5C = LT = |
| | | MA = M5C = MG = LT = MA = MG = MA = L5C = MA = MA = MG |
| 950 | CO-10782 | M5C = M5C = M5C = LG = M5C = M5C = MT = L5C = MT = |
| | | MA = M5C = LG = MT = MA = MG = LA = M5C = MA = MA = MG |
| 951 | CO-10783 | M5C = L5C = M5C = MG = L5C = M5C = MT = L5C = MT = |
| | | MA = L5C = MG = MT = LA = MG = MA = L5C = MA = MA = LG |
| 952 | CO-10784 | L5C = M5C = M5C = LG = M5C = M5C = LT = M5C = MT = |
| | | LA = M5C = MG = LT = MA = MG = LA = M5C = MA = LA = MG |
| 953 | CO-10785 | M5C = M5C = M5C = MG = M5C = d5C = dT = M5C = |
| | | dT = dA = M5C = dG = dT = MA = dG = MA = M5C = MA = MA = MG |
| 954 | CO-10786 | M5C = M5C = M5C-MG-M5C = M5C = MT = M5C = MT = |
| | | MA = M5C = MG = MT = MA = MG = MA-M5C-MA = MA = MG |
| 955 | CO-10787 | M5C = M5C-M5C-MG-M5C = M5C = MT = M5C = MT = |
| | | MA = M5C = MG = MT = MA = MG = MA-M5C-MA-MA = MG |
| 956 | CO-10788 | MT = M5C = M5C = M5C = MG = M5C = M5C = MT = M5C = MT = |
| | | MA = M5C = MG = MT = MA = MG = MA = M5C = MA = MA = MG = |
| | | MT |
| 957 | CO-10789 | MT = M5C = MT = MA = M5C = MG = MT = MA = MG = MA = |
| | | M5C = MA = MA = MG = MT = MA = MA = M5C = M5C = MA |
| 958 | CO-10790 | M5C = M5C = MT = M5C = MT = MA = M5C = MG = MT = |
| | | MA = MG = MA = M5C = MA = MA = MG = MT = MA = MA = M5C |
| 959 | CO-10791 | M5C = MG = M5C = M5C = MT = M5C = MT = MA = M5C = |
| | | MG = MT = MA = MG = MA = M5C = MA = MA = MG = MT = MA |
| 960 | CO-10792 | MG = MT = M5C = M5C = M5C = MG = M5C = M5C = MT = |
| | | M5C = MT = MA = M5C = MG = MT = MA = MG = MA = M5C = MA |
| 961 | CO-10793 | MG = MA = MG = MT = M5C = M5C = M5C = MG = M5C = |
| | | M5C = MT = M5C = MT = MA = M5C = MG = MT = MA = MG = MA |
| 962 | CO-10794 | MG = MA = MG = MA = MG = MT = M5C = M5C = M5C = |
| | | MG = M5C = M5C = MT = M5C = MT = MA = M5C = MG = MT = MA |

In some embodiments, the ASO comprises a sequence and/or chemical modification selected from a sequence provided in any one of SEQ ID NOs: 10-4916. In some embodiments, the ASO comprises a sequence selected from a sequence provided in any one of SEQ ID NOs: 1369-4738. In some embodiments, the ASO comprises a sequence and chemical modification selected from a sequence provided in any one of SEQ ID NOs: 10-1368, or 4734-4916. In some embodiments, the ASO comprises a sequence and chemical modification selected from a sequence provided in any one of SEQ ID NOs: 10-442, 691, 991-1368, or 4743-4915. In some embodiments, the ASO comprises a sequence and chemical modification selected from a sequence provided in any one of SEQ ID NOs: 443-690, 692-990, or 4916.

### High Affinity Modified Nucleotides

A high affinity modified nucleotide is a modified nucleotide which, when incorporated into the oligonucleotide enhances the affinity of the oligonucleotide for its complementary target, for example as measured by the melting temperature (Tₘ). A high affinity modified nucleotide of the present invention preferably result in an increase in melting temperature between +0.5 to +12° C., such as between +1.5 to +10° C. or +3 to +8° C. per modified nucleotide. Numerous high affinity modified nucleotides are known in the art and include for example, many 2′ substituted nucleotides as well as locked nucleic acids (LNA) (see e.g., Freier & Altmann (1997) Nucl. Acid Res. 25: 4429-43 and Uhlmann (2000) Curr. Opinion in Drug Development 3(2): 203-213, each of which are hereby incorporated by reference).

### Sugar Modifications

The ASOs described herein may comprise one or more nucleotides which have a modified sugar moiety, i.e., a modification of the sugar moiety when compared to the ribose sugar moiety found in DNA and RNA. Numerous nucleotides with modification of the ribose sugar moiety have been made, primarily with the aim of improving certain properties of oligonucleotides, such as affinity and/or nuclease resistance. Such modifications include those where the ribose ring structure is modified, e.g., by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g., UNA). Other sugar modified nucleotides include, for example, bicyclohexose nucleic acids (WO2011/017521) or tricyclic nucleic acids (WO2013/154798), both of which are hereby incorporated by reference. Modified nucleotides also include nucleotides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′ or 5′ positions.

In some embodiments, oligonucleotides comprise modified sugar moieties, such as any one of a 2′-O-methyl (2′OMe) moiety, a 2′-O-methoxyethyl moiety, a bicyclic sugar moiety, PNA (e.g., an oligonucleotide comprising one or more N-(2-aminoethyl)-glycine units linked by amide bonds or carbonyl methylene linkage as repeating units in place of a sugar-phosphate backbone), locked nucleotide (LNA) (e.g., an oligonucleotide comprising one or more locked ribose, and can be a mixture of 2′-deoxy nucleotides or 2′Ome nucleotides), cET (e.g., an oligonucleotide comprising one or more cET sugar), cMOE (e.g., an oligonucleotide comprising one or more cMOE sugar), morpholino oligomer (e.g., an oligonucleotide comprising a backbone comprising one or more phosphorodiamidate morpholiono oligomers), 2′-deoxy-2′-fluoro nucleotide (e.g., an oligonucleotide comprising one or more 2′-fluoro-β-D-arabinonucleotide), tcDNA (e.g., an oligonucleotide comprising one or more tcDNA modified sugar), constrained ethyl 2′-4′-bridged nucleic acid (cEt), S-cEt, ethylene bridged nucleic acid (ENA) (e.g., an oligonucleotide comprising one or more ENA modified sugar), hexitol nucleic acids (HNA) (e.g., an oligonucleotide comprising one or more HNA modified sugar), or tricyclic analog (tcDNA) (e.g., an oligonucleotide comprising one or more tcDNA modified sugar).

In some embodiments, oligonucleotides comprise nucleobase modifications selected from the group consisting of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, unstructured nucleic acid (“UNA”), isoguanine (“isoG”), and isocytosine (“isoC”), glycerol nucleic acid (GNA), thiomorpholino (C₄H9NS) or thiophosphoramidate morpholinos (TMOs). Synthesis of glycerol nucleic acid (GNA) (also known as glycol nucleic acids) is described in Zhang et al, Current Protocols in Nucleic Acid Chemistry 4.40.1-4.40.18, September 2010, hereby incorporated by reference. Synthesis of thiophosphoramidate morpholino oligonucleotides is described in Langer et al. J. Am. Chem. Soc. 2020, 142(38): 16240-253

### 2′ Sugar Modified Nucleotides

A 2′ sugar modified nucleotide is a nucleotide which has a substituent other than H or —OH at the 2′ position (2′ substituted nucleotide) or comprises a 2′ linked biradicle capable of forming a bridge between the 2′ carbon and a second carbon in the ribose ring, such as LNA (2′-4′ biradicle bridged) nucleotides.

Without wishing to be bound by theory, the 2′ modified sugar may provide enhanced binding affinity and/or increased nuclease resistance to the oligonucleotide. Examples of 2′ substituted modified nucleotides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-FANA nucleotide. For further examples, see e.g., Freier & Altmann 1997, supra; Uhlmann 2000, supra, and Deleavey and Damha (2012) Chemistry and Biology 19: 937, each of which are hereby incorporated by reference.

### Locked Nucleic Acid Nucleotides (LNA Nucleotide)

A “LNA nucleotide” is a 2′-sugar modified nucleotide which comprises a biradical linking the C2′ and C4′ of the ribose sugar ring of said nucleotide (also referred to as a “2′-4′ bridge”), which restricts or locks the conformation of the ribose ring. In other words, a locked nucleotide is a nucleotide comprising a bicyclic sugar moiety comprising a 4′-CH₂—O-2′ bridge. This structure effectively “locks” the ribose in the 3′-endo structural conformation. The addition of locked nucleotides to oligonucleotides has been shown to increase oligonucleotide stability in serum, and to reduce off-target effects (Grunweller, A. et al., (2003) Nucleic Acids Research 31(12):3185-3193). These nucleotides are also sometimes termed bridged nucleic acid or bicyclic nucleic acid (BNA). The locking of the conformation of the ribose is associated with an enhanced affinity of hybridization (duplex stabilization) when the LNA is incorporated into an oligonucleotide with complementarity to an RNA or a DNA molecule. This can be routinely determined by measuring the melting temperature of the oligonucleotide/complement duplex. Exemplary LNA nucleotides include beta-D-oxy-LNA, 6′-methyl-beta-D-oxy LNA such as (S)-6′-methyl-beta-D-oxy-LNA (ScET) and ENA.

Examples of bicyclic nucleotides for use in the polynucleotides of the disclosure include without limitation nucleotides comprising a bridge between the 4′ and the 2′ ribosyl ring atoms. In certain embodiments, the polynucleotide agents of the disclosure include one or more bicyclic nucleotides comprising a 4′ to 2′ bridge. Examples of such 4′ to 2′ bridged bicyclic nucleotides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof, see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof, see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleotides can be prepared having one or more stereochemical sugar configurations including for example α-L-ribofuranose and 3-D-ribofuranose (see PCT Application Publication No. WO 99/14226, contents of which are incorporated by reference herein).

An oligonucleotide of the disclosure can also be modified to include one or more constrained ethyl nucleotides. As used herein, a “constrained ethyl nucleotide” or “cEt” is a locked nucleotide comprising a bicyclic sugar moiety comprising a 4′-CH(CH₃)—O-2′ bridge. In one embodiment, a constrained ethyl nucleotide is in the S conformation referred to herein as “S-cEt.”

An oligonucleotide of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to an RNA (e.g., a regRNA or a mRNA). The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT Application Publication WO 2013/036868, the entire contents of each of which are hereby incorporated herein by reference.

In some embodiments, an oligonucleotide of the disclosure comprises one or more monomers that are UNA (unlocked nucleotide) nucleotides. UNA is unlocked acyclic nucleotide, wherein any of the bonds of the sugar has been removed, forming an unlocked “sugar” residue. In one example, UNA also encompasses monomers with bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose sugar may also be modified with a cyclopropane ring to produce a tricyclodeoxynucleic acid (tricyclo DNA). The ribose moiety may be substituted for another sugar such as 1,5,-anhydrohexitol, threose to produce a threose nucleotide (TNA), or arabinose to produce an arabino nucleotide. The ribose molecule can also be replaced with non-sugars such as cyclohexene to produce cyclohexene nucleotide or glycol to produce glycol nucleotides.

Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3′-phosphate, inverted base dT(idT) and others. Disclosure of this modification can be found in PCT Publication No. WO 2011/005861.

Other alternatives chemistries of an oligonucleotide of the disclosure include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Additional non-limiting, exemplary LNA nucleotides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667, each of which are hereby incorporated by reference.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the nucleotide sugar modification is 2′-O-C1-4alkyl such as 2′-O-methyl (2′-OMe), 2′-deoxy (2′-H), 2′-O-C1-3alkyl-O-C1-3alkyl such as 2′-methoxyethyl (“2′-MOE” or “MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), or 4′-thioribosyl nucleotide.

### Mixmers and Gapmers

The ASO can have a mixmer and/or gapmer structure, for example, in a pattern disclosed by the ASOs in FIG. 17 or FIG. 18.

In certain embodiments, the ASO is a mixmer. As used herein, the term “mixmer” refers to an oligonucleotide comprising an alternating composition of DNA monomers and nucleotide analogue monomers across at least a portion of the oligonucleotide sequence. In certain embodiments, the ASO is a mixmer based on the gapmer structure, comprising a mixture of DNA nucleotides and 2′-MOE nucleotides in the gap, flanked by RNA sequences (e.g., 2′-modified RNA sequences) in the wings. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) monomers, 2′-fluoro-ANA monomers, HNA monomers, INA monomers, 2′-MOE-RNA (2′-O-methoxyethyl-RNA), 2′Fluoro-DNA, and LNA. In some embodiments, the mixmer is incapable of recruiting RNase H. In some embodiments, the mixmer comprises one type of affinity enhancing nucleotide analogue together with DNA and/or RNA.

Multiple different modifications can be interspaced in a mixmer. For example, the ASO can comprise LNA modification in a plurality of nucleotides and a different modification in some or all of the rest of the nucleotides. In some embodiments, any two adjacent LNA-modified nucleotides are separated by at least 1, 2, 3, 4, or 5 nucleotides. Throughout the ASO, the distance between adjacent LNA-modified nucleotides can either be constant (e.g., any two adjacent LNA-modified nucleotides are separated by 1, 2, 3, 4, or 5 nucleotides) or variable. In some embodiments, the length of the ASO is 3×n, 3×n−1, or 3×n−2 nucleotides (n is an integer of 6 or greater), wherein (a) (i) the nucleotides at positions 3×m−2 (m is an integer from 1 to n) are nucleotides (e.g., ribonucleotides or deoxyribonucleotides) comprising a first modification (e.g., LNA), (ii) the nucleotides at positions 3×m−1 (m is an integer from 1 to n) are nucleotides (e.g., ribonucleotides or deoxyribonucleotides) comprising a first modification (e.g., LNA), or (iii) the nucleotides at positions 3×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl. In some embodiments, the length of the ASO is 2×n or 2×n−1 nucleotides (n is an integer of 9 or greater), wherein (a) (i) the nucleotides at positions 2×m−1 (m is an integer from 1 to n) are nucleotides (e.g., ribonucleotides or deoxyribonucleotides) comprising a first modification (e.g., LNA), or (ii) the nucleotides at positions 2×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). Similar modification patterns, for example, where the first modification is repeated every 4, 5, or more nucleotides, are also contemplated.

In certain embodiments, the ASO comprises a DNA sequence (e.g., having at least 1, 2, 3, 4, 5, 6, 7, 8, 9, 10, 11, 12, 13, 14, or 15 contiguous nucleotides of unmodified DNA) flanked on both sides by RNA sequences (e.g., 2′-modified RNA sequences or 2′-modified ribonucleotides). Such structure is known as “gapmer,” in which the DNA region is referred to as the “gap” and the RNA regions is referred to as the “wings” (see, e.g., PCT Application Publication No. WO2013/177248). Gapmers were known to facilitate degradation of the target RNA by recruiting nucleases (e.g., nuclear RNAses (e.g., RNase H)). Surprisingly, in some embodiments of the present disclosure, it has been discovered that a gapmer that binds to a regRNA, having the same sequence as having a parent ASO but having different chemical modifications, can also increase target gene expression.

In some embodiments, the ASO gapmer comprises an internal DNA region flanked by two external RNA “wings.” For example, the internal DNA gap can comprise at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s), while each of the external RNA wing(s) can independently comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Exemplary gapmer structures include, but are not limited to a 1-10-9, 2-10-8, 3-10-7, 4-10-6, 6-10-4, 7-10-3, 8-10-2, 9-10-1, 1-18-1, 2-16-2, 3-14-3, 4-12-4, 5-10-5, 6-8-6, 7-6-7, 8-5-7, 7-5-8, 8-4-8, or 9-2-9 structure where the first and third number indicate the number of external RNA nucleotides and the second number indicates the number of internal DNA nucleotides.

The ASO can also be a mixmer comprising one DNA region linked to one RNA region. In some embodiments, the mixmer comprises at least 10 DNA nucleotides linked to at least 10 RNA nucleotides, wherein the DNA nucleotides are at the 5′ end of the mixmer or the 3′ end of the mixmer. In some embodiments, the mixmer comprises at least 1, 2, 3, 4, 5, 6, 7, 8, 9, 10, 11, 12, 13, 14, 15, 16, 17, 18, or 19 DNA nucleotide(s) linked to at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 RNA nucleotide(s), wherein the DNA nucleotides are at the 5′ end of the mixmer or the 3′ end of the mixmer. In some embodiment, the RNA regions of the gapmer or mixmer can comprise any additional chemical modification as disclosed herein.

In certain embodiments, the ASO (e.g., the gapmer or mixmer) is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. In certain embodiments, the gap is about 1, 2, 3, 4, 5, 6, 7, 8, 9, 10, 11, 12, 13, 14, 15, 16, 17, 18, 19, or more nucleotides in length. In certain embodiments, one or both wings are about 2, 3, 4, 5, 6, 7, 8, 9, 10, 11, 12, 13, 14, 15, 16, 17, 18, or 19 or more nucleotides in length. In certain embodiments, one or both RNA region or wing comprises RNA modifications, for example, β-D-ribonucleotides, 2′-modified nucleotides (e.g., 2′-O-(2-methoxyethyl) (2′-MOE), 2′-O—CH₃, or 2′-fluoro-arabino (FANA)), and bicyclic sugar modified nucleotides (e.g., having a constrained ethyl or locked nucleic acid (LNA)). In certain embodiments, each ribonucleotide in the mixmer or gapmer is modified by 2′-MOE. In certain embodiments, the mixmer or gapmer comprises one or more modified internucleotide bonds, e.g., phosphorothioate (PS) internucleotide linkage. In certain embodiments, each two adjacent nucleotides in the mixmer or gapmer are linked by a phosphorothioate internucleotide bond.

In certain embodiments, the ASO does not comprise 1 or more, 2 or more, 3 or more, 4 or more, 5 or more, 6 or more, 7 or more, 8 or more, 9 or more, 10 or more, 10 or more, 11 or more, 12 or more, 13 or more, 14 or more, 15 or more, 20 or more, 25 or more, 30 or more, 35 or more, 40 or more, or 45 or more contiguous nucleotides of unmodified DNA. In some embodiments, such a DNA sequence is disrupted by modified (e.g., 2′-MOE modified) ribonucleotides every 2, 3, 4, 5, or more nucleotides. In some embodiments, the ASO comprises only ribonucleotides and no deoxyribonucleotides.

The structural features of mixmer and gapmer can be combined. In certain embodiments, the ASO has a structure similar to that of a mixmer disclosed herein (e.g., one having interspaced modifications), except that the second modification in the gap is changed to a third modification (e.g., deoxyribonucleotide). In certain embodiments, the ASO has a structure similar to that of a gapmer disclosed herein, except that in the gap the nucleotides are modified in a mixmer pattern.

In certain embodiments, the ASO further comprises a ligand moiety, e.g., a ligand moiety that specifically targets a tissue or organ in a subject. For example, N-Acetylgalactosamine (GalNAc) specifically targets liver. In certain embodiments, the ligand moiety comprises GalNAc. In certain embodiments, the ligand moiety comprises a three-cluster GalNAc moiety, commonly denoted GalNAc3. Other types of GalNAc moieties are one cluster, two cluster or four cluster GalNAc, denoted as GalNAc1, GalNAc2, or GalNAc4. In certain embodiments, the ligand moiety comprises GalNAc1, GalNAc2, GalNAc3, or GalNAc4.

In certain embodiments, the ligand moiety comprises biotin. In certain embodiments, the ligand moiety comprises palmitic acid. In certain embodiments, the ligand moiety comprises a Spacer 18 moiety (C18).

### III. Pharmaceutical Compositions

In certain embodiments, the ASOs disclosed herein can be present in pharmaceutical compositions. The pharmaceutical composition can be formulated for use in a variety of drug delivery systems. One or more pharmaceutically acceptable excipients or carriers can also be included in the composition for proper formulation. In some embodiments, the pharmaceutical acceptable carrier comprises sterile saline, sterile water, phosphate buffered saline (PBS), or aCSF. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Exemplary carriers and pharmaceutical formulations suitable for delivering nucleic acids are described in Durymanov and Reineke (2018) Front. Pharmacol. 9:971; Barba et al. (2019) Pharmaceutics 11(8): 360; Ni et al. (2019) Life (Basel) 9(3): 59, each of which is incorporated herein by reference. It is understood that the presence of a ligand moiety conjugated to the ASO may circumvent the need for a carrier for delivery to a tissue or organ targeted by the ligand moiety.

The delivery of an oligonucleotide of the disclosure to a cell e.g., a cell within a subject, such as a human subject e.g., a subject in need thereof, such as a subject having a GRN related disorder can be achieved in a number of different ways. For example, delivery may be performed by contacting a cell with an oligonucleotide of the disclosure ither in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the disclosure (see e.g., Akhtar S. and Julian R L., (1992) Trends Cell. Biol. 2(5):139-144 and WO 94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleotide linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively-charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the disclosure. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the disclosure are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

In some embodiments, the compounds described herein may be administered in combination with additional therapeutics. Examples of additional therapeutics include standard of care anti-epilepsy medications such as quinidine and/or sodium channel blockers. Additionally, the compounds described herein may be administered in combination with recommended lifestyle changes such as a ketogenic diet.

### Membranous Molecular Assembly Delivery Methods

Oligonucleotides of the disclosure can also be delivered using a variety of membranous molecular assembly delivery methods including polymeric, biodegradable microparticle, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively-charged liposomes which interact with the negatively charged nucleic acid molecules to form a stable complex. The positively-charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P. Pharma. Sci., 4(6):466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively-charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively-charged DOTMA liposomes which interact spontaneously with negatively charged polynucleotides to form complexes. When enough positively-charged liposomes are used, the net charge on the resulting complexes is also positive. Positively-charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present disclosure are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the disclosure can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides of in the disclosure may be fully encapsulated in a lipid formulation, e.g., a lipid nanoparticle (LNP), or other nucleic acid-lipid particle. LNPs are useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

Non-limiting examples of cationic lipids include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (Cis). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

The ASO may also be deliver in a lipidoid. The synthesis of lipidoids has been extensively described and formulations containing these compounds are particularly suited for delivery of modified nucleic acid molecules or ASOs (see Mahon et al, Bioconjug Chem. 2010 21: 1448-1454; Schroeder et al, J Intern Med. 2010 267:9-21; Akinc et al, Nat Biotechnol. 2008 26:561-569; Love et al, Proc Natl Acad Sci USA. 2010 107: 1864-1869; Siegwart et al, Proc Natl Acad Sci USA. 2011 108: 12996-3001; all of which are incorporated herein in their entireties).

Lipid compositions for RNA delivery are disclosed in WO2012170930A1, WO2013149141A1, and WO2014152211A1, each of which are hereby incorporated by reference.

### IV. Therapeutic Applications

The present disclosure provides methods for treating diseases and disorders associated with decreased GRN gene expression and other diseases and disorders. In some embodiments, the methods employ ASOs that hybridize with GRN regRNAs transcribed from a regulatory element of the GRN gene or a pharmaceutical composition comprising the ASO. The oligonucleotide compositions described herein are useful in the methods of the disclosure and, while not bound by theory, are believed to exert their desirable effects through their ability to modulate the level of PGRN protein (and its GRN peptide proteolytic products) and/or GRN mRNA, and/or the status or activity of GRN (e.g., by increasing the level of the PGRN protein in a cell in a subject (e.g., a mammal, a mouse, a hamster, a non-human primate (e.g., a monkey), or a human)).

An aspect of the present disclosure relates to methods of treating disorders related to GRN (e.g., a GRN-related disorder) in a subject in need thereof, including administering an ASO of the disclosure (or a pharmaceutical composition including the ASO) to thereby increase the expression of GRN in a cell of the subject. In some embodiments, the GRN-related disorder is frontotemporal dementia (FTD) (e.g., GRN-FTD, also known as FTD-GRN) or frontotemporal lobar degeneration (e.g., GRN-related frontotemporal lobar degeneration). In some embodiments, the subject comprises a progranulin haploinsufficiency.

Another aspect of the present disclosure relates to methods of treating a disease or disorder (e.g., a disease or disorder provided herein) in a subject in need thereof, including administering an ASO of the disclosure (or a pharmaceutical composition including the ASO), thereby treating the disease or the disorder in the subject. In some embodiments, the disease or disorder is selected from frontotemporal dementia (FTD), frontotemporal lobar degeneration (FTLD), neuroinflammation, myopathy, familial frontotemporal dementia with neuropathologic frontotemporal lobal degeneration associated with accumulation of TDP-43 inclusions (FTLD-TDP), Down syndrome, Huntington's disease, hippocampal sclerosis dementia, spinocerebellar ataxia 3, chronic traumatic encephalopathy, Alzheimer's disease (AD), amyotrophic lateral sclerosis (ALS), Gaucher disease (GD) and Parkinson's disease (PD), neuronal ceroid lipofuscinosis (NCL) type 11 (CLN11), limbic-predominant age-related TDP-43 encephalopathy (LATE), autism, ischemia-reperfusion injury in the brain, a lysosomal storage disease (LSD), rheumatoid arthritis (RA), inflammatory bowel disease (IBD), multiple sclerosis (MS), ischemic heart disease, intervertebral disc ceneration, and acute kidney injury. In some embodiments, the disease or disorder is a LSD selected from a sphingolipidosis disorder (e.g., GM2 gangliosidosis, Type A (also known as Tay Sachs disease); GM2 gangliosidosis, Type O (also known as Sandhoff disease); GM2 gangliosidosis, Type AB (also known as GM2 activator deficiency); Niemann-Pick disease (e.g., Niemann-Pick disease, Type A; Niemann-Pick disease, Type B; Niemann-Pick disease, Type C; Neimann-Pick disease, Type D; Neimann-Pick disease, Type E; and Neimann-Pick disease, Type F); Gaucher's disease (e.g., Gaucher's disease type 1; Gaucher's disease type 2; and Gaucher's disease type 3); Fabry disease (also known as Anderson-Fabry disease) (e.g., classic Fabry disease and late-onset Fabry Disease); metachromatic leukodystrophy; globoid leukodystrophy (also known as Krabbe disease); GM1 gangliosidosis (e.g., GM1 gangliosidosis Type 1, GM1 gangliosidosis Type 2, and GM1 gangliosidosis Type 3); and multiple sulfatase deficiency); an oligosaccharidosis disorder (e.g., alfa mannosidosis, Schindler disease, aspartylglucosaminuria, and Fucosidosis); a mucopolysaccharidosis (MPS) (e.g., Hurler syndrome, Scheie syndrome, Hurler-Scheie syndrome, Hunter syndrome, Sanfilippo syndrome (e.g., Sanfilippo syndrome Type A, Sanfilippo syndrome Type B, Sanfilippo syndrome Type C, and Sanfilippo syndrome Type D), Morquio syndrome (e.g., Morquio syndrome Type A and Morquio syndrome Type B), Maroteaux-Lamy syndrome, and Sly syndrome; a neuronal ceroid lipofuscinoses (NCLs; also known as Batten disease) (e.g., CLN1, CLN2, CLN3, CLN4, CLN5, CLN6, CLN7, CLN8, CLN9, CLN10, CLN11, CLN12, CLN13, and CLN 14); a sialic acid disorder (e.g., galactosialidosis, infantile sialic acid storage disease, Salla disease, and sialuria); a mucolipidosis (e.g., sialidosis I, sialidosis II, I-cell disease, Pseudo-Hurler-polydystrophy, and mucolipidosis IV); lysosomal acid lipase deficiency, Pompe disease, Danon disease, and cystinosis. Lysosomal storage diseases are generally described in Rajkumar and Dumpa, “Lysosomal Storage Disease,” In: StatPearls [Internet]. Treasure Island (FL): StatPearls Publishing; 2023 January, available at: ncbi.nlm.nih.gov/books/NBK563270/, hereby incorporated by reference in its entirety.

Another aspect of the disclosure includes methods of increasing the level of PGRN protein or GRN mRNA in a cell of a subject (e.g., a subject identified as having a GRN-related disorder or any other disease or disorder provided herein), including contacting the cell of the subject with an ASO of the disclosure (or a pharmaceutical composition including the ASO) to thereby increase the level of PGRN protein or GRN mRNA in the cell of the subject.

Another aspect of the disclosure includes methods of increasing the expression of a GRN gene in a cell of a subject (e.g., a subject having a disease or disorder described herein) comprising administering an ASO of the disclosure (or a pharmaceutical composition including the ASO) to thereby increase the expression of a GRN gene in a cell of the subject.

Another aspect of the present disclosure relates to methods of treating a disease or disorder in a subject in need thereof, comprising administering an ASO of the disclosure (or a pharmaceutical composition including the ASO), thereby treating the disease or disorder in the subject.

Still another aspect of the present disclosure includes methods of increasing expression of GRN in a cell in a subject, comprising administering an ASO of the disclosure (or a pharmaceutical composition including the ASO), thereby treating the disease or disorder in the subject.

In yet another aspect, the disclosure provides for an ASO of the disclosure (or a pharmaceutical composition including the ASO) for use as a medicament. Further, the disclosure provides for an ASO of the disclosure (or a pharmaceutical composition including the ASO) for use in therapy.

Yet another aspect of the disclosure includes methods of modulating (e.g., increasing or reducing) expression of a GRN gene in a cell (e.g., in vivo, ex vivo, or in vitro) including contacting the cell with an ASO of the disclosure (or a pharmaceutical composition including the ASO), thereby increasing the expression of a GRN gene in the cell. In some embodiments, the cell is a human cell or a mammalian cell. The methods may include contacting a cell with an ASO of the disclosure (or a pharmaceutical composition including the ASO), in an amount effective to modulate (e.g., increase) expression of GRN in the cell, thereby increasing expression of PGRN protein or GRN mRNA in the cell. In some embodiments, contacting the cell with the ASO (or a pharmaceutical composition including the ASO) modulates (e.g., increases) the amount of GRN mRNA in the cell. In some embodiments, contacting the cell with the ASO (or a pharmaceutical composition including the ASO) modulates (e.g., increases) the amount of PGRN protein in the cell.

Based on the above methods, further aspects of the present disclosure include an oligonucleotide of the disclosure, or a composition comprising such an oligonucleotide, for use in therapy, or for use as a medicament, or for use in treating a disease or disorder (e.g., a GRN-related disorder or FTD) in a subject in need thereof, or for use in increasing the level of PGRN in a cell of a subject (e.g., a subject identified as having a GRN-related disorder), or for use in increasing expression of GRN in a cell in a subject. The uses include the contacting of a cell with the oligonucleotide, in an amount effective to increase expression of GRN in the cell, thereby increasing expression of GRN in the cell. Embodiments described below in relation to the methods of the disclosure are also applicable to these further aspects.

Contacting of a cell with an ASO may be performed in vitro, ex vivo, or in vivo. Contacting a cell in vivo with the ASO includes contacting a cell or group of cells within a subject, e.g., a human subject, with the oligonucleotide. Combinations of in vitro, ex-vivo, and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest. The cell can be a CNS cell, for example a neuron or a brain cell, a microglial cell.

Administration of the ASOs or pharmaceutical compositions disclosed herein to a subject can be intravenous, intraarterial, intraperitoneal, intramuscular, subcutaneous, intrapleural, intrathecal, intracavitary, by perfusion through a catheter or by direct intralesional injection. In certain embodiments, the ASO or pharmaceutical composition is administered systemically. In certain embodiments, the ASO or pharmaceutical composition is administered by a parenteral route. For example, in certain embodiments, the ASO or pharmaceutical composition is administered intravenously (e.g., by intravenous infusion), for example, with a prefilled bag, a prefilled pen, or a prefilled syringe. In other embodiments, the ASO or pharmaceutical composition is administered locally to an organ or tissue in which an increase in the target gene expression is desirable (e.g., liver or brain tissue (e.g., cortex, hypothalamus, hippocampus, cerebellum, and coronal brain tissue)).

In some embodiments, the oligonucleotide is administered to a subject such that the oligonucleotide is delivered to a specific site within the subject. Such targeted delivery can be achieved by either systemic administration or local administration. The increase of expression of GRN may be assessed using measurements of the level or change in the level of GRN mRNA or PGRN protein in a sample (e.g., blood, tissue or CNS sample) derived from a specific site within the subject. In certain embodiments, the methods include a clinically relevant increase of expression of GRN, e.g., as demonstrated by a clinically relevant outcome after treatment of a subject with an agent to reduce the expression of GRN.

In other embodiments, the oligonucleotide is administered in an amount and for a time effective to result in a reduction (e.g., by 10%, 20%, 30%, 40%, 50%, 60%, 70%, 80%, 90%, or 100%) of one or more symptoms of a disease or disorder (e.g., a GRN-related disorder), such as deterioration in behavior or personality, language impairment, disturbances or alterations in muscle or motor function, memory loss, cognitive dysfunction, tremor, seizures, and dizziness.

### Increase of GRN Expression Level

In some embodiments, the therapeutic methods disclosed herein, using an ASO that targets a GRN regRNA, are designed to increase GRN expression in a subject. Increasing expression of a GRN gene includes any level of increasing of a GRN gene, e.g., at least a partial increase of the expression of a GRN gene. Increased expression may be assessed by an increase in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer-only (vehicle) control or inactive agent control). In certain embodiments, the method causes a clinically relevant increase of expression of GRN, e.g., as demonstrated by a clinically relevant outcome after treatment of a subject with an agent to increase the expression of GRN.

In certain embodiments, the method disclosed herein increases GRN gene expression by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose, pre-administration, or pre-exposure baseline level. In certain embodiments, the method disclosed herein increases GRN gene expression by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level. In certain embodiments, the subject has a deficiency in GRN expression, and the method disclosed herein restores the GRN expression level or activity to at least 20%, at least 30%, at least 40%, at least 50%, at least 60%, at least 70%, at least 80%, at least 90%, or at least 100% of the average GRN expression level or activity in subjects of the species of like age and gender.

In some embodiments, an ASO of the disclosure may enhance the production of GRN mRNA (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may enhance the production of GRN mRNA (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

In some embodiments, an ASO of the disclosure may enhance the production of PGRN protein (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may enhance the production of PGRN protein (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

The expression of a GRN gene may be assessed based on the level of any variable associated with GRN gene expression, e.g., GRN mRNA level or PGRN protein levels. In certain embodiments, the expression level or activity of GRN herein refers to the average expression level or activity in neuron cells or the brain (e.g., brain cells of a brain region described herein).

In certain embodiments, surrogate markers can be used to detect an increase of GRN expression level. For example, effective treatment of a GRN-related disorder, as demonstrated by acceptable diagnostic and monitoring criteria with an agent to increase GRN expression can be understood to demonstrate a clinically relevant increase in GRN.

Increase of the expression of a GRN gene may be manifested by an increase of the amount of GRN mRNA expressed by a first cell or group of cells (such cells may be present, for example, in a sample derived from a subject) in which a GRN gene is transcribed and which has or have been treated (e.g., by contacting the cell or cells with an oligonucleotide of the disclosure, or by administering an oligonucleotide of the disclosure to a subject in which the cells are or were present) such that the expression of a GRN gene is increased, as compared to a second cell or group of cells substantially identical to the first cell or group of cells but which has not or have not been so treated (control cell(s) not treated with an oligonucleotide or not treated with an oligonucleotide targeted to the gene of interest).

In other embodiments, increase of the expression of a GRN gene may be assessed in terms of an increase of a parameter that is functionally linked to GRN gene expression, e.g., PGRN protein expression, granulin peptide levels, or PGRN activity. An increase in GRN expression may be determined in any cell expressing GRN, either endogenous or heterologous from an expression construct, and by any assay known in the art.

An increase of GRN expression may be manifested by an increase in the level of the PGRN protein (or its proteolytic granulin peptide products) that is expressed by a cell or group of cells (e.g., the level of protein expressed in a sample derived from a subject), relative to a control cell or a control group of cells. An increase of GRN expression may also be manifested by an increase in the level of the GRN mRNA level in a treated cell or group of cells, relative to a control cell or a control group of cells.

A control cell or group of cells that may be used to assess the increase of the expression of a GRN gene includes a cell or group of cells that has not yet been contacted with an oligonucleotide of the disclosure. For example, the control cell or group of cells may be derived from an individual subject (e.g., a human or animal subject) prior to treatment of the subject with an oligonucleotide.

The level of GRN mRNA that is expressed by a cell or group of cells may be determined using any method known in the art for assessing mRNA expression. In one embodiment, the level of expression of GRN in a sample is determined by detecting a transcribed polynucleotide, or portion thereof, e.g., mRNA of the GRN gene. RNA may be extracted from cells using RNA extraction techniques including, for example, using acid phenol/guanidine isothiocyanate extraction (RNAzol™ B; Biogenesis), RNeasy™ RNA preparation kits (Qiagen) or PAXgene® (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating GRN mRNA may be detected using methods described in PCT Publication WO 2012/177906, the entire contents of which are hereby incorporated herein by reference. In some embodiments, the level of expression of GRN is determined using a nucleic acid probe. The term “probe,” as used herein, refers to any molecule that is capable of selectively binding to a specific GRN or PGRN sequence, e.g., to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

In some embodiments, the therapeutic methods disclosed herein, using an ASO that targets a GRN regRNA, are designed to decrease an immune response gene expression level in a subject. Such immune response genes include, but are not limited to, cytokines and chemokines. Exemplary cytokines and chemokines are IL-8, IL-6, CCL4, and CCL2. In some embodiments, the GRN ASO reduces the expression of IL-8, IL-6, CCL4, and CCL2 in a cell or a subject.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to GRN mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of GRN mRNA.

An alternative method for determining the level of expression of GRN in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of GRN is determined by quantitative fluorogenic RT-PCR (i.e., the TAQMAN™ System) or the DUAL-GLO® Luciferase assay.

The expression levels of GRN mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of GRN expression level may also comprise using nucleic acid probes in solution.

In some embodiments, the level of GRN mRNA expression is assessed using branched DNA (bDNA) assays or real time PCR (qPCR). Such methods can also be used for the detection of GRN nucleic acids.

The level of PGRN protein expression and the level of granulin peptides may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence of PGRN protein and granulin peptides.

### EXAMPLES

### Example 1: Synthesis and In Vitro Characterization of Human GRN regRNA-Targeting ASOs

Six GRN regRNAs were identified in the human genome, one paRNAs and five eRNAs. 177 steric ASOs were designed and synthesized targeting the GRN regRNAs. 29 ASOs were selected for tiling after first pass screening. 52 ASOs were designed and synthesized for fine tuning. Of these, 5 ASOS were gapmers, 27 had PO/PS bonds, 5 were mixmers, and 15 comprised LNAs.

To assess the expression of hGRN paRNA and mRNA in iPSC-derived neurons and iMGL cells, the following experiment was performed. Real time quantitative PCR (qPCR) was used to asses the expression of the hGRN paRNA and mRNA in these cells, following 24 hour exposure of the cells to the histone deacetylase (HDAC) inhibitor vorinostat (VOR) at either 1 uM or 3 uM, which is known to increase the expression of GRN mRNA. As control, cells were exposed to DMSO vehicle control. For this analysis, the qPCR reference gene was the geomean of GAPDH and PPIA, normalized to iPSC-dervied neurons treated with DMSO samples. n=3.

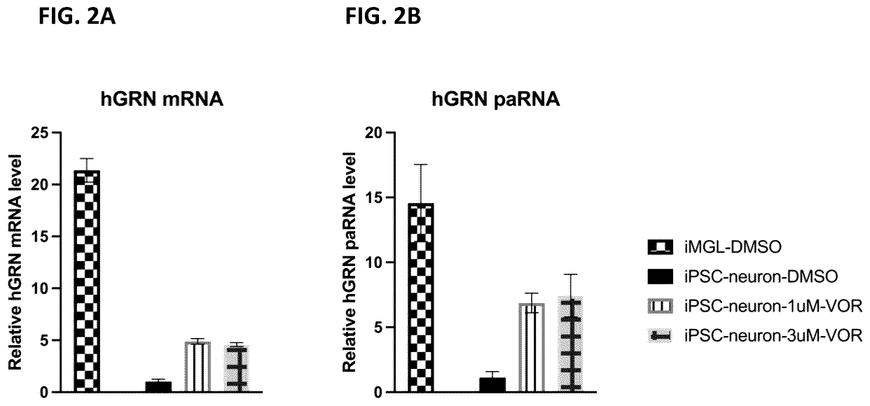

hGRN mRNA (FIG. 2A) and paRNA (FIG. 2B) was detected in iPSC-derived neurons and iMGL cells. hGRN mRNA and paRNA were 15- to 20-fold more abundant in microglia (iMGL cells) as compared to iPSC-derived neurons. Vorinostat treatment induced both hGRN mRNA and paRNA levels.

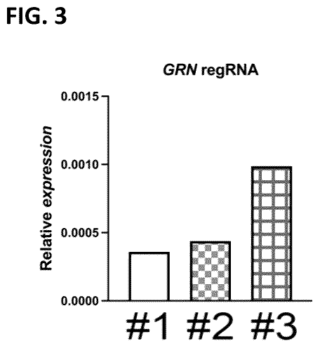

To detect whether hGRN paRNA was also expressed in human cortex tissue, a similar qPCR analysis was performed using human cortex tissue. As shown in FIG. 3, hGRN paRNA was detected in human cortex tissue.

To assess the ability of ASOs targeting hGRN paRNA to modulate the expression of human GRN mRNA, 110 ASOs targeting hGRN paRNA (− strand) were tested in the hepatocellular carcinoma cell lineHepG2. Briefly, HepG2 cells were transfected with 100 nM of the ASOs indicated in Table 2. 48 hours post-transfection, cells were collected for mRNA analysis via qPCR. The expression levels hGRN mRNA in cells treated with each ASOs is provided in Table 2.

TABLE 2

| | hGRN |
|---|---|
| ASO | mRNA |
| name | FC |
| CO-3413 | 0.9 |
| CO-3414 | 0.9 |
| CO-3415 | 1.0 |
| CO-3416 | 0.7 |
| CO-3417 | 0.7 |
| CO-3418 | 0.8 |
| CO-3419 | 0.9 |
| CO-3420 | 0.9 |
| CO-3421 | 0.8 |
| CO-3422 | 0.7 |
| CO-3423 | 1.2 |
| CO-3424 | 1.3 |
| CO-3425 | 1.1 |
| CO-3426 | 0.8 |
| CO-3427 | 1.0 |
| CO-3428 | 0.8 |
| CO-3429 | 0.9 |
| CO-3430 | 0.9 |
| CO-3431 | 0.5 |
| CO-3432 | 1.2 |
| CO-3433 | 0.7 |
| CO-3434 | 0.7 |
| CO-3435 | 0.6 |
| CO-3436 | 1.2 |
| CO-3437 | 0.8 |
| CO-3438 | 0.8 |
| CO-3439 | 0.7 |
| CO-3440 | 0.8 |
| CO-3441 | 0.7 |
| CO-3442 | 0.9 |
| CO-3443 | 1.1 |
| CO-3444 | 0.9 |
| CO-3445 | 0.8 |
| CO-3446 | 0.8 |
| CO-3447 | 0.7 |
| CO-3448 | 0.8 |
| CO-3449 | 1.1 |
| CO-3450 | 0.9 |
| CO-3451 | 0.9 |
| CO-3452 | 0.8 |
| CO-3453 | 0.9 |
| CO-3454 | 1.1 |
| CO-3455 | 0.9 |
| CO-3456 | 0.7 |
| CO-3457 | 0.9 |
| CO-3458 | 1.0 |
| CO-3459 | 1.2 |
| CO-3460 | 0.8 |
| CO-3461 | 0.7 |
| CO-3462 | 1.3 |
| CO-3463 | 1.6 |
| CO-3464 | 1.3 |
| CO-3465 | 0.8 |
| CO-3466 | 1.0 |
| CO-3467 | 1.2 |
| CO-3468 | 1.0 |
| CO-3469 | 1.0 |
| CO-3470 | 1.2 |
| CO-3471 | 0.8 |
| CO-3472 | 0.8 |
| CO-3473 | 1.0 |
| CO-3474 | 1.4 |
| CO-3475 | 1.1 |
| CO-3476 | 0.8 |
| CO-3477 | 0.8 |
| CO-3478 | 0.7 |
| CO-3479 | 1.0 |
| CO-3480 | 0.9 |
| CO-3481 | 1.4 |
| CO-3482 | 0.9 |
| CO-3483 | 0.9 |
| CO-3484 | 1.0 |
| CO-3485 | 0.9 |
| CO-3486 | 0.9 |
| CO-3487 | 0.8 |
| CO-3488 | 0.9 |
| CO-3489 | 1.0 |
| CO-3490 | 0.9 |
| CO-3491 | 0.9 |
| CO-3492 | 0.8 |
| CO-3493 | 1.0 |
| CO-3494 | 0.9 |
| CO-3495 | 0.9 |
| CO-3496 | 1.2 |
| CO-3497 | 1.3 |
| CO-3498 | 0.9 |
| CO-3499 | 0.8 |
| CO-3500 | 0.8 |
| CO-3501 | 0.8 |
| CO-3502 | 0.8 |
| CO-3503 | 1.5 |
| CO-3504 | 0.9 |
| CO-3505 | 0.9 |
| CO-3506 | 0.8 |
| CO-3507 | 0.8 |
| CO-3508 | 1.0 |
| CO-3509 | 1.2 |
| CO-3510 | 0.9 |
| CO-3511 | 1.1 |
| CO-3512 | 1.1 |
| CO-3513 | 1.7 |
| CO-3514 | 1.0 |
| CO-3515 | 1.0 |
| CO-3516 | 0.8 |
| CO-3517 | 1.2 |
| CO-3518 | 1.0 |
| CO-3519 | 0.8 |
| CO-3520 | 1.0 |
| CO-3521 | 1.3 |
| CO-3522 | 1.1 |

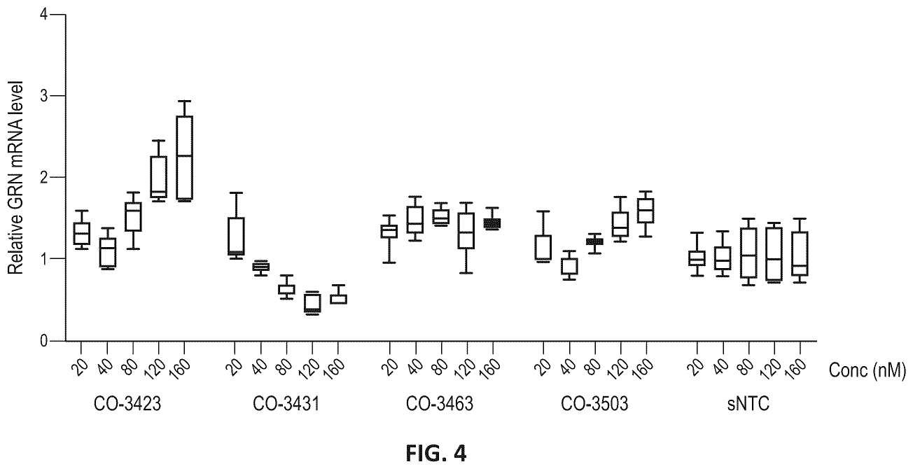

The best ASOs based on increased GRN mRNA fold-change were selected for further characterization in dose titration studies. Briefly, human neuroblastoma SK-N-AS cells were transfected with 20 to 160 nM of ASOs CO-3423, CO-3431, CO-3463, and CO-3503. A steric non-targeting control ASO (sNTC) was used as a control. Cells were collected for mRNA analysis after 48 hours and GRN mRNA quantified by qPCR. Housekeeping genes for normalization were GAPDH and PPIA, and mRNA fold change (FC) was normalized to sNTC. CO-3423 upregulated GRN mRNA by approximately 2.5-fold in a dose-dependent manner as compared to sNTC at the same dose (FIG. 4). CO-3431 downregulated GRN levels approximately 50%.

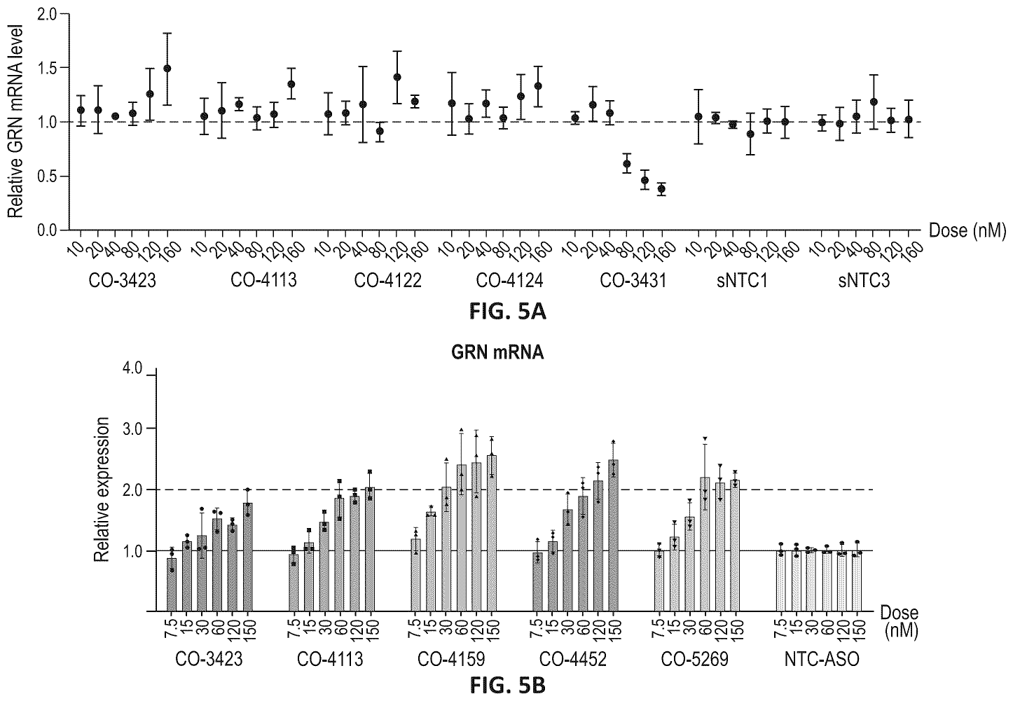

36 additional ASOs were designed with different modifications and base walking around selected ASOs. The effect of increasing doses of these ASOs was assessed in the human neuroblastoma cell line SK-N-AS following transfection. Relative GRN mRNA levels in SK-N-AS cells after treatment was assessed 48-hours post-transfection, and data from exemplary ASOs is shown in FIGS. 5A and 5B.

Additional chemical modifications, including LNA, modifications were made to CO-3423. SK-N-AS cells were transfected with increasing concentrations of the LNA/PS modified ASOs and GRN mRNA quantified by qPCR. ASOs including different LNA modifications of CO-3423 had similar efficacy (i.e, similar increased GRN mRNA expression). The GRN mRNA fold-change induced by each ASO is shown in Table 3. GRN mRNA was normalized to the average fold-change of cells treated with two steric non-targeting control ASOs (CO-3772 and CO-1589 (sNTC1)).

TABLE 3

| ASO | | | |
|---|---|---|---|
| name | 30 nM FC | 60 nM FC | 120 nM FC |
| CO-3423 | 1.2 | 1.4 | 1.7 |
| CO-4113 | 1.6 | 2.0 | 2.3 |
| CO-4353 | 1.4 | 1.4 | 1.8 |
| CO-4354 | 1.6 | 1.9 | 1.9 |
| CO-4355 | 1.6 | 1.8 | 1.9 |
| CO-4356 | 1.6 | 1.8 | 2.2 |
| CO-4357 | 1.5 | 1.7 | 2.4 |
| CO-4358 | 1.5 | 1.9 | 2.0 |
| CO-4359 | 1.7 | 2.2 | 2.5 |
| CO-4360 | 1.4 | 1.8 | 2.5 |

The effect of ASO length on GRN gene expression modulation was also investigated. SK-N-AS cells were transfected with increasing concentrations of the longer ASOs and GRN mRNA quantified by qPCR. Increasing the length of CO-3423 increased the potency of the ASO, as shown in Table 4. mRNA was normalized to the average fold change of cells treated with two steric non-targeting control ASOs (CO-3772 and CO-1589 (sNTC1)).

TABLE 4

| ASO name | 30 nM FC | 60 nM FC | 120 nM FC |
|---|---|---|---|
| CO-3423 | 1.2 | 1.4 | 1.7 |
| CO-4124 | 1.2 | 1.7 | 1.7 |
| CO-4361 | 1.1 | 1.3 | 1.6 |
| CO-4362 | 1.1 | 1.1 | 1.4 |
| CO-4363 | 1.3 | 2.2 | 1.8 |
| CO-4364 | 1.1 | 1.3 | 1.6 |

To determine whether variations in the PO/PS internucleotide bond linkages impacted the ability of CO-3423 and CO-3431 to modulate gene expression, several modified ASOs based on these parent ASOs were prepared. SK-N-AS cells were transfected with increasing concentrations of the mixed PO/PS bond ASOs described in Tables 5 and 6, and GRN mRNA quantified by qPCR. ASOs including mixed PO/PS internucleotide bond linkages did not affect the efficacy of CO-3423 or CO-3431.

GRN mRNA fold-change after treatment with mixed PO/PS bond versions of CO-3423 is shown in Table 5. mRNA was normalized to the average fold change of cells treated with steric nontargeting controls (CO-3772, CO-1589 (sNTC1), and CO-1929 (sNTC3)).

TABLE 5

| ASO name | 30 nM FC | 60 nM FC | 120 nM FC |
|---|---|---|---|
| CO-4113 | 1.6 | 2.0 | 2.3 |
| CO-3423 | 1.2 | 1.4 | 1.7 |
| CO-4452 | 1.2 | 1.6 | 1.8 |
| CO-4453 | 1.4 | 1.9 | 1.9 |

As shown in Table 5, the PO/PS mixed versions of CO-3423 had similar efficacy as the parent ASO, CO-3423.

GRN mRNA fold-change after treatment with mixed PO/PS bond versions of CO-3431 is shown in Table 6. mRNA was normalized as described above.

TABLE 6

| ASO name | 30 nM FC | 60 nM FC | 120 nM FC |
|---|---|---|---|
| CO-3431 | 0.8 | 0.6 | 0.5 |
| CO-4455 | 0.5 | 0.4 | 0.4 |
| CO-4456 | 0.7 | 0.5 | 0.4 |
| CO-4457 | 0.2 | 0.1 | 0.1 |

Similarly, the PO/PS mixed versions of CO-3431 had similar efficacy as CO-3431.

To further assess the effect of additional modifications on the ability of CO-3423 parent ASO to modulate GRN gene expression, additional LNA tiling and PO bond modifications were incorporated to CO-3423. SK-N-AS cells were transfected with increasing concentrations of the modified ASOs and GRN mRNA quantified by qPCR. CO-3423 was used for comparison. GRN mRNA was normalized to the average fold-change of cells treated with a steric non-targeting control ASO, CO-1589 (sNTC1). The GRN mRNA fold change induced by each ASO is shown in Table 7.

TABLE 7

| ASO name | 7.5 nM FC | 13 nM FC | 30 nM FC | 60 nM FC | 120 nM FC | 150 nM FC |
|---|---|---|---|---|---|---|
| CO-3423 | 0.9 | 1.1 | 1.3 | 1.5 | 1.4 | 1.8 |
| CO-4113 | 0.9 | 1.1 | 1.5 | 1.9 | 1.9 | 2.0 |
| CO-4359 | 1.2 | 1.6 | 2.0 | 2.4 | 2.4 | 2.6 |
| CO-4364 | 1.1 | 1.2 | 1.7 | 2.1 | 1.9 | 2.3 |
| CO-4452 | 1.0 | 1.1 | 1.7 | 1.9 | 2.1 | 2.5 |
| CO-5266 | 1.0 | 1.0 | 1.2 | 1.7 | 2.0 | 2.2 |
| CO-5267 | 1.0 | 1.0 | 1.2 | 1.7 | 1.9 | 2.3 |
| CO-5268 | 1.0 | 1.1 | 1.5 | 1.9 | 1.9 | 2.6 |
| CO-5269 | 1.0 | 1.2 | 1.6 | 2.2 | 2.1 | 2.2 |

ASOs with the additional modifications had similar efficacy as ASOs with LNA residues. CO-4452, CO-5268, and CO-5269 had similar or higher efficacy as compared to CO-4113.

To further assess the effect of modifications on the ability of CO-3463 parent ASO to modulate GRN gene expression, ASOs based on this ASOS incorporating additional LNA modifications were prepared and compared to CO-3462. SK-N-AS cells were transfected with increasing concentrations of the ASOs and GRN mRNA quantified by qPCR. GRN mRNA was normalized to the average fold-change of cells treated with a steric non-targeting control ASO, CO-1589 (sNTC1). The GRN mRNA fold change induced by each ASO is shown in Table 8.

TABLE 8

| ASO name | 7.5 nM FC | 13 nM FC | 30 nM FC | 60 nM FC | 120 nM FC | 150 nM FC |
|---|---|---|---|---|---|---|
| CO-3462 | 1.2 | 1.2 | 1.3 | 1.7 | 2.0 | 1.9 |
| CO-3463 | 1.3 | 1.5 | 1.4 | 1.6 | 1.4 | 1.4 |
| CO-5288 | 1.3 | 1.5 | 1.5 | 1.9 | 1.9 | 2.1 |
| CO-5289 | 1.4 | 1.4 | 1.5 | 1.9 | 1.9 | 1.9 |

ASOs CO-3462, CO-5288, and CO-5289 upregulated GRN mRNA 2-fold as compared to control ASO CO-1589.

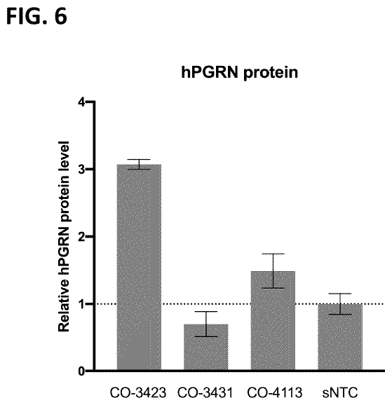

PGRN protein levels after ASO treatment were also assessed. To assess the effect of ASO treatment on PGRN protein expression, the following experiment was performed. 120 nM of various ASOs (CO-3423, CO-3431, CO-4113) were transfected into SK-N-AS cells. As control, cells treated with a steric non-targeting control ASO, CO-1589 (sNTC1). 48 hours-post transfection, protein was extracted using RIPA Lysis and Extraction Buffer (Thermo Fisher Scientific), and PGRN protein levels determined using a GRN ELISA (PGRN ELISA kit, AdipoGen® Life Sciences; Cat. No. AG-45A-0018). Values were normalized to lysates from cells treated with sNTC1. As shown in FIG. 6, CO-3423 and CO-4113 increased PGRN protein by 1.5- to 3-fold.

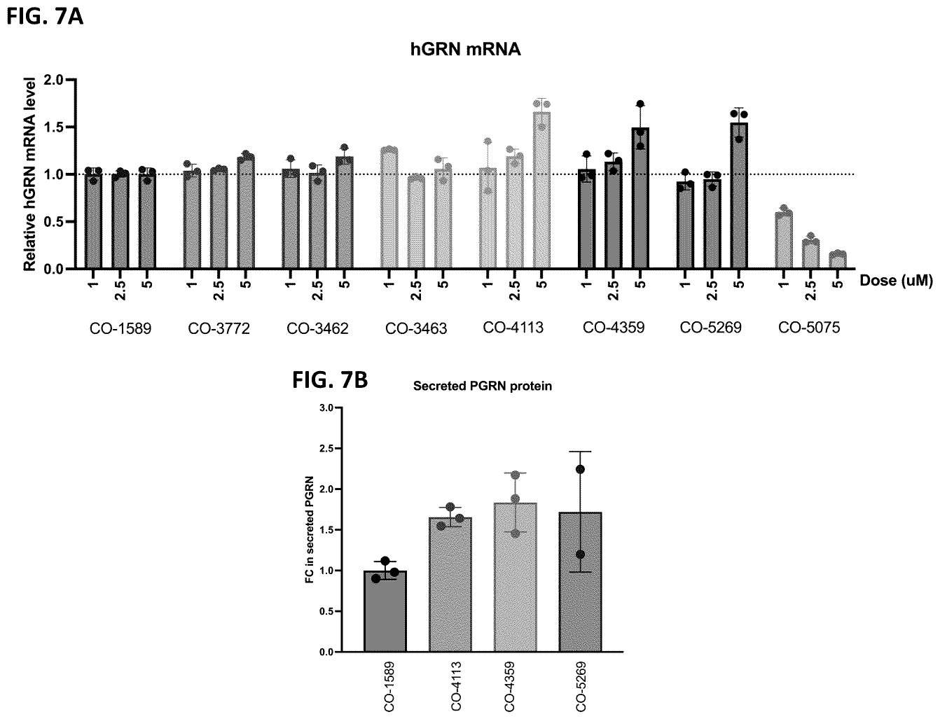

ASOs CO-3462, CO-3463, CO-41113, CO-4359, and CO-5269 also upregulate GRN mRNA in iMGL cells. To test the efficacy of GRN paRNA-targeting ASOs' ability to modulate GRN gene expression in iMGL cells, the following experiment was performed. Briefly, iMGL cells were nucleofected with ASOs CO-3462, CO-3463, CO-4113, CO-4359, CO-5269, as well as, two steric non-targeting control ASOs (CO-3772 and CO-1589 (sNTC1), and a negative control ASO, CO-5075 (described in Laudisi et al. (2019) Mol. Oncol. 13(10): 2142-59. iMGL cells were collected 72 hours post-nucleofection for mRNA quantification using qPCR. Cell supernatant was also collected to quantify secreted PGRN protein using the GRN ELISA described above. mRNA was normalized to cells treated with CO-1589. As shown in FIG. 7A, all tested ASOs (i.e., CO-3462, CO-3463, CO-4113, CO-4359, and CO-5269 upregulated GRN mRNA in iMGL cells as compared to control CO-1589. ASOs CO-4113, CO-4359, and CO-5269 also increased secreted PGRN protein in iMGL cells (FIG. 7B).

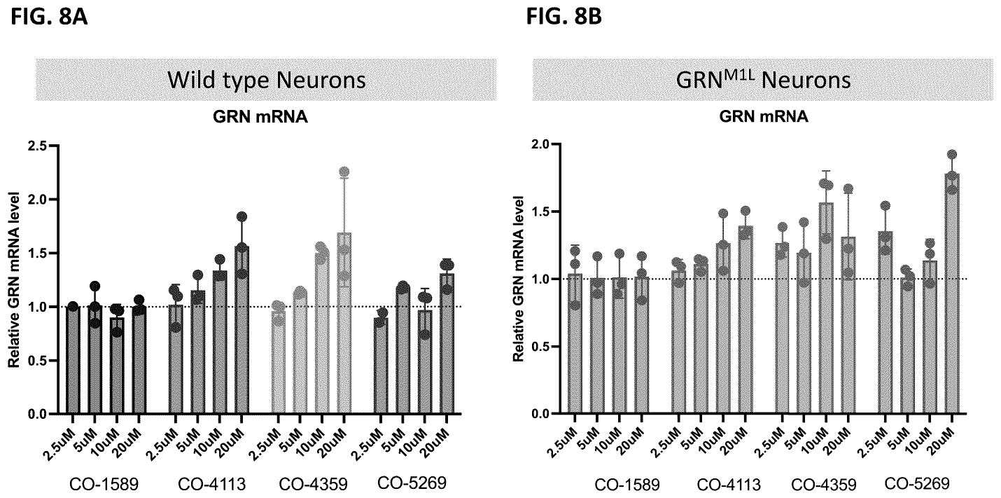

ASOs CO-3431, CO-3463, and CO-4113, CO-4359 and CO-5269 also upregulate GRN mRNA in iPSC wild type or GRN^{M1L} neurons. To test the efficacy of GRN paRNA-targeting ASOs' ability to modulate GRN gene expression in wild-type iPSC-derived neurons and iPSC-derived neurons including the GRN M1L missense mutation (GRN^{M1L}) neurons, the following experiment was performed. Briefly, iPSC-derived neuron cells and GRN^{M1L} neurons were nucleofected with the indicated ASO or a steric non-targeting control (sNTC1; CO-1589) as the negative control. Cells were collected 120 hours post-nucleofection for mRNA quantification using qPCR, and mRNA was normalized to cells treated with sNTC1. As shown in FIGS. 8A and 8B, the tested ASOs upregulated GRN mRNA in wild type-iPSC neurons and in GRN^{M1L} neurons.

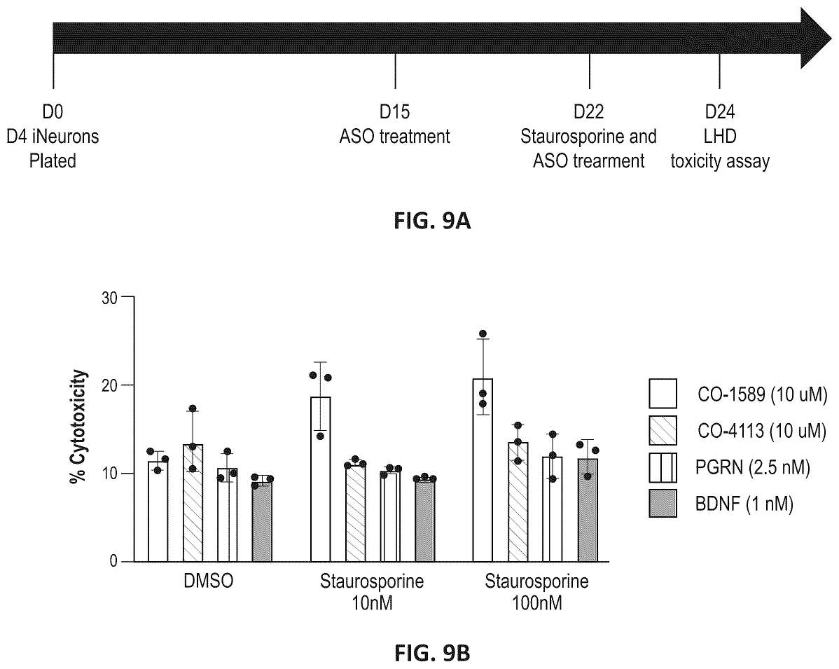

CO-4113 rescued staurosporine-induced toxicity in GRN-FTD patient-derived neurons. GRN-FTD patient-derived neurons were treated with 10 uM of CO-4113 or sNTC (CO-1589) ASOs. As positive controls, cells were treated with either recombinant human PGRN protein (2.5 nM) or brain derived neurotrophic factor (BDNF) protein (1 nM). One week after treatment, neurons were treated with DMSO, 10 nM or 100 nM staurosporine. Culture media was collected 24 hour later, and cell toxicity was measured using LDH-Glo™ Cytotoxicity Assay (Promega Corp.) as instructed by manufacturer. Triton™ X-100-treated cells were used as positive control. All values were normalized to positive control cells to calculate percent cytotoxicity. The assay timeline is provided in FIG. 9A. As shown in FIG. 9B, treatment with ASO CO-4113 reduced the cytotoxicity of GRN-FTD patient-derived neuron cells induced by staurosporine at 10 nm or 100 nM.

68 additional ASOs were designed to target intergenic enhancers (eRNA). SK-N-AS cells were transfected with 120 nM of the ASOs indicated in Table 9. 48 hours post-transfection, the cells were collected for GRN mRNA analysis using qPCR. CO-1589, a steric nontargeting control ASO, was used as a control. GRN mRNA was normalized to cells treated with CO-1589. The GRN mRNA fold change induced by each ASO is shown in Table 9 below.

TABLE 9

| ASO name | hGRN mRNA FC | ASO name | hGRN mRNA FC |
|---|---|---|---|
| CO-3423 | 1.6 | CO-4585 | 1.4 |
| CO-3431 | 1.2 | CO-4586 | 0.9 |
| CO-4113 | 1.1 | CO-4587 | 1.2 |
| CO-4124 | 1.3 | CO-4588 | 0.9 |
| CO-4619 | 1.3 | CO-4589 | 1.1 |
| CO-4620 | 1.0 | CO-4590 | 1.3 |
| CO-4621 | 1.2 | CO-4591 | 1.1 |
| CO-4622 | 1.0 | CO-4592 | 1.1 |
| CO-4623 | 1.1 | CO-4593 | 0.8 |
| CO-4624 | 1.3 | CO-4594 | 1.3 |
| CO-4625 | 1.1 | CO-4595 | 1.0 |
| CO-4626 | 1.0 | CO-4596 | 1.0 |
| CO-4627 | 1.3 | CO-4597 | 0.9 |
| CO-4628 | 1.0 | CO-4598 | 1.1 |
| CO-4629 | 1.1 | CO-4599 | 1.0 |
| CO-4630 | 1.0 | CO-4600 | 1.0 |
| CO-4631 | 1.1 | CO-4601 | 1.0 |
| CO-4632 | 1.1 | CO-4602 | 1.2 |
| CO-4633 | 1.3 | CO-4603 | 1.3 |
| CO-4634 | 1.1 | CO-4604 | 1.2 |
| CO-4635 | 1.0 | CO-4605 | 1.2 |
| CO-4636 | 0.9 | CO-4606 | 1.4 |
| CO-4637 | 0.9 | CO-4607 | 1.1 |
| CO-4638 | 1.0 | CO-4608 | 1.1 |
| CO-4639 | 1.1 | CO-4609 | 1.2 |
| CO-4640 | 1.0 | CO-4610 | 1.2 |
| CO-4641 | 0.9 | CO-4611 | 1.4 |
| CO-4642 | 1.0 | CO-4612 | 1.0 |
| CO-4643 | 0.9 | CO-4613 | 1.6 |
| CO-4644 | 1.1 | CO-4614 | 1.2 |
| CO-4645 | 1.4 | CO-4615 | 1.0 |
| CO-4646 | 1.3 | CO-4616 | 1.2 |
| CO-4647 | 0.8 | CO-4617 | 1.2 |
| CO-4648 | 1.6 | CO-4618 | 1.3 |
| CO-4649 | 1.2 | | |
| CO-4650 | 1.1 | | |
| CO-4651 | 1.3 | | |

The best hits from these ASOs were tested in a dose escalation study. Briefly, SK-N-AS cells were transfected with increasing concentrations of the ASOs indicated in Table 10 at 3.75 nM to 120 nM. Cells were incubated for 48 hours. Subsequently, the cells were collected for mRNA quantification via RT-qPCR. GRN paRNA-targeting ASOs CO-3431, CO-4113, and CO-4124 were used as positive controls. GRN mRNA was normalized to cells treated with CO-1589. The GRN mRNA fold change induced by each ASO is shown in Table 10 below.

TABLE 10

| ASO name | 3.75 nM FC | 7.5 nM FC | 15 nM FC | 30 nM FC | 60 nM FC | 120 nM FC |
|---|---|---|---|---|---|---|
| CO-3431 | 0.9 | 0.9 | 0.6 | 0.5 | 0.4 | 0.4 |
| CO-4113 | 0.9 | 1.1 | 1.5 | 1.9 | 1.9 | 2.0 |
| CO-4124 | 1.0 | 1.0 | 1.0 | 1.2 | 1.4 | 1.9 |
| CO-4606 | 1.1 | 1.1 | 1.2 | 1.3 | 1.4 | 1.7 |
| CO-4611 | 1.1 | 1.1 | 1.1 | 1.1 | 1.1 | 1.3 |
| CO-4613 | 1.0 | 1.1 | 1.2 | 1.1 | 1.2 | 1.4 |
| CO-4619 | 1.0 | 1.1 | 1.3 | 1.2 | 1.2 | 1.6 |
| CO-4622 | 1.0 | 1.0 | 1.0 | 1.0 | 1.1 | 1.1 |
| CO-4631 | 1.1 | 1.1 | 1.2 | 1.1 | 1.1 | 1.3 |
| CO-4637 | 1.1 | 1.1 | 1.2 | 1.1 | 1.2 | 1.3 |
| CO-4649 | 1.2 | 1.2 | 1.3 | 1.2 | 1.2 | 1.3 |

ASOs CO-4611, CO-4613, CO-4619, CO-4622, and CO-4631, CO-4637, CO-4649 were identified as initial hits. ASOs CO-4606 and CO-4619 showed greater than 1.3-fold GRN mRNA upregulation.

26 additional ASOs were designed to target an intragenic enhancer RNA (eRNA). SK-N-AS cells were treated with 120 nM of the ASOs indicated in Table 11 for 48 hours. The cells were collected for GRN mRNA analysis using qPCR. CO-1589 was used as a control. GRN mRNA was normalized to cells treated with CO-1589. The GRN mRNA fold change induced by each ASO is shown in Table 11 below.

TABLE 11

| ASO name | hGRN mRNA FC |
|---|---|
| CO-6430 | 1.0 |
| CO-6431 | 0.8 |
| CO-6432 | 1.0 |
| CO-6433 | 0.9 |
| CO-6434 | 0.9 |
| CO-6435 | 1.2 |
| CO-6436 | 1.1 |
| CO-6437 | 1.1 |
| CO-6438 | 1.2 |
| CO-6439 | 1.1 |
| CO-6440 | 1.0 |
| CO-6441 | 1.4 |
| CO-6442 | 1.2 |
| CO-6443 | 1.0 |
| CO-6444 | 1.0 |
| CO-6445 | 1.3 |
| CO-6446 | 1.1 |
| CO-6447 | 1.2 |
| CO-6448 | 1.1 |
| CO-6449 | 1.1 |
| CO-6450 | 0.9 |
| CO-6451 | 1.0 |
| CO-6452 | 1.4 |
| CO-6453 | 0.9 |
| CO-6454 | 1.0 |
| CO-6455 | 0.9 |

ASOs CO-6411, CO-6445 and CO-6452 showed greater than 1.3-fold GRN mRNA upregulation.

Additional ASOs based on parent ASO CO-3462 were designed. SK-N-AS cells were transfected with increasing concentrations of ASOs based on CO-3462, designed to include chemical modifications and additional antisense nucleotide sequences. GRN mRNA quantified using qPCR. CO-3462 and CO-4113 were used as controls. GRN mRNA was normalized to cells treated with CO-1589. The GRN mRNA fold change induced by each ASO is shown in Table 12.

TABLE 12

| ASO | | | | ASO | | | |
|---|---|---|---|---|---|---|---|
| name | 30 nM FC | 60 nM FC | 120 nM FC | name | 30 nM FC | 60 nM FC | 120 nM FC |
| CO-3462 | 0.9 | 1.2 | 1.7 | CO-6387 | 1.3 | 1.0 | 1.1 |
| CO-4113 | 1.4 | 1.4 | 2.1 | CO-6388 | 1.1 | 1.0 | 1.4 |
| CO-6380 | 0.8 | 0.8 | 0.8 | CO-6389 | 1.0 | 1.0 | 2.1 |
| CO-6381 | 0.8 | 0.7 | 1.0 | CO-6390 | 1.3 | 1.3 | 1.5 |
| CO-6382 | 0.8 | 0.8 | 0.9 | CO-6391 | 1.3 | 1.0 | 1.6 |
| CO-6383 | 1.2 | 1.0 | 1.2 | CO-6392 | 1.0 | 0.8 | 1.0 |
| CO-6384 | 1.0 | 1.0 | 1.5 | CO-6393 | 0.9 | 0.7 | 1.0 |
| CO-6385 | 1.1 | 1.2 | 1.3 | CO-6394 | 1.1 | 0.9 | 1.3 |
| CO-6386 | 1.2 | 0.9 | 1.1 | CO-6395 | 1.0 | 1.3 | 1.6 |
| CO-6396 | 1.4 | 1.9 | 1.9 | CO-6410 | 1.1 | 1.0 | 1.6 |
| CO-6397 | 1.4 | 1.8 | 1.8 | CO-6411 | 1.1 | 1.4 | 1.8 |
| CO-6398 | 1.3 | 1.5 | 1.8 | CO-6512 | 1.3 | 1.8 | 1.3 |
| CO-6399 | 1.3 | 1.6 | 1.9 | CO-6513 | 1.3 | 2.7 | 1.8 |
| CO-6400 | 1.1 | 1.3 | 1.3 | CO-6514 | 1.4 | 1.9 | 1.8 |
| CO-6401 | 1.2 | 1.5 | 1.7 | CO-6515 | 1.2 | 1.8 | 1.7 |
| CO-6402 | 1.5 | 1.6 | 1.8 | CO-6516 | 1.0 | 1.4 | 1.0 |
| CO-6403 | 1.6 | 2.2 | 2.4 | CO-6517 | 1.2 | 2.3 | 2.0 |
| CO-6404 | 1.3 | 1.3 | 1.5 | CO-6518 | 1.2 | 1.3 | 0.9 |
| CO-6405 | 1.1 | 1.3 | 1.3 | CO-6519 | 1.1 | 1.0 | 0.9 |
| CO-6406 | 1.4 | 1.4 | 1.7 | CO-6520 | 1.0 | 1.2 | 0.8 |
| CO-6407 | 1.1 | 1.2 | 1.6 | CO-6521 | 1.1 | 1.2 | 1.1 |
| CO-6408 | 1.0 | 1.1 | 1.5 | CO-6522 | 1.1 | 1.0 | 1.1 |
| CO-6409 | 1.1 | 1.1 | 1.5 | CO-6523 | 2.9 | 1.4 | 1.3 |

Additional ASOs based on parent ASOs CO-4363 and CO-4364 were designed. SK-N-AS cells were transfected with increasing concentrations of ASOs based on CO-4363 and CO-4364 designed to include additional chemical modifications, and GRN mRNA quantified using qPCR. GRN mRNA was normalized to cells treated with CO-1589. The GRN mRNA fold change (FC) induced by each ASO is shown in Table 13.

TABLE 13

| ASO | | | |
|---|---|---|---|
| name | 30 nM FC | 60 nM FC | 120 nM FC |
| CO-3423 | 1.1 | 1.6 | 1.4 |
| CO-4113 | 1.6 | 1.9 | 1.6 |
| CO-4363 | 1.6 | 1.7 | 1.7 |
| CO-4364 | 1.1 | 1.5 | 1.6 |
| CO-6412 | 1.0 | 0.8 | 0.9 |
| CO-6413 | 1.0 | 0.6 | 0.7 |
| CO-6414 | 1.6 | 1.4 | 1.3 |
| CO-6415 | 1.3 | 1.6 | 1.6 |
| CO-6416 | 1.4 | 1.6 | 2.2 |
| CO-6417 | 1.4 | 2.3 | 2.8 |
| CO-6418 | 1.6 | 1.6 | 1.9 |
| CO-6419 | 1.0 | 1.0 | 0.9 |
| CO-6420 | 1.4 | 2.7 | 2.2 |
| CO-6421 | 1.3 | 2.1 | 1.7 |
| CO-6422 | 1.9 | 2.0 | 2.1 |
| CO-6423 | 1.2 | 2.6 | 2.8 |
| CO-6424 | 1.7 | 2.9 | 3.1 |
| CO-6425 | 0.9 | 1.3 | 1.3 |
| CO-6426 | 0.8 | 1.5 | 1.3 |
| CO-6427 | 1.2 | 1.1 | 1.2 |
| CO-6428 | 1.1 | 1.4 | 1.0 |
| CO-6429 | 1.0 | 1.5 | 1.4 |

ASOs CO-6416, CO-6417, CO-6420, CO-6423 and CO-6224 showed greater than 2-fold GRN mRNA upregulation.

Select ASOs were assessed for IFNγ-induced immune suppression activity. To assess the ability of GRN regRNA-targeting ASOs to mitigate immunosuppression induced by IFNγ, the following experiment was performed. Briefly, iMGL cells were nucleofected with CO-4113, CO-4359, or CO-5269. A steric non-targeting control ASO was used as control. After ASO nucleofection, IFNγ was added to the culture media of the ASO-treated iMGL cells to induce an immune response. As additional control, cells treated with the steric non-targeting control ASO were solely treated with phosphate-buffered saline (PBS). Cells and supernatants were collected for qPCR and ELISA quantification of secreted protein (PGRN, IL-8, and CCL4) and gene expression (IL-6 mRNA, CCL4 mRNA, and CCL2 mRNA).

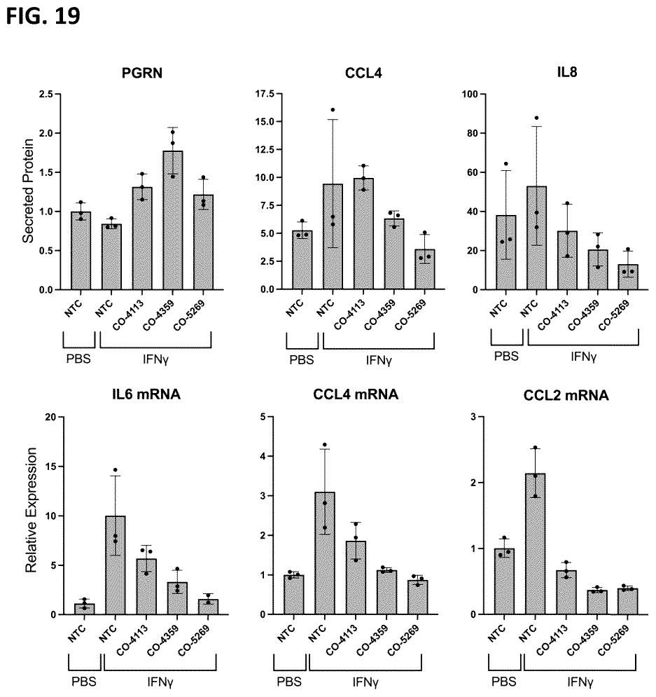

As shown in FIG. 19, treatment of the iMGL cells with the GRN regRNA-targeting ASOs upregulated secreted PGRN protein levels and significantly reduced the IFNγ-mediated expression of immune response genes IL-8 and CCL4, as shown by the reduced secretion of IL-8 and CCL4 protein. Treatment of the iMGL cells with the GRN regRNA-targeting ASOs also significantly reduced the IFNγ-mediated expression of immune response genes IL-6, CCL4, and CCL2, as shown by their respective reduced mRNA levels (FIG. 19).

### Example 2: Synthesis and In Vitro Characterization of Mouse GRN regRNA-Targeting ASOs

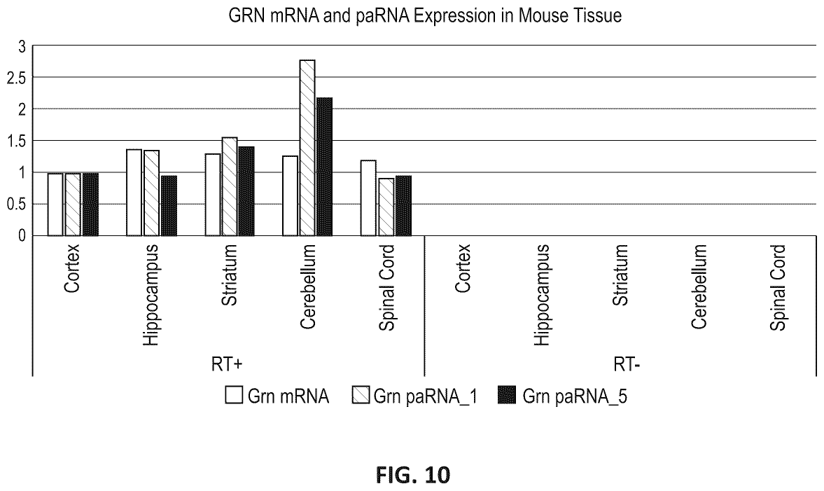

mGRN expression in various mouse tissues was determined. To determine whether GRN paRNA and mRNA is expressed in mouse CNS tissue, CNS tissue from a C57/BL6 mouse was lysed and RNA extracted using TRIzol™ reagent (Thermo Fisher Scientific) and analyzed using qPCR. cDNA was synthesized with and without reverse transcriptase (RT) to ensure that the amplified products were derived from RNA in the samples. As shown in FIG. 10, GRN mRNA and paRNA were detected and are expressed in the murine cortex, hippocampus, striatum, cerebellum, and spinal cord.

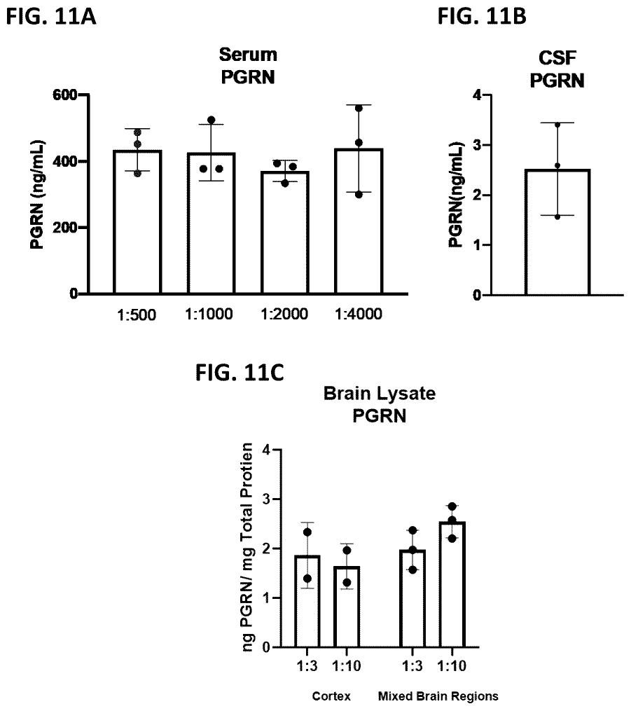

To assess the levels of Pgrn protein in mouse samples, the following experiment was performed. Briefly, mouse plasma, CSF, and brain tissue derived from C57/BL6 mice were obtained. Brain tissue (cortex or mixed brain regions) was homogenized in RIPA buffer containing protease inhibitors. Mouse Pgrn protein levels were quantified by ELISA (Mouse Pgrn ELISA Kit, AdipoGen® Life Sciences; Cat. No. AG-45A-0019) as instructed by manufacturer. Serum and brain samples were assessed at varying dilutions, and all sample results normalized to total protein. As shown in FIGS. 11A, 11B, and 11C, the assay detected mouse Pgrn in serum, CSF, and cortex and mixed brain regions lysate.

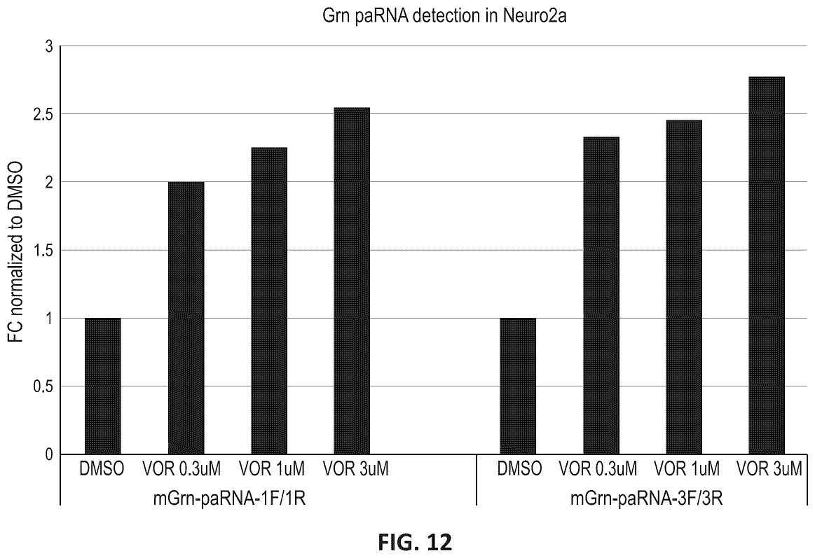

Mouse Grn paRNA is also expressed in mouse neuroblastoma Neuro2a cells. To assess whether mouse Grn paRNA is expressed in the immortalized neuroblastoma cell line Neuro2a, cells were exposed for 24 hour to vorinostat (VOR) at either 0.3 uM, 1 uM or 3 uM or DMSO control, and total RNA was extracted from Neuro2a cells using a Qiagen RNeasy Kit, and cDNA was synthesized using random hexamers. Mouse Grn paRNA was detected using two different primer sets (1F/1R or 3F/3R) by a real-time quantitive PCR (qPCR) assay. As shown in FIG. 12, mouse Grn paRNA levels increase with vorinostat treatment.

91 steric ASOs were designed and synthesized targeting the mGrn regRNAs. 33 ASOs were selected for tiling after a first pass screening. 88 ASOs were designed and synthesized for fine tuning. Of these, 5 ASOs were gapmers, 11 were sterics, 28 had PO/PS bonds, and 44 were mixmers.

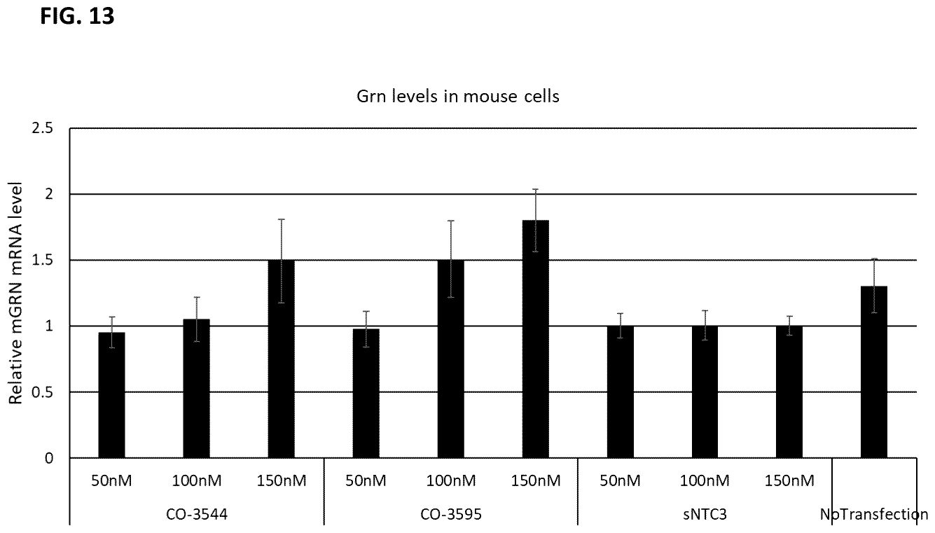

57 ASOs targeting the Grn promoter (− strand) were screened in Neuro2a cells. Neuro2a cells were transfected with 100 nM of each of the ASOs listed in Table 14 and cells were harvested 48 hours later for Grn mRNA quantification using qPCR. Housekeeping genes for normalization were Gapdh and Ppia. Relative Grn mRNA levels were normalized to mRNA extracted from cells treated with sNTC3 (steric nontargeting control). As shown in FIG. 13, Grn regRNA-targeting ASOs CO-3544 and CO-3595 upregulated mGrn mRNA in a dose-dependent manner and upregulated mGrn mRNA up to 1.5-fold and 1.8-fold, respectively.

The mGm mRNA fold change (FC) induced by each ASO at 100 nM is shown in Table 14.

TABLE 14

| ASO | mGRN mRNA |
|---|---|
| name | FC |
| CO-3544 | 1.1 |
| CO-3545 | 1.7 |
| CO-3546 | 1.4 |
| CO-3547 | 1.7 |
| CO-3548 | 1.5 |
| CO-3549 | 1.4 |
| CO-3550 | 0.9 |
| CO-3551 | 1.5 |
| CO-3552 | 1.1 |
| CO-3553 | 1.3 |
| CO-3554 | 1.2 |
| CO-3555 | 1.5 |
| CO-3556 | 1.3 |
| CO-3557 | 1.6 |
| CO-3558 | 1.2 |
| CO-3559 | 1.4 |
| CO-3560 | 1.2 |
| CO-3561 | 1.3 |
| CO-3562 | 1.1 |
| CO-3563 | 1.5 |
| CO-3564 | 1.1 |
| CO-3565 | 1.6 |
| CO-3566 | 1.1 |
| CO-3567 | 1.1 |
| CO-3568 | 1.2 |
| CO-3569 | 1.2 |
| CO-3570 | 1.2 |
| CO-3571 | 1.4 |
| CO-3572 | 1.3 |
| CO-3573 | 1.4 |
| CO-3574 | 1.3 |
| CO-3575 | 1.2 |
| CO-3576 | 1.2 |
| CO-3577 | 1.1 |
| CO-3578 | 1.0 |
| CO-3579 | 1.2 |
| CO-3580 | 1.2 |
| CO-3581 | 1.1 |
| CO-3582 | 1.1 |
| CO-3583 | 1.2 |
| CO-3584 | 1.1 |
| CO-3585 | 1.7 |
| CO-3586 | 1.4 |
| CO-3587 | 1.9 |
| CO-3588 | 0.9 |
| CO-3589 | 1.3 |
| CO-3590 | 1.2 |
| CO-3591 | 1.4 |
| CO-3592 | 1.2 |
| CO-3593 | 1.1 |
| CO-3594 | 1.6 |
| CO-3595 | 2.3 |
| CO-3596 | 1.4 |
| CO-3597 | 1.2 |
| CO-3598 | 1.3 |
| CO-3599 | 1.0 |
| CO-3600 | 1.5 |

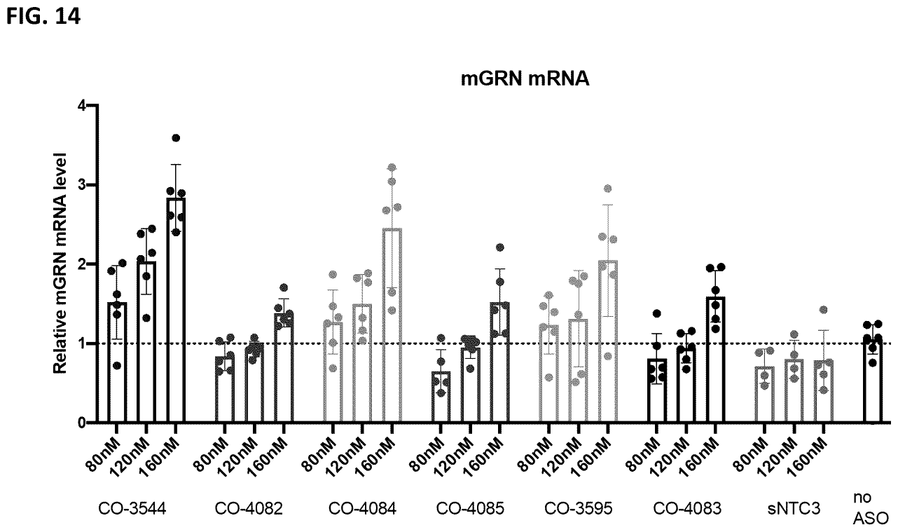

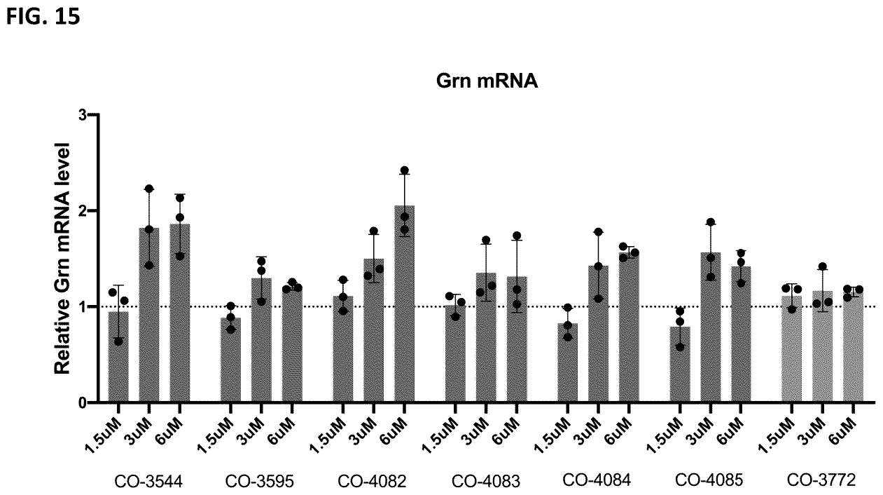

Next, 26 ASOs based on parent ASOs CO-3544 and CO-3595 were designed with different modifications and base walking. Neuro2a cells were transfected with either 80, 120 or 160 nM of each of the ASOs listed in Table 15, and cells were harvested 48 hours later for mGrn mRNA quantification using qPCR. As controls, cells were treated with sNTC3 or no ASO. Housekeeping genes for normalization were Gapdh and Ppia. Relative Grn mRNA levels were normalized to mRNA extracted from cells treated with sNTC3. Four new ASOs (CO-4082, CO-4083, CO-4084, CO-4085) upregulated mGRN mRNA in Neuro2a cells by 1.5- to 2.5-fold (FIG. 14). Similar upregulation was observed with these ASOs when used to treat primary mouse neuron cells (1.5-6 μM by free uptake, FIG. 15).

CO-3544 was modified to include PO/PS bonds and the efficacy in upregulating mGrn mRNA was determined. As shown in Table 15 below, CO-3544 with up to 9 PO to PS internucleotide bond substitutions was effective in upregulating mGrn mRNA after transfection in Neuro2a cells at 40, 80 and 160 nM following transfection.

TABLE 15

| ASO name | 40 nM FC | 80 nM FC | 160 nM FC |
|---|---|---|---|
| CO-3544 | 1.2 | 1.3 | 1.4 |
| CO-4082 | 1.4 | 1.6 | 1.4 |
| CO-5290 | 1.1 | 1.0 | 1.0 |
| CO-5291 | 0.9 | 1.0 | 0.9 |
| CO-5292 | 1.1 | 1.1 | 1.1 |
| CO-5293 | 1.1 | 1.0 | 1.1 |
| CO-5294 | 1.1 | 1.1 | 1.3 |
| CO-5295 | 1.1 | 1.3 | 1.4 |
| CO-5296 | 1.1 | 1.1 | 1.0 |
| CO-5297 | 1.0 | 1.1 | 1.2 |
| CO-5298 | 1.3 | 1.3 | 1.5 |
| CO-5299 | 1.2 | 1.3 | 1.5 |
| CO-5300 | 1.3 | 1.4 | 1.4 |
| CO-5301 | 0.9 | 1.0 | 0.7 |
| CO-5302 | 1.3 | 1.2 | 1.0 |

Similarly, CO-3595 was modified to include varying PO/PS internucleotide bonds and the efficacy of these ASOs in upregulating mGrn mRNA was determined in Neuro2A cells. As shown in Table 16 below, ASOs based on CO-3595 including up to 13 PO/PS internucleotide bond substitutions were effective in upregulating mGrn mRNA at 40, 80 and 160 nM following transfection.

TABLE 16

| ASO name | 40 nM FC | 80 nM FC | 160 nM FC |
|---|---|---|---|
| CO-3595 | 1.2 | 1.3 | 1.4 |
| CO-4083 | 1.3 | 1.4 | 1.6 |
| CO-5303 | 1.3 | 1.6 | 1.8 |
| CO-5304 | 1.4 | 1.6 | 2.2 |
| CO-5305 | 1.2 | 1.4 | 1.6 |
| CO-5306 | 1.2 | 1.0 | 1.3 |
| CO-5307 | 1.0 | 1.1 | 1.1 |
| CO-5308 | 1.3 | 1.4 | 2.0 |
| CO-5309 | 1.3 | 1.7 | 2.0 |
| CO-5310 | 1.1 | 1.1 | 1.3 |
| CO-5311 | 1.5 | 1.7 | 1.7 |
| CO-5312 | 1.5 | 1.7 | 1.8 |
| CO-5313 | 1.3 | 1.4 | 1.5 |
| CO-5314 | 1.5 | 1.7 | 1.5 |
| CO-5315 | 1.3 | 1.4 | 1.6 |

### Example 3: In Vivo Modulation of Mouse GRN Expression with regRNA-Targeting ASOs

ASO-mediated upregulation of progranulin in different brain regions in vivo was determined. ASO CO-3544 (300 μg) in PBS was injected into right lateral ventricle of 8-week-old C57/BL6 mice. A steric nontargeting control ASO (CO-1929), and vehicle (PBS) control were used as controls. Mice were sacrificed at day 28 post-injection. Tissue samples (cortex, hippocampus and striatum) were collected and processed for total RNA and protein extraction as described above. Mouse Grn mRNA quantification was performed using real time qPCR assay.

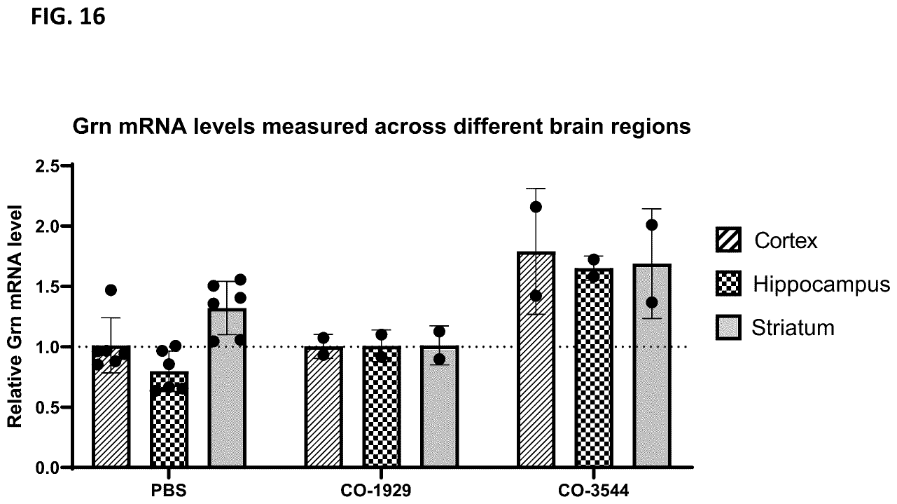

As shown in FIG. 16, treatment with CO-3544 increased Grn mRNA levels by greater than 1.5-fold across different brain regions. Thus, this ASO showed in vivo therapeutic efficacy as noted by its ability to upregulate Grn mRNA levels in mouse CNS tissue.

### Example 4: In Vivo Modulation of PGRN in hGRNT^{Tg} Mice

To assess the ability of hGRN paRNA-targeting ASOs to upregulate hGRN in vivo, experiments using a human GRN transgenic mouse model were performed as described below.

### Materials

CO-4359 was modified to remove the 3′ and 5′ terminal nucleotides resulting in CO-8178. CO-8178 was characterized in SK-N-AS, HEK293T and Vero-76 cell lines, as well as NGN2 neurons and iMGL cells, as previously described (data not shown).

B6.Cg-Grn^{tm1.2Blrl}Hprt1^{tml(GRN)Blrl}/J mice (hGRN^{Tg}; The Jackson Laboratory, Strain No. 036240) were used to assess the efficacy of the ASO CO-8178 in upregulating human GRN in vivo. These mice express an X-linked transgenic human GRN gene (Hprttm1(GRN)Blrl) and have knockouts of exons 3 and 4 in the mouse Grn gene (Grntm1.2Blrl).

Mice were injected intracerebroventricular (ICV) with 5 μL of aCSF (vehicle control) or 100 μg CO-8178 at a rate of 1 μl/min. Animals were sacrificed 3 weeks post-dosing for analysis. Mouse brain sections were processed to analyze PGRN protein and mRNA levels. GRN mRNA and intracellular and secreted PGRN were quantified using the methods described above.

The in vivo assay with the hGRN^{Tg} mice was repeated with ASOs CO-4452, CO-8883, CO-8903, CO-8879, CO-8871, CO-8873, CO-8873, CO-3462, and CO-6424 as described above. aCSF and a steric nontargeting (NTC) were used as controls. Mouse brain sections were processed for measuring PGRN protein and mRNA. GRN mRNA and intracellular and secreted PGRN protein were quantified using the methods described above.

### Results

CO-8178 upregulated GRN mRNA in SK-N-AS, HEK293T and Vero-76 cell lines as well as NGN2 neurons and iMGL cells (data not shown). CO-8178 also suppressed the IFNγ-induced immune response in iMGL cells (data not shown). Without wishing to be bound by theory, CO-8178 is an 18mer so it may be better distributed across tissues and more likely to escape from the endosome.

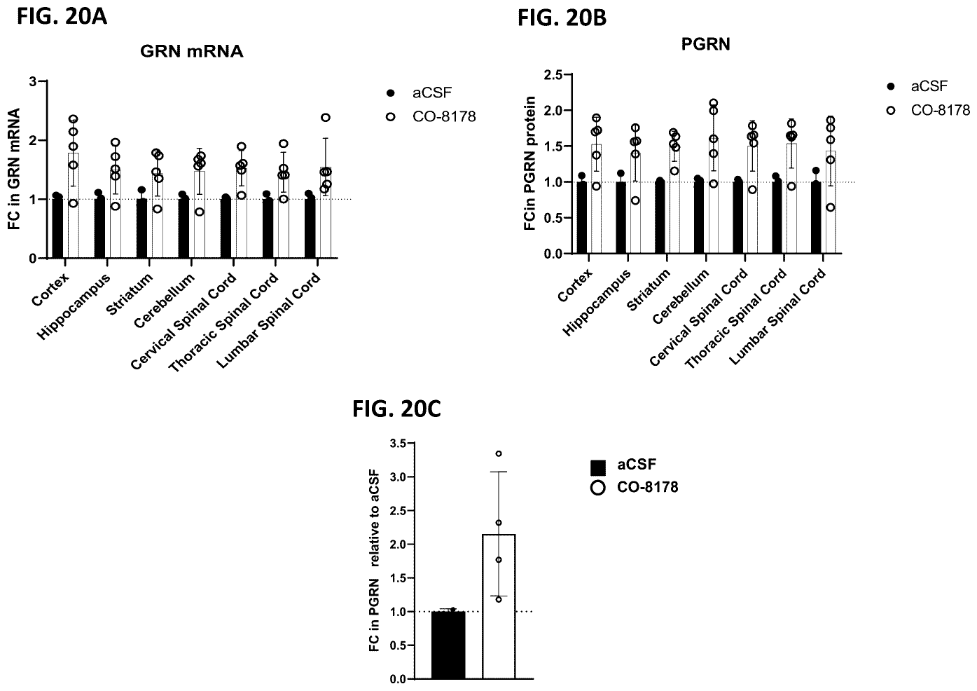

As shown in FIGS. 20A and 20B, in vivo treatment with CO-8178 upregulated GRN mRNA and protein across all CNS tissues assessed. FIG. 20A provides hGRN mRNA quantifications and FIG. 20B provides hPGRN protein quantification in hGRNT^{Tg} mice. Samples from aCSF-treated control mice are shown in the bars on the left of each tissue, samples from CO-8178 treat mice are shown in the bars on the right of each tissue. The GRN mRNA and protein expression levels correlated post-treatment with CO-8178. r=0.8158, 95% CI was 0.7079-0.8899, r²=0.6699. P value <0.0001. In addition, secreted PGRN protein in the CSF was also upregulated post-CO-8178 treatment (FIG. 20C). Thus, CO-8178 upregulated GRN mRNA and PGRN protein in human GRN mice across all measured CNS tissue. CO-8178 upregulated the secreted PGRN protein in the CSF. In addition, upregulation of GRN mRNA and PGRN protein were significantly correlated.

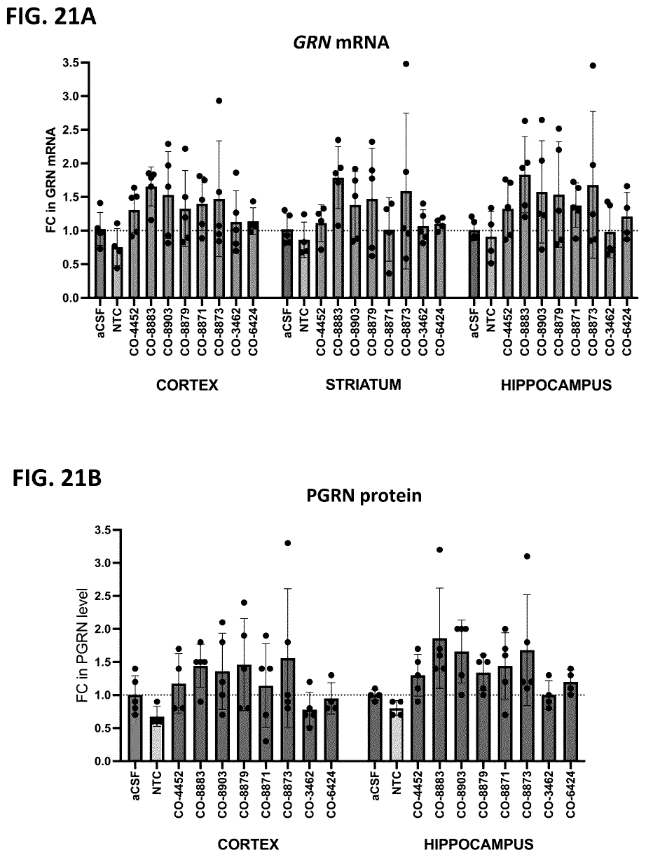

As shown in FIGS. 21A and 21B, in vivo treatment with CO-4452, CO-8883, CO-8903, CO-8879, CO-8873 upregulated human GRN mRNA and PGRN protein across all tissues examined. FIG. 21A provides hGRN mRNA quantifications and FIG. 21B provides human PGRN protein quantification in hGRNT^{Tg} mice brain tissues

### Example 5: In Vivo Modulation of Grn mRNA in Grn^{+/-} Haploinsufficient Mice

To assess a mouse model analog of human GRN haploinsufficiency, the following experiments were performed using a Grn heterozygous knockout mouse.

### Materials and Methods

Grn^{tm1.1Far} (B6.129S4(FVB)-Grn^{tm1.1Far}/Mmjax; The Jackson Laboratory, Cat. No. MMRRC Strain #036771-JAX) heterozygous mice were used to assess the efficacy of CO-3544 and CO-10691 to upregulate mGrn in vivo. These mice have deletion of exon 2-13 of the mouse Grn gene. CO-3544 and CO-10691 target mouse Grn paRNA.

Mice were injected ICV with 5 μL of artificial CSF (aCSF; vehicle control), 300 μg of ASO CO-3544, or 200 μg of ASO CO-10691, each in aCSF, at a rate of 1 μL/min. For ASO CO-3544, mice were sacrificed 4 weeks post-dose for analyses. For CO-10691, mice were sacrificed 2 and 4 weeks post-dose for analyses. Mouse brain sections were processed, and Pgrn protein and Grn mRNA quantified, as described above.

### Results

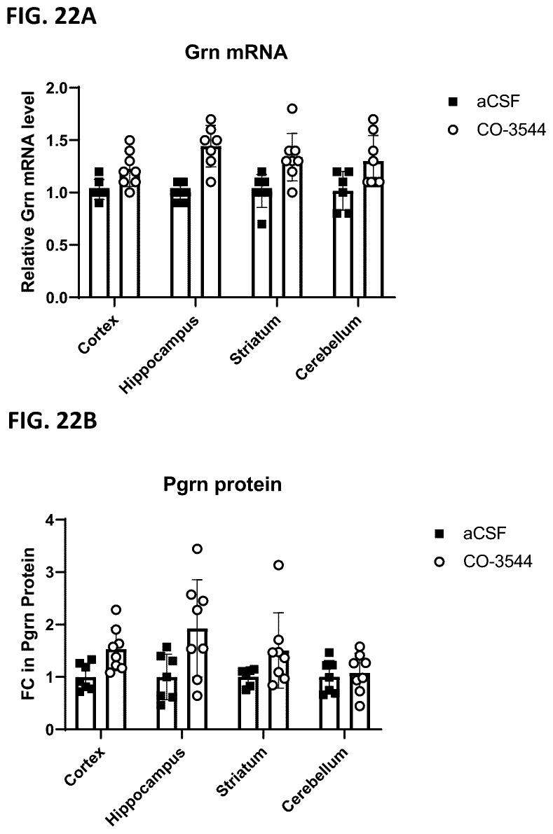

As shown in FIGS. 22A and 22B, in vivo treatment of heterozygous Grn^{tm1.1Far} mice with ASO CO-3544 resulted in a 1.5- to 2.0-fold increase in Pgrn protein expression in the hippocampus, striatum, and cerebellum brain regions, as compared to aCSF vehicle control (FIG. 22B). Treatment with ASO CO-3544 resulted in up to 1.5-fold increase in Grn mRNA expression as compared to aCSF vehicle control (FIG. 22A).

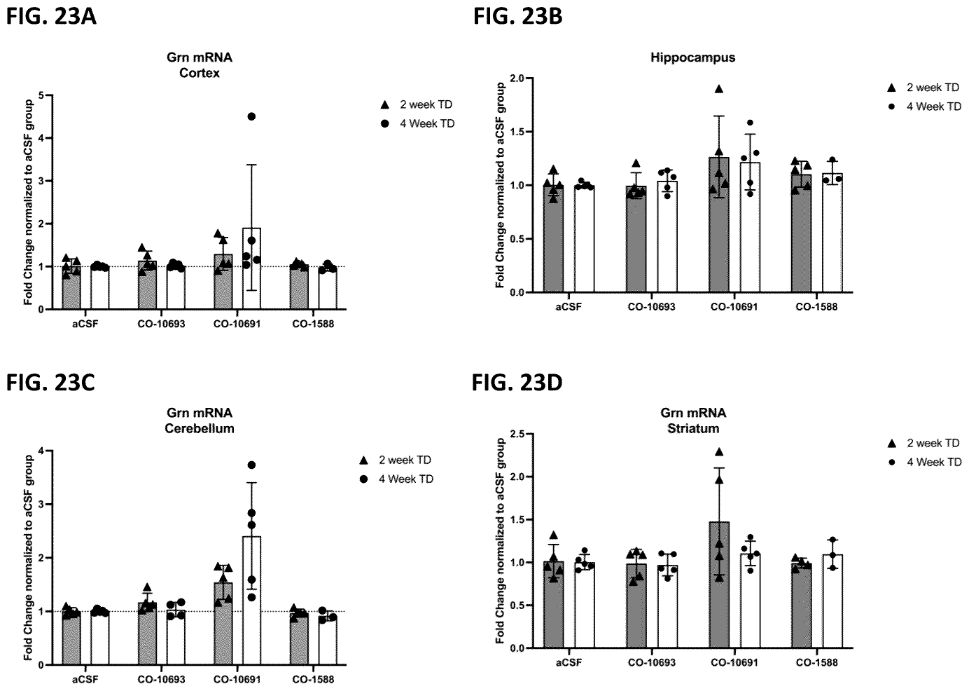

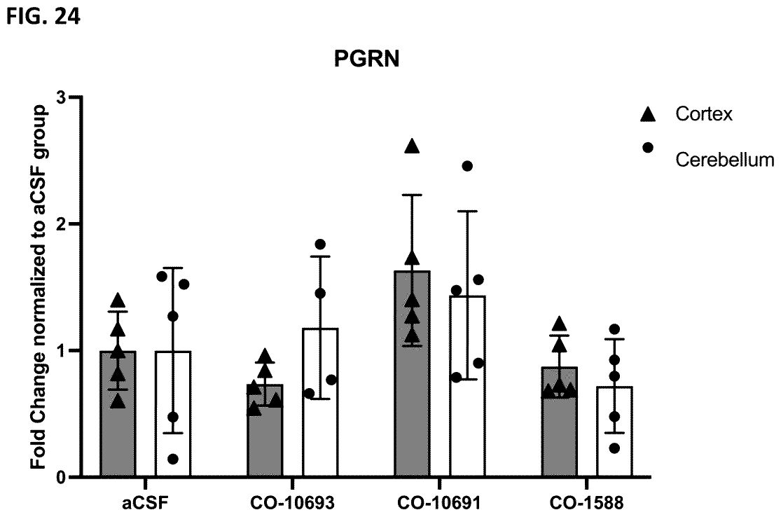

ASO CO-10691 also upregulated mGm mRNA (FIGS. 23A, 23B, 23C, and 23D) and murine Pgrn protein (FIG. 24) expression in the cortex, hippocampus, striatum, and cerebellum brain regions, as compared to aCSF vehicle control.

### Example 6: Synthesis and In Vitro Characterization of Additional Human GRN regRNA-Targeting ASOs

Further hGRN regRNA-targeting ASOs were designed and synthesized with additional chemical modifications, and characterized in vitro.

### Materials and Methods

### Secreted and Intracellular GRN Levels

SK-N-AS cells were transfected with 90 nM of the ASOs CO-4359, CO-4452, CO-5268, CO-5269, CO-6424, and CO-8178. A steric nontargeting control ASO (sNTC) was used as a control. Cells were collected and analyzed via ELISA 48 hours post-transfection, as described in Example 1 above. Protein levels were normalized to total protein and cells treated with sNTC.

mRNA Expression Assay

SK-N-AS cells were transfected with 3.75 to 90 nM of selected ASOs. Scrambled ASO (sNTC) was used as a control. Cells were collected for mRNA 48 hours post-transfection. Housekeeping genes were GAPDH and PPIA, mRNA fold change was normalized to cells treated with sNTC.

iPSC-derived neurons were nucleofected with 20 μM of ASOs CO-8865, CO-8866, CO-8871, CO-8873, CO-8875, CO-8877, CO-8879, CO-8883, CO-8889, CO-8901, and CO-8903. Two non-targeting control ASOs (NTC-ASO-1 and NTC-ASO-2) were used as control. Cells were harvested 5 days post-nucleofection for GRN mRNA quantification. The qPCR reference genes were GAPDH and PPIA. Relative GRN mRNA levels were normalized to cells treated with either of the two non-targeting control ASOs.

### Chemokine Assay

iMGL cells were nucleofected with 5 uM of CO-4452, CO-8865, CO-8866, and CO-8883. A non-targeting control ASO (NTC) was used as a control. After treatment, IFNγ was added to the ASO-treated iMGL cells to induce an immune response. Cells and supernatants were collected for qPCR and ELISA quantification GRN, CCL3 and CCL4 gene expression.

### Results

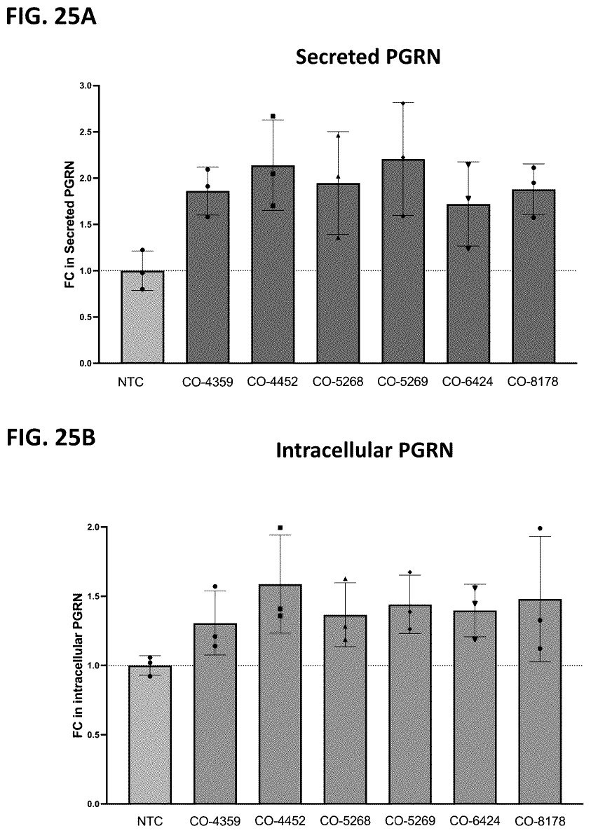

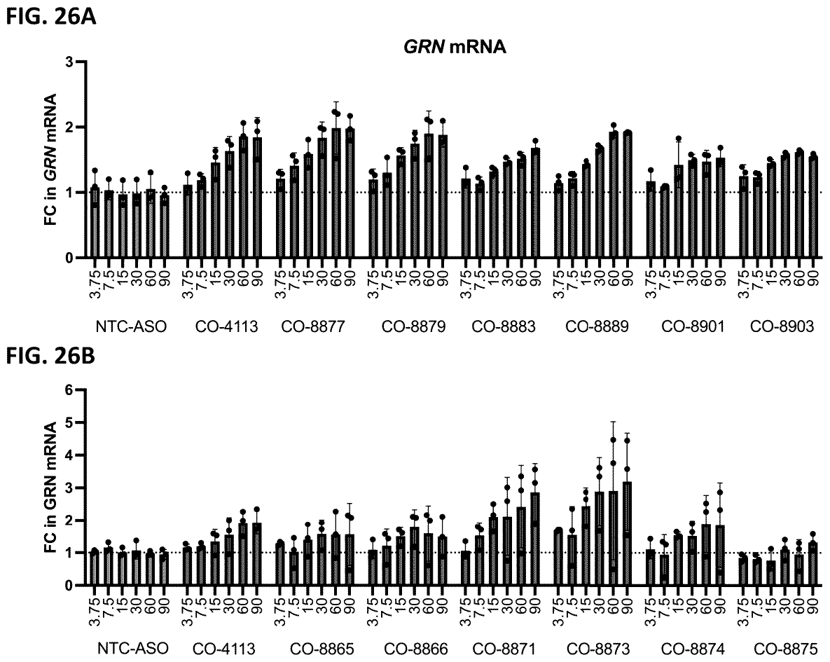

As shown in FIGS. 25A and 25B, ASOs CO-4359, CO-4452, CO-5268, CO-5269, CO-6424 and CO-8178 upregulated both secreted (FIG. 25A) and intracellular (FIG. 25B) PGRN protein in SK-N-AS cells. A dose-dependent increase in GRN mRNA expression in SK-N-AS cells was also observed with CO-4113, CO-8877, CO-8879, CO-8883, CO-8889, CO-8901, and CO-8903 (FIGS. 26A and 26B).

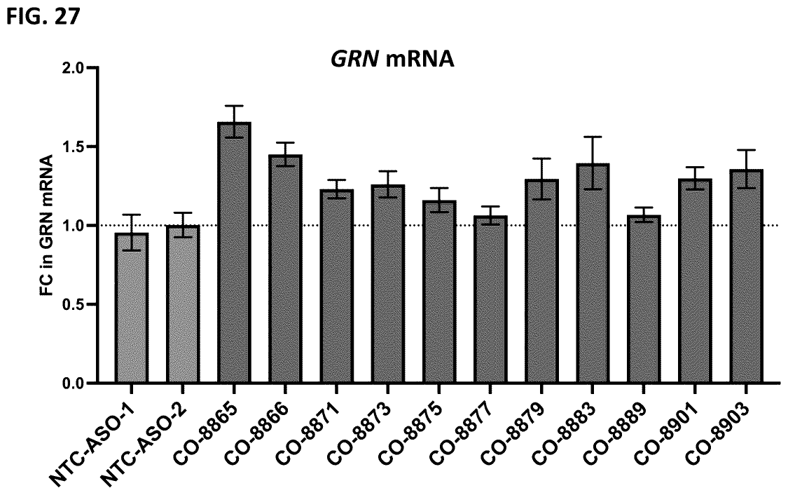

Upregulation of GRN mRNA in iPSC-derived neuron cells was also observed with CO-8865, CO-8866, CO-8871, CO-8873, CO-8875, CO-8879, CO-8883, CO-8889, CO-8901, and CO-8903 (FIG. 27).

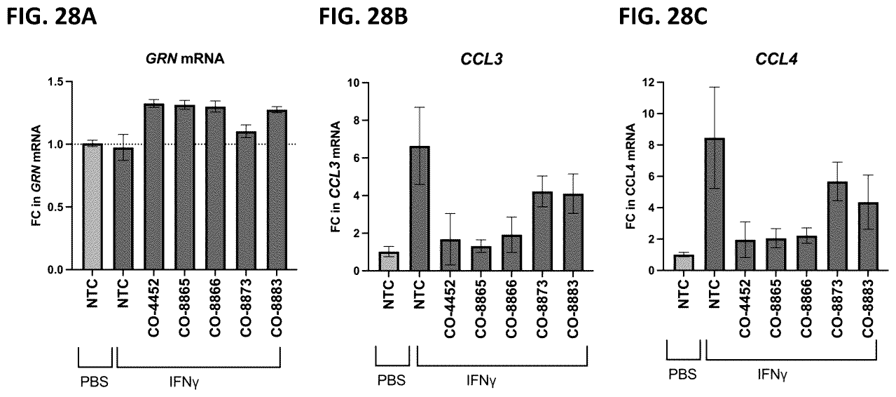

As shown in FIGS. 28A, 28B, and 28C, ASOs CO-4452, CO-8865, CO-8866, CO-8873, and CO-8883 also upregulate GRN mRNA expression (FIG. 28A) and reduced IFNγ-induced chemokine (CCL3 and CCL4) expression in iMGL cells (FIGS. 28B and 28C, respectively).

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. An antisense oligonucleotide (ASO) targeting a regulatory RNA (regRNA) of progranulin (pGRN), comprising a nucleotide sequence complementary to at least 5 contiguous nucleotides of any one of SEQ ID NOs: 1-9.

2. (canceled)

3. (canceled)

4. The ASO of claim 1, wherein the ASO comprises a nucleotide sequence of any one of SEQ ID NOs: 1543, 1684-1688, 1902, 2292-2296, 2767, 3464, 3564, 1369-1542, 1544-1683, 1689-2291, 2297-2766, 2768-3463, 3465-3563, or 3565-4738.

5. The ASO of claim 1, wherein the regRNA has a nucleotide sequence of SEQ ID NO: 1, and the ASO comprises a nucleotide sequence of any one of SEQ ID NOs: 20, 59, 1085, 10-19, 21-58, 60-268, 691, 991-1084, 1086-1368, or 4743-4915.

6. The ASO of claim 1, wherein
a. the regRNA has a nucleotide sequence of SEQ ID NO: 2, and the ASO comprises the nucleotide sequence of any one of SEQ ID NOs: 269-279,
b. the regRNA has a nucleotide sequence of SEQ ID NO: 3, and the ASO comprises the nucleotide sequence of any one of SEQ ID NOs: 280-291 or 336-359,
c. the regRNA has a nucleotide sequence of SEQ ID NO: 4, and the ASO comprises the nucleotide sequence of any one of SEQ ID NOs: 292-313 or 360-380,
d. the regRNA has a nucleotide sequence of SEQ ID NO: 5, and the ASO comprises the nucleotide sequence of any one of SEQ ID NOs: 314-335 or 381-416, or
e. the regRNA has a nucleotide sequence of SEQ ID NO: 6, and the ASO comprises the nucleotide sequence of any one of SEQ ID NOs: 417-442.

7. (canceled)

8. (canceled)

9. (canceled)

10. (canceled)

11. The ASO of claim 1, wherein the ASO comprises a nucleotide comprising one or more chemical modifications.

12. (canceled)

13. The ASO of claim 11, wherein at least 3, 4, or 5 nucleotides at the 5′ end and at least 3, 4, or 5 nucleotides at the 3′ end of the ASO comprise ribonucleotides with one or more chemical modifications.

14. The ASO of claim 11, wherein the one or more chemical modifications comprise a nucleotide sugar modification comprising one or more of 2′-O-C1-4alkyl such as 2′-O-methyl (2′-OMe), 2′-deoxy (2′-H), 2′-OC1-3alkyl-O-C1-3alkyl such as 2′-methoxyethyl (“2′-MOE” or “MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

15. The ASO of claim 11, wherein the one or more chemical modifications comprise an internucleotide linkage modification comprising one or more of phosphorothioate (“PS” or (P(S))), phosphoramidate (P(NR₁R₂) such as dimethylaminophosphoramidate (P(N(CH₃)₂)), phosphonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂COO⁻)), alkylphosphonate (P(C₁₋₃alkyl) such as methylphosphonate —P(CH₃), boranophosphonate (P(BH₃)), or phosphorodithioate (P(S)₂).

16. The ASO of claim 11, wherein the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, unstructured nucleic acid (“UNA”), isoguanine (“isoG”), isocytosine (“isoC”), a glycerol nucleic acid (GNA), glycerol nucleic acid (GNA), or thiophosphoramidate morpholinos (TMOs).

17. The ASO of claim 11, wherein the one or more chemical modifications comprise a biotin, a palmitic acid, or a C18 moiety linked to the 5′ end or the 3′ end of the ASO.

18. The ASO of claim 11, wherein the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

19. The ASO of claim 11, wherein the ASO does not comprise 10 or more contiguous nucleotides of unmodified DNA.

20. The ASO of claim 19, wherein the ASO does not comprise a deoxyribonucleotide.

21. The ASO of claim 11, wherein the ASO does not comprise an unmodified ribonucleotide.

22. (canceled)

23. (canceled)

24. (canceled)

25. (canceled)

26. (canceled)

27. (canceled)

28. (canceled)

29. (canceled)

30. (canceled)

31. The ASO of claim 11, wherein the ASO comprises at least one phosphodiester bond.

32. (canceled)

33. The ASO of claim 11, wherein each cytidine in the ASO is modified by 5-methyl.

34. The ASO of claim 11, wherein the ASO comprises 2 or more contiguous nucleotides of unmodified DNA flanked by at least 3 nucleotides of modified ribonucleotides at each of the 5′ end and the 3′ end.

35. (canceled)

36. (canceled)

37. A pharmaceutical composition comprising the ASO of claim 1 and a pharmaceutically acceptable carrier.

38. A method of increasing transcription of GRN in a human cell, the method comprising contacting the cell with the ASO of claim 1.

39. (canceled)

40. (canceled)

41. (canceled)

42. (canceled)

43. (canceled)

44. A method of treating a disease or disorder in a subject, the method comprising administering to a subject in need thereof an effective amount of the ASO of claim 1.

45. The method of claim 44, wherein the disease or disorder is selected from the group consisting of frontotemporal dementia (FTD), frontotemporal lobar degeneration (FTLD), neuroinflammation, myopathy, familial frontotemporal dementia with neuropathologic frontotemporal lobal degeneration associated with accumulation of TDP-43 inclusions (FTLD-TDP), Down syndrome, Huntington's disease, hippocampal sclerosis dementia, spinocerebellar ataxia 3, chronic traumatic encephalopathy, Alzheimer's disease (AD), amyotrophic lateral sclerosis (ALS), Gaucher disease (GD) and Parkinson's disease (PD), neuronal ceroid lipofuscinosis (NCL) type 11 (CLN11), limbic-predominant age-related TDP-43 encephalopathy (LATE), autism, ischemia-reperfusion injury in the brain, a lysosomal storage disease (LSD), rheumatoid arthritis (RA), inflammatory bowel disease (IBD), multiple sclerosis (MS), ischemic heart disease, intervertebral disc ceneration, and acute kidney injury.

46. (canceled)

47. (canceled)

48. (canceled)

49. (canceled)

50. (canceled)

51. (canceled)
